(19) United States
(12) Patent Application Publication
(10) Pub. No.: US 2013/0324530 A1
(43) Pub. Date: Dec. 5, 2013

(54) 3-(AMINOARYL)-PYRIDINE COMPOUNDS

(71) Applicants: William R. Antonios-McCrea, Moraga, CA (US); Paul A. Barsanti, Pleasant Hill, CA (US); Cheng Hu, Menlo Park, CA (US); Xianming Jin, San Ramon, CA (US); Eric J. Martin, El Cerrito, CA (US); Yue Pan, Moraga, CA (US); Keith B. Pfister, San Ramon, CA (US); Martin Sendzik, San Mateo, CA (US); James Sutton, Pleasanton, CA (US); Lifeng Wan, Union City, CA (US)

(72) Inventors: William R. Antonios-McCrea, Moraga, CA (US); Paul A. Barsanti, Pleasant Hill, CA (US); Cheng Hu, Menlo Park, CA (US); Xianming Jin, San Ramon, CA (US); Eric J. Martin, El Cerrito, CA (US); Yue Pan, Moraga, CA (US); Keith B. Pfister, San Ramon, CA (US); Martin Sendzik, San Mateo, CA (US); James Sutton, Pleasanton, CA (US); Lifeng Wan, Union City, CA (US)

(73) Assignee: Novartis AG

(21) Appl. No.: 13/885,640

(22) PCT Filed: Nov. 17, 2011
(86) PCT No.: PCT/EP11/70339

§ 371 Date: Aug. 21, 2013

Related U.S. Application Data
(60) Provisional application No. 61/414,797, filed on Nov. 17, 2010.

(51) Int. Cl. C07D 213/74; C07D 401/14; C07D 409/14; C07D 405/14

(52) U.S. Cl. CPC C07D 213/74; C07D 405/14; C07D 401/14; C07D 409/14
USPC 514/235.5; 514/333; 546/256; 514/334; 546/258; 544/124; 514/318; 546/194; 546/193

(57) ABSTRACT

The present invention provides a compound of formula (I):

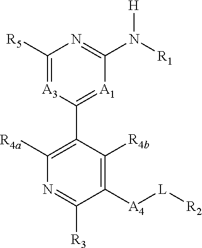

and pharmaceutically acceptable salts, enantiomers, stereoisomers, rotamers, tautomers, diastereomers, or racemates thereof. Also provided are pharmaceutical compositions containing these compounds and methods of treating a disease or condition mediated by CDK9 using these compounds and compositions.

### BACKGROUND

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. (Hardie, G. and Hanks, S., THE PROTEIN KINASE FACTS BOOK, I AND II, Academic Press, San Diego, Calif.: 1995). Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.). Sequence motifs have been identified that generally correspond to each of these kinase families (See, for example, Hanks, S. K., Hunter, T., FASEB J. 1995, 9, 576-596; Knighton et al., Science 1991, 253, 407-414; Hiles et al., Cell 1992, 70, 419-429; Kunz et al., Cell 1993, 73, 585-596; Garcia-Bustos et al., EMBO J. 1994, 13, 2352-2361).

Many diseases are associated with abnormal cellular responses triggered by the protein kinase-mediated events described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, viral diseases, and hormone-related diseases. Accordingly, there has been a substantial effort in medicinal chemistry to find protein kinase inhibitors that are effective as therapeutic agents.

The cyclin-dependent kinase (CDK) complexes are a class of kinases that are targets of interest. These complexes comprise at least a catalytic (the CDK itself) and a regulatory (cyclin) subunit. Some of the more important complexes for cell cycle regulation include cyclin A (CDK1—also known as cdc2, and CDK2), cyclin B1-B3 (CDK1) and cyclin D1-D3 (CDK2, CDK4, CDK5, CDK6), cyclin E (CDK2). Each of these complexes is involved in a particular phase of the cell cycle. Additionally, CDKs 7, 8, and 9 are implicated in the regulation of transcription.

The activity of CDKs is regulated post-translationally, by transitory associations with other proteins, and by alterations of their intracellular localization. Tumor development is closely associated with genetic alteration and deregulation of CDKs and their regulators, suggesting that inhibitors of CDKs may be useful anti-cancer therapeutics. Indeed, early results suggest that transformed and normal cells differ in their requirement for, e.g., cyclin A/CDK2 and that it may be possible to develop novel antineoplastic agents devoid of the general host toxicity observed with conventional cytotoxic and cytostatic drugs. While inhibition of cell cycle-related CDKs is clearly relevant in, e.g., oncology applications, inhibition of RNA polymerase-regulating CDKs may also be highly relevant in cancer indications.

The CDKs have been shown to participate in cell cycle progression and cellular transcription, and loss of growth control is linked to abnormal cell proliferation in disease (see e.g., Malumbres and Barbacid, Nat. Rev. Cancer 2001, 1:222). Increased activity or temporally abnormal activation of cyclin-dependent kinases has been shown to result in the development of human tumors (Sherr C. J., Science 1996, 274: 1672-1677). Indeed, human tumor development is commonly associated with alterations in either the CDK proteins themselves or their regulators (Cordon-Cardo C., Am. J. Pat. 1995; vol. 147: 545-560; Karp J. E. and Broder S., Nat. Med. 1995; 1: 309-320; Hall M. et al., Adv. Cancer Res. 1996; 68: 67-108).

Naturally occurring protein inhibitors of CDKs such as p16 and p27 cause growth inhibition in vitro in lung cancer cell lines (Kamb A., Curr. Top. Microbiol. Immunol. 1998; 227: 139-148).

CDKs 7 and 9 seem to play key roles in transcription initiation and elongation, respectively (see, e.g., Peterlin and Price, Cell 23: 297-305, 2006; Shapiro, J. Clin. Oncol. 24: 1770-83, 2006;). Inhibition of CDK9 has been linked to direct induction of apoptosis in tumor cells of hematopoietic lineages through down-regulation of transcription of antiapoptotic proteins such as Mcl1 (Chao, S.-H. et al. J. Biol. Chem. 2000; 275:28345-28348; Chao, S.-H. et al. J. Biol. Chem. 2001; 276:31793-31799; Lam et. al. Genome Biology 2: 0041.1-11, 2001; Chen et al. Blood 2005; 106:2513; MacCallum et al. Cancer Res. 2005; 65:5399; and Alvi et al. Blood 2005; 105:4484). In solid tumor cells, transcriptional inhibition by downregulation of CDK9 activity synergizes with inhibition of cell cycle CDKs, for example CDK1 and 2, to induce apoptosis (Cai, D.-P., Cancer Res 2006, 66:9270. Inhibition of transcription through CDK9 or CDK7 may have selective non-proliferative effect on the tumor cell types that are dependent on the transcription of mRNAs with short half lives, for example Cyclin D1 in Mantle Cell Lymphoma. Some transcription factors such as Myc and NF-kB selectively recruit CDK9 to their promoters, and tumors dependent on activation of these signalling pathways may be sensitive to CDK9 inhibition.

Small molecule CDK inhibitors may also be used in the treatment of cardiovascular disorders such as restenosis and atherosclerosis and other vascular disorders that are due to aberrant cell proliferation. Vascular smooth muscle proliferation and intimal hyperplasia following balloon angioplasty are inhibited by over-expression of the cyclin-dependent kinase inhibitor protein. Moreover, the purine CDK2 inhibitor CVT-313 (Ki=95 nM) resulted in greater than 80% inhibition of neointima formation in rats.

CDK inhibitors can be used to treat diseases caused by a variety of infectious agents, including fungi, protozoan parasites such as Plasmodium falciparum, and DNA and RNA viruses. For example, cyclin-dependent kinases are required for viral replication following infection by herpes simplex virus (HSV) (Schang L. M. et al., J. Virol. 1998; 72: 5626) and CDK homologs are known to play essential roles in yeast.

Inhibition of CDK9/cyclin T function was recently linked to prevention of HIV replication and the discovery of new CDK biology thus continues to open up new therapeutic indications for CDK inhibitors (Sausville, E. A. Trends Molec. Med. 2002, 8, S32-S37).

CDKs are important in neutrophil-mediated inflammation and CDK inhibitors promote the resolution of inflammation in animal models. (Rossi, A. G. et al, Nature Med. 2006, 12:1056). Thus CDK inhibitors, including CDK9 inhibitors, may act as anti-inflammatory agents.

Selective CDK inhibitors can be used to ameliorate the effects of various autoimmune disorders. The chronic inflammatory disease rheumatoid arthritis is characterized by synovial tissue hyperplasia; inhibition of synovial tissue proliferation should minimize inflammation and prevent joint destruction. In a rat model of arthritis, joint swelling was substantially inhibited by treatment with an adenovirus expressing a CDK inhibitor protein p 16. CDK inhibitors are effective against other disorders of cell proliferation including psoriasis (characterized by keratinocyte hyperproliferation), glomerulonephritis, chronic inflammation, and lupus.

Certain CDK inhibitors are useful as chemoprotective agents through their ability to inhibit cell cycle progression of normal untransformed cells (Chen, et al. J. Natl. Cancer Institute, 2000; 92: 1999-2008). Pre-treatment of a cancer patient with a CDK inhibitor prior to the use of cytotoxic agents can reduce the side effects commonly associated with chemotherapy. Normal proliferating tissues are protected from the cytotoxic effects by the action of the selective CDK inhibitor.

Accordingly, there is a great need to develop inhibitors of protein kinases, such as CDK1, CDK2, CDK3, CDK4, CDK5, CDK6, CDK7, CDK8 and CDK9, as well as combinations thereof.

### SUMMARY OF THE INVENTION

There remains a need for new treatments and therapies for protein kinase-associated disorders. There is also a need for compounds useful in the treatment or prevention or amelioration of one or more symptoms of cancer, inflammation, cardiac hypertrophy, and HIV. Furthermore, there is a need for methods for modulating the activity of protein kinases, such as CDK1, CDK2, CDK3, CDK4, CDK5, CDK6, CDK7, CDK8 and CDK9, and combinations thereof, particularly modulation of CDK9. The present invention provides novel compounds that inhibit CDK9, and are thus useful for treatment of disorders mediated by excessive or undesired levels of CDK9 activity.

In one aspect, the invention provides a compound of Formula (I):

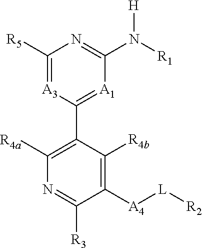

or a pharmaceutically acceptable salt or deuterated version thereof, wherein:

A₁ is N or CR₆;

A₃ is N or CR₈;

A₄ is selected from the group consisting of a bond, SO₂, CO—NR₉, NR₉, —SO₂—NR₉—, and O;

L is selected from the group consisting of a bond, optionally substituted C₁₋₄alkyl, C₃₋₆ cycloalkyl, C₃₋₆ heterocycloalkyl, and C₂₋₄ alkenyl;

R₁ is —X—R₁₆;

as more fully described below.

In certain embodiments, the compound is a compound of Formula II:

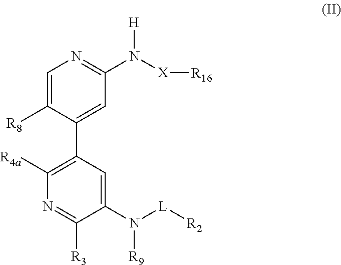

The compounds of Formulas (I) and (II) are inhibitors of CDK9. Accordingly, they are useful to treat conditions mediated by excessive or undesired levels of CDK9 activity. The invention also, in another aspect, provides a pharmaceutical composition comprising a compound of Formula (I) or (II) in combination with at least one pharmaceutically acceptable excipient and/or carrier.

In another aspect the invention provides methods to use the compounds of Formula I or II or a pharmaceutical composition comprising such compounds to treat conditions associated with CDK9 activity, such as cancer and other conditions described herein.

In other embodiments, the present invention provides a method for inhibiting the activity of a protein kinase. The method includes contacting a cell with any of the compounds of the present invention. In a related embodiment, the method further provides that the compound is present in an amount effective to selectively inhibit the activity of a protein kinase.

In other embodiments, the present invention provides a use of any of the compounds of the invention for manufacture of a medicament to treat cancer, inflammation, cardiac hypertrophy, and HIV infection in a subject.

In other embodiments, the invention provides a method of manufacture of a medicament, including formulating any of the compounds of the present invention for treatment of a subject.

### DETAILED DESCRIPTION OF CERTAIN EMBODIMENTS

The term “treat,” “treated,” “treating” or “treatment” includes the diminishment or alleviation of at least one symptom associated or caused by the state, disorder or disease being treated. In certain embodiments, the treatment comprises the induction of a protein kinase-associated disorder, followed by the activation of the compound of the invention, which would in turn diminish or alleviate at least one symptom associated or caused by the protein kinase-associated disorder being treated. For example, treatment can be diminishment of one or several symptoms of a disorder or complete eradication of a disorder.

The term “use” includes any one or more of the following embodiments of the invention, respectively: the use in the treatment of protein kinase-associated disorders; the use for the manufacture of pharmaceutical compositions for use in the treatment of these diseases, e.g., in the manufacture of a medicament; methods of use of compounds of the invention in the treatment of these diseases; pharmaceutical preparations having compounds of the invention for the treatment of these diseases; and compounds of the invention for use in the treatment of these diseases; as appropriate and expedient, if not stated otherwise. In particular, diseases to be treated and are thus preferred for use of a compound of the present invention are selected from cancer, inflammation, cardiac hypertrophy, and HIV infection, as well as those diseases that depend on the activity of protein kinases. The term “use” further includes embodiments of compositions herein which bind to a protein kinase sufficiently to serve as tracers or labels, so that when coupled to a fluor or tag, or made radioactive, can be used as a research reagent or as a diagnostic or an imaging agent.

The term “subject” is intended to include organisms, e.g., prokaryotes and eukaryotes, which are capable of suffering from or afflicted with a disease, disorder or condition associated with the activity of a protein kinase. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from cancer, inflammation, cardiac hypertrophy, and HIV infection, and other diseases or conditions described herein (e.g., a protein kinase-associated disorder). In another embodiment, the subject is a cell.

The language “protein kinase-modulating compound,” “modulator of protein kinase” or “protein kinase inhibitor” refers to compounds that modulate, e.g., inhibit, or otherwise alter, the activity of a protein kinase. Examples of protein kinase-modulating compounds include compounds of the invention, i.e., Formula I and Formula II, as well as the compounds of Table 1 and Table 1B, including pharmaceutically acceptable salts thereof, as well as enantiomers, stereoisomers, rotamers, tautomers, diastereomers, atropisomers or racemates thereof.

Additionally, a method of the invention includes administering to a subject an effective amount of a protein kinase-modulating compound of the invention, e.g., protein kinase-modulating compounds of Formula I and Formula II, as well as Table 1 and Table 1B, including pharmaceutically acceptable salts thereof, as well as enantiomers, stereoisomers, rotamers, tautomers, diastereomers, atropisomers or racemates thereof.

Where linking groups are specified by their conventional chemical formula herein, written from left to right, they equally encompass the chemically identical substituents that would result from writing the structure from right to left, e.g., —CH₂O— is intended to include —OCH₂— for this purpose only.

The term “alkyl,” by itself or as part of another substituent, means, unless otherwise stated, a fully saturated straight-chain (linear; unbranched) or branched chain, or a combination thereof, having the number of carbon atoms specified, if designated (i.e. C₁-C₁₀ means one to ten carbons). Examples include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like. If no size is designated, the alkyl groups mentioned herein contain 1-10 carbon atoms, typically 1-8 carbon atoms, and often 1-6 or 1-4 carbon atoms, and preferably 1-2 carbon atoms. If the alkyl group is a branched alkyl group, and the number of carbon atoms is not mentioned, the branched alkyl group will consist of 3-8 carbon atoms, typically about 3-6 carbon atoms, and particularly 3-4 carbon atoms.

The term “alkenyl” refers to unsaturated aliphatic groups including straight-chain (linear; unbranched), branched-chain groups, and combinations thereof, having the number of carbon atoms specified, if designated, which contain at least one double bond (—C═C—). All double bonds may be independently either (E) or (Z) geometry, as well as mixtures thereof. Examples of alkenyl groups include, but are not limited to, —CH₂—CH═CH—CH₃; —CH═CH—CH═CH₂ and —CH₂—CH═CH—CH(CH₃)—CH₂—CH₃. If no size is specified, the alkenyl groups discussed herein contain 2-6 carbon atoms.

The term “alkynyl” refers to unsaturated aliphatic groups including straight-chain (linear; unbranched), branched-chain groups, and combinations thereof, having the number of carbon atoms specified, if designated, which contain at least one carbon-carbon triple bond (—C≡C—). Examples of alkynyl groups include, but are not limited to, —CH₂—C≡C—CH₃; —C≡C—C≡CH and —CH₂—C≡C—CH(CH₃)—CH₂—CH₃. If no size is specified, the alkynyl groups discussed herein contain 2-6 carbon atoms. Alkynyl and alkenyl groups can contain more than one unsaturated bond, or a mixture of double and triple bonds, and can be otherwise substituted as described for alkyl groups.

Where an alkyl, alkenyl or alkynyl or cycloalkyl or heterocycloalkyl group is shown by its context to function as a linking group connecting two features together, e.g., groups such as L and X and R₂₂ in Formula I, the alkyl, alkenyl or alkynyl group is divalent, as will be recognized be a person of ordinary skill. Examples of such groups include methylene, (CH₂)ₙ where n=1-4, —CH(CH₃)—, 1,1-cyclopropane-diyl, and the like.

The terms “alkoxy,” “alkenyloxy,” and “alkynyloxy” refer to —O-alkyl, —O-alkenyl, and —O-alkynyl, respectively.

The term “cycloalkyl” by itself or in combination with other terms, represents, unless otherwise stated, cyclic versions of alkyl, alkenyl, or alkynyl, or mixtures thereof. Additionally, cycloalkyl may contain fused rings, but excludes fused aryl and heteroaryl groups, and cycloalkyl groups can be substituted unless specifically described as unsubstituted. Examples of cycloalkyl include, but are not limited to, cyclopropyl, cyclobutyl, cyclopentyl, cyclohexyl, 1-cyclohexenyl, 3-cyclohexenyl, cyclohexynyl, cyclohexynyl, cyclohexadienyl, cyclopentadienyl, cyclopentenyl, cycloheptyl, norbornyl, and the like. If no ring size is specified, the cycloalkyl groups described herein contain 3-8 ring members, or 3-6 ring members.

The term “heterocyclic” or “heterocycloaklyl” or “heterocyclyl,” by itself or in combination with other terms, represents a cycloalkyl radical containing at least one annular carbon atom and at least one annular heteroatom selected from the group consisting of O, N, P, Si and S, preferably from N, O and S, wherein the ring is not aromatic but can contain unsaturations. The nitrogen and sulfur atoms in a heterocyclic group may optionally be oxidized and the nitrogen heteroatom may optionally be quaternized. In many embodiments, the annular heteroatoms are selected from N, O and S. The heterocyclic groups discussed herein, if not otherwise specified, contain 3-10 ring members, and at least one ring member is a heteroatom selected from N, O and S; commonly not more than three of these heteroatoms are included in a heterocyclic group, and generally not more than two of these heteroatoms are present in a single ring of the heterocyclic group. The heterocyclic group can be fused to an additional carbocyclic, heterocyclic, or aryl ring. A heterocyclic group can be attached to the remainder of the molecule at an annular carbon or annular heteroatom, and the heterocyclic groups can be substituted as described for alkyl groups. Additionally, heterocyclic may contain fused rings, but excludes fused systems containing a heteroaryl group as part of the fused ring system. Examples of heterocyclic groups include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, 1,2,3,4-tetrahydropyridyl, dihydroindole (indoline), tetrahydrofuran-3-yl, tetrahydrothien-2-yl, tetrahydrothien-3-yl, 1-piperazinyl, 2-piperazinyl, and the like.

As with other moieties described herein, heterocycloalkyl moieties can be unsubstituted, or substituted with various substituents known in the art, e.g., hydroxy, halo, oxo (C═O), alkylimino (RN═, wherein R is a loweralkyl or loweralkoxy group), amino, alkylamino, dialkylamino, acylaminoalkyl, alkoxy, thioalkoxy, polyalkoxy, loweralkyl, cycloalkyl or haloalkyl. Non-limiting examples of substituted heterocycloalkyl groups include the following, where each moiety may be attached to the parent molecule at any available valence, and in some of these substructures, a preferred attachment point is indicated by a bond having a wavy line across it:

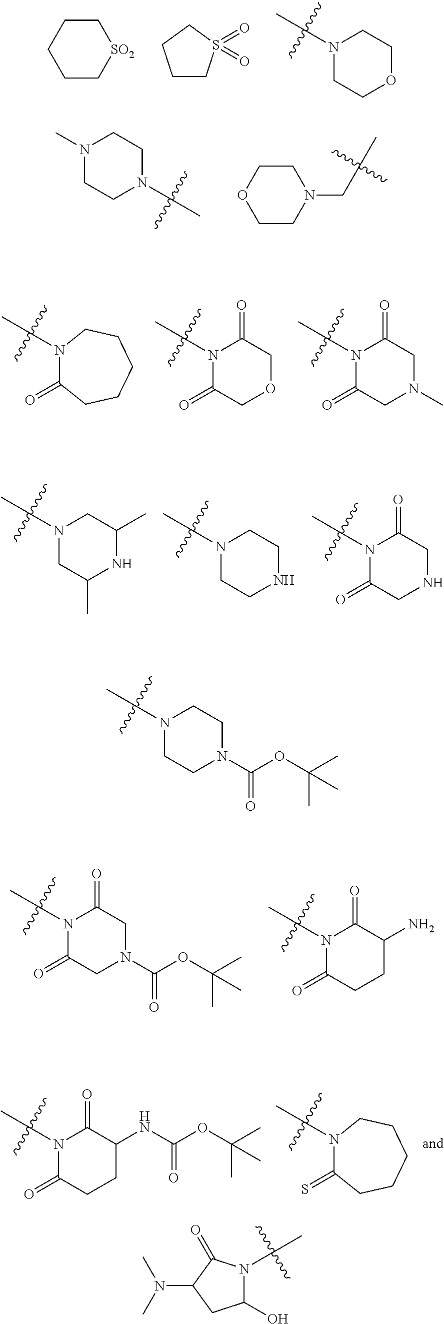

Also included within heterocyclic are piperidine, morpholine, thiomorpholine, piperazine, pyrrolidine, tetrahydrofuran, oxetane, oxepane, oxirane, tetrahydrothiofuran, thiepane, thiirane, and optionally substituted versions of each of these.

The terms “cycloalkyloxy” and “heterocycloalkyloxy” refer to —O-cycloalkyl and —O-heterocycloalkyl groups, respectively (e.g., cyclopropoxy, 2-piperidinyloxy, and the like).

The term ‘acyl’ as used herein takes its conventional meaning, and refers to a group of the formula —C(═O)R, where R represents an alkyl group or other group of suitable size and composition. For example, a C₁₋₆ acyl would include R═C₁-C₅ alkyl, wherein the alkyl may be substituted as for typical alkyl groups.

The term “aryl” means, unless otherwise stated, an aromatic hydrocarbon group which can be a single ring or multiple rings (e.g., from 1 to 3 rings) which are fused together. Aryl may contain fused rings, wherein one or more of the rings is optionally cycloalkyl, but not including heterocyclic or heteroaromatic rings; a fused system containing at least one heteroaromatic ring is described as a heteroaryl group, and a phenyl ring fused to a heterocyclic ring is described herein as a heterocyclic group. An aryl group will include a fused ring system wherein a phenyl ring is fused to a cycloalkyl ring. Examples of aryl groups include, but are not limited to, phenyl, 1-naphthyl, tetrahydro-naphthalene, dihydro-1H-indene, 2-naphthyl, tetrahydronaphthyl and the like.

The term “heteroaryl” as used herein refers to groups comprising a single ring or two or three fused rings, where at least one of the rings is an aromatic ring that contain from one to four heteroatoms selected from N, O, and S as ring members (i.e., it contains at least one heteroaromatic ring), wherein the nitrogen and sulfur atoms are optionally oxidized, and the nitrogen atom(s) are optionally quaternized. A heteroaryl group can be attached to the remainder of the molecule through an annular carbon or annular heteroatom, and it can be attached through any ring of the heteroaryl moiety, if that moiety is bicyclic or tricyclic. Heteroaryl may contain fused rings, wherein one or more of the rings is optionally cycloalkyl or heterocycloalkyl or aryl, provided at least one of the rings is a heteroaromatic ring. Non-limiting examples of heteroaryl groups are 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Aryl and/or heteroaryl groups commonly contain up to four substituents per ring (0-4), and sometimes contain 0-3 or 0-2 substituents. The terms “aryloxy” and “heteroaryloxy” refer to aryl and heteroaryl groups, respectively, attached to the remainder of the molecule via an oxygen linker (—O—).

The term “arylalkyl” or “aralkyl” designates an alkyl-linked aryl group, where the alkyl portion is attached to the parent structure and the aryl is attached to the alkyl portion of the arylalkyl moiety. Examples are benzyl, phenethyl, and the like. “Heteroarylalkyl” or “heteroaralkyl” designates a heteroaryl moiety attached to the parent structure via an alkyl residue. Examples include furanylmethyl, pyridinylmethyl, pyrimidinylethyl, and the like. Aralkyl and heteroaralkyl also include substituents in which at least one carbon atom of the alkyl group is present in the alkyl group and wherein another carbon of the alkyl group has been replaced by, for example, an oxygen atom (e.g., phenoxymethyl, 2-pyridylmethoxy, 3-(1-naphthyloxy)propyl, and the like).

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and perhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like. The prefix “perhalo” refers to the respective group wherein all available valences are replaced by halo groups. For example “perhaloalkyl” includes —CCl₃, —CF₃, —CCl₂CF₃, and the like. The terms “perfluoroalkyl” and “perchloroalkyl” are a subsets of perhaloalkyl wherein all available valences are replaced by fluoro and chloro groups, respectively. Non limiting examples of perfluoroalkyl include —CF₃ and —CF₂CF₃. Non limiting examples of perchloroalkyl include —CCl₃ and —CCl₂CCl₃.

“Amino” refers herein to the group —NH₂ or —NRR′, where R and R′ are each independently selected from hydrogen or an alkyl (e.g, lower alkyl). The term “arylamino” refers herein to the group —NRR′ where R is aryl and R′ is hydrogen, alkyl, or an aryl. The term “aralkylamino” refers herein to the group —NRR′ where R is an aralkyl and R′ is hydrogen, an alkyl, an aryl, or an aralkyl. “Substituted amino” refers to an amino wherein at least one of R and R′ is not H, i.e., the amino has at least one substituent group on it. The term alkylamino refers to -alkyl-NRR′ where R and R′ are each independently selected from hydrogen or an alkyl (e.g, lower alkyl).

The term “aminocarbonyl” refers herein to the group —C(O)—NH₂, i.e., it is attached to the base structure through the carbonyl carbon atom. “Substituted aminocarbonyl” refers herein to the group —C(O)—NRR′ where R is alkyl and R′ is hydrogen or an alkyl. The term “arylaminocarbonyl” refers herein to the group —C(O)—NRR′ where R is an aryl and R′ is hydrogen, alkyl or aryl. “Aralkylaminocarbonyl” refers herein to the group —C(O)—NRR′ where R is aralkyl and R′ is hydrogen, alkyl, aryl, or aralkyl.

“Aminosulfonyl” refers herein to the group —S(O)₂—NH₂. “Substituted aminosulfonyl” refers herein to the group —S(O)₂—NRR′ where R is alkyl and R′ is hydrogen or an alkyl. The term “aralkylaminosulfonylaryl” refers herein to the group -aryl-S(O)₂—NH-aralkyl.

“Carbonyl” refers to the divalent group —C(O)—.

The term “sulfonyl” refers herein to the group —SO₂—. “Alkylsulfonyl” refers to a substituted sulfonyl of the structure —SO₂R in which R is alkyl. Alkylsulfonyl groups employed in compounds of the present invention are typically loweralkylsulfonyl groups having from 1 to 6 carbon atoms in R. Thus, exemplary alkylsulfonyl groups employed in compounds of the present invention include, for example, methylsulfonyl (i.e., where R is methyl), ethylsulfonyl (i.e., where R is ethyl), propylsulfonyl (i.e., where R is propyl), and the like. The term “arylsulfonyl” refers herein to the group —SO₂-aryl. The term “aralkylsulfonyl” refers herein to the group —SO₂-aralkyl. The term “sulfonamido” refers herein to —SO₂NH₂, or to —SO₂NRR′ if substituted.

Unless otherwise stated, each radical/moiety described herein (e.g., “alkyl,” “cycloalkyl,” “heterocycloalkyl,” “aryl,” “heteroaryl,” “alkoxy,” etc.) is meant to include both substituted and unsubstituted forms.

“Optionally substituted” as used herein indicates that the particular group or groups being described may have no non-hydrogen substituents (i.e., it can be unsubstituted), or the group or groups may have one or more non-hydrogen substituents. If not otherwise specified, the total number of such substituents that may be present is equal to the number of H atoms present on the unsubstituted form of the group being described. Typically, an optionally substituted group will contain up to three (0-3) substituents. Where an optional substituent is attached via a double bond, such as a carbonyl oxygen (═O), the group takes up two available valences on the group being substituted, so the total number of substituents that may be included is reduced according to the number of available valences. Suitable substituent groups include, for example, hydroxyl, nitro, amino, imino, cyano, halo, thio, sulfonyl, thioamido, amidino, imidino, oxo, oxamidino, methoxamidino, imidino, guanidino, sulfonamido, carboxyl, formyl, loweralkyl, loweralkoxy, loweralkoxyalkyl, alkylcarbonyl, aminocarbonyl, arylcarbonyl, aralkylcarbonyl, carbonylamino, heteroarylcarbonyl, heteroaralkylcarbonyl, alkylthio, aminoalkyl, cyanoalkyl, aryl, alkylamino, alkylsulfonyl, aralkylamino, alkylcarbonylamino, carbonyl, piperidinyl, morpholinyl, pyrrolidinyl and the like.

Deuterium, when introduced into a compound at levels at least 5× above natural abundance, can also be considered a substituent for purposes of describing the compounds herein. Note that because deuterium is an isotope of hydrogen that does not substantially change the shape of the molecule, deuterium is exempt from the typical numerical limitations placed on numbers of substituents: deuterium (D) can be included in place of hydrogen (H) in addition to other substituents and should not be counted in the numerical limitations that apply to other substituents.

A substituent group can itself be substituted by the same groups described herein for the corresponding type of structure. The group substituted onto the substituted group can be carboxyl, halo, nitro, amino, cyano, hydroxyl, loweralkyl, loweralkenyl, loweralkynyl, loweralkoxy, aminocarbonyl, —SR, thioamido, —SO₃H, —SO₂R, COOR, N-methylpyrrolidinyl, piperidinyl, piperazinyl, N-methylpiperazinyl, 4-chloropyrimidinyl, pyridinyl, tetrahydropyranyl, heterocycloalkyl, heteroaryl, or cycloalkyl, where R is typically hydrogen or loweralkyl.

When the substituted substituent includes a straight chain group, the substituent can occur either within the chain (e.g., 2-hydroxypropyl, 2-aminobutyl, and the like) or at the chain terminus (e.g., 2-hydroxyethyl, 3-cyanopropyl, and the like). Substituted substituents can be straight chain, branched or cyclic arrangements of covalently bonded carbon or heteroatoms (N, O or S).

The term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “isomer” includes all stereoisomers of the compounds referred to in the formulas herein, including enantiomers, diastereomers, as well as all conformers, rotamers, and tautomers, unless otherwise indicated. The invention includes all enantiomers of any chiral compound disclosed, in either substantially pure levorotatory or dextrorotatory form, or in a racemic mixture, or in any ratio of enantiomers. For compounds disclosed as an (R)-enantiomer, the invention also includes the (S)-enantiomer; for compounds disclosed as the (S)-enantiomer, the invention also includes the (R)-enantiomer. The invention includes any diastereomers of the compounds referred to in the above formulas in diastereomerically pure form and in the form of mixtures in all ratios.

Unless stereochemistry is explicitly indicated in a chemical structure or chemical name, the chemical structure or chemical name is intended to embrace all possible stereoisomers, conformers, rotamers, and tautomers of the compound depicted. For example, a compound containing a chiral carbon atom is intended to embrace both the (R) enantiomer and the (S) enantiomer, as well as mixtures of enantiomers, including racemic mixtures; and a compound containing two chiral carbons is intended to embrace all enantiomers and diastereomers (including (R,R), (S,S), (R,S), and (R,S) isomers). In all uses of the compounds of the formulas disclosed herein, the invention also includes use of any or all of the stereochemical, enantiomeric, diastereomeric, conformational, rotomeric, tautomeric, solvate, hydrate, polymorphic, crystalline form, non-crystalline form, salt, pharmaceutically acceptable salt, metabolite and prodrug variations of the compounds as described.

The term “heteroatom” includes atoms of any element other than carbon or hydrogen. Preferred heteroatoms are nitrogen, oxygen, sulfur and phosphorus. Additionally, the phrase “any combination thereof” implies that any number of the listed functional groups and molecules may be combined to create a larger molecular architecture. For example, the terms “phenyl,” “carbonyl” (or “═O”), “—O—,” “—OH,” and C₁₋₆ (i.e., —CH₃ and —CH₂CH₂CH₂—) can be combined to form a 3-methoxy-4-propoxybenzoic acid substituent. It is to be understood that when combining functional groups and molecules to create a larger molecular architecture, hydrogens can be removed or added, as required to satisfy the valence of each atom.

The description of the disclosure herein should be construed in congruity with the laws and principals of chemical bonding. For example, it may be necessary to remove a hydrogen atom in order accommodate a substituent at any given location. Furthermore, it is to be understood that definitions of the variables (i.e., “R groups”), as well as the bond locations of the generic formulae of the invention (e.g., formulas I or II), will be consistent with the laws of chemical bonding known in the art. It is also to be understood that all of the compounds of the invention described above will further include bonds between adjacent atoms and/or hydrogens as required to satisfy the valence of each atom. That is, bonds and/or hydrogen atoms are added to provide the following number of total bonds to each of the following types of atoms: carbon: four bonds; nitrogen: three bonds; oxygen: two bonds; and sulfur: two-six bonds. Preferably, the compounds do not include any oxygen-oxygen bonds.

As used herein, “isomer” includes all stereoisomers of the compounds referred to in the formulas herein, including enantiomers, diastereomers, as well as all conformers, rotamers, and tautomers, unless otherwise indicated. The invention includes all enantiomers of any chiral compound disclosed, in either substantially pure levorotatory or dextrorotatory form, or in a racemic mixture, or in any ratio of enantiomers. For compounds disclosed as an (R)-enantiomer, the invention also includes the (S)-enantiomer; for compounds disclosed as the (S)-enantiomer, the invention also includes the (R)-enantiomer. The invention includes any diastereomers of the compounds referred to in the above formulas in diastereomerically pure form and in the form of mixtures in all ratios.

Unless stereochemistry is explicitly indicated in a chemical structure or chemical name, the chemical structure or chemical name is intended to embrace all possible stereoisomers, conformers, rotamers, and tautomers of the compound depicted. For example, a compound containing a chiral carbon atom is intended to embrace both the (R) enantiomer and the (5) enantiomer, as well as mixtures of enantiomers, including racemic mixtures; and a compound containing two chiral carbons is intended to embrace all enantiomers and diastereomers (including (R,R), (S,S), (R,S), and (R,S) isomers). In all uses of the compounds of the formulas disclosed herein, the invention also includes use of any or all of the stereochemical, enantiomeric, diastereomeric, conformational, rotomeric, tautomeric, solvate, hydrate, polymorphic, crystalline form, non-crystalline form, salt, pharmaceutically acceptable salt, metabolite and prodrug variations of the compounds as described.

It will also be noted that the substituents of some of the compounds of this invention include isomeric cyclic structures. It is to be understood accordingly that constitutional isomers of particular substituents are included within the scope of this invention, unless indicated otherwise. For example, the term “tetrazole” includes tetrazole, 2H-tetrazole, 3H-tetrazole, 4H-tetrazole and 5H-tetrazole.

In one aspect, the invention provides a compound of Formula (I):

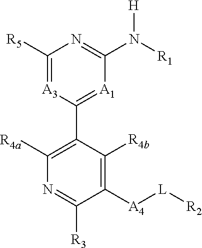

or a pharmaceutically acceptable salt or deuterated version thereof, wherein:

A₁ is N or CR₆;

A₃ is N or CR₈;

A₄ is selected from the group consisting of a bond, SO₂, CO—NR₉, NR₉, —SO₂—NR₉—, and O;

L is selected from the group consisting of a bond and an optionally substituted group selected from C₁₋₄alkyl, C₃₋₆ cycloalkyl, C₃₋₆ heterocycloalkyl, and C₂₋₄ alkenyl;

R₁ is —X—R₁₆;

X is a bond or C₁₋₄ alkyl;

R₁₆ is selected from the group consisting of C₁₋₆ alkyl, C₃₋₆ branched alkyl, C₃₋₈cycloalkyl, heterocycloalkyl, C₃₋₁₀ heterocycloalkyl, C₃₋₈-partially unsaturated cycloalkyl, C₆₋₁₀ aryl, C₆₋₁₀ aryl- or C₅₋₆-heteroaryl-fused C₅₋₇ heterocycloalkyl, and C₅₋₁₀ heteroaryl,
wherein R₁₆ is optionally substituted with up to three groups independently selected from halogen, oxo (═O), C₁₋₆alkyl, C₁₋₆haloalkyl, C₃₋₆ branched alkyl, C₃₋₆ branched haloalkyl, OH, C₁₋₆alkoxy, C₄₋₈ heterocycloalkyl, C₁₋₂alkyl-heterocycloalkyl, C₁₋₂alkyl-heteroaryl, —R₂₂—OR₁₂, —S(O)₀₋₂R₁₂, —R₂₂—S(O)₀₋₂R₁₂, —S(O)₂NR₁₃R₁₄, —R₂₂—S(O)₂NR₁₃R₁₄, —C(O)OR₁₂, —R₂₂—C(O)OR₁₂, —C(O)R₁₉, —R₂₂—C(O)R₁₉, —O—C₁₋₃ alkyl, —OC₁₋₃ haloalkyl, —OC(O)R₁₉, —R₂₂—OC(O)R₁₉, —C(O)NR₁₃R₁₄, —R₂₂—C(O)NR₁₃R₁₄, —NR₁₅S(O)₂R₁₂, —R₂₂—NR₁₅S(O)₂R₁₂, —NR₁₇R₁₈, —R₂₂—NR₁₇R₁₈, —NR₁₅C(O)R₁₉, —R₂₂—NR₁₅C(O)R₁₉, —NR₁₅C(O)OCH₂Ph, —R₂₂—NR₁₅C(O)OCH₂Ph, —NR₁₅C(O)OR₁₂, —R₂₂—NR₁₅C(O)OR₁₂, —NR₁₅C(O)NR₁₃R₁₄, and —R₂₂—NR₁₅C(O)NR₁₃R₁₄,
   wherein said C₁₋₆alkyl and C₃₋₆ branched alkyl are optionally substituted with up to three R₂₀;

R₁₇ and R₁₈ are each, independently, selected from the group consisting of hydrogen, hydroxyl, C₁₋₆alkyl, C₁₋₆haloalkyl, C₃₋₆ branched alkyl, C₃₋₈ cycloalkyl, C₁₋₄-alkyl-C₃₋₈-cycloalkyl, C₃₋₈ heterocycloalkyl, C₁₋₄-alkyl-C₃₋₈ heterocycloalkyl, —R₂₂—OR₁₂, —R₂₂—S(O)₀₋₂R₁₂, —R₂₂—S(O)₂NR₁₃R₁₄, —R₂₂—C(O)OR₁₂, —R₂₂—C(O)R₁₉, —R₂₂—OC(O)R₁₉, —R₂₂—C(O)NR₁₃R₁₄, —R₂₂—NR₁₅S(O)₂R₁₂, —R₂₂—NR₂₃R₂₄, —R₂₂—NR₁₅C(O)R₁₉, —R₂₂—NR₁₅C(O)OCH₂Ph, —R₂₂—NR₁₅C(O)OR₁₂, —R₂₂—NR₁₅C(O)NR₁₃R₁₄, C₆₋₁₀ aryl, C₅₋₁₀ heteroaryl, —C₁₋₂alkyl-C₃₋₈-cycloalkyl, —C₁₋₂ alkyl-aryl, —C₁₋₂ alkyl-heterocycloalkyl and —C₁₋₂ alkyl-heteroaryl,
wherein each of said C₁₋₆alkyl, C₁₋₆haloalkyl, C₃₋₆ branched alkyl, C₁₋₄ alkyl-, C₃₋₈ heterocycloalkyl, and C₃₋₈ cycloalkyl, groups are optionally substituted with up to three R₂₀,
and each of said aryl and heteroaryl groups is optionally substituted with up to three R₂₁, halo or C₁₋₆ alkoxy;
alternatively, R₁₇ and R₁₈ along with the nitrogen atom to which they are attached to can be taken together to form a four to six, seven or eight-membered heterocyclic ring containing up to one additional N, O or S as a ring member, which can be optionally fused with a 5-6-membered optionally-substituted aryl or heteroaryl,
wherein the carbon atoms of said heterocyclic, aryl and heteroaryl rings are optionally substituted with R₂₀, and the nitrogen atom of said rings are optionally substituted with R₂₁;

R₁₉ is selected from optionally substituted alkyl, optionally substituted cycloalkyl, optionally substituted heterocycloalkyl, optionally substituted aryl, and optionally substituted heteroaryl;

R₂₀ is selected from the group consisting of halo, hydroxy, amino, CN, CONR₁₃R₁₄, oxo (═O), C₁₋₆alkoxy, C₁₋₆ alkyl, C₂₋₄ alkenyl, C₂₋₄ alkynyl, and C₁₋₆ haloalkyl;
and two R²⁰ on the same or adjacent connected atoms can be taken together with the atoms to which they are attached to form a 3-8 membered carbocyclic or heterocyclic ring containing up to 2 heteroatoms selected from N, O and S as ring members and optionally substituted with up to two groups selected from halo, oxo, Me, OMe, CN, hydroxy, amino, and dimethylamino;
R₂₁ is selected from the group consisting of C₁₋₆alkyl, C₁₋₆haloalkyl, —C(O)R₁₂, C(O)OR₁₂, and —S(O)₂R₁₂;
R₂₂ is selected from the group consisting of C₁₋₆ alkyl, C₁₋₆haloalkyl, C₃₋₆ branched alkyl, C₃₋₆ branched haloalkyl;

R₂₃ and R₂₄ are each, independently, selected from the group consisting of hydrogen, C₁₋₆ alkyl, C₁₋₆haloalkyl, C₃₋₆ branched alkyl, C₃₋₆ branched haloalkyl;

R₂ is selected from hydrogen, C₁₋₆ alkyl, C₃₋₈ cycloalkyl, C₃₋₈ branched alkyl, C₄₋₈ heterocycloalkyl, C₆₋₁₀ aryl and C₅₋₁₀ heteroaryl wherein said C₁₋₆ alkyl, C₃₋₈ cycloalkyl, C₃₋₈ branched alkyl, and C₄₋₈ heterocycloalkyl groups are optionally substituted with up to three R₂₀, and said aryl and heteroaryl groups are optionally substituted with up to three groups selected from halo, C₁₋₆alkoxy, and R₂₁;

R₄ₐ, R_{4b}, R₅, and R₆ are each, independently, selected from the group consisting of hydrogen, hydroxyl, cyano, halogen, C₁₋₄ alkyl, C₁₋₄haloalkyl, C₂₋₄ alkenyl, C₂₋₄ alkynyl, amino, NR₁₀R₁₁, C₁₋₄alkoxy and C₁₋₄haloalkoxy;

R₃ and R₈ are each, independently, selected from the group consisting of hydrogen, hydroxyl, cyano, halogen, optionally substituted C₁₋₄ alkyl, tetrazolyl, morpholino, C₁₋₄ haloalkyl, optionally substituted C₂₋₄ alkenyl, optionally substituted C₂₋₄ alkynyl, C₁₋₄ alkoxy, NR₁₀R₁₁, C(O)R₁₂, C(O)OR₁₂, C(O)NR₁₃R₁₄, S(O)₀₋₂R₁₂, S(O)₀₋₂NR₁₃R₁₄, and optionally substituted C₃₋₄ cycloalkyl;

R₉ is selected from the group consisting of hydrogen, C₁₋₄ alkyl, alkoxy, C(O)R₁₂, C(O)OR₁₅, C(O)NR₁₃R₁₄, S(O)₀₋₂R₁₂, S(O)₀₋₂NR₁₃R₁₄, optionally substituted C₃₋₄ cycloalkyl, and optionally substituted heterocycloalkyl;

R₁₀ and R₁₁ are each, independently, selected from the group consisting of hydrogen, hydroxyl, alkyl, alkoxy, C(O)R₁₂, C(O)OR₁₂, C(O)NR₁₃R₁₄, S(O)₀₋₂R₁₂, and S(O)₀₋₂NR₁₃R₁₄;
alternatively, R₁₀ and R₁₁ along with the nitrogen atom to which they are attached to can be taken together to form an optionally substituted four to six membered heteroaromatic, or a non-aromatic heterocyclic ring containing up to one additional heteroatom selected from N, O and S as a ring member;

R₁₂ and R₁₅ are each, independently selected from the group consisting of hydrogen, alkyl, branched alkyl, haloalkyl, branched haloalkyl, (CH₂)₀₋₃-cycloalkyl, (CH₂)₀₋₃-heterocycloalkyl, (CH₂)₀₋₃-aryl, and heteroaryl;

R₁₃ and R₁₄ are each, independently, selected from the group consisting of hydrogen, hydroxyl, alkyl, branched alkyl, haloalkyl, branched haloalkyl, alkoxy, cycloalkyl or heterocycloalkyl; and alternatively, R₁₃ and R₁₄ along with the nitrogen atom to which they are attached to can be taken together to form an optionally substituted four to six membered heteroaromatic, or non-aromatic heterocyclic ring that can contain an additional heteroatom selected from N, O and S as a ring member.

In some embodiments, the compound is a compound of Formula I, or a pharmaceutically acceptable salt or deuterated version thereof, wherein:

A₁ is N or CR₆;

A₃ is N or CR₈;

A₄ is selected from the group consisting of a bond, SO₂, CO—NR₉, NR₉, —SO₂—NR₉—, and O;

L is selected from the group consisting of a bond, optionally substituted C₁₋₄alkyl, C₃₋₆ cycloalkyl, C₃₋₆ heterocycloalkyl, and C₂₋₄ alkenyl;

R₁ is —X—R₁₆;

X is a bond or C₁₋₄ alkyl;

R₁₆ is selected from the group consisting of C₁₋₆ alkyl, C₃₋₆ branched alkyl, C₃₋8cycloalkyl, heterocycloalkyl, C₃₋₈-partially unsaturated cycloalkyl, aryl, and heteroaryl,
wherein R₁₆ is optionally substituted with up to three groups independently selected from halogen, oxo (═O), C₁₋₆alkyl, C₁₋₆haloalkyl, C₃₋₆ branched alkyl, C₃₋₆ branched haloalkyl, OH, C₁₋₆alkoxy, C₄₋₈ heterocycloalkyl, C₁₋₂alkyl-heterocycloalkyl, C₁₋₂alkyl-heteroaryl, —R₂₂—OR₁₂, —S(O)₀₋₂R₁₂, —R₂₂—S(O)₀₋₂R₁₂, —S(O)₂NR₁₃R₁₄, —R₂₂—S(O)₂NR₁₃R₁₄, —C(O)OR₁₂, —R₂₂—C(O)OR₁₂, —C(O)R₁₉, —R₂₂—C(O)R₁₉, —O—C₁₋₃ alkyl, —OC₁₋₃ haloalkyl, —OC(O)R₁₉, —R₂₂—OC(O)R₁₉, —C(O)NR₁₃R₁₄, —R₂₂—C(O)NR₁₃R₁₄, —NR₁₅S(O)₂R₁₂, —R₂₂—NR₁₅S(O)₂R₁₂; —NR₁₇R₁₈, —R₂₂—NR₁₇R₁₈, —NR₁₅C(O)R₁₉, —R₂₂—NR₁₅C(O)R₁₉, —NR₁₅C(O)OCH₂Ph, —R₂₂—NR₁₅C(O)OCH₂Ph, —NR₁₅C(O)OR₁₂, —R₂₂—NR₁₅C(O)OR₁₂, —NR₁₅C(O)NR₁₃R₁₄, and —R₂₂—NR₁₅C(O)NR₁₃R₁₄,
   wherein said C₁₋₆alkyl and C₃₋₆ branched alkyl are optionally substituted with up to three R₂₀;

R₁₇ and R₁₈ are each, independently, selected from the group consisting of hydrogen, hydroxyl, C₁₋₆alkyl, C₁₋₆haloalkyl, C₃₋₆ branched alkyl, C₃₋₆ cycloalkyl, —R₂₂—OR₁₂, —R₂₂—S(O)₀₋₂R₁₂, —R₂₂—S(O)₂NR₁₃R₁₄, —R₂₂—C(O)OR₁₂, —R₂₂—C(O)R₁₉, —R₂₂—OC(O)R₁₉, —R₂₂—C(O)NR₁₃R₁₄, —R₂₂—NR₁₅S(O)₂R₁₂, —R₂₂—NR₂₃R₂₄, —R₂₂—NR₁₅C(O)R₁₉, —R₂₂—NR₁₅C(O)OCH₂Ph, —R₂₂—NR₁₅C(O)OR₁₂, —R₂₂—NR₁₅C(O)NR₁₃R₁₄, heterocycloalkyl, aryl, heteroaryl, —C₁₋₂alkyl-cycloalkyl, —C₁₋₂ alkyl-aryl, —C₁₋₂ alkyl-heterocycloalkyl and —C₁₋₂ alkyl-heteroaryl,
wherein each of said C₁₋₆alkyl, C₁₋₆haloalkyl, C₃₋₆ branched alkyl, heterocycloalkyl, and C₃₋₆ cycloalkyl groups are optionally substituted with up to three R₂₀,
and each of said aryl and heteroaryl groups is optionally substituted with up to three R₂₁, halo or C₁₋₆ alkoxy;
alternatively, R₁₇ and R₁₈ along with the nitrogen atom to which they are attached to can be taken together to form a four to six membered heterocyclic ring containing up to one additional N, O or S as a ring member,
wherein the carbon atoms of said heterocyclic ring are optionally substituted with R₂₀, and the additional nitrogen atom of said ring are optionally substituted with R₂₁;

R₁₉ is selected from optionally substituted alkyl, optionally substituted cycloalkyl, optionally substituted heterocycloalkyl, optionally substituted aryl, and optionally substituted heteroaryl;

R₂₀ is selected from the group consisting of halo, hydroxy, amino, CN, CONR₁₃R₁₄, oxo (═O), C₁₋₆ alkoxy, C₁₋₆ alkyl and C₁₋₆ haloalkyl;
R₂₁ is selected from the group consisting of C₁₋₆alkyl, C₁₋₆haloalkyl, —C(O)R₁₂, C(O)OR₁₂, and —S(O)₂R₁₂;
R₂₂ is selected from the group consisting of C₁₋₆ alkyl, C₁₋₆haloalkyl, C₃₋₆ branched alkyl, C₃₋₆ branched haloalkyl;

R₂₃ and R₂₄ are each, independently, selected from the group consisting of hydrogen, C₁₋₆ alkyl, C₁₋₆haloalkyl, C₃₋₆ branched alkyl, C₃₋₆ branched haloalkyl;

R₂ is selected from hydrogen, C₁₋₆ alkyl, C₃₋₈ cycloalkyl, C₃₋₈ branched alkyl, C₄₋₈ heterocycloalkyl, aryl and heteroaryl wherein said C₁₋₆ alkyl, C₃₋₈ cycloalkyl, C₃₋₈ branched alkyl, and C₄₋₈ heterocycloalkyl groups are optionally substituted with up to three R₂₀, and said aryl and heteroaryl groups are optionally substituted with up to three groups selected from halo, C₁₋₆ alkoxy, and R₂₁;

R₄ₐ, R_{4b}, R₅, and R₆ are each, independently, selected from the group consisting of hydrogen, hydroxyl, cyano, halogen, C₁₋₄ alkyl, C₁₋₄haloalkyl, C₂₋₄ alkenyl, C₂₋₄ alkynyl, amino, NR₁₀R₁₁, C₁₋₄ alkoxy and C₁₋₄haloalkoxy;

R₃ and R₈ are each, independently, selected from the group consisting of hydrogen, hydroxyl, cyano, halogen, optionally substituted C₁₋₄ alkyl, tetrazolyl, morpholino, C₁₋₄ haloalkyl, optionally substituted C₂₋₄ alkenyl, optionally substituted C₂₋₄ alkynyl, C₁₋₄ alkoxy, NR₁₀R₁₁, C(O)R₁₂, C(O)OR₁₂, C(O)NR₁₃R₁₄, S(O)₀₋₂R₁₂, S(O)₀₋₂NR₁₃R₁₄, and optionally substituted C₃₋₄ cycloalkyl;

R₉ is selected from the group consisting of hydrogen, C₁₋₄ alkyl, alkoxy, C(O)R₁₂, C(O)OR₁₅, C(O)NR₁₃R₁₄, S(O)₀₋₂R₁₂, S(O)₀₋₂NR₁₃R₁₄, optionally substituted C₃₋₄ cycloalkyl, and optionally substituted heterocycloalkyl;

R₁₀ and R₁₁ are each, independently, selected from the group consisting of hydrogen, hydroxyl, alkyl, alkoxy, C(O)R₁₂, C(O)OR₁₂, C(O)NR₁₃R₁₄, S(O)₀₋₂R₁₂, and S(O)₀₋₂NR₁₃R₁₄;
alternatively, R₁₀ and R₁₁ along with the nitrogen atom to which they are attached to can be taken together to form an optionally substituted four to six membered heteroaromatic, or a non-aromatic heterocyclic ring containing up to one additional heteroatom selected from N, O and S as a ring member;

R₁₂ and R₁₅ are each, independently selected from the group consisting of hydrogen, alkyl, branched alkyl, haloalkyl, branched haloalkyl, (CH₂)₀₋₃-cycloalkyl, (CH₂)₀₋₃-heterocycloalkyl, (CH₂)₀₋₃-aryl, and heteroaryl;

R₁₃ and R₁₄ are each, independently, selected from the group consisting of hydrogen, hydroxyl, alkyl, branched alkyl, haloalkyl, branched haloalkyl, alkoxy, cycloalkyl or heterocycloalkyl; and alternatively, R₁₃ and R₁₄ along with the nitrogen atom to which they are attached to can be taken together to form an optionally substituted four to six membered heteroaromatic, or non-aromatic heterocyclic ring that can contain an additional heteroatom selected from N, O and S as a ring member.

In certain embodiments of these compounds, A₁ is CR₆; and A₃ is CR₈.

In alternative embodiments, A₁ is N; and A₃ is CR₈.

In alternative embodiments, A₁ is CR₆; and A₃ is N.

In some embodiments of the above compounds, R₈ is selected from halogen, hydrogen, CN, CF₃, O—C₁₋₃-alkyl, and C₁₋₃-alkyl. In some embodiments, R₈ is selected from hydrogen, Cl, F, and methyl. In preferred embodiments, R₈ is Cl or F, and most preferably it is Cl.

In some embodiments of the above compounds, R₆ is selected from halogen, hydrogen, CN, CF₃, O—C₁₋₃-alkyl, and C₁₋₃-alkyl. In some embodiments, R₆ is selected from hydrogen, Cl, F, and methyl. In preferred embodiments, R₆ is H.

In these compounds of Formula I, X can be a bond or a C₁₋₄ alkyl linker, such as —CH₂—. In some embodiments, X is a bond, particularly when R₁₆ is a cyclic group such as an optionally substituted C₃₋₈cycloalkyl, heterocycloalkyl, C₃₋₈-partially unsaturated cycloalkyl, aryl, or heteroaryl

R₁₆ can be any of the groups described above. In some embodiments, it is a C₁₋₂ alkyl, C₃₋₆ cycloalkyl, or C₄₋₈ heterocyclyl containing one or two heteroatoms selected from N, O and S as ring members. In specific embodiments, it is a C5 or C6 cycloalkyl or a C₅₋₆ heterocycloalkyl containing one heteroatom. These alkyl, cycloalkyl and heterocycloalkyl groups can be substituted; preferably, they have at least one substituent. Typically, R₁₆ is substituted with up to three groups, preferably 1-3 groups, independently selected from halogen, C₁₋₃alkyl, C₃₋₆ branched alkyl, OH, C₁₋₂alkoxy, —R₂₂—OR₁₂, S(O)₁₋₂R₁₂, —C(O)OR₁₂, —R₂₂—C(O)OR₁₂, —C(O)R₁₉, —R₂₂—OC(O)R₁₉, —C(O)NR₁₃R₁₄, —NR₁₅S(O)₂R₁₂, —NR₁₇R₁₈, —R₂₂—NR₁₇R₁₈, —NR₁₅C(O)R₁₉, —R₂₂—NR₁₅C(O)R₁₉, and —NR₁₅C(O)OCH₂Ph. In some embodiments, R₁₆ is substituted with amino, hydroxy, oxo C₁₋₄ alkyl, C₁₋₄ aminoalkyl, C₁₋₄ hydroxyalkylamine, or —NR₁₇R₁₈, e.g., —OMe.

R₅ and R_{4b} can be as described above; in many embodiments, each of them is H. R₆ when present is often H, also. R₄ₐ can be various groups; in some embodiments, it is H, F, Cl or Me.

R₈ when present can be as described above; advantageously it is a group other than H, particularly F, Cl, Me, or CF₃.

R₃ in the compounds of the invention can be H, Cl, F, Me, OMe, CN, COOR, OH, CF3, or tetrazole. In some embodiments, it is H, Cl or F. In a preferred embodiment, R₃ is H.

A₄ can be any of the groups described above; in some embodiments, it is O, SO₂ or NR₉. In certain embodiments, A₄ is O. In a preferred embodiment, A₄ is NH.

L can be a bond or various linking groups as described above. In some embodiments, L is a divalent alkyl group such as —(CH₂)₁₋₄—. In preferred embodiments, L is —CH₂— or —CH₂CH₂—.

R₂ can be any of the groups described above; in some embodiments, it is a 6-membered ring such as cycloalkyl, heterocycloalkyl or phenyl, and is optionally substituted. Exemplary six-membered rings include cyclohexyl, piperidinyl, morpholinyl, tetrahydropyranyl, dioxolanyl, and phenyl. Suitable substituents include one or more halo, C₁₋₄ alkyl, hydroxy, C₁₋₄ alkoxy, C₂₋₄ alkenyl, C₂₋₄ alkynyl, amino, CN, CONH₂, CONHMe, CONMe₂, and the like; and for non-aromatic rings, the suitable substituents further include oxo. Some preferred selections for this R₂ ring include phenyl, piperidinyl, and tetrahydropyranyl, e.g., 4-tetrahydropyranyl. Phenyl groups are typically substituted, while the heteroaryl groups can be substituted or unsubstituted.

These cyclic R₂ groups can be unsubstituted or they can be substituted, typically with up to two groups selected from halo, OH, COOMe, CN, CONH₂, ethyl, vinyl, ethynyl, CONHMe, CONMe₂, Me, OMe, and CF₃.

In some embodiments, six-membered rings are preferred for R₂, e.g., phenyl, piperidinyl, tetrahydropyranyl, and pyridinyl. Preferred embodiments of R₂ when -A₄-L- is a group of the formula —NH—(CH₂)—, include phenyl, pyridinyl, piperidinyl, and tetrahydropyranyl, each of which can be substituted with up to two groups selected from halo, Me, OMe, OH, CN, and CONH₂; particularly phenyl or 4-pyridinyl substituted with up to 2 halo substituents, preferably F or Cl; and piperidin-4-yl or tetrahydropyran-4-yl, each of which is unsubstituted or is substituted with Me, OMe, OH, CN or CONH₂, frequently at position 4.

In some embodiments, R₂ is a cyclopropyl ring that can be unsubstituted or it can be substituted, typically with up to two groups selected from halo, OH, COOMe, CN, CONH₂, CONHMe, CONMe₂, Me, OMe, ethynyl, vinyl, and CF₃. In some embodiments, the cyclopropyl ring is unsubstituted, or it is substituted at Cl with Me, OMe, F, OH, CN or CONH₂.

In specific embodiments, -L-R₂ is

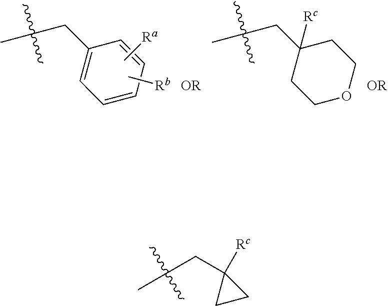

where R^{a} and R^{b} and R^{c} each independently represent H, F, Cl, —OCHF₂, —C(O)-Me, —OH, CF₃, Me, —OMe, —CN, —C≡CH, vinyl, -Ethyl, COOMe, COOH, NH₂, NMe₂, —CONH₂, or —NH—C(O)-Me. Preferably, R^{a} and R^{b} are selected from H, F, Cl, OMe, CF₃, and Me. Preferably, R^{c} is H, F, CN, Me, or OMe.

In some embodiments, -L-R₂ is a group of the formula:

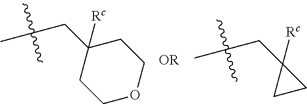

wherein R^{c} is CN, Me, H, OMe, or CF₃.

In some embodiments of the compounds of any of the foregoing embodiments, R₁ is —X—R₁₆ wherein X is a bond or C₁₋₂ alkyl; and

R₁₆ is selected from the group consisting of C₁₋₂-alkyl, C₄₋₆cycloalkyl, C₄₋₈ heterocycloalkyl, phenyl, and C₅₋₁₀ heteroaryl,

wherein R₁₆ is substituted with up to three groups independently selected from halogen, C₁₋₃alkyl, C₃₋₆ branched alkyl, OH, C₁₋₂alkoxy, —R₂₂—OR₁₂, S(O)₁₋₂R₁₂, —C(O)OR₁₂, —R₂₂—C(O)OR₁₂, —C(O)R₁₉, —R₂₂—OC(O)R₁₉, —C(O)NR₁₃R₁₄, —NR₁₅S(O)₂R₁₂, —R₂₂—NR₁₇R₁₈, —NR₁₅C(O)R₁₉, —R₂₂—NR₁₅C(O)R₁₉, and —NR₁₅C(O)OCH₂Ph.

In some embodiments of the compounds of any of the foregoing embodiments, R₁₆ is selected from the group consisting of C₁₋₂-alkyl, cyclopentyl, cyclohexyl, piperidine, piperazine, morpholine, pyridine, pyrrolidine, cyclohexenyl, and tetrahydro-2H-pyran;

wherein R₁₆ is substituted with one to three groups selected from amino, hydroxyl, —NHCH₂-phenyl, —CH₂-amino, —COO-t-butyl, methoxy, —NH—SO₂-ethyl, —CH₂—NHSO₂-ethyl, —SO₂-ethyl, t-butyl, methyl, —CH₂—COOH, —CO—NHCH₃, —CON(CH₃)₂, —NHC(CH₃)—CH₂—SO₂—CH₃, —NH—COO—CH₂-phenyl, hydroxy-methyl, —CH₂—NH—CH₃, CH₂—NH-ethyl, —NH—CH₂—CH₂-methoxy, —CH₂—NH—CO—CH₃, —NH—CH₂—CH₂OH, —NH—CO—CH₂—N(CH₃)₂, —NH—CO-methylpyrrolidine, —NH—CH₂—C(CH₃)-dioxolane, pyridyl, NH-ethyl, pyrrolidine, —CH₂—NH—CO-pyridyl, —NH-tetrahydropyran, —COCH₂—N(CH₃)₂, —NH—CH₂—C(CH₃)-dimethyldioxolane, tetrahydropyran, —CO-methylpyrrolidine, —CH₂-methylpiperidine, —NH—CO—CH₃, —NH—SO₂—CH₃, —NH—CH(CH₂—OCH₃)₂, —NH—CH₂-tetrahydrofuran, —NH—CH₂-oxetane, —NH—CH₂-tetrahydropyran, CH₂-dioxane, —N(CH₃)—CH₂CH₂—OCH₃, —CH(OH)—CH₂-amino, —NH—CH₂CH₂—OCF₃, —NHCH₂—OCH₃, —NH—CH₂—CH(CF₃)—OCH₃, —NH—CH(CH₃)—CH₂—OH, F, —NH-oxetane, —CH₂—CH₂—OCH₃, —CH₂—OCH₃, —CH₂-tetrahydropyran, —CH₂-methylpiperazine, —NH—CH₂—CH(OH)—CF₃, piperidine, —CH₂-pyrrolidine, —NH—CH(CH₃)CH₂OCH₃, —NH-tetrahydrofuran, —(CH₂)₃—NH₂, hydroxyethyl, propyl, —CH₂-pyridyl, —CH₂-piperidine, morpholine, —NH-chloropyrimidine, —NH—CH₂CH₂—SO₂-methyl, —(CH₃)₃—N(CH₃)₂, piperazine,

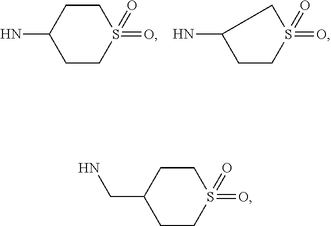

and —CH₂-morpholine.

In some embodiments of the compounds of Formula I, R¹ is substituted cyclohexyl. In some such embodiments, R₁ is cyclohexyl substituted with —NR₁₇R₁₈,
wherein R₁₇ and R₁₈ are each, independently, selected from the group consisting of hydrogen, hydroxyl, C₁₋₆alkyl, C₁₋₆haloalkyl C₃₋₆ branched alkyl, C₃₋₆ cycloalkyl, —R₂₂—OR₁₂, —R₂₂—S(O)₀₋₂R₁₂, —R₂₂—S(O)₂NR₁₃R₁₄, —R₂₂—C(O)OR₁₂, —R₂₂—C(O)R₁₉, —R₂₂—OC(O)R₁₉, —R₂₂—C(O)NR₁₃R₁₄, —R₂₂—NR₁₅S(O)₂R₁₂, —R₂₂—NR₂₃R₂₄, —R₂₂—NR₁₅C(O)R₁₉, —R₂₂—NR₁₅C(O)OCH₂Ph, —R₂₂—NR₁₅C(O)OR₁₂, —R₂₂—NR₁₅C(O)NR₁₃R₁₄, cycloalkyl, heterocycloalkyl and heteroaryl;
or R₁₇ and R₁₈ along with the nitrogen atom to which they are attached can be taken together to form a four to six or seven membered heterocyclic ring that can contain an additional O, N or S as a ring member, wherein the carbon atoms of said ring are optionally substituted with R₂₀, and the nitrogen atoms of said ring are optionally substituted with R₂₁.
In some preferred embodiments, R₁ is

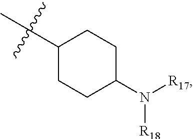

where R₁₇ is H. Suitably, —NR₁₇R₁₈ is a group of the formula:

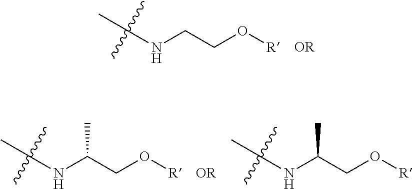

wherein R′ is H, Me, or Et.

In some embodiments of the foregoing compounds, R₃ is selected from H, methyl, cyano, chloro, CONH₂, amino, tetrazolyl, cyclopropyl, ethyl, and fluoro;

R₄ₐ and R_{4b} are independently selected from halogen, methyl, hydrogen, and halo-methyl;

R₆ is H if A₁ is CR₆;

R₈ is Cl if A₃ is CR₈;

R₁₆ is C₁₋₆ alkyl or C₃₋₈ cycloalkyl, and R₁₆ is substituted with one to three groups independently selected from hydroxyl, C₁₋₆ alkyl, —NR₁₇R₁₈ and —R₂₂—NR₁₇R₁₈;
wherein R₁₇ and R₁₈ are each, independently, selected from the group consisting of hydrogen, C₁₋₃alkyl, C₁₋₄haloalkyl, C₃₋₆ branched alkyl, —R₂₂—OR₁₂, —R₂₂—S(O)₂R₁₂, —R₂₂—NR₁₅S(O)₂R₁₂, heterocycloalkyl and heteroaryl;
alternatively, R₁₇ and R₁₈ along with the nitrogen atom to which they are attached to can be taken together to form a four to six membered heterocyclic ring containing up to one additional heteroatom selected from N, O and S as a ring member and wherein said ring carbon atoms are optionally substituted with R₂₀, and the additional nitrogen atom is optionally substituted with R₂₁;
R₁₉ is selected from optionally substituted C₁₋₃-alkyl, optionally substituted heterocycloalkyl, optionally substituted aryl and optionally substituted heteroaryl;
R₂₀ represents the group C₁₋₃alkyl; and
R₂₂ is selected from the group consisting of C₁₋₄alkyl, and C₃₋₆ branched alkyl.

In other embodiments of the compounds described above, A₄ is selected from NR₉, O, and a bond; L is selected from a bond, C₁₋₄-alkyl, and cyclopropyl;

R₂ is selected from the group consisting of C₃₋₇ cycloalkyl, C₅₋₇ heterocycloalkyl, phenyl, and pyridyl, wherein said C₃₋₇ cycloalkyl and C₅₋₇ heterocycloalkyl are optionally substituted with up to three substituents independently selected from halogen, methoxy, dihalo-methoxy, trihalo-methoxy, trihalo alkyl, C₁₋₃-alkyl, and hydroxyl, and said phenyl and pyridyl are optionally substituted with up to three groups selected from halogen, cyano, oxo, CONH₂, CONHMe, CONMe₂, methoxy, dihalo-methoxy, trihalo-methoxy, trihalo C₁₋₆-alkyl, and C₁₋₃-alkyl; and

R₉ represents methyl, hydrogen, or ethyl.

In yet other embodiments of the compounds described above, X is a bond;

R₁₆ is selected from cyclohexyl, and C₂₋₅-alkyl, CH(CH₂OH)₂, CH₂—CH(OH)—CH₂NH₂; CH₂—C(CH₃)₂—CH₂NHCH₃, CH(CH₃)OH, CH₂—C(CH₃)₂—CH₂NH₂, cyclopentyl, and cyclopropyl, wherein each said cyclohexyl, cyclopentyl, cyclopropyl and C₂₋₅-alkyl group is substituted with 1 to 2 substituents selected from amino, methyl-amino, hydroxy, amino-ethyl, dimethyl-amino, —NH—(CH₂)₂—O-ethyl, —NH—SO₂-methyl, —CH₂—NH—SO₂-methyl, piperidinyl, pyrrolidinyl, —NH—CH₂—CF₃, —NH—(CH₂)₂—O-methyl, —N(CH₃)—(CH₂)₁₋₂-methoxy, —NH—CH₂—CH(CH₃)—OH, NH—CH₂-tetrahydrofuranyl, —NH—(CH₂)₂—OH, —NH—CH₂—CONH₂, —NH(CH₂)₂—CF₃, methylpyrrolidin-3-ol, NH—(CH₂)₂-pyrrolidinyl, —NH—CH₂—COOH, —NH—CH₂-dioxane, —NH-oxetane, —NH-tetrahydrofuranyl, morpholinyl, —NH—(CH₂)₂—O—(CH₂)₂—OCH₃, —NH—(CH₂)₂—CONH₂, and —N(CH₂CH₂OCH₃)₂;

R₂ is selected from pyridyl, phenyl, tetrahydropyranyl, cyclopropyl, cyclohexyl, cycloheptyl, 1,4-dioxane, morpholinyl, alkyl substituted dioxane, tetrahydrofuranyl, dioxepane, piperidinyl and

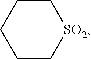

wherein each R₂ is substituted with one, two, or three groups independently selected from hydrogen, Cl, Br, F, methoxy, hydroxy-methyl, hydrogen, —CONR′₂, —SO₂R′, —SR′, —C(O)—R′, —COOR′, —NR′₂, cyano, dihalo-methoxy, trihalo-methoxy, trifluoro-methyl, hydroxyl and methyl; where each R′ is independently H or C1-C4 alkyl, and wherein two R′ on N can optionally cyclise to form a 5-7 membered heterocyclic ring that can optionally contain an additional heteroatom selected from N, O and S as a ring member;

A₄ is NH;

L is a bond, C₁₋₂alkyl or C₃₋₄ cycloalkyl;

R₃ is selected from H, CONH₂, hydroxyethyl, chloro, tetrazolyl, hydroxy, morpholino, cyano, fluoro, and methoxy;

R₄ₐ and R_{4b} are independently selected from H, Cl, and fluoro;

R₅ represents H;

R₆ represents hydrogen; and

R₈ is selected from hydrogen, chloro and fluoro.

In other embodiments of the compounds described above:

X represents a bond;

R₁₆ is selected from cyclohexyl, and C₂₋₅-alkyl, —CH(CH₂OH)₂, —CH₂—CH(OH)—CH₂NH₂; —CH₂—C(CH₃)₂—CH₂NHCH₃, —CH(CH₃)OH, —CH₂—C(CH₃)₂—CH₂NH₂, cyclopentyl, and cyclopropyl, wherein each said cyclohexyl, cyclopentyl, cyclopropyl and C₂₋₅-alkyl group is substituted with 1 to 2 substituents selected from amino, methyl-amino, hydroxy, amino-ethyl, dimethyl-amino, —NH—(CH₂)₂—O-ethyl, —NH—SO₂-methyl, —CH₂—NH—SO₂-methyl, piperidinyl, pyrrolidinyl, —NH—CH₂—CF₃, —NH—(CH₂)₂—O-methyl, —N(CH₃)—(CH₂)₁₋₂-methoxy, —NH—CH₂—CH(CH₃)—OH, —NH—CH₂-tetrahydrofuranyl, —NH—(CH₂)₂—OH, —NH—CH₂—CONH₂, —NH(CH₂)₂—CF₃, methylpyrrolidin-3-ol, —NH—(CH₂)₂-pyrrolidinyl, —NH—CH₂—COOH, —NH—CH₂-dioxane, —NH-oxetane, —NH-tetrahydrofuranyl, morpholinyl, —NH—(CH₂)₂—O—(CH₂)₂—OCH₃, —NH—(CH₂)₂—CONH₂, and —N(CH₂CH₂OCH₃)₂;

-L-R₂ is selected from —CH₂-fluorophenyl, —CH₂-difluorophenyl, —CH₂-chlorophenyl, —CH₂-pyridyl, —CH₂-cyclohexyl, —CH₂-piperidinyl, —CH₂-cyano-phenyl,

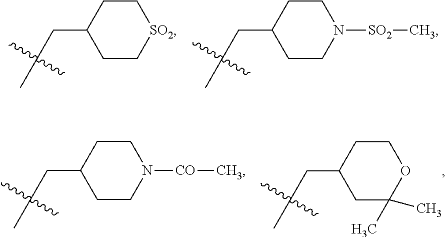

—CH₂-tetrahydropyran, benzyl, —CH₂-toluoyl, and —CH₂-methoxy-phenyl;

A₄ is NH;

R₃ is selected from H, CONH₂, hydroxyethyl, chloro, tetrazolyl, hydroxy, morpholino, cyano, fluoro, and methoxy;

R₄ₐ and R_{4b} are independently selected from H, Cl and fluoro;

R₅ represents H;

R₆ represents hydrogen; and

R₈ is selected from hydrogen, chloro and fluoro.

In preferred embodiments, the invention provides a compound selected from those depicted in Table 1 or Table 1B herein. For the compounds in Table 1, when the word ‘Chiral’ appears along with the structure, the structure shows the absolute stereochemistry. Where the word ‘chiral’ is not present, the compound is racemic (or is not optically active, due to a plane of symmetry, for example) and indications of stereochemistry are used to clarify relative stereochemistry rather than absolute stereochemistry.

In certain embodiments of the compounds of Formula I, the compound is of Formula II:

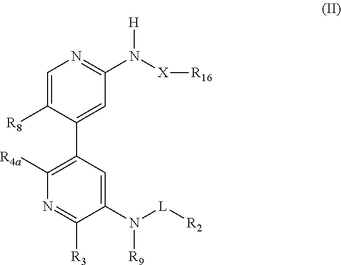

wherein:
X is a bond, —CH₂—, or —(CH₂)₂—,
   R₁₆ is selected from C₃-C₆ cycloalkyl and C₁₋₄ alkyl, each of which is optionally substituted with one to three groups independently selected from C₁₋₆ haloalkyl, halo, amino, oxo, —OR, —(CH₂)₂₋₄OR, —NR—(CH₂)₂₋₄—OR, —O—(CH₂)₂₋₄—OR, and C₁₋₄ aminoalkyl, wherein each R is independently C₁₋₄ alkyl or H;
   L is —CH₂— or a bond;
   R₈ is F or Cl;
   R₄ₐ is H, F or Cl;
   R₃ is H, F, Cl, OH, CN, or 4-morpholinyl;
   R₉ is H or Me; and
   R₂ is selected from cycloalkyl, heterocycloalkyl, heteroaryl and aryl, each of which is optionally substituted with up to three groups independently selected from halo, hydroxy, amino, CONH₂, haloalkyl, CN, C₁₋₄ alkyl, C₂₋₄ alkenyl, C₂₋₄alkynyl, and C₁₋₄ haloalkyl.

In the compounds of Formula II, in some embodiments R₈ is Cl; and R₄ₐ is H.

In some embodiments of the foregoing compounds of Formula II, R₃ is H and R₉ is H.

In some embodiments of the foregoing compounds of Formula II, L is —CH₂— and

R₂ is C₅₋₇ heterocycloalkyl,

wherein the heterocycloalkyl contains 1-2 heteroatoms selected from N, O and S as ring members, and is optionally substituted with up to three groups independently selected from halo, hydroxy, amino, haloalkyl, CN, CONH₂, C₁₋₄ alkyl, C₁₋₄ alkoxy, and C₁₋₄ haloalkyl. Suitable heterocycloalkyls include tetrahydropyran and piperidine.

In some preferred embodiments, -LR₂ is —CH₂-phenyl, where the phenyl is optionally substituted with one to three groups selected from halo, hydroxy, amino, methyl CF₃, and methoxy, or -LR₂ is a group of this formula, where the wavy line bisects the point of attachment of L to the rest of the Formula II structure:

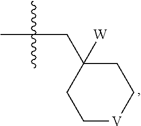

where V is O, NR, S or SO₂, where R is H or C₁₋₄ alkyl, and W is selected from H, Me, F, CN, OH, OMe, and CONH₂. In some such embodiments, V is O or NH, and W is H or CN.

In some embodiments of the compounds of Formula II, L is a bond and R₂ is aryl or heteroaryl, each of which is optionally substituted with up to three groups independently selected from halo, hydroxy, amino, haloalkyl, CN, C₁₋₄ alkyl, and C₁₋₄ haloalkyl. In some such embodiments, R₂ is phenyl and is optionally substituted with up to three groups independently selected from halo, hydroxy, amino, haloalkyl, CN, C₁₋₄ alkyl, and C₁₋₄ haloalkyl.

In other embodiments of the compounds of Formula II, L is CH₂ and R₂ is cyclopropyl, optionally substituted with Me, OMe, F, OH, CN or CONH₂; in certain substituted embodiments, one of these substituents is present at C-1 of the cyclopropyl ring.

In some preferred embodiments, -L-R₂ is a group of the formula

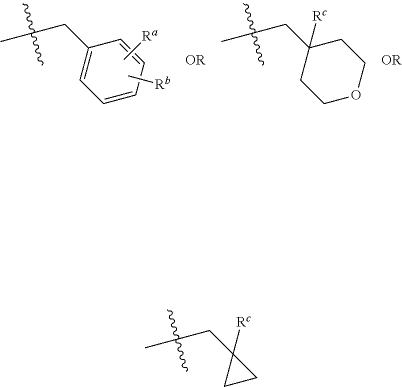

where R^{a} and R^{b} and R^{c} each independently represent H, F, Cl, CF₃, —OCHF₂, —C(O)-Me, —OH, Me, —OMe, —CN, —C≡CH, vinyl, -Ethyl, —CONH₂, or —NH—C(O)-Me. In particular embodiments, -L-R₂ is a group of the formula:

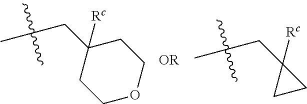

wherein R^{c} is CN, Me, H, OMe, or CF₃.

In some preferred embodiments of the foregoing compounds of Formula II, —X—R₁₆ is a C₅₋₆ cycloalkyl or heterocycloalkyl substituted with an amine-containing group such as NR₁₇R₁₈ as described above for Formula I. For example, —X—R₁₆ can be a group of this formula:

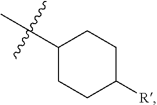

wherein R′ is selected from C₁₋₆ haloalkyl, halo, hydroxy, amino, oxo, C₁₋₄ aminoalkyl, —(CH₂)₁₋₄OR, —NR—(CH₂)₂₋₄—OR, and —O—(CH₂)₂₋₄—OR, wherein each R is independently C₁₋₄ alkyl or H. In preferred embodiments, R′ is a group of the formula:

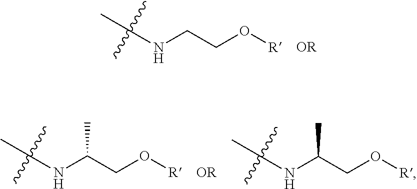

wherein R″ is H, Me, or Et.

Some specific embodiments of the invention described herein are enumerated below:

1. A compound of Formula (I):

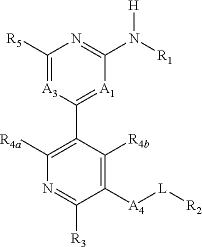

or a pharmaceutically acceptable salt thereof, wherein:

A₁ is N or CR₆;

A₃ is N or CR₈;

A₄ is selected from the group consisting of a bond, SO₂, CO—NR₉, NR₉, —SO₂—NR₉—, and O;

L is selected from the group consisting of a bond, optionally substituted C₁₋₄alkyl, C₃₋₆ cycloalkyl, C₃₋₆ heterocycloalkyl, and C₂₋₄ alkenyl;

R₁ is —X—R₁₆;

X is a bond or C₁₋₄ alkyl;

R₁₆ is selected from the group consisting of C₁₋₆ alkyl, C₃₋₆ branched alkyl, C₃₋₈cycloalkyl, heterocycloalkyl, C₃₋₈-partially unsaturated cycloalkyl, aryl, and heteroaryl,
wherein R₁₆ is optionally substituted with up to three groups independently selected from halogen, oxo (═O), C₁₋₆alkyl, C₁₋₆haloalkyl, C₃₋₆ branched alkyl, C₃₋₆ branched haloalkyl, OH, C₁₋₆alkoxy, C₄₋₈ heterocycloalkyl, C₁₋₂alkyl-heterocycloalkyl, C₁₋₂alkyl-heteroaryl, —R₂₂—OR₁₂, —S(O)₀₋₂R₁₂, —R₂₂—S(O)₀₋₂R₁₂, —S(O)₂NR₁₃R₁₄, —R₂₂—S(O)₂NR₁₃R₁₄, —C(O)OR₁₂, —R₂₂—C(O)OR₁₂, —C(O)R₁₉, —R₂₂—C(O)R₁₉, —O—C₁₋₃ alkyl, —OC₁₋₃ haloalkyl, —OC(O)R₁₉, —R₂₂—OC(O)R₁₉, —C(O)NR₁₃R₁₄, —R₂₂—C(O)NR₁₃R₁₄, —NR₁₅S(O)₂R₁₂, —R₂₂—NR₁₅S(O)₂R₁₂, —NR₁₇R₁₈, —R₂₂—NR₁₇R₁₈, —NR₁₅C(O)R₁₉, —R₂₂—NR₁₅C(O)R₁₉, —NR₁₅C(O)OCH₂Ph, —R₂₂—NR₁₅C(O)OCH₂Ph, —NR₁₅C(O)OR₁₂, —R₂₂—NR₁₅C(O)OR₁₂, —NR₁₅C(O)NR₁₃R₁₄, and —R₂₂—NR₁₅C(O)NR₁₃R₁₄,
   wherein said C₁₋₆alkyl and C₃₋₆ branched alkyl are optionally substituted with up to three R₂₀;

R₁₇ and R₁₈ are each, independently, selected from the group consisting of hydrogen, hydroxyl, C₁₋₆alkyl, C₁₋₆haloalkyl, C₃₋₆ branched alkyl, C₃₋₆ cycloalkyl, —R₂₂—OR₁₂, —R₂₂—S(O)₀₋₂R₁₂, —R₂₂—S(O)₂NR₁₃R₁₄, —R₂₂—C(O)OR₁₂, —R₂₂—C(O)R₁₉, —R₂₂—OC(O)R₁₉, —R₂₂—C(O)NR₁₃R₁₄, —R₂₂—NR₁₅S(O)₂R₁₂, —R₂₂—NR₂₃R₂₄, —R₂₂—NR₁₅C(O)R₁₉, —R₂₂—NR₁₅C(O)OCH₂Ph, —R₂₂—NR₁₅C(O)OR₁₂, —R₂₂—NR₁₅C(O)NR₁₃R₁₄, heterocycloalkyl, aryl, heteroaryl, —C₁₋₂alkyl-cycloalkyl, —C₁₋₂ alkyl-aryl, —C₁₋₂ alkyl-heterocycloalkyl and —C₁₋₂ alkyl-heteroaryl,
wherein each of said C₁₋₆alkyl, C₁₋₆haloalkyl, C₃₋₆ branched alkyl, heterocycloalkyl, and C₃₋₆ cycloalkyl groups are optionally substituted with up to three R₂₀,
and each of said aryl and heteroaryl groups is optionally substituted with up to three R₂₁, halo or C₁₋₆ alkoxy;
alternatively, R₁₇ and R₁₈ along with the nitrogen atom to which they are attached to can be taken together to form a four to six membered heterocyclic ring containing up to one additional N, O or S as a ring member,
wherein the carbon atoms of said heterocyclic ring are optionally substituted with R₂₀, and the additional nitrogen atom of said ring is optionally substituted with R₂₁;

R₁₉ is selected from optionally substituted alkyl, optionally substituted cycloalkyl, optionally substituted heterocycloalkyl, optionally substituted aryl, and optionally substituted heteroaryl;

R₂₀ is selected from the group consisting of halo, hydroxy, amino, CN, CONR₁₃R₁₄, oxo (═O), C₁₋₆ alkoxy, C₁₋₆ alkyl and C₁₋₆ haloalkyl;
R₂₁ is selected from the group consisting of C₁₋₆alkyl, C₁₋₆haloalkyl, —C(O)R₁₂, C(O)OR₁₂, and —S(O)₂R₁₂;
R₂₂ is selected from the group consisting of C₁₋₆alkyl, C₁₋₆haloalkyl, C₃₋₆ branched alkyl, C₃₋₆ branched haloalkyl;

R₂₃ and R₂₄ are each, independently, selected from the group consisting of hydrogen, C₁₋₆ alkyl, C₁₋₆haloalkyl, C₃₋₆ branched alkyl, C₃₋₆ branched haloalkyl;

R₂ is selected from hydrogen, C₁₋₆ alkyl, C₃₋₈ cycloalkyl, C₃₋₈ branched alkyl, C₄₋₈ heterocycloalkyl, aryl and heteroaryl wherein said C₁₋₆ alkyl, C₃₋₈ cycloalkyl, C₃₋₈ branched alkyl, and C₄₋₈ heterocycloalkyl groups are optionally substituted with up to three R₂₀, and said aryl and heteroaryl groups are optionally substituted with up to three groups selected from halo, C₁₋₆ alkoxy, and R₂₁;

R₄ₐ, R_{4b}, R₅, and R₆ are each, independently, selected from the group consisting of hydrogen, hydroxyl, cyano, halogen, C₁₋₄ alkyl, C₁₋₄haloalkyl, C₂₋₄ alkenyl, C₂₋₄ alkynyl, amino, NR₁₀R₁ₗ, C₁₋₄ alkoxy and C₁₋₄haloalkoxy;

R₃ and R₈ are each, independently, selected from the group consisting of hydrogen, hydroxyl, cyano, halogen, optionally substituted C₁₋₄ alkyl, tetrazolyl, morpholino, C₁₋₄ haloalkyl, optionally substituted C₂₋₄ alkenyl, optionally substituted C₂₋₄ alkynyl, C₁₋₄ alkoxy, NR₁₀R₁₁, C(O)R₁₂; C(O)OR₁₂, C(O)NR₁₃R₁₄, S(O)₀₋₂R₁₂, S(O)₀₋₂NR₁₃R₁₄, and optionally substituted C₃₋₄ cycloalkyl;

R₉ is selected from the group consisting of hydrogen, C₁₋₄ alkyl, alkoxy, C(O)R₁₂, C(O)OR₁₅, C(O)NR₁₃R₁₄, S(O)₀₋₂R₁₂, S(O)₀₋₂NR₁₃R₁₄, optionally substituted C₃₋₄ cycloalkyl, and optionally substituted heterocycloalkyl;

R₁₀ and R₁₁ are each, independently, selected from the group consisting of hydrogen, hydroxyl, alkyl, alkoxy, C(O)R₁₂, C(O)OR₁₂, C(O)NR₁₃R₁₄, S(O)₀₋₂R₁₂, and S(O)₀₋₂NR₁₃R₁₄;
alternatively, R₁₀ and R₁₁ along with the nitrogen atom to which they are attached to can be taken together to form an optionally substituted four to six membered heteroaromatic, or a non-aromatic heterocyclic ring containing up to one additional heteroatom selected from N, O and S as a ring member;

R₁₂ and R₁₅ are each, independently selected from the group consisting of hydrogen, alkyl, branched alkyl, haloalkyl, branched haloalkyl, (CH₂)₀₋₃-cycloalkyl, (CH₂)₀₋₃-heterocycloalkyl, (CH₂)₀₋₃-aryl, and heteroaryl;

R₁₃ and R₁₄ are each, independently, selected from the group consisting of hydrogen, hydroxyl, alkyl, branched alkyl, haloalkyl, branched haloalkyl, alkoxy, cycloalkyl or heterocycloalkyl; and alternatively, R₁₃ and R₁₄ along with the nitrogen atom to which they are attached to can be taken together to form an optionally substituted four to six membered heteroaromatic, or non-aromatic heterocyclic ring that can contain an additional heteroatom selected from N, O and S as a ring member.

2. The compound of embodiment 1, wherein:

A₁ is CR₆; and

A₃ is CR₈.

3. The compound of embodiment 1, wherein:

A₁ is N; and

A₃ is CR₈.

4. The compound of embodiment 1, wherein:

A₁ is CR₆; and

A₃ is N.

5. The compound of any one of embodiments 1-3, wherein:

R₈ is selected from halogen, hydrogen, CN, CF₃, O—C₁₋₃-alkyl, and C₁₋₃-alkyl.

6. The compound of any one of embodiments 1-3, wherein:

R₈ is selected from hydrogen, Cl, F, and methyl.

7. The compound of any one of embodiments 1-3, wherein R₈ is Cl or F.
8. The compound of any one of embodiments 1-7, wherein:

R₁ is —X—R₁₆ wherein X is a bond or C₁₋₂ alkyl; and

R₁₆ is selected from the group consisting of C₁₋₂-alkyl, C₄₋₆cycloalkyl, C₄₋₈ heterocycloalkyl, phenyl, and C₅₋₁₀ heteroaryl,

wherein R₁₆ is substituted with up to three groups independently selected from halogen, C₁₋₃alkyl, C₃₋₆ branched alkyl, OH, C₁₋₂alkoxy, —R₂₂—OR₁₂, S(O)₁₋₂R₁₂, —C(O)OR₁₂, —R₂₂—C(O)OR₁₂, —C(O)R₁₉, —R₂₂—OC(O)R₁₉, —C(O)NR₁₃R₁₄, —NR₁₅S(O)₂R₁₂, —NR₁₇R₁₈, —R₂₂—NR₁₇R₁₈, —NR₁₅C(O)R₄₉, —R₂₂—NR₁₅C(O)R₁₉, and —NR₁₅C(O)OCH₂Ph.

9. A compound of embodiments 1-3 or 5-8, wherein:

R₁₆ is selected from the group consisting of C₁₋₂-alkyl, cyclopentyl, cyclohexyl, piperidine, piperazine, morpholine, pyridine, pyrrolidine, cyclohexenyl, and tetrahydro-2H-pyran;

wherein R₁₆ is substituted with one to three groups selected from amino, hydroxyl, —NHCH₂-phenyl, —CH₂-amino, —COO-t-butyl, methoxy, —NH—SO₂-ethyl, —CH₂—NHSO₂-ethyl, —SO₂-ethyl, t-butyl, methyl, —CH₂—COOH, —CO—NHCH₃, —CON(CH₃)₂, —NHC(CH₃)—CH₂—SO₂—CH₃, —NH—COO—CH₂-phenyl, hydroxy-methyl, —CH₂—NH—CH₃, CH₂—NH-ethyl, —NH—CH₂—CH₂-methoxy, —CH₂—NH—CO—CH₃, —NH—CH₂—CH₂OH, —NH—CO—CH₂—N(CH₃)₂, —NH—CO-methylpyrrolidine, —NH—CH₂—C(CH₃)-dioxolane, —NH—CO-pyridyl, NH-ethyl, pyrrolidine, —CH₂—NH—CO-pyridyl, —NH-tetrahydropyran, —COCH₂—N(CH₃)₂, —NH—CH₂—C(CH₃)-dimethyldioxolane, tetrahydropyran, —CO-methylpyrrolidine, —CH₂-methylpiperidine, —NH—CO—CH₃, —NH—SO₂—CH₃, —NH—CH(CH₂—OCH₃)₂, —NH—CH₂-tetrahydrofuran, —NH—CH₂-oxetane, —NH—CH₂-tetrahydropyran, —NH—CH₂-dioxane, —N(CH₃)—CH₂CH₂—OCH₃, —CH(OH)—CH₂-amino, —NH—CH₂CH₂—OCF₃, —NHCH₂—OCH₃, —NH—CH₂—CH(CF₃)—OCH₃, —NH—CH(CH₃)—CH₂—OH, F, —NH-oxetane, —CH₂—CH₂—OCH₃, —CH₂—OCH₃, —CH₂-tetrahydropyran, —CH₂-methylpiperazine, —NH—CH₂—CH(OH)—CF₃, piperidine, —CH₂-pyrrolidine, —NH—CH(CH₃)CH₂OCH₃, —NH-tetrahydrofuran, —(CH₂)₃—NH₂, hydroxyethyl, propyl, —CH₂-pyridyl, —CH₂-piperidine, morpholine, —NH-chloropyrimidine, —NH—CH₂CH₂—SO₂-methyl, —(CH₃)₃—N(CH₃)₂, piperazine,

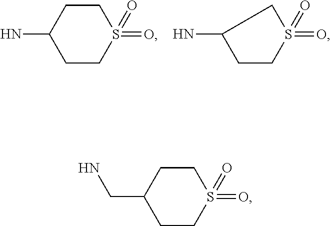

and —CH₂-morpholine.
   10. A compound of any one of embodiments 1-7, wherein:

R₃ is selected from H, methyl, cyano, chloro, CONH₂, amino, tetrazolyl, cyclopropyl, ethyl, and fluoro;

R₄ₐ and R_{4b} are independently selected from halogen, methyl, hydrogen, and halo-methyl;

R₆ is H if A₁ is CR₆;

R₈ is Cl if A₃ is CR₈;

R₁₆ is C₁₋₆ alkyl or C₃₋₈ cycloalkyl, and R₁₆ is substituted with one to three groups independently selected from hydroxyl, C₁₋₆ alkyl, —NR₁₇R₁₈ and —R₂₂—NR₁₇R₁₈;
wherein R₁₇ and R₁₈ are each, independently, selected from the group consisting of hydrogen, C₁₋₃alkyl, C₁₋₄haloalkyl, C₃₋₆ branched alkyl, —R₂₂—OR₁₂, —R₂₂—S(O)₂R₁₂, —R₂₂—NR₁₅S(O)₂R₁₂, heterocycloalkyl and heteroaryl;
alternatively, R₁₇ and R₁₈ along with the nitrogen atom to which they are attached to can be taken together to form a four to six membered heterocyclic ring containing up to one additional heteroatom selected from N, O and S as a ring member and wherein said ring carbon atoms are optionally substituted with R₂₀, and the additional nitrogen atom is optionally substituted with R₂₁;
R₁₉ is selected from optionally substituted C₁₋₃-alkyl, optionally substituted heterocycloalkyl, optionally substituted aryl and optionally substituted heteroaryl;
R₂₀ represents the group C₁₋₃alkyl; and
R₂₂ is selected from the group consisting of C₁₋₄alkyl, and C₃₋₆ branched alkyl.
   11. A compound of any one of embodiments 1-10, wherein:

A₄ is selected from NR₉, O, and a bond;

L is selected from a bond, C₁₋₄-alkyl, and cyclopropyl;

R₂ is selected from the group consisting of C₃₋₇ cycloalkyl, C₅₋₇ heterocycloalkyl, phenyl, and pyridyl, wherein said C₃₋₇ cycloalkyl and C₅₋₇ heterocycloalkyl are optionally substituted with up to three substituents independently selected from halogen, methoxy, dihalo-methoxy, trihalo-methoxy, trihalo alkyl, C₁₋₃-alkyl, and hydroxyl, and said phenyl and pyridyl are optionally substituted with up to three groups selected from halogen, cyano, oxo, CONH₂, CONHMe, CONMe₂, methoxy, dihalo-methoxy, trihalo-methoxy, trihalo C₁₋₆-alkyl, and C₁₋₃-alkyl; and

R₉ represents methyl, hydrogen, or ethyl.

12. A compound of embodiment 1, wherein:

X represents a bond;

R₁₆ is selected from cyclohexyl, and C₂₋₅-alkyl, CH(CH₂OH)₂, CH₂—CH(OH)—CH₂NH₂; CH₂—C(CH₃)₂—CH₂NHCH₃, CH(CH₃)OH, CH₂—C(CH₃)₂—CH₂NH₂, cyclopentyl, and cyclopropyl, wherein each said cyclohexyl, cyclopentyl, cyclopropyl and C₂₋₅-alkyl group is substituted with 1 to 2 substituents selected from amino, methyl-amino, hydroxy, amino-ethyl, dimethyl-amino, —NH—(CH₂)₂—O-ethyl, —NH—SO₂-methyl, —CH₂—NH—SO₂-methyl, piperidinyl, pyrrolidinyl, —NH—CH₂—CF₃, —NH—(CH₂)₂—O-methyl, —N(CH₃)—(CH₂)₁₋₂-methoxy, —NH—CH₂—CH(CH₃)—OH, NH—CH₂-tetrahydrofuranyl, —NH—(CH₂)₂—OH, —NH—CH₂—CONH₂, —NH(CH₂)₂—CF₃, methylpyrrolidin-3-ol, NH—(CH₂)₂-pyrrolidinyl, —NH—CH₂—COOH, —NH—CH₂-dioxane, —NH-oxetane, —NH-tetrahydrofuranyl, morpholinyl, —NH—(CH₂)₂—O—(CH₂)₂—OCH₃, —NH—(CH₂)₂—CONH₂, and —N(CH₂CH₂OCH₃)₂;

R₂ is selected from pyridyl, phenyl, tetrahydropyranyl, cyclopropyl, cyclohexyl, cycloheptyl, 1,4-dioxane, morpholinyl, alkyl substituted dioxane, tetrahydrofuranyl, dioxepane, piperidinyl and

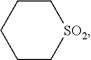

wherein each R₂ is substituted with one, two, or three groups independently selected from hydrogen, Cl, Br, F, methoxy, hydroxy-methyl, hydrogen, —CONR′₂, —SO₂R′, —SR′, —C(O)—R′, —COOR′, —NR′₂, cyano, dihalo-methoxy, trihalo-methoxy, trifluoro-methyl, hydroxyl and methyl; where each R′ is independently H or C1-C4 alkyl, and wherein two R′ on N can optionally cyclise to form a 5-7 membered heterocyclic ring that can optionally contain an additional heteroatom selected from N, O and S as a ring member;

A₄ is NH;

L is a bond, C₁₋₂alkyl or C₃₋₄ cycloalkyl;

R₃ is selected from H, CONH₂, hydroxyethyl, chloro, tetrazolyl, hydroxy, morpholino, cyano, fluoro, and methoxy;

R₄ₐ and R_{4b} are independently selected from H, Cl, and fluoro;

R₅ represents H;

R₆ represents hydrogen; and

R₈ is selected from hydrogen, chloro and fluoro.

13. A compound of embodiment 1, wherein:

X represents a bond;

R₁₆ is selected from cyclohexyl, and C₂₋₅-alkyl, —CH(CH₂OH)₂, —CH₂—CH(OH)—CH₂NH₂; —CH₂—C(CH₃)₂—CH₂NHCH₃, —CH(CH₃)OH, —CH₂—C(CH₃)₂—CH₂NH₂, cyclopentyl, and cyclopropyl, wherein each said cyclohexyl, cyclopentyl, cyclopropyl and C₂₋₅-alkyl group is substituted with 1 to 2 substituents selected from amino, methyl-amino, hydroxy, amino-ethyl, dimethyl-amino, —NH—(CH₂)₂—O-ethyl, —NH—SO₂-methyl, —CH₂—NH—SO₂-methyl, piperidinyl, pyrrolidinyl, —NH—CH₂—CF₃, —NH—(CH₂)₂—O-methyl, —N(CH₃)—(CH₂)₁₋₂-methoxy, —NH—CH₂—CH(CH₃)—OH, —NH—CH₂-tetrahydrofuranyl, —NH—(CH₂)₂—OH, —NH—CH₂—CONH₂, —NH(CH₂)₂—CF₃, methylpyrrolidin-3-ol, —NH—(CH₂)₂-pyrrolidinyl, —NH—CH₂—COOH, —NH—CH₂-dioxane, —NH-oxetane, —NH-tetrahydrofuranyl, morpholinyl, —NH—(CH₂)₂—O—(CH₂)₂—OCH₃, —NH—(CH₂)₂—CONH₂, and —N(CH₂CH₂OCH₃)₂;

-L-R₂ is selected from —CH₂-fluorophenyl, —CH₂-difluorophenyl, —CH₂-chlorophenyl, —CH₂-pyridyl, —CH₂-cyclopropyl, —CH₂-cyclohexyl, —CH₂-cyano-phenyl,

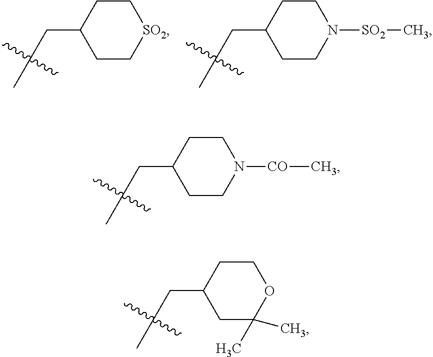

—CH₂-tetrahydropyran, benzyl, —CH₂-toluoyl, and —CH₂-methoxy-phenyl;

A₄ is NH;

R₃ is selected from H, CONH₂, hydroxyethyl, chloro, tetrazolyl, hydroxy, morpholino, cyano, fluoro, and methoxy;

R₄ₐ and R_{4b} are independently selected from H, Cl and fluoro;

R₅ represents H;

R₆ represents hydrogen; and

R₈ is selected from hydrogen, chloro and fluoro.

14. The compound of embodiments 1, wherein the compound is selected from the compounds of Table 1.
15. A compound of Formula (II):

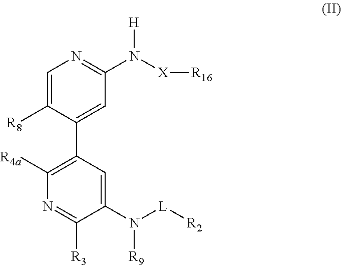

wherein:
X is a bond, —CH₂—, or —(CH₂)₂—,
   R₁₆ is selected from C₃-C₆ cycloalkyl and C₁₋₄ alkyl, each of which is optionally substituted with one to three groups independently selected from C₁₋₆ haloalkyl, halo, amino, oxo, —OR, —(CH₂)₂₋₄OR, —NR—(CH₂)₂₋₄—OR, —O—(CH₂)₂₋₄—OR, and C₁₋₄ aminoalkyl, wherein each R is independently C₁₋₄ alkyl or H;
   L is —CH₂— or a bond;
   R₈ is F or Cl;
   R₄ₐ is H, F or Cl;
   R₃ is H, F, Cl, OH, CN, or 4-morpholinyl;
   R₉ is H or Me; and
   R₂ is selected from cycloalkyl, heterocycloalkyl, heteroaryl and aryl, each of which is optionally substituted with up to three groups independently selected from halo, hydroxy, amino, CONH₂, haloalkyl, CN, C₁₋₄ alkyl, and C₁₋₄ haloalkyl.
      16. The compound of embodiment 15, wherein: R₈ is Cl; and R₄ₐ is H.
      17. The compound of embodiments 15 or 16, wherein R₃ is H and R₉ is H.
      18. The compound of embodiments 16 or 17, wherein L is —CH₂— and R₂ is C₅₋₇ heterocycloalkyl,

wherein said heterocycloalkyl contains 1-2 heteroatoms selected from N, O and S as ring members, and is optionally substituted with up to three groups independently selected from halo, hydroxy, amino, haloalkyl, CN, C₁₋₄ alkyl, C₁₋₄ alkoxy, CONH₂, and C₁₋₄ haloalkyl.

19. The compound of any of embodiments 15-18, wherein -LR₂ is

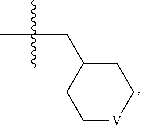

wherein V is O, NR, S or SO₂, where R is H or C₁₋₄ alkyl.
   20. The compound of embodiments 16 or 17, wherein L is a bond and R₂ is cyclopropyl, aryl or heteroaryl, each of which is optionally substituted with up to three groups independently selected from halo, hydroxy, amino, haloalkyl, CN, C₁₋₄ alkyl, and C₁₋₄ haloalkyl.
   21. The compound of embodiment 20, wherein R₂ is phenyl and is optionally substituted with up to three groups independently selected from halo, hydroxy, amino, haloalkyl, CN, C₁₋₄ alkyl, and C₁₋₄ haloalkyl.
   22. The compound of any of embodiments 15-21, wherein —X—R₁₆ is

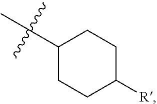

wherein R′ is selected from C₁₋₆ haloalkyl, halo, hydroxy, amino, oxo, C₁₋₄ aminoalkyl, —(CH₂)₁₋₄OR, —NR—(CH₂)₂₋₄—OR, and —O—(CH₂)₂₋₄—OR, wherein each R is independently C₁₋₄alkyl or H.

23. A compound according to any one of embodiments 1 to 22 or a pharmaceutically acceptable salt thereof, for use in therapy.
24. The compound according to embodiment 23, wherein the use in therapy is a use to treat cancer.
25. A pharmaceutical composition comprising a compound according to any one of embodiments 1-22 admixed with at least one pharmaceutically acceptable excipient.
26. The pharmaceutical composition of embodiment 25, wherein said compound is admixed with at least one pharmaceutically acceptable carrier and at least one additional pharmaceutically acceptable excipient.
27. Use of a compound according to any of embodiments 1-22, or a pharmaceutically acceptable salt thereof, for preparation of a medicament for treating a disease or condition mediated by CDK9.
28. The use of embodiment 27, wherein the disease or condition mediated by CDK9 is selected from cancer, cardiac hypertrophy, HIV and inflammatory diseases.
29. A method to treat a disease or condition mediated by CDK9 comprising administering to a subject in need thereof a therapeutically effective amount of a compound according to any one of embodiments 1-22, or a pharmaceutically acceptable salt thereof.
30. The method of embodiment 29, wherein the disease or condition mediated by CDK9 is selected from cancer, cardiac hypertrophy, HIV and inflammatory diseases.
31. The method of embodiment 30 wherein the disease or condition mediated by CDK9 is cancer.
32. The method of embodiment 31, wherein the cancer is selected from the group consisting of bladder, head and neck, breast, stomach, ovary, colon, lung, brain, larynx, lymphatic system, hematopoietic system, genitourinary tract, gastrointestinal, ovarian, prostate, gastric, bone, small-cell lung, glioma, colorectal, and pancreatic cancer.
33. The method of embodiment 29, wherein the compound of Formula I or II is administered, simultaneously or sequentially, with an antiinflammatory, antiproliferative, chemotherapeutic agent, immunosuppressant, anti-cancer, cytotoxic agent or kinase inhibitor or salt thereof.

The pharmaceutical compositions of the invention contain at least one compound according to any of the embodiments disclosed herein, including the pharmaceutically acceptable salts of these compounds, admixed with at least one pharmaceutically acceptable excipient, carrier or diluent. Preferably, the pharmaceutical compositions are sterile compositions, or compositions that consist essentially of or only of the above-described compounds and one or more pharmaceutically acceptable excipients, carriers and/or diluents. In some embodiments, the pharmaceutical composition comprises at least two pharmaceutically acceptable carriers and/or excipients described herein.

Certain compounds of the present invention can exist in unsolvated forms as well as solvated forms (i.e., solvates). Compounds of the invention may also include hydrated forms (i.e., hydrates). In general, the solvated and hydrated forms are equivalent to unsolvated forms for purposes of biological utility and are encompassed within the scope of the present invention. The invention also includes all polymorphs, including crystalline and non-crystalline forms. In general, all physical forms are useful for the uses contemplated by the present invention and are intended to be within the scope of the present invention.

The present invention includes all salt forms of the compounds described herein, as well as methods of using such salts. The invention also includes all non-salt forms of any salt of a compound named herein, as well as other salts of any salt of a compound named herein. In one embodiment, the salts of the compounds comprise pharmaceutically acceptable salts. “Pharmaceutically acceptable salts” are those salts which retain the biological activity of the free compounds and which can be administered as drugs or pharmaceuticals to humans and/or animals. The desired salt of a basic functional group of a compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, hippuric, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. The desired salt of an acidic functional group of a compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts.

Pharmaceutically acceptable metabolites and prodrugs of the compounds referred to in the formulas herein are also embraced by the invention. The term “pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, PRO-DRUGS AS NOVEL DELIVERY SYSTEMs, Vol. 14 of the A.C.S. Symposium Series, and in Edward B. Roche, ed., BIOREVERSIBLE CARRIERS IN DRUG DESIGN, American Pharmaceutical Association and Pergamon Press, 1987.

Pharmaceutically acceptable esters of the compounds referred to in the formulas herein are also embraced by the invention. As used herein, the term “pharmaceutically acceptable ester” refers to esters, which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

The invention further provides deuterated versions of the above-described compounds. As used herein, “deuterated version” refers to a compound in which at least one hydrogen atom is enriched in the isotope deuterium beyond the natural rate of deuterium occurrence. Typically, the hydrogen atom is enriched to be at least 50% deuterium, frequently at least 75% deuterium, and preferably at least about 90% deuterium. Optionally, more than one hydrogen atom can be replaced by deuterium. For example, a methyl group can be deuterated by replacement of one hydrogen with deuterium (i.e., it can be —CH₂D), or it can have all three hydrogen atoms replaced with deuterium (i.e., it can be —CD₃). In each case, D signifies that at least 50% of the corresponding H is present as deuterium.

A substantially pure compound means that the compound is present with no more than 15% or no more than 10% or no more than 5% or no more than 3% or no more than 1% of the total amount of compound as impurity and/or in a different form. For instance, substantially pure S,S compound means that no more than 15% or no more than 10% or no more than 5% or no more than 3% or no more than 1% of the total R,R; S,R; and R,S forms are present.

As used herein, “therapeutically effective amount” indicates an amount that results in a desired pharmacological and/or physiological effect for the condition. The effect may be prophylactic in terms of completely or partially preventing a condition or symptom thereof and/or may be therapeutic in terms of a partial or complete cure for the condition and/or adverse effect attributable to the condition. Therapeutically effective amounts of the compounds of the invention generally include any amount sufficient to detectably inhibit a CDK or CDK9 kinase activity by any of the assays described herein, by other CDK or CDK9 kinase activity assays known to those having ordinary skill in the art or by detecting an inhibition or alleviation of symptoms of cancer.

As used herein, the term “pharmaceutically acceptable carrier,” and cognates thereof, refers to adjuvants, binders, diluents, etc. known to the skilled artisan that are suitable for administration to an individual (e.g., a mammal or non-mammal). Combinations of two or more carriers are also contemplated in the present invention. The pharmaceutically acceptable carrier(s) and any additional components, as described herein, should be compatible for use in the intended route of administration (e.g., oral, parenteral) for a particular dosage form. Such suitability will be easily recognized by the skilled artisan, particularly in view of the teaching provided herein. Pharmaceutical compositions described herein include at least one pharmaceutically acceptable carrier or excipient; preferably, such compositions include at least one carrier or excipient other than or in addition to water.

As used herein, the term “pharmaceutical agent” or “additional pharmaceutical agent,” and cognates of these terms, are intended to refer to active agents other than the claimed compounds of the invention, for example, drugs, which are administered to elicit a therapeutic effect. The pharmaceutical agent(s) may be directed to a therapeutic effect related to the condition that a claimed compound is intended to treat or prevent (e.g., conditions mediated by a CDK kinase such as CDK9, including, but not limited to those conditions described herein (e.g., cancer)) or, the pharmaceutical agent may be intended to treat or prevent a symptom of the underlying condition (e.g., tumor growth, hemorrhage, ulceration, pain, enlarged lymph nodes, cough, jaundice, swelling, weight loss, cachexia, sweating, anemia, paraneoplastic phenomena, thrombosis, etc.) or to further reduce the appearance or severity of side effects of administering a claimed compound.

Another aspect of the present invention provides a compound of Formula I or II, or pharmaceutically acceptable salt or solvate thereof, for use in therapy. Yet another aspect of the present invention provides a compound of Formula I or II, or a pharmaceutically acceptable salt or solvate thereof, for use in a method of treating a disease or condition mediated by CDK9.

Yet another aspect of the present invention provides a method of treating a disease or condition mediated by CDK9 comprising administration to a subject in need thereof a therapeutically effective amount of a compound of Formula I or II, or a pharmaceutically acceptable salt thereof. Provided in yet another aspect of the present invention is a compound of Formula I or II for use in a method of treating a disease or condition mediated by CDK9 is selected from cancer, cardiac hypertrophy, HIV and inflammatory diseases.

Another aspect of the present invention provides a method of treating a cancer selected from the group consisting of bladder, head and neck, breast, stomach, ovary, colon, lung, brain, larynx, lymphatic system, hematopoietic system, genitourinary tract, gastrointestinal, ovarian, prostate, gastric, bone, small-cell lung, glioma, colorectal and pancreatic cancer. This method comprises administering an effective amount of a compound of Formula I or II to a subject diagnosed with at least one such condition.

Yet another aspect of the present invention provides a pharmaceutical composition comprising a compound of Formula I or II, or a pharmaceutically acceptable salt thereof, and a pharmaceutically acceptable carrier, diluent or excipient. In some embodiments, the pharmaceutical composition comprises at least two pharmaceutically acceptable carriers, diluents or excipients. In a preferred embodiment, the composition consists of a compound of Formula I or II, or a pharmaceutically acceptable salt thereof, and at least one pharmaceutically acceptable carrier, diluent or excipient.

In another aspect, the invention provides a method of regulating, modulating, or inhibiting protein kinase activity which comprises contacting a protein kinase with a compound of the invention. In one embodiment, the protein kinase is selected from the group consisting of CDK1, CDK2, CDK3, CDK4, CDK5, CDK6, CDK7, CDK8 and CDK9, or any combination thereof. In another embodiment, the protein kinase is selected from the group consisting of CDK1, CDK2 and CDK9, or any combination thereof. In still another embodiment, the protein kinase is in a cell culture. In yet another embodiment, the protein kinase is in a mammal.

In another aspect, the invention provides a method of treating a protein kinase-associated disorder comprising administering to a subject in need thereof a pharmaceutically acceptable amount of a compound of the invention such that the protein kinase-associated disorder is treated. In one embodiment, the protein kinase is selected from the group consisting of CDK1, CDK2, CDK3, CDK4, CDK5, CDK6, CDK7, CDK8 and CDK9. In a particularly preferred embodiment, the protein kinase is selected from the group consisting of CDK9.

In one embodiment, the protein kinase-associated disorder is cancer. In still another embodiment, the cancer is selected from the group consisting of bladder, head and neck, breast, stomach, ovary, colon, lung, brain, larynx, lymphatic system, hematopoietic system, genitourinary tract, gastrointestinal, ovarian, prostate, gastric, bone, small-cell lung, glioma, colorectal and pancreatic cancer.

In one embodiment, the protein kinase-associated disorder is inflammation. In another embodiment, the inflammation is related to rheumatoid arthritis, lupus, type 1 diabetes, diabetic nephropathy, multiple sclerosis, glomerulonephritis, chronic inflammation, and organ transplant rejections.

In another embodiment, the protein kinase-associated disorder is a viral infection. In one embodiment, the viral infection is associated with the HIV virus, human papilloma virus, herpes virus, poxvirus virus, Epstein-Barr virus, Sindbis virus, or adenovirus. In still another embodiment, the protein kinase-associated disorder is cardiac hypertrophy.

In another aspect, the invention provides a method of treating cancer comprising administering to a subject in need thereof a pharmaceutically acceptable amount of a compound of the invention such that the cancer is treated. In one embodiment, the cancer is selected from the group consisting of bladder, head and neck, breast, stomach, ovary, colon, lung, brain, larynx, lymphatic system, hematopoietic system, genitourinary tract, gastrointestinal, ovarian, prostate, gastric, bone, small-cell lung, glioma, colorectal and pancreatic cancer.

In another aspect, the invention provides a method of treating inflammation comprising administering to a subject in need thereof a pharmaceutically acceptable amount of a compound such that the inflammation is treated, wherein the compound is a compound of the invention. In one embodiment, the inflammation is related to rheumatoid arthritis, lupus, type 1 diabetes, diabetic nephropathy, multiple sclerosis, glomerulonephritis, chronic inflammation, and organ transplant rejections.

In another aspect, the invention provides a method of treating cardiac hypertrophy comprising administering to a subject in need thereof a pharmaceutically acceptable amount of a compound such that the cardiac hypertrophy is treated, wherein the compound is a compound of the invention.

In another aspect, the invention provides a method of treating a viral infection comprising administering to a subject in need thereof a pharmaceutically acceptable amount of a compound such that the viral infection is treated, wherein the compound is a compound of the invention. In one embodiment, the viral infection is associated with the HIV virus, human papilloma virus, herpes virus, poxvirus virus, Epstein-Barr virus, Sindbis virus, or adenovirus.

In one embodiment, the subject to be treated by the compounds of the invention is a mammal. In another embodiment, the mammal is a human.

In another aspect, the compounds of the invention is administered, simultaneously or sequentially, with an antiinflammatory, antiproliferative, chemotherapeutic agent, immunosuppressant, anti-cancer, cytotoxic agent or kinase inhibitor or salt thereof. In one embodiment, the compound, or salt thereof, is administered, simultaneously or sequentially, with one or more of a PTK inhibitor, cyclosporin A, CTLA4-Ig, antibodies selected from anti-ICAM-3, anti-IL-2 receptor, anti-CD45RB, anti-CD2, anti-CD3, anti-CD4, anti-CD80, anti-CD86, and monoclonal antibody OKT3, CVT-313, agents blocking the interaction between CD40 and gp39, fusion proteins constructed from CD40 and gp39, inhibitors of NF-kappa B function, non-steroidal antiinflammatory drugs, steroids, gold compounds, FK506, mycophenolate mofetil, cytotoxic drugs, TNF-α inhibitors, anti-TNF antibodies or soluble TNF receptor, rapamycin, leflunimide, cyclooxygenase-2 inhibitors, paclitaxel, cisplatin, carboplatin, doxorubicin, caminomycin, daunorubicin, aminopterin, methotrexate, methopterin, mitomycin C, ecteinascidin 743, porfiromycin, 5-fluorouracil, 6-mercaptopurine, gemcitabine, cytosine arabinoside, podophyllotoxin, etoposide, etoposide phosphate, teniposide, melphalan, vinblastine, vincristine, leurosidine, epothilone, vindesine, leurosine, or derivatives thereof.

In another aspect, the invention provides a packaged protein kinase-associated disorder treatment, comprising a protein kinase-modulating compound of the Formula I or Formula II, packaged with instructions for using an effective amount of the protein kinase-modulating compound to treat a protein kinase-associated disorder.

In certain embodiments, the compound of the present invention is further characterized as a modulator of a protein kinase, including, but not limited to, protein kinases selected from the group consisting of abl, ATK, Bcr-abl, Blk, Brk, Btk, c-fms, e-kit, c-met, c-src, CDK, cRafl, CSFIR, CSK, EGFR, ErbB2, ErbB3, ErbB4, ERK, Fak, fes, FGFRI, FGFR2, FGFR3, FGFR4, FGFRS, Fgr, FLK-4, fit-1, Fps, Frk, Fyn, GSK, Gst-Flkl, Hck, Her-2, Her-4, IGF-1R, INS-R, Jak, JNK, KDR, Lck, Lyn, MEK, p38, panHER, PDGFR, PLK, PKC, PYK2, Raf, Rho, ros, SRC, TRK, TYK2, UL97, VEGFR, Yes, Zap70, Aurora-A, GSK3-alpha, HIPK1, HIPK2, HIPS, IRAK1, JNK1, JNK2, JNK3, TRKB, CAMKII, CK1, CK2, RAF, GSK3Beta, MAPK1, MKK4, MKK7, MST2, NEK2, AAK1, PKCalpha, PKD, RIPK2 and ROCK-II.

In a preferred embodiment, the protein kinase is selected from the group consisting of CDK1, CDK2, CDK3, CDK4, CDK5, CDK6, CDK7, CDK8 and CDK9 and any combination thereof, as well as any other CDK, as well as any CDK not yet identified. In a particularly preferred embodiment, the protein kinase is selected from the group consisting of CDK1, CDK2 and CDK9. In a particularly preferred embodiment, the protein kinase is selected from the group consisting of CDK9.

In a particular embodiment, CDK combinations of interest include CDK4 and CDK9; CDK1, CDK2 and CDK9; CDK9 and CDK7; CDK9 and CDK1; CDK9 and CDK2; CDK4, CDK6 and CDK9; CDK1, CDK2, CDK3, CDK4, CDK6 and CDK9. In some embodiments, the compounds of the invention are active on at least one of these combinations with IC-50 levels below about 1 micromolar on each CDK and preferably below about 100 nM on each CDK in one of these combinations.

In other embodiments, the compounds of the present invention are used for the treatment of protein kinase-associated disorders. As used herein, the term “protein kinase-associated disorder” includes disorders and states (e.g., a disease state) that are associated with the activity of a protein kinase, e.g., the CDKs, e.g., CDK1, CDK2 and/or CDK9. Non-limiting examples of protein kinase-associated disorders include abnormal cell proliferation (including protein kinase-associated cancers), viral infections, fungal infections, autoimmune diseases and neurodegenerative disorders.

Non-limiting examples of protein-kinase associated disorders include proliferative diseases, such as viral infections, auto-immune diseases, fungal disease, cancer, psoriasis, vascular smooth cell proliferation associated with atherosclerosis, pulmonary fibrosis, arthritis glomerulonephritis, chronic inflammation, neurodegenerative disorders, such as Alzheimer's disease, and post-surgical stenosis and restenosis. Protein kinase-associated diseases also include diseases related to abnormal cell proliferation, including, but not limited to, cancers of the breast, ovary, cervix, prostate, testis, esophagus, stomach, skin, lung, bone, colon, pancreas, thyroid, biliary passages, buccal cavity and pharynx (oral), lip, tongue, mouth, pharynx, small intestine, colon-rectum, large intestine, rectum, brain and central nervous system, glioblastoma, neuroblastoma, keratoacanthoma, epidermoid carcinoma, large cell carcinoma, adenocarcinoma, adenocarcinoma, adenoma, adenocarcinoma, follicular carcinoma, undifferentiated carcinoma, papillary carcinoma, seminoma, melanoma, sarcoma, bladder carcinoma, liver carcinoma, kidney carcinoma, myeloid disorders, lymphoid disorders, Hodgkin's, hairy cells, and leukemia. Additional non-limiting examples of protein kinase-associated cancers include carcinomas, hematopoietic tumors of lymphoid lineage, hematopoietic tumors of myeloid lineage, tumors of mesenchymal origin, tumors of the central and peripheral nervous system, melanoma, seminoma, teratocarcinoma, osteosarcoma, xeroderoma pigmentosum, keratoctanthoma, thyroid follicular cancer and Kaposi's sarcoma. Protein kinase-associated disorders include diseases associated with apoptosis, including, but not limited to, cancer, viral infections, autoimmune diseases and neurodegenerative disorders.

Non-limiting examples of protein-kinase associated disorders include viral infections in a patient in need thereof, wherein the viral infections include, but are not limited to, HIV, human papilloma virus, herpes virus, poxvirus, Epstein-Barr virus, Sindbis virus and adenovirus.

Non-limiting examples of protein-kinase associated disorders include tumor angiogenesis and metastasis. Non-limiting examples of protein-kinase associated disorders also include vascular smooth muscle proliferation associated with atherosclerosis, postsurgical vascular stenosis and restenosis, and endometriosis.

Further non-limiting examples of protein-kinase associated disorders include those associated with infectious agents, including yeast, fungi, protozoan parasites such as Plasmodium falciparum, and DNA and RNA viruses.

In another embodiment, the compound of the present invention is further characterized as a modulator of a combination of protein kinases, e.g., the CDKs, e.g., CDK1, CDK2 and/or CDK9. In certain embodiments, a compound of the present invention is used for protein kinase-associated diseases, and/or as an inhibitor of any one or more protein kinases. It is envisioned that a use can be a treatment of inhibiting one or more isoforms of protein kinases.

The compounds of the invention are inhibitors of cyclin-dependent kinase enzymes. Without being bound by theory, inhibition of the CDK4/cyclin D1 complex blocks phosphorylation of the Rb/inactive E2F complex, thereby preventing release of activated E2F and ultimately blocking E2F-dependent DNA transcription. This has the effect of inducing G₁ cell cycle arrest. In particular, the CDK4 pathway has been shown to have tumor-specific deregulation and cytotoxic effects. Accordingly, the ability to inhibit the activity of combinations of CDKs will be of beneficial therapeutic use.

Furthermore, the cell's ability to respond and survive chemotherapeutic assault may depend on rapid changes in transcription or on activation of pathways which are highly sensitive to CDK9/cyclinT1 (PTEF-b) activity. CDK9 inhibition may sensitize cells to TNFalpha or TRAIL stimulation by inhibition of NF-kB, or may block growth of cells by reducing myc-dependent gene expression. CDK9 inhibition may also sensitize cells to genotoxic chemotherapies, HDAC inhibition, or other signal transduction based therapies.

As such, the compounds of the invention can lead to depletion of anti-apoptotic proteins, which can directly induce apoptosis or sensitize to other apoptotic stimuli, such as cell cycle inhibition, DNA or microtubule damage or signal transduction inhibition. Depletion of anti-apoptotic proteins by the compounds of the invention may directly induce apoptosis or sensitize to other apoptotic stimuli, such as cell cycle inhibition, DNA or microtubule damage or signal transduction inhibition.

The compounds of the invention can be effective in combination with chemotherapy, DNA damage arresting agents, or other cell cycle arresting agents. The compounds of the invention can also be effective for use in chemotherapy-resistant cells. The present invention includes treatment of one or more symptoms of cancer, inflammation, cardiac hypertrophy, and HIV infection, as well as protein kinase-associated disorders as described above, but the invention is not intended to be limited to the manner by which the compound performs its intended function of treatment of a disease. The present invention includes treatment of diseases described herein in any manner that allows treatment to occur, e.g., cancer, inflammation, cardiac hypertrophy, and HIV infection.

In certain embodiments, the invention provides a pharmaceutical composition of any of the compounds of the present invention. In a related embodiment, the invention provides a pharmaceutical composition of any of the compounds of the present invention and a pharmaceutically acceptable carrier or excipient of any of these compounds. In certain embodiments, the invention includes the compounds as novel chemical entities.

In one embodiment, the invention includes a packaged protein kinase-associated disorder treatment. The packaged treatment includes a compound of the invention packaged with instructions for using an effective amount of the compound of the invention for an intended use.

The compounds of the present invention are suitable as active agents in pharmaceutical compositions that are efficacious particularly for treating protein kinase-associated disorders, e.g., cancer, inflammation, cardiac hypertrophy, and HIV infection. The pharmaceutical composition in various embodiments has a pharmaceutically effective amount of the present active agent along with other pharmaceutically acceptable excipients, carriers, fillers, diluents and the like. In certain embodiments, the excipient is selected from the group consisting of corn starch, potato starch, tapioca starch, starch paste, pre-gelatinized starch, sugars, gelatin, natural gums, synthetic gums, sodium alginate, alginic acid, tragacanth, guar gum, cellulose, ethyl cellulose, cellulose acetate, carboxymethyl cellulose calcium, sodium carboxymethylcellulose, methyl cellulose, hydroxypropyl methylcellulose, microcrystalline cellulose, magnesium aluminum silicate, polyvinyl pyrrolidone, talc, calcium carbonate, powdered cellulose, dextrates, kaolin, mannitol, silicic acid, sorbitol, agar-agar, sodium carbonate, croscarmellose sodium, crospovidone, polacrilin potassium, sodium starch glycolate, clays, sodium stearate, calcium stearate, magnesium stearate, stearic acid, mineral oil, light mineral oil, glycerin, sorbitol, mannitol, polyethylene glycol, other glycols, sodium lauryl sulfate, hydrogenated vegetable oil, peanut oil, cottonseed oil, sunflower oil, sesame oil, olive oil, corn oil, soybean oil, zinc stearate, sodium oleate, ethyl oleate, ethyl laureate, silica, and combinations thereof.

The formulations described herein will generally be used in an amount effective to achieve the intended result, for example in an amount effective to treat or prevent the particular condition being treated or prevented. The formulations may be administered therapeutically to achieve therapeutic benefit. By therapeutic benefit is meant eradication or amelioration of the underlying condition being treated and/or eradication or amelioration of one or more of the symptoms associated with the underlying condition such that the individual reports an improvement in feeling or condition, notwithstanding that the individual may still be afflicted with the underlying condition. Therapeutic benefit also includes halting or slowing the progression of the condition, regardless of whether improvement is realized.

The amount of the formulation administered in order to administer an effective amount will depend upon a variety of factors, including, for example, the particular condition being treated, the frequency of administration, the particular formulation being administered, the severity of the condition being treated and the age, weight and general health of the individual, the adverse effects experienced by the individual being treated, etc. Determination of an effective dosage is within the capabilities of those skilled in the art, particularly in view of the teachings provided herein. Dosages may also be estimated using in vivo animal models.

The compounds of the invention may be administered enterally (e.g., orally or rectally), parenterally (e.g., sublingually, by injection, or by inhalation (e.g., as mists or sprays)), or topically, in dosage unit formulations containing conventional nontoxic pharmaceutically acceptable carriers, adjuvants, and vehicles as desired. For example, suitable modes of administration include oral, subcutaneous, transdermal, transmucosal, iontophoretic, intravenous, intraarterial, intramuscular, intraperitoneal, intranasal (e.g., via nasal mucosa), subdural, rectal, gastrointestinal, and the like, and directly to a specific or affected organ or tissue. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous injections, intravenous, intramuscular, intrasternal injection, or infusion techniques.

The compounds may be mixed with pharmaceutically acceptable carriers, adjuvants, and vehicles appropriate for the desired route of administration. In some embodiments, the route of administration is orally. In other embodiments, formulations are suitable for oral administration. The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The route of administration may vary according to the condition to be treated. Additional methods of administration are known in the art.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols that are solid at room temperature but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such formulations may also comprise adjuvants, such as wetting agents, emulsifying and suspending agents, cyclodextrins, and sweetening, flavoring, and perfuming agents.

The compounds of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present formulations in liposome form can contain, in addition to a compound of the present invention, stabilizers, preservatives, excipients, and the like. Suitable lipids are the phospholipids and phosphatidyl cholines (lecithins), both natural and synthetic.

Methods to form liposomes are known in the art. See, for example, Prescott, Ed., METHODS IN CELL BIOLOGY, Volume XIV, Academic Press, New York, N.W., p. 33 et seq (1976).

The compounds can be administered in prodrug form. Suitable prodrug formulations include, but are not limited to, peptide conjugates of the compounds of the invention and esters of compounds of the inventions. Further discussion of suitable prodrugs is provided in H. Bundgaard, DESIGN OF PRODRUGS, New York: Elsevier, 1985; in R. Silverman, THE ORGANIC CHEMISTRY OF DRUG DESIGN AND DRUG ACTION, Boston: Elsevier, 2004; in R. L. Juliano (ed.), BIOLOGICAL APPROACHES TO THE CONTROLLED DELIVERY OF DRUGS (Annals of the New York Academy of Sciences, v. 507), New York: New York Academy of Sciences, 1987; and in E. B. Roche (ed.), DESIGN OF BIOPHARMACEUTICAL PROPERTIES THROUGH PRODRUGS AND ANALOGS (Symposium sponsored by Medicinal Chemistry Section, APhA Academy of Pharmaceutical Sciences, November 1976 national meeting, Orlando, Fla.), Washington: The Academy, 1977. In some variations, the compounds are administered in a form of pharmaceutically acceptable esters.

The frequency and duration of administration of the formulation will depend on the condition being treated, the condition of the individual, and the like. The formulation may be administered to the individual one or more times, for example, 2, 3, 4, 5, 10, 15, 20, or more times. The formulation may be administered to the individual, for example, once a day, 2 times a day, 3 times a day, or more than 3 times a day. The formulation may also be administered to the individual, for example, less than once a day, for example, every other day, every third day, every week, or less frequently. The formulation may be administered over a period of days, weeks, or months.

The amount of active ingredient that may be combined with the carrier materials to produce a single dosage form will vary depending upon the host to which the active ingredient is administered and the particular mode of administration. It will be understood, however, that the specific dose level for any particular individual will depend upon a variety of factors including the activity of the specific compound employed, the age, body weight, body area, body mass index (BMI), general health, sex, diet, time of administration, route of administration, rate of excretion, drug combination, and the type, progression, and severity of the particular disease undergoing therapy. The pharmaceutical unit dosage chosen is usually fabricated and administered to provide a defined final concentration of drug in the blood, tissues, organs, or other targeted region of the body. The therapeutically effective amount for a given situation can be readily determined by routine experimentation and is within the skill and judgment of the ordinary clinician.

Examples of dosages which can be used are a therapeutically effective amount within the dosage range of about 0.1 μg/kg to about 300 mg/kg, or within about 1.0 μg/kg to about 40 mg/kg body weight, or within about 1.0 μg/kg to about 20 mg/kg body weight, or within about 1.0 μg/kg to about 10 mg/kg body weight, or within about 10.0 μg/kg to about 10 mg/kg body weight, or within about 100 μg/kg to about 10 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Other dosages which can be used are about 0.01 mg/kg body weight, about 0.1 mg/kg body weight, about 1 mg/kg body weight, about 10 mg/kg body weight, about 20 mg/kg body weight, about 30 mg/kg body weight, about 40 mg/kg body weight, about 50 mg/kg body weight, about 75 mg/kg body weight, about 100 mg/kg body weight, about 125 mg/kg body weight, about 150 mg/kg body weight, about 175 mg/kg body weight, about 200 mg/kg body weight, about 225 mg/kg body weight, about 250 mg/kg body weight, about 275 mg/kg body weight, or about 300 mg/kg body weight. Compounds of the present invention may be administered in a single daily dose, or the total daily dosage may be administered in divided dosage of two, three or four times daily.

For topical application, the formulation may be administered, for example transdermally at about 5 mg to about 100 mg over 24 hours. For IV administration, the formulation may be administered at a dosage of, for example, from about 0.1 mg per day to about 500 mg per day, typically from about 1 to about 200 mg/day. For oral administration, the formulation may be administered at a dosage of, for example, from about 1 mg per day to about 1500 mg per day, often from about 5 to about 250 mg/day.

As used herein, the term “pharmaceutically acceptable carrier,” and cognates thereof, refers to adjuvants, binders, diluents, etc., known to the skilled artisan that are suitable for administration to an individual (e.g., a mammal or non-mammal). As used herein, the term “pharmaceutically acceptable carriers, diluents or excipients” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329).

Combinations of two or more carriers or diluents are also contemplated in the present invention. In some embodiments, the pharmaceutical compositions comprise at least two pharmaceutically acceptable carriers, diluents or excipients selected from those disclosed herein.

The pharmaceutically acceptable carrier(s) and any additional components, as described herein, should be compatible for use in the intended route of administration (e.g., oral, parenteral) for a particular dosage form. Such suitability will be easily recognized by the skilled artisan, particularly in view of the teaching provided herein. Pharmaceutical compositions described herein include at least one pharmaceutically acceptable carrier or excipient; preferably, such compositions include at least one carrier or excipient other than or in addition to water.

The pharmaceutical composition can be formulated for particular routes of administration such as oral administration, and parenteral administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminium silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.
   Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil. Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

The invention further provides pharmaceutical compositions and dosage forms that may comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

The invention also includes compounds of any of the above embodiments for use in therapy. The use can be to treat a condition selected from the group consisting of cancer, cardiac hypertrophy, HIV, and inflammatory diseases. Use to treat cancer is preferred, and the cancer can be selected from the group consisting of bladder, head and neck, breast, stomach, ovary, colon, lung, brain, larynx, lymphatic system, hematopoietic system, genitourinary tract, gastrointestinal, ovarian, prostate, gastric, bone, small-cell lung, glioma, colorectal, and pancreatic cancer.

The invention also includes use of a compound of any of the above-described embodiments for the manufacture of a medicament for treatment of any of the conditions described herein as suitably treated by a CDK9 modulator, including cancers such as bladder, head and neck, breast, stomach, ovary, colon, lung, brain, larynx, lymphatic system, hematopoietic system, genitourinary tract, gastrointestinal, ovarian, prostate, gastric, bone, small-cell lung, glioma, colorectal, and pancreatic cancer.

When used with respect to methods of treatment/prevention and the use of the compounds and formulations thereof described herein, an individual “in need thereof” may be an individual who has been diagnosed with or previously treated for the condition to be treated. With respect to prevention, the individual in need thereof may also be an individual who is at risk for a condition (e.g., a family history of the condition, life-style factors indicative of risk for the condition, etc.). Typically, when a step of administering a compound of the invention is disclosed herein, the invention further contemplates a step of identifying an individual or subject in need of the particular treatment to be administered or having the particular condition to be treated.

In some embodiments, the individual is a mammal, including, but not limited to, bovine, horse, feline, rabbit, canine, rodent, or primate. In some embodiments, the mammal is a primate. In some embodiments, the primate is a human. In some embodiments, the individual is human, including adults, children and premature infants. In some embodiments, the individual is a non-mammal. In some variations, the primate is a non-human primate such as chimpanzees and other apes and monkey species. In some embodiments, the mammal is a farm animal such as cattle, horses, sheep, goats, and swine; pets such as rabbits, dogs, and cats; laboratory animals including rodents, such as rats, mice, and guinea pigs; and the like. Examples of non-mammals include, but are not limited to, birds, and the like. The term “individual” does not denote a particular age or sex.

In some variations, the individual has been identified as having one or more of the conditions described herein. Identification of the conditions as described herein by a skilled physician is routine in the art (e.g., via blood tests, X-rays, CT scans, endoscopy, biopsy, etc.) and may also be suspected by the individual or others, for example, due to tumor growth, hemorrhage, ulceration, pain, enlarged lymph nodes, cough, jaundice, swelling, weight loss, cachexia, sweating, anemia, paraneoplastic phenomena, thrombosis, etc.

In some embodiments, the individual has been identified as susceptible to one or more of the conditions as described herein. The susceptibility of an individual may be based on any one or more of a number of risk factors and/or diagnostic approaches appreciated by the skilled artisan, including, but not limited to, genetic profiling, family history, medical history (e.g., appearance of related conditions), lifestyle or habits.

As used herein and in the appended claims, the singular forms “a”, “an” and “the” include plural forms, unless the context clearly dictates otherwise. Unless defined otherwise or clearly indicated by context, all technical and scientific terms used herein have the same meaning as commonly understood by one of ordinary skill in the art to which this invention belongs.

### General Synthetic Methods

The compounds disclosed herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds disclosed herein may contain one or more chiral centers. Accordingly, if desired, such compounds can be prepared or isolated as pure stereoisomers, i.e., as individual enantiomers or diastereomers, or as stereoisomerenriched mixtures. All such stereoisomers (and enriched mixtures) are included within the scope of the embodiments, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

The various starting materials, intermediates, and compounds of the embodiments may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

Compounds of the embodiments may generally be prepared using a number of methods familiar to one of skill in the art, and may generally be made in accordance with the following reaction Schemes 1a, 1b, and 2, which are described in detail in the Examples below.

### EXAMPLES

Referring to the examples that follow, compounds of the embodiments were synthesized using the methods described herein, or other methods known to one skilled in the art.

The compounds and/or intermediates were characterized by high performance liquid chromatography (HPLC) using a Waters Millenium chromatography system with a 2695 Separation Module (Milford, Mass.). The analytical columns were reversed phase Phenomenex Luna C18 5μ, 4.6×50 mm, from Alltech (Deerfield, Ill.). A gradient elution was used (flow 2.5 mL/min), typically starting with 5% acetonitrile/95% water and progressing to 100% acetonitrile over a period of 10 minutes. All solvents contained 0.1% trifluoroacetic acid (TFA). Compounds were detected by ultraviolet light (UV) absorption at either 220 or 254 nm. HPLC solvents were from Burdick and Jackson (Muskegan, Mich.), or Fisher Scientific (Pittsburgh, Pa.).

In some instances, purity was assessed by thin layer chromatography (TLC) using glass or plastic backed silica gel plates, such as, for example, Baker-Flex Silica Gel 1B2-F flexible sheets. TLC results were readily detected visually under ultraviolet light, or by employing well known iodine vapor and other various staining techniques.

Mass spectrometric analysis was performed on LCMS instruments: Waters System (Acuity UPLC and a Micromass ZQ mass spectrometer; Column: Acuity HSS C18 1.8-micron, 2.1×50 mm; gradient: 5-95% acetonitrile in water with 0.05% TFA over a 1.8 min period; flow rate 1.2 mL/min; molecular weight range 200-1500; cone Voltage 20 V; column temperature 50° C.). All masses were reported as those of the protonated parent ions.

GCMS analysis is performed on a Hewlett Packard instrument (HP6890 Series gas chromatograph with a Mass Selective Detector 5973; injector volume: 1 μL; initial column temperature: 50° C.; final column temperature: 250° C.; ramp time: 20 minutes; gas flow rate: 1 mL/min; column: 5% phenyl methyl siloxane, Model No. HP 190915-443, dimensions: 30.0 m×25 m×0.25 m).

Nuclear magnetic resonance (NMR) analysis was performed on some of the compounds with a Varian 300 MHz NMR (Palo Alto, Calif.) or Varian 400 MHz MR NMR (Palo Alto, Calif.). The spectral reference was either TMS or the known chemical shift of the solvent. Some compound samples were run at elevated temperatures (e.g., 75° C.) to promote increased sample solubility.

The purity of some of the compounds is assessed by elemental analysis (Desert Analytics, Tucson, Ariz.).

Melting points are determined on a Laboratory Devices MeI-Temp apparatus (Holliston, Mass.).

Preparative separations are carried out using a Combiflash Rf system (Teledyne Isco, Lincoln, Nebr.) with RediSep silica gel cartridges (Teledyne Isco, Lincoln, Nebr.) or SiliaSep silica gel cartridges (Silicycle Inc., Quebec City, Canada) or by flash column chromatography using silica gel (230-400 mesh) packing material, or by HPLC using a Waters 2767 Sample Manager, C-18 reversed phase column, 30×50 mm, flow 75 mL/min. Typical solvents employed for the Combiflash Rf system and flash column chromatography are dichloromethane, methanol, ethyl acetate, hexane, heptane, acetone, aqueous ammonia (or ammonium hydroxide), and triethyl amine. Typical solvents employed for the reverse phase HPLC are varying concentrations of acetonitrile and water with 0.1% trifluoroacetic acid.

The examples below as well as throughout the application, the following abbreviations have the following meanings. If not defined, the terms have their generally accepted meanings.

### Abbreviations

### ACN: Acetonitrile

BINAP: 2,2′-bis(diphenylphosphino)-1,1′-binapthyl

### DCM: Dichloromethane

DIEA: diisopropylethylamine

### DIPEA: N,N-diisopropylethylamine

DME: 1,2-dimethoxyethane

### DMF: N,N-dimethylformamide

DMSO dimethyl sulfoxide
DPPF 1,1′-bis(diphenylphosphino)ferrocene
eq equivalent
EtOAc ethyl acetate
EtOH ethanol
HATU 2-(7-aza-1H-benzotriazole-1-yl)-1,1,3,3-tetramethyluronium hexafluorophosphate
HPLC high performance liquid chromatography
MCPBA meta-chloroperoxybenzoic acid
MeOH methanol

### NBS N-bromosuccinimide

NMP N-methyl-2-pyrrolidone
Rt rentention time
THF tetrahydrofuran

### Synthetic Examples

Compounds of the present invention can be synthesized by the schemes outlined below.

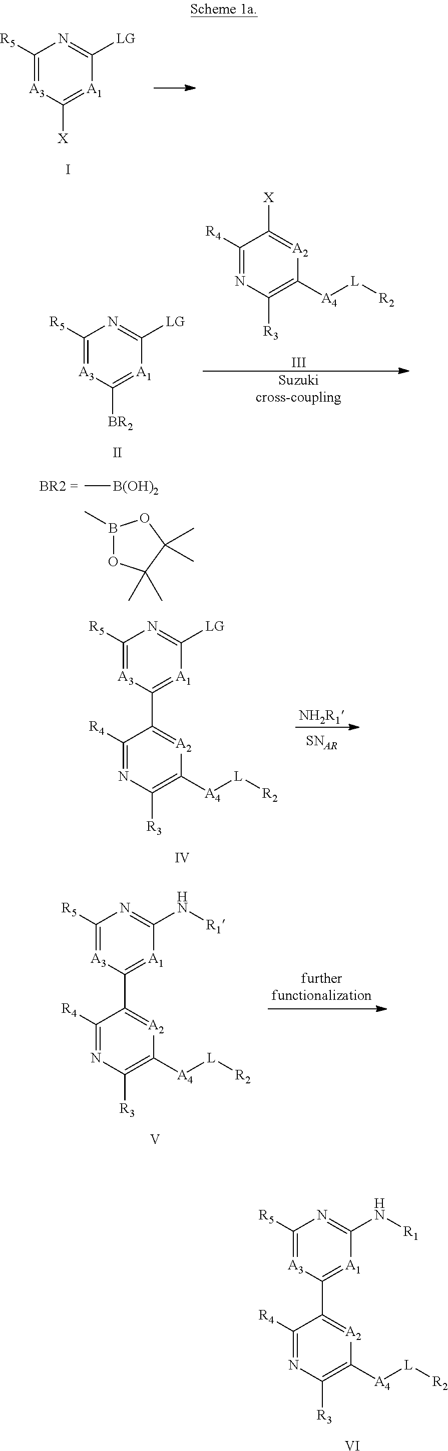

As shown in Scheme 1a, synthesis can start with a functionalized pyridine or pyrimidine I wherein LG is a leaving group such as F, Cl, OTf, and the like. X can be a functional group like Cl, Br, I or OTf. Compound I can be converted into boronic acid or boronic ester II by:

1) PdCl₂(dppf) DCM adduct, potassium acetate, bis(pinacolato)diboron heating from 30-120° C. in solvents such as THF, DMF, DME, DMA, toluene and dioxane; and 2) In a solvent such as THF or diethylether, anion halogen exchange by addition of nBuLi or LDA followed by quenching the anion with triisopropyl borate. Upon hydrolysis a boronic acid can be obtained. Suzuki cross-coupling reaction between compound II and pyridine or pyrazine III then gives bi-heteroaryl intermediate IV. The SN_{A} reaction between IV and a functionalized amine NH₂R₁′ under basic condition (DIEA, TEA, lutidine, pyridine) in a solvent such as DMF, THF, DMSO, NMP, dioxane with heating (30-130° C.) can give compound V. When R₁′ is not identical to R₁, further functional manipulation is needed to obtain VI. When R₁′ is identical to R₁, compound V will be the same as compound VI. Alternatively, VI can be obtained by following Scheme 1b. In which the Suzuki cross-coupling step is carried out between I and VII. Boronic acid or ester VII is synthesized from III in the same fashion as described above.

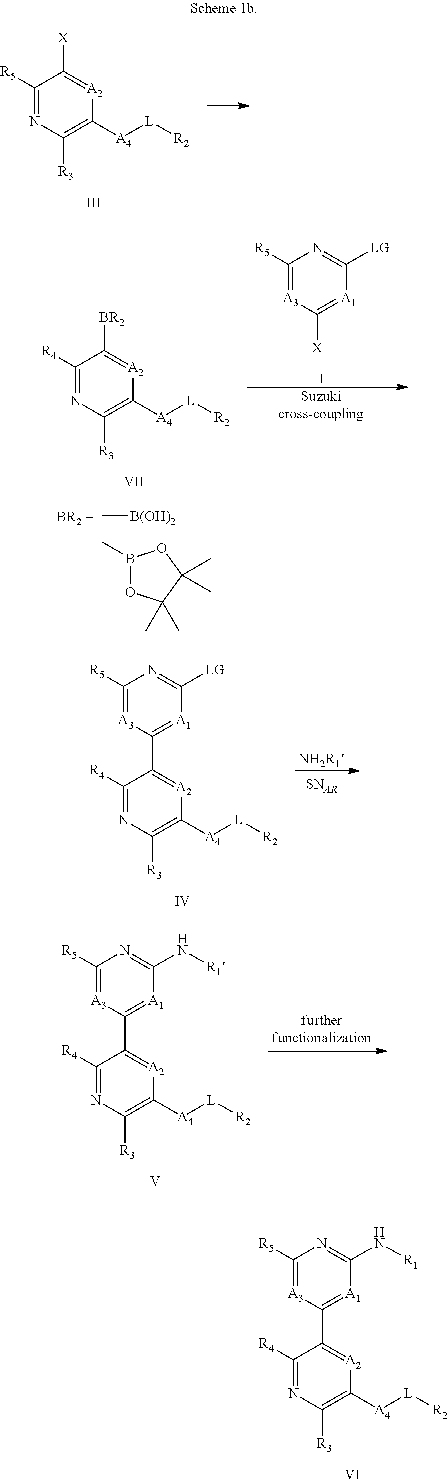

Another alternative route is illustrated in Scheme 2. As described in Scheme 1a, boronic ester or acid, X, can be prepared from aminopyridine or aminopyrimidine IX. Suzuki cross-coupling reaction between compound X and pyridine or pyrazine XI then can give the bi-heteroaryl intermediate XII. The SN_{AR} reaction between XII and functionalized amine HA₄LR₂ under basic condition (DIEA, TEA, lutidine, pyridine) in a solvent such as DMF, THF, DMSO, NMP, dioxane with heating (30-130° C.) can give compound V. When R₁′ is not identical to R₁, further functional manipulation will be needed to obtain VI. When R₁′ is identical with R₁, compound V will be the same as compound VII.

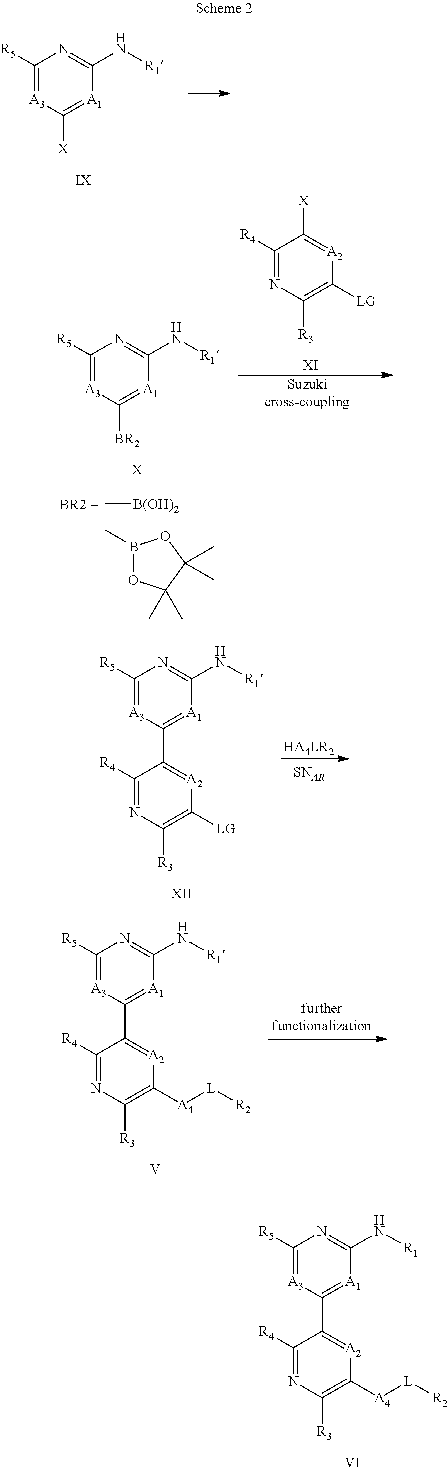

### Intermediates:

### Synthesis of N-(2-methoxyethyl)cyclohexane-trans-1,4-diamine (Intermediate A)

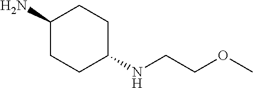

### Step 1. Synthesis of tert-butyl trans-4-(2-methoxyethylamino)cyclohexylcarbamate

A mixture of BOC 1,4-trans-diaminocyclohexane.HCl (2.178 g, 8.69 mmol), DIEA (2.275 mL, 13.03 mmol), p-toluenesulfonic acid 2-methoxyethyl ester (1.0 g, 4.34 mmol) in DMSO (20 mL) was heated in 100° C. oil bath (slightly cloudy colorless suspension) for about 16 hours. The resulting mixture was cooled to room temperature (red solution with solids floating on top), diluted in 200 mL water, added 20 mL 2 M Na₂CO₃ and extracted with EtOAc (2×150 mL). To the aqueous layer was added solid NaCl and extracted with EtOAc (1×150 mL). The organic extracts were combined, dried over Na₂SO₄, and concentrated to give a dark orange oil (3.2 g). This oil was diluted in 10 mL DCM, filtered off solids (impurity), then concentrated to yield a dark orange oil (3.1 g crude). The crude material was purified by column chromatography (ISCO system, 120 g column, Eluted with 100% DCM for 1 min, then 50% DCM to 100% (solution of 90% DCM/10% MeOH/0.5% NH₄OH) over 25 min, held for 10 min). Fractions containing pure product were combined and concentrated in vacuo to give 0.956 g (3.51 mmol, 81% yield) of a dark orange oil that solidified to a yellow solid upon standing. LC/MS (mass only): 273.1 (MH⁺), retention time=0.50 min.

### Step 2. N-(2-methoxyethyl)cyclohexane-trans-1,4-diamine

To compound obtained in step 1 (956 mg, 3.51 mmol) dissolved in 3 mL DCM was added TFA (1.0 mL, 12.98 mmol). The mixture was stirred at room temperature with oil bubbler for off-gassing. One hour later additional 1.0 mL of TFA was added and the reaction continued stirring for about 18 hours. The reaction was worked up by concentrating in vacuo to give a brown thick oil. This was dissolved in MeOH (30 mL) and neutralized with PL-HCO3 MP resin (18 g, 1.87 mmol/g, 100 angustron). Upon addition of the resin, off-gas was seen. The resin was filtered and the filtrate was concentrated in vacuo to give 0.5 g of brown oil. LC/MS showed as desired product. LC/MS: 173 (MH⁺), retention time=0.15 min.

### Synthesis of 5-bromo-6-chloro-N-((tetrahydro-2H-pyran-4-yl)methyl)pyridin-3-amine (Intermediate B)

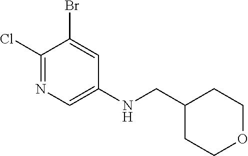 [SMILES: Clc1ncc(NCC2CCOCC2)cc1Br]

To a solution of 5-Amino-3-bromo-2-chloropyridine (3.00 g, 14.46 mmol) in DCM (90 ml) was add tetrahydro-2H-pyran-4-carbaldehyde (1.981 g, 17.35 mmol), acetic acid (0.828 ml, 14.46 mmol) and sodium triacetoxyborohydride (4.60 g, 21.69 mmol). The resulting slightly cloudy reddish solution was stirred at room temperature for about 2 hours. Additional sodium triacetoxyborohydride (˜1.3 g) was added and the reaction was stirred for about 18 hours. The reddish mixture was then concentrated, redissolved in EtOAc, and washed with water, NaHCO₃, and brine. The organic extracts were combined, dried over Na₂SO₄, filtered through silica plug, and the filtrate was concentrated in vacuo to give a yellow solid (4.3 g, 14.07 mmol, 97% yield). LC/MS: 306.9 (MH⁺), retention time=0.85 min.

### Synthesis of N—((R)-1-methoxypropan-2-yl)cyclohexane-trans-1,4-diamine (Intermediate C)

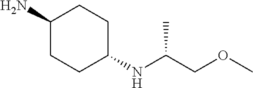

Step 1

To sodium hydride (0.488 g, 12.21 mmol) in 5 mL of THF was added via syringe (S)-(+)-3-methoxy-2-propanol (1.000 ml, 11.10 mmol) in 25 mL of THF at room temperature. The mixture was stirred for 20 min. and followed by addition of p-toluenesulfonyl chloride (2.327 g, 12.21 mmol). The white cloudy solution was stirred at room temperature for 18 hours. The reaction was diluted with sat. NaHCO₃ aq. and extracted with EtOAc. The organic extracts were combined, washed with brine, dried with sodium sulfate and concentrated in vacuo to give 2 g of colorless liquid. The crude mixture was purified by Analogix system (silica gel column 40 g, gradient: 100% n-heptane to 30% EtOAc in Heptane; 30 min.). The pure fractions were concentrated in vacuo to give 1.22 g of colorless oil. LC-MS (m/z): 245 (MH⁺), retention time=0.83 min.

### Step 2

To the tosylate obtained from step 1 (0.6 g, 2.45 mmol) in DMSO (6 ml) at room temperature was added cyclohexane-trans-1,4-diamine (0.84 g, 7.37 mmol). The light brown mixture was heated to 99° C. in a capped glass vial for 1 hour. LC/MS showed nearly complete conversion of the starting material. The mixture was diluted with water and extracted with DCM. The organic extracts were combined, washed with brine, dried with sodium sulfate and concentrated in vacuo to give 0.39 g of light brown liquid. This was used in the next step without further purification. LC-MS (m/z): 187 (MH⁺), retention time=0.14 min.

### Synthesis of N-(1,3-dimethoxypropan-2-yl)cyclohexane-trans-1,4-diamine (Intermediate D)

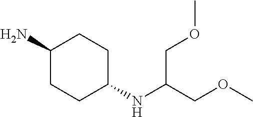

### Step 1

To NaH (0.366 g, 9.16 mmol) in THF (12 mL) at 0° C. was added 1,3-dimethoxy-2-propanol (1 g, 8.32 mmol) in THF (8 mL) solution. The mixture was warmed to room temperature and stirred for 0.5 hour. To this was added tosyl chloride (1.587 g, 8.32 mmol) in one portion. The white cloudy mixture was stirred at room temperature for 16 hours. LC/MS showed complete conversion. The reaction mixture was poured into water and extracted with EtOAc. The organic extracts were combined, washed with brine, dried with sodium sulfate and concentrated in vacuo to give 2 g of colorless oil. The crude mixture was purified by Analogix system (silica gel column 80 g, gradient: 0 min, 100% n-heptane; 5-12 min, 20% EtOAc in Heptane; 12-15 min. 30% EtOAc in Heptane and hold until 30 min). The pure fractions were combined and concentrated in vacuo to give 1.25 g of product as colorless oil which solidified upon standing.

### Step 2

To the tosylate obtained in Step 1 (0.8 g, 2.92 mmol) in DMSO (8 ml) was added 1,4-trans-cyclohexane diamine (0.999 g, 8.75 mmol). The brown mixture in a capped vial was heated to 95° C. with stirring for 2 hours. The reaction mixture was poured into 10% HCl in water (10 mL) at 0° C. (ice cubes in HCl) and extracted with DCM (1×20 mL). The aqueous (light pink) was basified with 6N NaOH to pH>12 and extracted with DCM (2×20 mL). The organic extracts were combined, dried with sodium sulfate and concentrated in vacuo to give a purple liquid. LC-MS (m/z): 217 (MH⁺), retention time=0.32 min., no UV absorption at 214 nm wavelength. This was used in the next step without further purification.

### Synthesis of (R)-2-methyl-2-(trifluoromethyl)oxirane

(Reference: A. Harada, Y. Fujiwara, T. Katagiri, Tetrahedron: Asymmetry (2008) 1210-1214.)

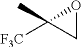

To a solution of (R)-2-(trifluoromethyl)oxirane (0.5 g, 4.46 mmol) under argon at −100° C. was added n-BuLi (1.89 mL, 4.91 mmol) and the mixture was stirred at this temperature for 10 min. To the solution was added iodomethane (0.558 mL, 8.92 mmol) and the mixture was stirred at −80° C. for 3 hours. The mixture was allowed to warm to 0° C. and directly used in the next reaction. Total volume: ˜24.8 mL; 0.18 M solution. To 1 mL of this solution was added triethylamine (139 μL, 0.997 mmol). The mixture was stirred for ˜30 min and the formed precipitate was removed over a syringe filter. The clear solution was directly used.

### Synthesis of 2,5-difluoropyridin-4-ylboronic acid

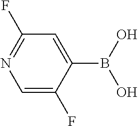 [SMILES: OB(O)c1cc(F)ncc1F]

To a solution of diisopropylamine (1.74 mL, 12.20 mmol) in anhydrous tetrahydrofuran (22 mL) under argon at −20° C. was added n-butyllithium (7.66 mL, 1.6M in hexanes) slowly over 10 min. The newly formed LDA was then cooled to −78° C. A solution of 2,5-difluoropyridine (1.05 mL, 11.5 mmol) in anhydrous tetrahydrofuran (3 mL) was added slowly over 30 min and the mixture was stirred at −78° C. for 4 hrs. A solution of triisopropyl borate (5.90 mL, 25.4 mmol) in anhydrous tetrahydrofuran (8.6 mL) was added dropwise. Once the addition was complete the reaction mixture was warmed to room temperature and stirring was continued for an additional hour. The reaction mixture was diluted with aqueous sodium hydroxide solution (4 wt. %, 34 mL). The separated aqueous layer was cooled to 0° C. and then slowly acidified to pH=4 with 6N aqueous hydrochloride solution (˜10 mL). The mixture was extracted with EtOAc (3×50 mL). The combined organic layers washed with brine (50 mL), dried over sodium sulfate, filtered off and concentrated under reduced pressure. The residue was triturated with diethylether to give 2,5-difluoropyridin-4-ylboronic acid (808 mg).

### Synthesis of (1-cyanocyclopropyl)methyl methanesulfonate

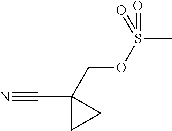

### Step 1: Preparation of methyl 1-cyanocyclopropanecarboxylate

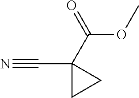

In a 100 mL, flask at 0° C., 1-cyanocyclopropanecarboxylic acid (3 g, 27.0 mmol) was dissolved in toluene (45 mL) and MeOH (5 mL). Reaction was treated dropwise with TMS-Diazomethane (27.0 mL, 27.0 mmol) and reaction stirred at 0° C. for 2 hr. Reaction was concentrated under reduced pressure providing a yellow oil, which was used without further purification (3.21 g, 25.7 mmol) GC/MS Rt=5.0 min, m/z=125.

### Step 2: Preparation of 1-(hydroxymethyl)cyclopropanecarbonitrile

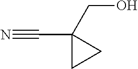

In a 100 mL, flask at 0° C., methyl 1-cyanocyclopropanecarboxylate (1 g, 7.99 mmol) was dissolved in 1,2-Dimethoxyethane (20 ml) and MeOH (2 mL). Reaction was treated portion wise with NaBH₄ (0.605 g, 15.98 mmol) and reaction stirred at 0° C. for 2 hr and then 20 hrs overnight. Reaction was quenched with 20 mL of saturated NH₄Cl solution. Reaction was diluted with Et₂O and stirred vigorously for 2 hrs. Organics were isolated, dried (MgSO4), filtered and concentrated under reduced pressure to provide the title compound as a yellow oil which was used without further purification (755 mg) GC/MS Rt=4.8 min, m/z=98.

### Step 3: Preparation of (1-cyanocyclopropyl)methyl methanesulfonate

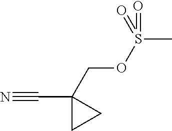

In a 250 mL, RBR at 0° C., 1-(hydroxymethyl)cyclopropanecarbonitrile (400 mg, 4.12 mmol) was dissolved in methylene chloride (15 mL) and triethylamine (1.148 mL, 8.24 mmol). Reaction was treated drop wise with methanesulfonyl chloride (0.353 mL, 4.53 mmol) and reaction stirred at 0° C. for 2 hr. Reaction was quenched with 20 mL of saturated aqueous Na₂CO₃ solution. Reaction mixture was diluted with Et₂O and stirred vigorously for 30 minutes. Organics were isolated, dried (MgSO4), filtered and concentrated under reduced pressure providing the title compound as a yellow oil which was used without further purification (622 mg).

### Synthesis of (S)-1-(tetrahydro-2H-pyran-4-yl)ethanamine

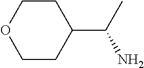

### Step 1: Preparation of (R,E)-2-methyl-N-((tetrahydro-2H-pyran-4-yl)methylene)propane-2-sulfinamide

A mixture of tetrahydro-2H-pyran-4-carbaldehyde (2.0 g, 17.52 mmol), (R)-2-methylpropane-2-sulfinamide (1.062 g, 8.76 mmol), pyridine 4-methylbenzenesulfonate (0.110 g, 0.438 mmol) and magnesium sulfate (5.27 g, 43.8 mmol) in dichloroethane (13 mL) was stirred at room temperature for 18 hrs. The solids were filtered off and the filtrate was concentrated to dryness under reduced pressure. The residue was purified by column chromatography [silica gel] providing (R,E)-2-methyl-N-((tetrahydro-2H-pyran-4-yl)methylene)propane-2-sulfinamide (1.9 g). LCMS (m/z): 218.1 [M+H]+; Retention time=0.58 min.

### Step 2: Preparation of (R)-2-methyl-N—((S)-1-(tetrahydro-2H-pyran-4-yl)ethyl)propane-2-sulfinamide

To a solution of (R,E)-2-methyl-N-((tetrahydro-2H-pyran-4-yl)methylene)propane-2-sulfinamide (0.93 g, 4.28 mmol) in dichloromethane (21.4 mL) at 0° C. was added slowly methylmagnesium bromide (2.0 M in tetrahydrofuran, 4.28 mL, 8.56 mmol). The reaction mixture was warmed to room temperature and stirred for 3 hrs. The mixture was diluted with saturated aqueous ammonium chloride solution (5 mL). The separated organic layer was washed with water and brine, dried over sodium sulfate and concentrated to dryness under reduced pressure. The residue was purified by column chromatography providing (R)-2-methyl-N—((S)-1-(tetrahydro-2H-pyran-4-yl)ethyl)propane-2-sulfinamide (910 mg). LCMS (m/z): 234.0 [M+H]+; Retention time=0.58 min.

### Step 3: Preparation of (S)-1-(tetrahydro-2H-pyran-4-yl)ethanamine

To a solution of (R)-2-methyl-N—((S)-1-(tetrahydro-2H-pyran-4-yl)ethyl)propane-2-sulfinamide (400 mg, 1.714 mmol) in MeOH (5 mL) was added 4M hydrochloride in dioxane (5 mL). The reaction mixture was stirred at room temperature for 30 min. The mixture was concentrated under reduced pressure and the residue was diluted with diethylether (10 mL). The precipitate was collected by filtration and washed with diethylether providing crude (S)-1-(tetrahydro-2H-pyran-4-yl)ethanamine hydrochloride salt. The hydrochloride salt was dissolved in water (10 mL) and neutralized with saturated aqueous sodium bicarbonate solution. The mixture was extracted with dichloromethane. The organic layer was dried over sodium sulfate, filtered off and concentrated under reduced pressure providing crude (S)-1-(tetrahydro-2H-pyran-4-yl)ethanamine (212 mg), which was directly used in the next reaction without further purification. LCMS (m/z): 130.1 [M+H]+; Retention time=0.34 min.

### Synthesis of (R)-1-(tetrahydro-2H-pyran-4-yl)ethanamine

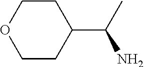

### Step 1: Preparation of (S,E)-2-methyl-N-((tetrahydro-2H-pyran-4-yl)methylene)propane-2-sulfinamide

A mixture of tetrahydro-2H-pyran-4-carbaldehyde (2.0 g, 17.52 mmol), (S)-2-methylpropane-2-sulfinamide (1.062 g, 8.76 mmol), pyridine 4-methylbenzenesulfonate (0.110 g, 0.438 mmol) and magnesium sulfate (5.27 g, 43.8 mmol) in dichloroethane (13 mL) was stirred at room temperature for 18 hrs. The solids were filtered off and the filtrate was concentrated to dryness under reduced pressure. The residue was purified by column chromatography [silica gel] providing (S,E)-2-methyl-N-((tetrahydro-2H-pyran-4-yl)methylene)propane-2-sulfinamide (1.50 g). LCMS (m/z): 218.1 [M+H]+; Retention time=0.58 min.

### Step 2: Preparation of (S)-2-methyl-N—((R)-1-(tetrahydro-2H-pyran-4-yl)ethyl)propane-2-sulfinamide

To a solution of (S,E)-2-methyl-N-((tetrahydro-2H-pyran-4-yl)methylene)propane-2-sulfinamide (1.5 g, 6.90 mmol) in dichloromethane (34.5 mL) at 0° C. was slowly added methylmagnesium bromide (1.646 g, 13.80 mmol). The reaction mixture was warmed to room temperature and stirred for 3 hrs. The mixture was diluted with saturated aqueous ammonium chloride solution (5 mL). The separated organic layer was washed with water and brine, dried over sodium sulfate and concentrated to dryness under reduced pressure. The residue was purified by column chromatograph providing (S)-2-methyl-N—((R)-1-(tetrahydro-2H-pyran-4-yl)ethyl)propane-2-sulfinamide (1.40 g).

LCMS (m/z): 234.3 [M+H]+; Retention time=0.57 min.

### Step 3: Preparation of (R)-1-(tetrahydro-2H-pyran-4-yl)ethanamine

To a solution of (S)-2-methyl-N—((R)-1-(tetrahydro-2H-pyran-4-yl)ethyl)propane-2-sulfinamide (400 mg, 1.714 mmol) in MeOH (5 mL) was added 4M hydrochloride in dioxane (5 mL). The reaction mixture was stirred at room temperature for 30 min. The mixture was concentrated under reduced pressure and the residue was diluted with diethylether (10 mL). The precipitate was collected by filtration and washed with diethylether providing crude (R)-1-(tetrahydro-2H-pyran-4-yl)ethanamine hydrochloride salt. The hydrochloride salt was dissolved in water (10 mL) and neutralized with saturated aqueous sodium bicarbonate solution. The mixture was extracted with dichloromethane (2×). The combined organic layers were dried over sodium sulfate, filtered off and concentrated under reduced pressure providing crude (R)-1-(tetrahydro-2H-pyran-4-yl)ethanamine (200 mg), which was directly used in the next reaction without further purification. LCMS (m/z): 130.1 [M+H]+; Retention time=0.34 min.

### Synthesis of (2,2-dimethyltetrahydro-2H-pyran-4-yl)methanamine

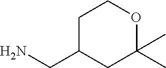

### Step 1: Preparation of (2,2-dimethyltetrahydro-2H-pyran-4-yl)methyl 4-methylbenzenesulfonate

To a solution of (2,2-dimethyltetrahydro-2H-pyran-4-yl)methanol (1 g, 6.93 mmol) in dichloromethane (5 mL) and pyridine (5 mL, 61.8 mmol) was added para-toluenesulfonyl chloride (1.586 g, 8.32 mmol) and DMAP (0.042 g, 0.347 mmol). The resulting mixture was stirred for 18 hrs at room temperature. The reaction mixture was concentrated under reduced pressure and the residue was diluted with water and dichloromethane. The separated organic phase was washed with 0.2N aqueous hydrochloride solution (1×), 1N aqueous hydrochloride solution (2×), brine, dried over sodium sulfate, filtered off and concentrated under reduced pressure. The residue was purified by column chromatography [silica gel, 40 g, EtOAc/hexane=0/100 to 50/50] providing (2,2-dimethyltetrahydro-2H-pyran-4-yl)methyl 4-methylbenzenesulfonate (2.05 g) as a colorless oil. LCMS (m/z): 299.1 [M+H]+; Retention time=0.96 min.

### Step 2: Preparation of (2,2-dimethyltetrahydro-2H-pyran-4-yl)methanamine

Into a solution of (2,2-dimethyltetrahydro-2H-pyran-4-yl)methyl 4-methylbenzenesulfonate (3 g, 10.05 mmol) in tetrahydrofuran (25 mL) in a steel bomb was condensed ammonia (˜5.00 mL) at −78° C. The mixture was heated in the steel bomb at 125° C. for ˜18 hrs. The mixture was cooled to −78° C., the steel bomb was opened, and the mixture was allowed to warm up to room temperature under a stream of nitrogen. The mixture was concentrated under reduced pressure and the residue was partitioned between a aqueous sodium hydroxide solution (5 wt. %) and dichloromethane. The separated aqueous layer was extracted with dichloromethane (1×). The combined organic layers were washed with aqueous sodium hydroxide solution (5 wt. %), dried over sodium sulfate, filtered off and concentrated under reduced pressure providing crude (2,2-dimethyltetrahydro-2H-pyran-4-yl)methanamine (˜2.36 g) as yellow liquid, which was directly used in the next reaction without further purification. LCMS (m/z): 144.1 [M+H]+; Retention time=0.26 min.

### Synthesis of (6,6-dimethyl-1,4-dioxan-2-yl)methanamine

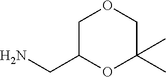

### Step 1: Preparation of 1-(allyloxy)-2-methylpropan-2-ol

To allylic alcohol (57.4 mL, 844 mmol) was added sodium hydride (60 wt. % in mineral oil, 2.43 g, 101 mmol) at 0° C. After stirring for 20 min 2,2-dimethyloxirane (15 mL, 169 mmol) was added and the solution was refluxed overnight. The mixture was allowed to cool to room temperature, diluted with saturated aqueous ammonium chloride solution and extracted with diethylether (3×). The combined organic layers were dried over sodium sulfate, filtered off and concentrated under reduced pressure to remove diethylether. The residue was distilled providing 1-(allyloxy)-2-methylpropan-2-ol (12.3 g, 42 torr, by 58-60° C.) as a colorless oil. ¹H NMR (400 MHz, chloroform-d) δ [ppm]: 5.87-5.96 (m, 1H) 5.26-5.31 (m, 1H) 5.18-5.21 (m, 1H) 4.03-4.05 (m, 2H) 3.28 (s, 2H) 2.31 (br. s, ¹H) 1.23 (s, 3H) 1.22 (s, 3H).

### Step 2: Preparation of 6-(iodomethyl)-2,2-dimethyl-1,4-dioxane

To a solution of 1-(allyloxy)-2-methylpropan-2-ol (5.0 g, 38 mmol) in acetonitrile (400 mL) was added sodium bicarbonate (19.5 g, 77 mmol) and the mixture was cooled to 0° C. Iodine (11.7 g, 46.1 mmol) was added and the reaction mixture was allowed to warm up to room temperature and stirred overnight. To the mixture was added triethylamine (6.42 mL, 46.1 mmol) and additional iodine (7.8 g, 30.7 mmol) and stirring was continued for additional 5 hrs at 0° C. To the mixture was added potassium carbonate (6.37 g, 46.1 mmol) and the suspension was stirred at room temperature for ˜3 days. The reaction mixture was diluted with saturated aqueous sodium thiosulfate solution (200 mL) and EtOAc (300 mL). The separated aqueous layer was extracted with EtOAc (2×) and the combined organic layers were dried over sodium sulfate, filtered off and concentrated under reduced pressure. The residue was purified by column chromatography [silica gel, EtOAc/hexane=10/100 to 10/40] providing 6-(iodomethyl)-2,2-dimethyl-1,4-dioxane as a yellow oil (2.07 g). ¹H NMR (400 MHz, chloroform-d) δ [ppm]: 4.01 (dd, J=11.2, 2.8 Hz, 1H) 3.81-3.88 (m, 1H) 3.44 (d, J=11.2 Hz, 1H) 3.22 (dd, J=11.6, 0.8 Hz, 1H) 2.97-3.13 (m, 3H) 1.33 (s, 3H) 1.14 (s, 3H). 1-(Allyloxy)-2-methylpropan-2-ol (1.63 g) was recovered.

### Step 3: Preparation of 6-(azidomethyl)-2,2-dimethyl-1,4-dioxane

To a solution of 6-(iodomethyl)-2,2-dimethyl-1,4-dioxane (1.80 g, 7.03 mmol) in anhydrous DMF (9 mL) was added sodium azide (0.685 g, 10.5 mmol) and the suspension was heated at 80° C. for 2.5 hrs. The mixture was diluted with water (30 mL) and EtOAc (30 mL). The separated organic layer was washed with water (3×). The aqueous layers were combined and extracted with EtOAc (1×). The combined organic layers, dried over sodium sulfate, filtered off and concentrated under reduced pressure.

The residue was purified by column chromatography [silica gel, EtOAc/hexane=10/40 to 20/40] providing 6-(azidomethyl)-2,2-dimethyl-1,4-dioxane (0.93 g) as a colorless oil. ¹H NMR (400 MHz, chloroform-d) δ [ppm]: 4.00-4.06 (m, 1H) 3.75 (ddd, J=11.2, 2.4, 0.4 Hz, 1H) 3.49 (d, J=11.2 Hz, 1H) 3.14-3.29 (m, 4H) 1.35 (s, 3H), 1.14 (s, 3H).

### Step 4: Preparation of (6,6-dimethyl-1,4-dioxan-2-yl)methanamine

To a solution of 6-(azidomethyl)-2,2-dimethyl-1,4-dioxane (502 mg, 2.93 mmol) in anhydrous tetrahydrofuran (15 mL) was added slowly a solution of lithium aluminumhydride (1M in tetrahydrofuran, 3.81 mL) 0° C. and the mixture was stirred at 0° C. for 1 hr and at room temperature for 0.5 hr. The reaction mixture was cooled to 0° C. and sodium sulfate decahydrate (excess) was slowly added and the suspension was vigorously stirred overnight. The suspension was filtered through cotton and the filtrate was concentrated under reduced pressure providing crude (6,6-dimethyl-1,4-dioxan-2-yl)methanamine (410 mg) as a colorless oil, which was directly used in the next step without purification. LCMS (m/z): 146.1 [M+H]+; Retention time=0.42 min.

### Synthesis of (5,5-dimethyl-1,4-dioxan-2-yl)methanamine

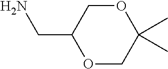

### Step 1: Preparation of 2-(allyloxy)-2-methylpropan-1-ol

To a solution of 2,2-dimethyloxirane (15.0 mL, 169 mmol) in allylic alcohol (57.4 mL) was added perchloric acid (70 wt. %, 7.26 mL, 84 mmol) slowly at 0° C. The solution was warmed to room temperature and stirred for 1.5 hrs. The reaction mixture was diluted with saturated aqueous sodium bicarbonate solution and extracted with diethylether (3×). The combined organic layers were dried over sodium sulfate, filtered off and concentrated under reduced pressure to remove diethylether. The residue was distilled providing 2-(allyloxy)-2-methylpropan-1-ol (9.70 g, 38 torr, by 74-76° C.) as a colorless oil. ¹H NMR (400 MHz, chloroform-d) δ [ppm]: 5.87-5.97 (m, 1H) 5.25-5.31 (m, 1H) 5.12-5.16 (m, 1H) 3.92-3.94 (m, 2H) 3.45 (m, 2H) 1.19 (s, 6H).

### Step 2: Preparation of 5-(iodomethyl)-2,2-dimethyl-1,4-dioxane

To a solution of 2-(allyloxy)-2-methylpropan-1-ol (5.0 g, 38.4 mmol) in acetonitrile (350 mL) was added sodium bicarbonate (9.68 g, 115 mmol) and the mixture was cooled to 0° C. Iodine (29.2 g, 115 mmol) was added and the reaction mixture was allowed to warm up to room temperature and stirred for 6 hrs. The reaction mixture was diluted with saturated aqueous sodium thiosulfate solution and concentrated under reduced pressure removing most of the organic solvent. The residue was extracted with EtOAc (2×) and the combined organic layers were dried over sodium sulfate, filtered off and concentrated under reduced pressure. The residue was purified by column chromatography [silica gel, EtOAc/hexane=10/100 to 10/40] providing 6-(iodomethyl)-2,2-dimethyl-1,4-dioxane as a colorless oil (7.04 g). ¹H NMR (400 MHz, chloroform-d) δ [ppm]: 3.70-3.73 (m, 1H) 3.57-3.64 (m, 2H) 3.43-3.50 (m, 2H) 3.13-3.15 (m, 2H) 1.32 (s, 3H) 1.13 (s, 3H).

### Step 3: Preparation of 5-(azidomethyl)-2,2-dimethyl-1,4-dioxane

To a solution of 5-(iodomethyl)-2,2-dimethyl-1,4-dioxane (2.58 g, 10.1 mmol) in anhydrous DMF (13 mL) was added sodium azide (0.982 g, 15.1 mmol) and the suspension was heated at 80° C. for 2.5 hrs. The mixture was diluted with water (40 mL) and EtOAc (40 mL). The separated organic layer was washed with water (3×). The aqueous layers were combined and extracted with EtOAc (1×). The combined organic layers, dried over sodium sulfate, filtered off and concentrated under reduced pressure. The residue was purified by column chromatography [silica gel, EtOAc/hexane=10/40 to 50/50] providing 6-(azidomethyl)-2,2-dimethyl-1,4-dioxane (1.61 g) as a colorless oil. NMR (400 MHz, chloroform-d) δ [ppm]: 3.63-3.72 (m, 2H) 3.52-3.59 (m, 2H) 3.42 (d, J=11.6 Hz, 1H) 3.29 (d, J=4.4 Hz, 2H) 1.33 (s, 3H) 1.13 (s, 3H).

### Step 4: Preparation of (5,5-dimethyl-1,4-dioxan-2-yl)methanamine

To a solution of 5-(azidomethyl)-2,2-dimethyl-1,4-dioxane (810 mg, 4.73 mmol) in anhydrous tetrahydrofuran (20 mL) was added slowly a solution of lithium aluminumhydride (1.0 M tetrahydrofuran, 6.2 mL) 0° C. and the mixture was stirred at 0° C. for 1 hr and at room temperature for 0.5 hr. The reaction mixture was cooled to 0° C. and sodium sulfate decahydrate (excess) was slowly added and the suspension was vigorously stirred overnight. The suspension was filtered through cotton and the filtrate was concentrated under reduced pressure providing crude (5,5-dimethyl-1,4-dioxan-2-yl)methanamine (673 mg) as a colorless oil, which was directly used in the next step without purification. LCMS (m/z): 146.1 [M+H]+; Retention time=0.42 min.

### Synthesis of (4-methyltetrahydro-2H-pyran-4-yl)methanamine

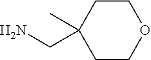

### Step 1: Preparation of 4-methyltetrahydro-2H-pyran-4-carbonitrile

To a solution of tetrahydro-2H-pyran-4-carbonitrile (2 g, 18.00 mmol) in tetrahydrofuran (10 mL) at 0-5° C. was added slowly LHMDS (21.59 mL, 21.59 mmol). The mixture was stirred for 1.5 hrs at 0° C. Iodomethane (3.37 mL, 54.0 mmol) was added slowly and stirring was continued for 30 min at ˜0° C. and then for ˜2 hrs at room temperature. The mixture was cooled to 0° C. and carefully diluted with 1N aqueous hydrochloride solution (30 mL) and EtOAc (5 mL) and concentrated under reduced pressure. The residue was taken up in diethylether and the separated organic layer was washed with brine, dried over sodium sulfate, filtered off and concentrated under reduced pressure providing crude 4-methyltetrahydro-2H-pyran-4-carbonitrile (1.8 g) as an orange oil, which was directly used in the next reaction without further purification. LCMS (m/z): 126.1 [M+H]+; Retention time=0.44 min.

### Step 2: Preparation of (4-methyltetrahydro-2H-pyran-4-yl)methanamine

To a solution of 4-methyltetrahydro-2H-pyran-4-carbonitrile (1.8 g, 14.38 mmol) in tetrahydrofuran (30 mL) was carefully added lithium aluminum hydride (1M solution in tetrahydrofuran, 21.57 mL, 21.57 mmol) at 0° C. The reaction mixture was stirred for 15 min at 0° C., allowed to warm to room temperature and stirred for additional 3 hrs at room temperature. To the reaction mixture was carefully added water (0.9 mL) [Caution: gas development!], 1N aqueous sodium hydroxide solution (2.7 mL) and water (0.9 mL). The mixture was vigorously stirred for 30 min. The precipitate was filtered off and rinsed with tetrahydrofuran. The solution was concentrated under reduced pressure providing crude (4-methyltetrahydro-2H-pyran-4-yl)methanamine (1.54 g) as a yellowish solid, which was directly used in the next step without further purification. LCMS (m/z): 130.1 [M+H]+; Retention time=0.21 min.

### Synthesis of 4-(aminomethyl)tetrahydro-2H-pyran-4-carbonitrile

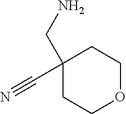

### Step 1: Preparation of dihydro-2H-pyran-4,4(3H)-dicarbonitrile

A mixture of malononitrile (0.991 g, 15 mmol), 1-bromo-2-(2-bromoethoxy)ethane (3.83 g, 16.50 mmol) and DBU (4.97 mL, 33.0 mmol) in DMF (6 mL) was heated at 85° C. for 3 hrs. The reaction mixture was cooled to room temperature and concentrated under reduced pressure. The residue was diluted with EtOAc (25 mL), washed with water (2×10 mL), dried over sodium sulfat, filtered off and concentrated under reduced pressure and further dried in high vacuo providing crude dihydro-2H-pyran-4,4(3H)-dicarbonitrile (1.65 g) as a light brown solid, which was directly used in the next step without further purification. GCMS: 136 [M]; Retention time=5.76 min. ¹H NMR (300 MHz, chloroform-d) δ [ppm]: 2.14-2.32 (m, 4H) 3.77-3.96 (m, 4H).

### Step 2: Preparation of 4-(aminomethyl)tetrahydro-2H-pyran-4-carbonitrile

To a solution of dihydro-2H-pyran-4,4(3H)-dicarbonitrile (450 mg, 3.31 mmol in EtOH (15 mL) was added sodium borohydride (375 mg, 9.92 mmol) in portions and the mixture was stirred at room temperature for 4 hrs. The mixture was concentrated under reduced pressure and the residue was diluted with EtOAc (30 mL), washed with water (10 mL), dried over sodium sulfate, filtered off and concentrated under reduced pressure providing crude 4-(aminomethyl)tetrahydro-2H-pyran-4-carbonitrile (388 mg), which was directly used in the next step without further purification. LCMS (m/z): 141.0 [M+H]+; Retention time=0.18 min.

### Synthesis of 4-(hydroxymethyl)tetrahydro-2H-pyran-4-carbonitrile

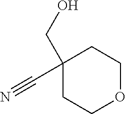

### Step 1: Preparation of methyl 4-cyanotetrahydro-2H-pyran-4-carboxylate

To methylcyanoacetate (7.87 ml, 101 mmol) in DMF (60 mL) at room temperature was added a solution of 1-bromo-2-(2-bromoethoxy)ethane (25.7 g, 111 mmol) in 20 mL DMF. To this mixture was added a solution of DBU (33.2 mL, 222 mmol) in 20 mL DMF dropwise via an addition funnel. The brown mixture was heated to 85° C. under argon for 3 hours. The reaction mixture was allowed to cool to room temperature, poured into water and extracted with EtOAc. The organic extracts were combined, washed with water and brine, dried over sodium sulfate and concentrated under reduced pressure. The residue was purified by column chromatography [SiO₂, 120 g, EtOAc/heptane]. Fractions were combined and concentrated under reduced pressure providing methyl 4-cyanotetrahydro-2H-pyran-4-carboxylate (11.2 g) as a nearly colorless oil.

### Step 2: Preparation of 4-(hydroxymethyl)tetrahydro-2H-pyran-4-carbonitrile

To a solution of methyl 4-cyanotetrahydro-2H-pyran-4-carboxylate (11.2 g, 66.2 mmol) in DME (60 mL) and MeOH (6 mL) at 0° C. was added sodium borohydride (1.454 g, 38.4 mmol) in one portion. The reaction mixture was stirred under argon at room temperature for 16 hrs. The resulting mixture was poured into saturated aqueous ammonium chloride solution (30 mL) and extracted with EtOAc (2×20 mL). The organic extracts were combined, washed with brine, dried over sodium sulfate and concentrated under reduced pressure providing crude 4-(hydroxymethyl)tetrahydro-2H-pyran-4-carbonitrile (7.8 g) as a nearly colorless oil, which was directly used without further purification. ¹H NMR (400 MHz, chloroform-d3) δ ppm 1.58-1.70 (m, 2H) 1.91 (dd, J=13.69, 1.96 Hz, 2H) 2.31 (br. s., 1H) 3.64-3.76 (m, 4H) 3.94-4.06 (m, 2H).

### Synthesis of toluene-4-sulfonic acid 4-methoxy-tetrahydro-pyran-4-ylmethyl ester

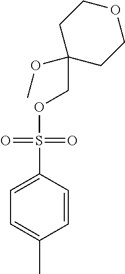 [SMILES: COC1(COS(=O)(=O)c2ccc(C)cc2)CCOCC1]

### Step 1: Preparation of 1,6-dioxaspiro[2.5]octane

To a solution of trimethylsulfonium iodide (3.27 g, 16 mmol) in DMSO (20 mL) under nitrogen atmosphere was added dihydro-2H-pyran-4(3H)-one (1.0 g, 10 mmol). To the mixture was added slowly a solution of tert-butoxide (1.68 g, 15 mmol) in DMSO (15 mL) and the solution was stirred at room temperature overnight. The reaction mixture was diluted slowly with water (50 mL) and extracted with diethylether (3×20 mL). The combined organic layers were dried over sodium sulfate, filtered off and concentrated under reduced pressure providing crude 1,6-dioxaspiro[2.5]octane (650 mg), which was directly used without further purification. ¹H NMR (300 MHz, chloroform-d) δ [ppm]: 1.44-1.62 (m, 2H) 1.76-1.98 (m, 2H) 2.70 (s, 2H) 3.70-3.98 (m, 4H).

### Step 2: Preparation of (4-methoxytetrahydro-2H-pyran-4-yl) MeOH

To a solution of 1,6-dioxaspiro[2.5]octane (600 mg, 5.26 mmol) in MeOH (10 mL) under nitrogen was added camphorsulfonic acid (50 mg, 0.21 mmol) at 0° C. and the mixture was stirred at 0° C. for 2 hrs. The mixture was concentrated under reduced pressure providing crude (4-methoxytetrahydro-2H-pyran-4-yl)methanol (707 mg) as a light yellow oil, which was directly used in the next step without further purification. ¹H NMR (300 MHz, chloroform-d) δ [ppm]: 1.89-2.08 (m, 4H), 3.18-3.30 (m, 3H), 3.47-3.59 (m, 2H), 3.64-3.78 (m, 4H).

### Step 3: Preparation of toluene-4-sulfonic acid 4-methoxy-tetrahydro-pyran-4-ylmethyl ester

To a solution of (4-methoxytetrahydro-2H-pyran-4-yl) MeOH (300 mg, 2.05 mmol) in pyridine (4 mL) was added toluenesulfonic chloride (430 mg, 2.25 mmol) at room temperature and the mixture was stirred at 25° C. overnight. The mixture was concentrated under reduced pressure and the residue was dissolved in dichloromethane (2 mL). Purification by column chromatography [silica gel, 12 g, EtOAc/hexane=0/100 to 30/70] provided toluene-4-sulfonic acid 4-methoxy-tetrahydro-pyran-4-ylmethyl ester (360 mg) as a light yellow solid. ¹H NMR (300 MHz, chloroform-d) δ [ppm]: 1.45-1.63 (m, 2H) 1.61-1.79 (m, 2H) 2.46 (s, 3H), 3.16 (s, 3H) 3.53-3.75 (m, 4H) 3.93 (s, 2H), 7.36 (d, J=8.20 Hz, 2H) 7.81 (d, J=8.20 Hz, 2H).

### Synthesis of (4-methoxytetrahydro-2H-pyran-4-yl)methanamine

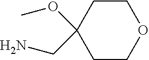

### Step 1: Preparation of 4,4-dimethoxytetrahydro-2H-pyran

A mixture of dihydro-2H-pyran-4(3H)-one (501 mg, 5 mmol), trimethyl orthoformate (0.608 mL, 5.50 mmol) and toluenesulfonic acid monohydrate (2.85 mg, 0.015 mmol) in MeOH (1 mL) was stirred in a sealed tube at 80° C. for 30 min. The reaction mixture was allowed to cool to room temperature and was concentrated under reduced pressure providing crude 4,4-dimethoxytetrahydro-2H-pyran (703 mg), which was used in the next step without further purification. ¹H NMR (400 MHz, chloroform-d) δ [ppm]: 1.61-1.90 (m, 4H) 3.20 (s, 6H) 3.60-3.78 (m, 4H).

### Step 2: Preparation of 4-methoxytetrahydro-2H-pyran-4-carbonitrile

To a solution of 4,4-dimethoxytetrahydro-2H-pyran (0.703 g, 4.81 mmol) and tin(IV)chloride (0.564 mL, 4.81 mmol) in dichloromethane (15 mL) was added slowly 2-isocyano-2-methylpropane (0.400 g, 4.81 mmol) at −70° C. and the mixture was allowed to warm to room temperature over 2-3 hrs. The mixture was diluted with aqueous sodium bicarbonate solution (10 mL) and dichloromethane (20 mL). The separated organic layer was washed with water (3×10 mL) and dried over sodium sulfate, filtered off and concentrated under reduced pressure providing crude 4-methoxytetrahydro-2H-pyran-4-carbonitrile (511 mg), which was used in the next step without further purification. GCMS: 109 [M-MeOH]; Retention time=5.44 min.

### Step 3: Preparation of (4-methoxytetrahydro-2H-pyran-4-yl)methanamine

To a mixture of LiAlH₄ (275 mg, 7.24 mmol) in tetrahydrofuran (10 mL) at room temperature was slowly added a solution of 4-methoxytetrahydro-2H-pyran-4-carbonitrile (511 mg, 3.62 mmol) in tetrahydrofuran (10 mL). The mixture was stirred at room temperature for 1 hr and heated to reflux for 3 hrs. The reaction mixture was cooled to 0° C. and water (3 mL) was carefully added dropwise. The resulting mixture was stirred for additional 30 min and filtered to remove all solids. The filtrate was dried over sodium sulfate for 2 hrs, filtered off and concentrated under reduced pressure providing crude (4-methoxytetrahydro-2H-pyran-4-yl)methanamine (370 mg), which was used in the next step without further purification. LCMS (m/z): 146.1 [M+H]+, 114.0 [M-MeOH]; Retention time=0.19 min.

### Synthesis of toluene-4-sulfonic acid 1′,1′-dioxo-hexahydro-1-thiopyran-4-yl-methyl ester

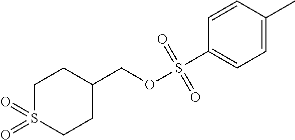 [SMILES: Cc1ccc(S(=O)(=O)OCC2CCS(=O)(=O)CC2)cc1]

A mixture of (1′,1′-dioxo-hexahydro-1-thiopyran-4-yl)-methanol (2.5 g, 15.22 mmol) [Organic Process Research & Development 2008, 12, 892-895.], pyridine (25 mL) and tosyl-Cl (2.90 g, 15.22 mmol) was stirred for 18 hrs at 50° C. The reaction mixture was concentrated under reduced pressure. The residue was purified by column chromatography [silica gel, EtOAc/hexane=0/100 to 70/30]. Fractions were combined and concentrated under reduced pressure providing toluene-4-sulfonic acid 1′,1′-dioxo-hexahydro-1-thiopyran-4-yl-methyl ester (3.78 g). LCMS (m/z): 319.0 [M+H]+; Retention time=0.71 min.

### Synthesis of (2R,6S)-2,6-dimethyltetrahydro-2H-pyran-4-carbaldehyde

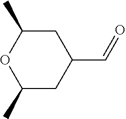

### Step 1: Preparation of (2R,6S)-2,6-dimethyldihydro-2H-pyran-4(3H)-one

A solution of 2,6-dimethyl-4H-pyran-4-one (2 g, 16.1 mmol) in EtOH (20 mL) was stirred over Pd/C (10 wt. %, 0.2 g) under hydrogen (15 psi) for 16 hrs at ambient temperature. The suspension was filtered off and the filtrate was concentrated under reduced pressure. The residue was dissolved in dichloromethane (15 mL) and treated with Dess-Martin periodinane (2.3 g) at ambient temperature for 16 hrs. To the suspension was added saturated aqueous sodium thiosulfate solution (˜3 mL) and the mixture was stirred for 1 hr. The mixture was diluted with saturated aqueous sodium bicarbonate solution (20 mL) and stirred for an additional 1 hr. The separated organic phase was washed with water and brine, dried over sodium sulfate, filtered through celite and concentrated under reduced pressure. The residue was purified by column chromatography [silica gel, EtOAc/heptane=10/90]. Fractions were combined and concentrated under reduced pressure providing (2R,6S)-2,6-dimethyldihydro-2H-pyran-4(3H)-one (600 mg). GCMS: 128 [M]; Retention time=4.25 min. ¹H NMR (400 MHz, DMSO-d6) δ [ppm]: 1.18 (d, J=6.26 Hz, 6H) 2.11-2.25 (m, 4H) 3.58-3.77 (m, 2H).

### Step 2: Preparation of (2R,6S)-4-(methoxymethylene)-2,6-dimethyltetrahydro-2H-pyran

To a suspension of (methoxymethyl)triphenyl phosphine chloride (1.5 g, 4.45 mmol) in tetrahydrofuran (8 mL) was added slowly sodium bis(trimethylsilyl)amide (1M solution in tetrahydrofuran, 4.45 mL) at −10° C. The reaction mixture was stirred for 1 hr and a solution of (2R,6S)-2,6-dimethyldihydro-2H-pyran-4(3H)-one (380 mg, 2.96 mmol) in tetrahydrofuran (2 mL) was added slowly. The resulting mixture was allowed to warm to ambient temperature and stirred for 3 hrs. The reaction mixture was diluted with water (15 mL) and extracted with diethylether (2×30 mL). The combined organic layers were washed with brine, dried over sodium sulfate, filtered off and concentrated under reduced pressure. The residue was purified by column chromatography [silica gel, EtOAc/heptane=10/90] providing (2R,6S)-4-(methoxymethylene)-2,6-dimethyltetrahydro-2H-pyran (240 mg) as a colorless oil. GCMS: 156 [M]; Retention time=5.40 min. ¹H NMR (400 MHz, DMSO-d6) δ [ppm]: 1.07 (t, J=6.06 Hz, 6H) 1.18-1.29 (m, 1H) 1.31-1.46 (m, 1H) 1.61 (t, J=12.13 Hz, 1H) 1.93 (d, J=13.30 Hz, 1H) 3.17-3.28 (m, 2H) 3.46 (s, 3H) 5.89 (s, 1H).

### Step 3: Preparation of (2R,6S)-2,6-dimethyltetrahydro-2H-pyran-4-carbaldehyde

A mixture of (2R,6S)-4-(methoxymethylene)-2,6-dimethyltetrahydro-2H-pyran (240 mg, 1.53 mmol) and formic acid (˜88 wt. % in water, 1.5 mL, 34.4 mmol) under argon was heated at 90° C. for 1 hr. The reaction mixture was cooled to 0° C., neutralized with 1N aqueous sodium hydroxide solution until pH-6 and extracted with diethylether. The organic layer were dried over sodium sulfate, filtered off and concentrated under reduced pressure providing crude (2R,6S)-2,6-dimethyltetrahydro-2H-pyran-4-carbaldehyde (120 mg) as a yellow oil, which was directly used in the next reaction without further purification. GCMS: 142 [M]; Retention time=5.0 min. ¹H NMR (400 MHz, DMSO-d6) δ [ppm]: 0.89-1.00 (m, 2H) 1.09 (d, J=6.26 Hz, 6H) 1.77 (ddd, J=12.33, 1.96, 1.76 Hz, 2H) 3.35 (t, J=7.04 Hz, 1H) 3.38-3.48 (m, 2H) 9.51 (s, 1H).

### Synthesis of 2,5′-dichloro-2′-fluoro-N-((tetrahydro-2H-pyran-4-yl)methyl)-3,4′-bipyridin-5-amine. (Intermediate E)

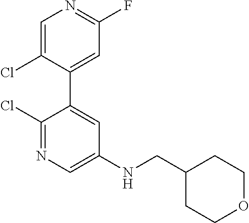 [SMILES: Fc1cc(-c2cc(NCC3CCOCC3)cnc2Cl)c(Cl)cn1]

A mixture of Intermediate B (3 g, 8.84 mmol), 5-chloro-2-fluoropyridin-4-ylboronic acid (4.88 g, 27.8 mmol), 2 M Na₂CO₃ (17.67 mL, 35.3 mmol) in DME (48 mL) was purged with argon for 5 min in a glass bomb with stir bar followed by addition of PdCl₂(dppf).CH₂Cl₂ adduct (0.722 g, 0.884 mmol). The mixture was capped and heated at 100° C. for about 3 hours. LC/MS showed as a mixture of ˜50% starting material (M+H=305/307, retention time=0.86 min.), 30% product (M+H=356/358, retention time=0.89 min.), and 15% des-bromo starting material (M+H=227.0, retention time=0.64 min.). The reaction was diluted in 250 mL EtOAc, then washed with water (250 mL), sat NaHCO₃ (250 mL), and brine (200 mL). Organic layer was dried over Na₂SO₄, filtered, and concentrated in vacuo to give 6.2 g of dark brown oil. The residue was purified by column chromatography (ISCO, SiO2, 120 g, eluted with 100% heptane for 1 min, 20-50% EtOAc in heptane over 55 min, hold for 20 min). The product and des-bromo fractions were combined and concentrated in vacuo to yield 0.659 g brown gum which contained 66% product and 33% des-bromo starting material based upon LC/MS analysis. LC/MS of the product: 356/358 (MH⁺), retention time=0.89 min.

### Synthesis of N2′-(trans-4-aminocyclohexyl)-2,5′-dichloro-N5-((tetrahydro-2H-pyran-4-yl)methyl)-3,4′-bipyridine-2′,5-diamine (Intermediate F)

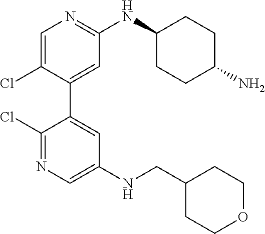 [SMILES: N[C@H]1CC[C@H](Nc2cc(-c3cc(NCC4CCOCC4)cnc3Cl)c(Cl)cn2)CC1]

To a scintillation vial containing 2,5′-dichloro-2′-fluoro-N-((tetrahydro-2H-pyran-4-yl)methyl)-3,4′-bipyridin-5-amine Intermediate E (70 mg, 0.197 mmol) was added DMSO (2 ml) and trans-cyclohexane-1,4-diamine (224 mg, 1.965 mmol). The homogenous reaction mixture was capped and heated to 100° C. in oil bath for 3 hours. The resulting solution was purified by reverse phase preparative HPLC to yield N2′-(trans-4-aminocyclohexyl)-2,5′-dichloro-N5-((tetrahydro-2H-pyran-4-yl)methyl)-3,4′-bipyridine-2′,5-diamine (38 mg, 0.067 mmol, 34.3% yield), LCMS (m/z): 450.0 (MH⁺), retention time=0.59 min, as a yellow solid.

### Synthesis of 5-bromo-3-((tetrahydro-2H-pyran-4-yl)methyl)aminopicolinonitrile (Intermediate G)

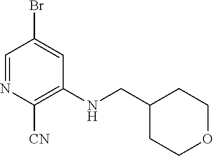 [SMILES: N#Cc1ncc(Br)cc1NCC1CCOCC1]

### Step 1. Synthesis of 5-bromo-3-fluoropicolinonitrile

Tetrabutylammonium fluoride (26.3 ml, 26.3 mmol, 1M in THF) was charged to a round bottom flask and cooled to −40° C. This was treated with sulfuric acid (0.04 ml, 0.074 g, 0.750 mmol). It was then treated with DMF (18 ml) until the suspension became homogenous. To this mixture was added slowly a solution of 5-bromo-3-nitropicolinonitrile (2.0 g, 8.77 mmol) dissolved in DMF (32 ml). Once the addition was complete the reaction was allowed to stir at −40° C. for 90 minutes and at room temperature for another 1 hour. The reaction was then quenched with 2 N HCl (20 ml), then diluted with H₂O (100 ml) and extracted with EtOAc (3×100 ml). The extracts were washed with H₂O (4×100 ml) followed by brine (1×100 ml). The organic layer was dried (Na₂SO₄), filtered, and concentrated in vacuo to give 2.15 g of crude material. The material was purified using the ISCO system (40 g of silica gel column. Eluted using 10 EtOAc/90 heptane to 50 EtOAc 50 Heptane over 20 min). The pure fractions were combined and concentrated to yield 0.7458 g (42%) of pure desired product. ¹H NMR (300 MHz, METHANOL-d4) δ ppm 8.27 (dd, J=8.20, 1.76 Hz, 1H) 8.70 (s, 1H).

### Step 2. Synthesis of 5-bromo-3-((tetrahydro-2H-pyran-4-yl)methyl)aminopicolinonitrile

Compound obtained from the above step (0.100 g, 0.498 mmol), (tetrahydro-2H-pyran-4-yl)methanamine (0.073 ml, 0.069 g, 0.597 mmol), and triethylamine (0.073 ml, 0.069 g, 0.597 mmol) were dissolved in DMA (1.0 ml). The reaction was then heated at 80° C. for 3 hours. It was allowed to cool to room temperature. The reaction mixture was diluted with H₂O (25 ml) and was extracted with EtOAc (3×25 ml). The combined extracts were washed with H₂O (2×25 ml) and brine (1×25 ml). The organic layer was dried (Na₂SO₄), filtered, and concentrated in vacuo to give 0.1708 g of crude material. The material was purified using the ISCO system (12 g of SiO₂ column. Eluted using 25 EtOAc/75 heptane to 100 EtOAc over 15 min). The pure fractions were combined and concentrated to yield 0.0657 g (45%) of the title compound. LC/MS of the product: 296.0/297.9 (MH⁺), retention time=0.81 min. ¹H NMR (300 MHz, CHLOROFORM-d) δ ppm 1.31-1.48 (m, 2H) 1.50-1.53 (m, 0H) 1.56 (s, 1H) 1.72 (d, J=12.89 Hz, 2H) 1.78-1.98 (m, 1H) 3.10 (t, J=6.30 Hz, 2H) 3.42 (td, J=11.87, 1.76 Hz, 2H) 4.02 (dd, J=11.28, 3.66 Hz, 2H) 4.64-4.79 (m, 1H) 7.19 (d, J=1.76 Hz, 1H) 8.01 (d, J=1.76 Hz, 1H).

### Synthesis of N1-((R)-3,3,3-trifluoro-2-methoxypropyl)cyclohexane-trans-1,4-diamine (Intermediate H)

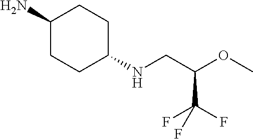 [SMILES: CO[C@H](CN[C@H]1CC[C@H](N)CC1)C(F)(F)F]

### Step 1. Preparation of (R)-3-(benzyloxy)-1,1,1-trifluoropropan-2-ol

(R)-(+)-3,3,3-Trifluoro-1,2-epoxypropane (700 μL, 8.08 mmol) and benzyl alcohol (1.68 mL, 16.17 mmol) were dissolved in DCM (20 ml). Boron trifluoride diethyl etherate (102 μL, 0.808 mmol) was added. The reaction mixture was stirred for about 16 hours at 60° C. in a sealed vessel. The reaction was judged to be complete by TLC (2:1 heptanes:ethyl acetate). The reaction mixture was cooled to ambient temperature, diluted with DCM, and washed sequentially with saturated sodium bicarbonate and brine. The organic phase was dried over sodium sulfate, filtered, and concentrated. The crude material was purified by flash chromatography (heptanes/ethyl acetate gradient) to give 998 mg (56.1% yield) of (R)-3-(benzyloxy)-1,1,1-trifluoropropan-2-ol as a colorless oil.

### Step 2. Preparation of (R)-((3,3,3-trifluoro-2-methoxypropoxy)methyl)benzene

(R)-3-(benzyloxy)-1,1,1-trifluoropropan-2-ol (998 mg, 4.53 mmol) was dissolved in THF (20 ml) at ambient temperature. Sodium hydride (190 mg, 4.76 mmol) was added. The mixture was stirred for 10 minutes at ambient temperature and 20 minutes at 50° C. Iodomethane (0.312 ml, 4.99 mmol) was added. The reaction vessel was sealed and stirred at 50° C. for about 16 hours. TLC (2:1 heptanes:ethyl acetate) showed clean conversion to product. The cooled reaction was quenched by the addition of saturated aqueous sodium bicarbonate. The mixture was extracted with ethyl acetate. The combined organic layers were washed with brine, dried over sodium sulfate, filtered, and concentrated to give 1.05 g (99% yield) of crude (R)-((3,3,3-trifluoro-2-methoxypropoxy)methyl)benzene which was used without further purification.

### Step 3. Preparation of (R)-3,3,3-trifluoro-2-methoxypropan-1-ol

(R)-((3,3,3-trifluoro-2-methoxypropoxy)methyl)benzene (1.05 g, 4.48 mmol) was dissolved in methanol (90 ml). Argon was bubbled through the solution for 5 minutes, and 20% palladium hydroxide on carbon (0.079 g, 0.112 mmol) was added. The flask was purged and flushed twice with hydrogen. The mixture was stirred for about 16 hours at ambient temperature under a hydrogen balloon. The mixture was filtered through a pad of celite. The filter cake was rinsed with additional methanol. The filtrate was concentrated at ambient temperature to give 495 mg (77%) of (R)-3,3,3-trifluoro-2-methoxypropan-1-ol as a colorless oil. This was used in the next step without further purification.

### Step 4. Preparation of (R)-3,3,3-trifluoro-2-methoxypropyl 4-methylbenzenesulfonate

Sodium hydride (412 mg, 10.31 mmol) was added to a solution of (R)-3,3,3-trifluoro-2-methoxypropan-1-ol (495 mg, 3.44 mmol) in THF (10 ml) at ambient temperature. The mixture was stirred for 30 minutes. P-Toluenesulfonyl chloride (1965 mg, 10.31 mmol) was added. The white cloudy solution was stirred at ambient temperature for 18 hours. The reaction mixture was diluted with saturated aqueous sodium bicarbonate and extracted with EtOAc. The organic extracts were combined, washed with brine, dried with sodium sulfate and concentrated in vacuo. The crude mixture was purified by flash chromatography (heptanes:EtOAc gradient) to give 0.51 g of (R)-3,3,3-trifluoro-2-methoxypropyl 4-methylbenzenesulfonate as a colorless crystalline solid. LCMS (m/z): 298.9 (MR); retention time=1.01 min.

### Step 5. Preparation of N1-((R)-3,3,3-trifluoro-2-methoxypropyl)cyclohexane-trans-1,4-diamine

(R)-3,3,3-trifluoro-2-methoxypropyl 4-methylbenzenesulfonate (510 mg, 1.71 mmol) and trans-1,4-diaminocyclohexane (586 mg, 5.13 mmol) were suspended in DMSO (4 ml). The reaction mixture was stirred at 100° C. for 3 hours. The cooled reaction mixture was diluted with water (40 mL) and extracted with DCM. The combined extracts were washed sequentially with water and brine, dried over sodium sulfate, filtered, and concentrated to give 400 mg (97% yield) of crude N1-((R)-3,3,3-trifluoro-2-methoxypropyl)cyclohexane-trans-1,4-diamine which was used without further purification. LCMS (m/z): 241.1 (MH⁺); retention time=0.33 min. ¹H NMR (400 MHz, CHLOROFORM-d) δ ppm 0.93-1.20 (m, 4H) 1.83 (br. s., 4H) 2.25-2.41 (m, 2H) 2.65-2.85 (m, 4H) 3.52 (s, 3H) 3.54-3.66 (m, 2H).

### Synthesis of cis- and trans-4-(2,2-dimethylmorpholino)cyclohexanamine

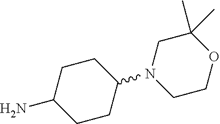

### Step 1: Preparation of tert-butyl cis/trans-4-(2,2-dimethylmorpholino)cyclohexylcarbamate

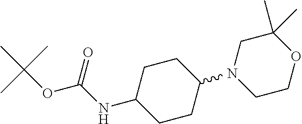 [SMILES: CC(C)(C)OC(=O)NC1CCC(N2CCOC(C)(C)C2)CC1]

To a solution of tert-butyl 4-oxocyclohexylcarbamate (350 mg, 1.641 mmol) in methylene chloride (8 mL) was added 2,2-dimethylmorpholine (189 mg, 1.641 mmol) followed by sodium triacetoxyborohydride (1.739 g, 8.21 mmol). Reaction mixture was stirred at 25° C. for 6 hr. Reaction mixture was diluted with EtOAc and washed with water. Organics were isolated, dried (MgSO4), filtered and concentrated under reduced pressure. The residue was purified by column chromatography [SiO₂; 12 g] to provide the title compound as a yellow oil. LCMS (m/z): 313.1 [M+H]+; Retention time=0.60 min.

### Step 2: Preparation of cis- and trans-4-(2,2-dimethylmorpholino)cyclohexanamine

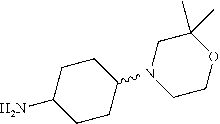

To a solution of tert-butyl cis/trans-4-(2,2-dimethylmorpholino)cyclohexylcarbamate (419 mg, 1.341 mmol) in methylene chloride (10 mL) was added trifluoroacetic acid (0.103 mL, 1.341 mmol). Reaction was stirred at 25° C. for 2 hr. Reaction was concentrated to provide the title compounds as trifluoroacetic acid salts as a white solid which was used without further purification. (400 mg, 1.884 mmol). LCMS (m/z): 213.1 [M+H]+; Retention time=0.19 min LC/MS Rt=0.19 min, m/z (H+)=213.1

### Synthesis of trans-N1-((R)-1-methoxypropan-2-yl)cyclohexane-1,4-diamine

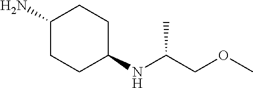

### Step 1: Preparation of (S)-1-methoxypropan-2-yl 4-methylbenzenesulfonate

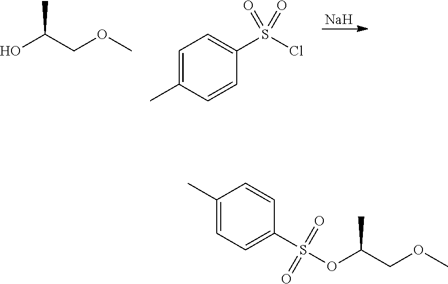

To sodium hydride (5.99 g, 150 mmol) in THF (200 mL) at 0° C. was added (S)-1-methoxypropan-2-ol (13.5 g, 150 mmol) dropwise. The mixture was warmed to room temperature and stirred under argon for 1 hr. The resulting white cloudy mixture was cooled to 0° C. To this was added 4-methylbenzene-1-sulfonyl chloride (28.6 g, 150 mmol) in THF (200 mL). The reaction mixture was stirred at room temperature for 18 hr. The reaction mixture was poured into water and extracted with EtOAc (3×150 mL). The organic extracts were combined, washed with brine, dried over sodium sulfate, filtered and concentrated under reduced pressure to give 45 g of oil. The crude mixture was purified by column chromatography [SiO₂, 330 g, EtOAc/heptane=0/100 for 10 min, 10/90 for 20 min, then 30/70], providing 27.33 g of (S)-1-methoxypropan-2-yl 4-methylbenzenesulfonate as colorless oil. ¹H NMR (400 MHz, chloroform-d) δ ppm 1.28 (d, 3H) 2.45 (s, 3H) 3.25 (s, 3H) 3.33-3.47 (m, 2H) 4.72 (td, 1H) 7.34 (d, 2H) 7.82 (d, 2H).

### Step 2: Preparation of trans-N1-((R)-1-methoxypropan-2-yl)cyclohexane-1,4-diamine

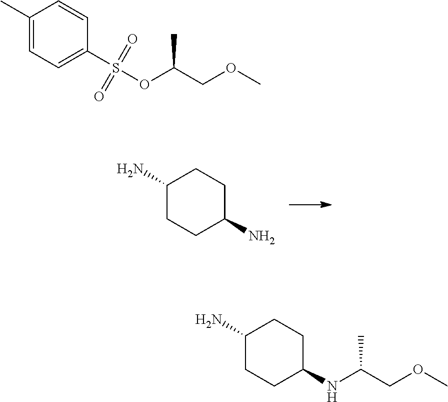

To (S)-1-methoxypropan-2-yl 4-methylbenzenesulfonate (15 g, 61.4 mmol) in acetonitrile (100 mL) at room temperature was added 1,4-trans-cyclohexane-diamine (17.53 g, 153 mmol). The light brown mixture was heated to 90° C. in a sealed steel bomb for 18 hr. The resulting mixture was cloudy light brown. LC/MS showed formation of desired product and side bis-alkylated product. A second batch of the same reaction mixture was set up in a similar fashion (12.33 g of (S)-1-methoxypropan-2-yl 4-methylbenzenesulfonate, 14.41 g of 1,4-trans-cyclohexane-diamine) and the two reactions were cooled to room temperature, combined and worked up as below. To the cooled reaction mixture, ether (˜200 mL) was added. The solid was removed by filtration. The filtrate was concentrated then heptane (80 mL) and EtOAc (15 mL) were added. The precipitates were removed by filtration. The filtrate was concentrated under reduced pressure to give brown oil and some solid. The residue was dissolved with 100 mL of water and extracted with ether (1×100 mL) and DCM (4×45 mL). Ether extract was discarded. The DCM extracts were combined, dried with sodium sulfate and concentrated under reduced pressure to give 10.4 g (50% yield) of brown oil. LC/MS showed this contained trans-N1-((R)-1-methoxypropan-2-yl)cyclohexane-1,4-diamine (major) along with bis-alkylated side product (˜5%). This was used in the next step without further purification. LCMS (m/z): 187.1 [M+H]+; Retention time=0.15 min. ¹H NMR (400 MHz, chloroform-d) δ ppm 1.02 (d, 3H) 1.05-1.23 (m, 4H) 1.77-2.03 (m, 4H) 2.49 (br. s., 1H) 2.65 (d, 1H) 2.95-3.06 (m, 1H) 3.18-3.31 (m, 2H) 3.34 (s, 3H).

### Synthesis of trans-N1-(1-(trideuteromethoxy)propan-2-yl)cyclohexane-1,4-diamine

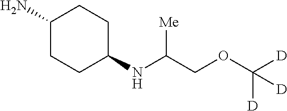 [SMILES: [2H]C([2H])([2H])OCC(C)N[C@H]1CC[C@H](N)CC1]

### Step 1: Preparation of 1-(trideuteromethoxy)propan-2-yl 4-methylbenzenesulfonate

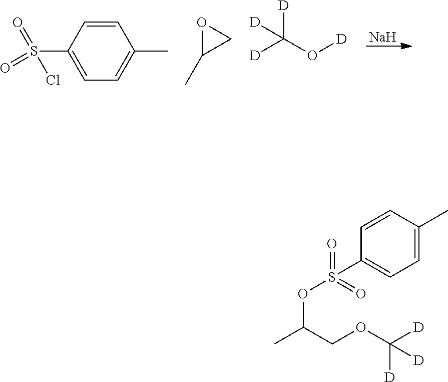

To 2-methyloxirane (0.603 mL, 8.61 mmol) in DMF (10 mL) at room temperature was added methanol-d4 (0.310 g, 8.61 mmol) dropwise. The resulting grey cloudy mixture was stirred at room temperature under argon for 30 min followed by addition of 2-methyloxirane (0.603 mL, 8.61 mmol). The mixture was heated to 50° C. in a sealed scintillation vial for 18 hr. The resulting mixture was dark brown and cloudy. To this was added tosyl-Cl (1.641 g, 8.61 mmol) in one portion and the mixture was stirred at room temperature for 3 hr. The reaction mixture was poured into aqueous saturated NaHCO₃ solution (50 mL) and extracted with EtOAc (2×50 mL). The organic extracts were combined, washed with brine, dried with sodium sulfate, filtered and concentrated under reduced pressure to give a brown oil. The crude mixture was purified by column chromatography [SiO₂, 40 g, EtOAc/heptane=0/100 for 4 min, 30/70 for 4-8 min, then 50/50 for 20 min] providing 0.77 g of 1-(trideuteromethoxy)propan-2-yl 4-methylbenzenesulfonate as a light yellow oil.

### Step 2: Preparation of trans-N1-(1-(trideuteromethoxy)propan-2-yl)cyclohexane-1,4-diamine

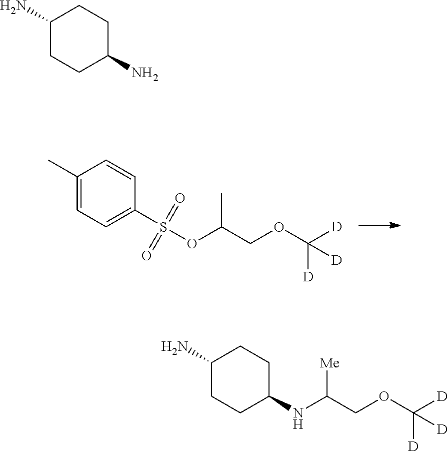 [SMILES: N[C@H]1CC[C@H](N)CC1 ; [2H]C([2H])([2H])OCC(C)OS(=O)(=O)c1ccc(C)cc1 ; [2H]C([2H])([2H])OCC(C)N[C@H]1CC[C@H](N)CC1]

To 1-(trideuteromethoxy)propan-2-yl 4-methylbenzenesulfonate (0.77 g, 3.11 mmol) in acetonitrile (10 mL) at room temperature was added 1,4-trans-cyclohexane-diamine (0.711 g, 6.23 mmol). The light brown mixture was heated to 90° C. in a sealed steel bomb for 18 hr. The resulting mixture was cloudy light brown. LC/MS showed formation of desired product and side bis-alkylated product in a ratio about 2:1. The reaction mixture was cooled to room temperature and ether was added. The solid was removed by filtration. The filtrate was concentrated under reduced pressure to give a brown oil. The residue was dissolved with saturated aqueous sodium bicarbonate solution (5 mL) and extracted with ether (1×10 mL) and DCM (4×5 mL). LC/MS showed ether extract mainly contained bis-alkylated side product and little product, this was discarded. The DCM extracts were combined, dried with sodium sulfate, filtered and concentrated under reduced pressure to give 0.19 g of trans-N1-(1-(trideuteromethoxy)propan-2-yl)cyclohexane-1,4-diamine as a brown oil. LC/MS showed this contained desired product (major) along with bis-alkylated side product and other impurity (with UV absorption). This was used in the next step without further purification. LCMS (m/z): 188.1 [M+H]+; Retention time=0.17 min.

### Synthesis of trans-N1-(2-deutero-1-methoxypropan-2-yl)cyclohexane-1,4-diamine

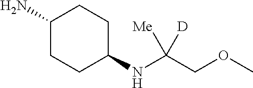

### Step 1: Preparation of 2-deutero-1-methoxypropan-2-ol

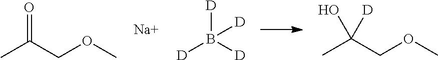

To 1-methoxypropan-2-one (5.26 mL, 56.8 mmol) in MeOH-d4 (10 mL) and THF (50.00 mL) at 0° C. was added NaBD₄ (2.375 g, 56.8 mmol) portion wise. Vigorous off-gassing was seen. The reaction mixture was warmed to room temperature and stirred under argon for 5 hrs. The reaction mixture was worked up by pouring saturated aqueous NaHCO₃ solution (10 mL) and stirred for 1 hr. The product was extracted with diethyl ether (100 mL), washed with brine, dried with sodium sulfate and concentrated under reduced pressure to give 3.53 g of colorless liquid. This was used in the next step without further purification.

### Step 2: Preparation of 2-deutero-1-methoxypropan-2-yl 4-methylbenzenesulfonate

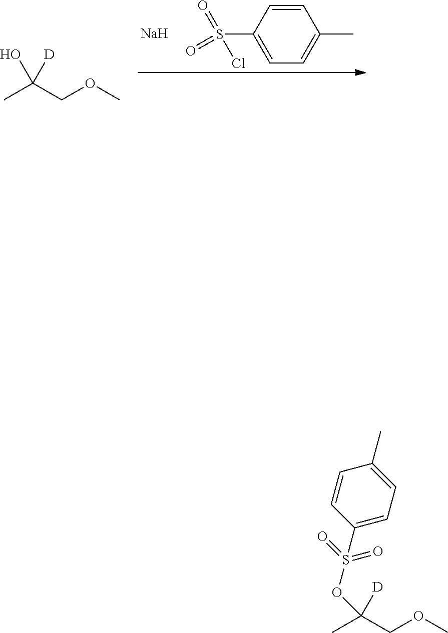

To NaH (1.549 g, 38.7 mmol) in THF (10 mL) was added 2-deutero-1-methoxypropan-2-ol (3.53 g, 38.7 mmol) in THF (10 mL) dropwise. The mixture was stirred at room temperature for 10 min to give a grey cloudy mixture. To this was added tosyl-Cl (7.39 g, 38.7 mmol) in one portion. The reaction mixture was stirred under argon at room temperature for 2 days. The reaction mixture was poured into water and extracted with ethyl acetate. The organic extracts were combined, washed with brine, dried with sodium sulfate, filtered, and concentrated under reduced pressure to give 7.2 g of colorless oil. The crude mixture was purified by column chromatography [SiO₂, 120 g, EtOAc/heptane=0/100 for 4 min, 30/70 until 12 min, then 50/50 until 20 min] providing 4.3 g of 2-deutero-1-methoxypropan-2-yl 4-methylbenzenesulfonate as a colorless oil. ¹H NMR (400 MHz, chloroform-d) δ ppm 1.27 (s, 3H) 2.45 (s, 3H) 3.25 (s, 3H) 3.33-3.46 (m, 2H) 7.34 (d, 2H) 7.81 (d, 2H).

### Step 3: Preparation of trans-N1-(2-deutero-1-methoxypropan-2-yl)cyclohexane-1,4-diamine

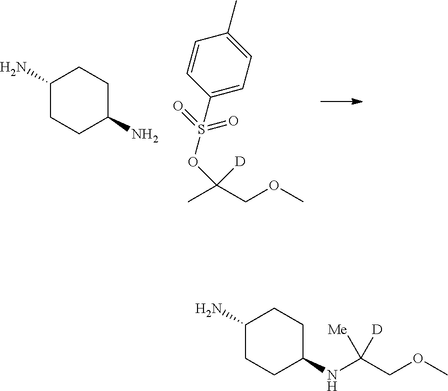 [SMILES: N[C@H]1CC[C@H](N)CC1 ; [2H]C(C)(COC)OS(=O)(=O)c1ccc(C)cc1 ; [2H]C(C)(COC)N[C@H]1CC[C@H](N)CC1]

To 2-deutero-1-methoxypropan-2-yl 4-methylbenzenesulfonate (4.3 g, 17.53 mmol) in acetonitrile (80 mL) at room temperature was added 1,4-trans-cyclohexane-diamine (4.00 g, 35.1 mmol). The light brown mixture was heated to 90° C. in a sealed steel bomb for 18 hr. The resulting mixture was cloudy light brown. LC/MS showed formation of desired product and side bis-alkylated product in a ratio of 2:1. The reaction mixture was cooled to room temperature and ether was added. The solid was removed by filtration. The filtrate was concentrated under reduced pressure to give a brown oil. To this was added ether (80 mL) and heptane (80 mL). A lot of precipitates formed which were removed by filtration. The filtrate was concentrated under reduced pressure to give 2.85 g of brown oil. The residue was dissolved with 20 mL of saturated aqueous sodium bicarbonate solution and extracted with ether (lx 40 mL) and DCM (4×20 mL). LC/MS showed ether extract only contained bis-alkylated side product and little product. The DCM extracts were combined, dried with sodium sulfate and concentrated under reduced pressure to give 1.19 g of brown oil. LC/MS showed this contained desired product (major) along with bis-alkylated side product. This was used in the next step without further purification. LCMS (m/z): 188.1 [M+H]+; Retention time=0.17 min. ¹H NMR (400 MHz, chloroform-d) δ ppm 0.97-1.27 (m, 7H) 1.81-2.03 (m, 4H) 2.42-2.55 (m, 1H) 2.59-2.71 (m, 1H) 3.19-3.31 (m, 2H) 3.34 (s, 3H).

### Synthesis of trans-N1-cyclopropyl-N1-(2-methoxyethyl)cyclohexane-1,4-diamine

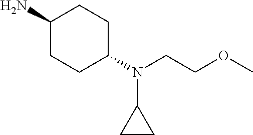

### Step 1: Preparation of tert-butyl (trans-4-((2-ethoxyethyl)amino)cyclohexyl)carbamate

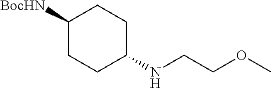 [SMILES: COCCN[C@H]1CC[C@H](NC(=O)OC(C)(C)C)CC1]

To 2-methoxyethyl 4-methylbenzenesulfonate (2.68 g, 11.64 mmol) in acetonitrile (50 mL) at room temperature was added N-Boc-trans-cyclohexane-1,4-diamine (4.99 g, 23.28 mmol). The off-white suspension was heated to 95° C. in a sealed glass bomb for 18 hr. The resulting mixture was light brown with white precipitate. LC/MS showed no starting materials with desired product and side product in a ratio of ˜1:1. The reaction mixture was cooled to room temperature and filtered. The filtrate was concentrated under reduced pressure to give 3 g of brown oil. The crude product was purified by column chromatography [silica gel, 40 g, MeOH/DCM=0/100 for 5 min, 5/95 for 5 min, then 1/9 for 30 min]. The pure fractions were combined and concentrated under reduced pressure to give 2.08 g of product as white foam. LC/MS showed the material was not very clean, but contains desired product as main component, showed no UV absorption. LCMS (m/z): 273.1 [M+H]+; Retention time=0.45 min. ¹H NMR showed as mono-tosylate salt. ¹H NMR (400 MHz, methanol-d4) δ ppm 1.17-1.51 (m, 13H) 1.93-2.19 (m, 4H) 2.37 (s, 3H) 2.88-3.03 (m, 1H) 3.10-3.17 (m, 2H) 3.40 (s, 3H) 3.55-3.64 (m, 2H) 7.16-7.27 (m, 2H) 7.67-7.75 (m, 2H).

### Synthesis of 2,5′-dichloro-N-((2,2-dimethyltetrahydro-2H-pyran-4-yl)methyl)-2′-fluoro-3,4′-bipyridin-5-amine (Intermediate I)

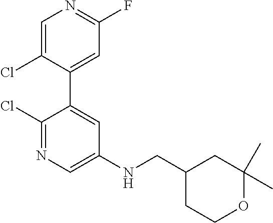 [SMILES: CC1(C)CC(CNc2cnc(Cl)c(-c3cc(F)ncc3Cl)c2)CCO1]

### Step 1. Preparation of 5-bromo-6-chloropyridin-3-amine

In a 250 mL round bottom flask, 3-bromo-2-chloro-5-nitropyridine (3 g, 12.63 mmol), ammonium chloride (1.35 g, 25.2 mmol) and zinc dust (8.79 g, 134 mmol) were suspended in MeOH (50 ml). The reaction mixture was heated for 2 hrs at 90° C. The reaction was cooled to room temperature, filtered over celite, and concentrated in vacuo. The resulting solid was adsorbed onto silica and purified by silica gel chromatography (25-55% EtOAc/Heptane). The title compound (1.85 g, 8.92 mmol, 70.6% yield) was obtained as a yellow solid. LCMS (m/z): 219.1 (MH⁺); retention time=0.77 min.

### Step 2. Preparation of 2,2-dimethyltetrahydro-2H-pyran-4-carbaldehyde

To a solution of (2,2-dimethyltetrahydro-2H-pyran-4-yl)methanol (1 g, 6.93 mmol) in DCM (20 ml) was added tetrapropylammonium perruthenate (0.122 g, 0.347 mmol), 4-methylmorpholine 4-oxide (1.218 g, 10.40 mmol), and 4 A powdered molecular sieves (3.5 g). The reaction vessel was purged with argon, capped, and stirred at room temperature for 2 hours. TLC showed that there was no starting material (Rf=0.233, 50% EtOAc/Heptanes), only the desired product (Rf=0.416, 50% EtOAc/Heptanes). The reaction mixture was diluted with 20 mL DCM and filtered through a plug of silica, which was washed with additional DCM (100 mL). The filtrate was combined and the DCM was distilled off under atmospheric pressure to yield a final volume of about 30 mL (986 mg, 6.93 mmol). This solution was used in the next step without further purification.

### Step 3. Preparation of 5-bromo-6-chloro-N-((2,2-dimethyltetrahydro-2H-pyran-4-yl)methyl)pyridin-3-amine

To a solution of 5-bromo-6-chloropyridin-3-amine (450 mg, 2.169 mmol) in DCM (15 ml) was added 2,2-dimethyltetrahydro-2H-pyran-4-carbaldehyde (300 mg, 2.110 mmol), acetic acid (0.121 ml, 2.110 mmol) and sodium triacetoxyborohydride (671 mg, 3.16 mmol). The reaction mixture was stirred at room temperature for 2 hours. The solvent was removed and the crude material was redissolved in EtOAc (30 mL) which was washed with saturated sodium bicarbonate aqueous solution (30 mL), water (30 mL) and brine (30 mL), dried over sodium sulfate, filtrated and concentrated in vacuo. The resulting residue was purified by silica gel chromatography (eluted with 20-50% ethyl acetate:hexanes). The pure fractions were combined and concentrated in vacuo to yield the title compound (318 mg, 0.955 mmol, 45.2% yield). LCMS (m/z): 334.9 (MH⁺); retention time=0.96 min.

### Step 4. Preparation of 2,5′-dichloro-N-((2,2-dimethyltetrahydro-2H-pyran-4-yl)methyl)-2′-fluoro-3,4′-bipyridin-5-amine

A mixture of 5-bromo-6-chloro-N-((2,2-dimethyltetrahydro-2H-pyran-4-yl)methyl)pyridin-3-amine (400 mg, 1.199 mmol), 5-chloro-2-fluoropyridin-4-ylboronic acid (420 mg, 2.398 mmol) and 1,3-Bis(2,6-di-1-propylphenyl)imidazol-2-ylidene(1,4-naphthoquinone)palladium (0) dimer (157 mg, 0.120 mmol) in DME (5 ml) and sodium carbonate (2M aqueous solution, 2 mL, 4.00 mmol) was purged with argon and then heated at 120° C. for 2 hours. The reaction was cooled to room temperature and concentrated in vacuo to dryness. The resulting residue was redissolved in EtOAc (50 mL), washed with saturated sodium bicarbonate solution (50 mL), water (50 mL) and brine (50 mL). The organic layer was dried over Na₂SO₄ and concentrated in vacuo. The crude material was purified by silica gel chromatography (eluted with 10-50% EtOAc/Heptanes). The pure fractions were combined and concentrated in vacuo to yield the title compound (250 mg, 0.651 mmol, 54% yield). LCMS (m/z): 384.1 (MH⁺); retention time=0.97 min.

### EXAMPLES

### Example 1

### Compound 2

### Synthesis of 2,5′-dichloro-N2′-(trans-4-(2-methoxyethylamino)cyclohexyl)-N5-((tetrahydro-2H-pyran-4-yl)methyl)-3,4′-bipyridine-2′,5-diamine

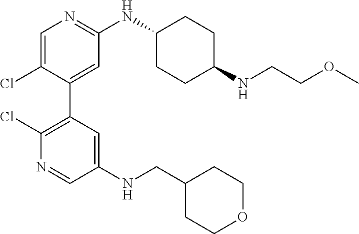 [SMILES: COCCN[C@H]1CC[C@H](Nc2cc(-c3cc(NCC4CCOCC4)cnc3Cl)c(Cl)cn2)CC1]

### Step 1. Synthesis of 2,5′-dichloro-2′-fluoro-N-((tetrahydro-2H-pyran-4-yl)methyl)-3,4′-bipyridin-5-amine

A mixture of Intermediate B (3 g, 8.84 mmol), 5-chloro-2-fluoropyridin-4-ylboronic acid (4.88 g, 27.8 mmol), 2 M Na₂CO₃ (17.67 mL, 35.3 mmol) in DME (48 mL) was purged with argon for 5 min in a glass bomb with stir bar followed by addition of PdCl₂(dppf).CH₂Cl₂ adduct (0.722 g, 0.884 mmol). The mixture was capped and heated at 100° C. for about 3 hours. LC/MS showed as a mixture of ˜50% starting material (M+H=305/307, retention time=0.86 min.), 30% product (M+H=356/358, retention time=0.89 min.), and 15% des-bromo starting material (M+H=227.0, retention time=0.64 min.). The reaction was diluted in 250 mL EtOAc, then washed with water (250 mL), sat NaHCO₃ (250 mL), and brine (200 mL). Organic layer was dried over Na₂SO₄, filtered, and concentrated in vacuo to give 6.2 g of dark brown oil. The residue was purified by column chromatography (ISCO, SiO2, 120 g, eluted with 100% heptane for 1 min, 20-50% EtOAc in heptane over 55 min, hold for 20 min). The product and des-bromo fractions were combined and concentrated in vacuo to yield 0.659 g brown gum which contained 66% product and 33% des-bromo starting material based upon LC/MS analysis. LC/MS of the product: 356/358 (MH⁺), retention time=0.89 min.

### Step 2

To the compound obtained in step 1 (659 mg, 1.110 mmol) at room temperature was added DIEA (0.388 ml, 2.220 mmol) and Intermediate A (300 mg, 1.741 mmol) in DMSO sequestially. The brown mixture was heated to 105° C. in a sealed glass flask for 16 hours. LC/MS showed as a mixture of desired product, starting material fluoropyridine and des-Br side product from the starting material in a ratio about 1.2:1:1. To the mixture was added additional DIEA (0.4 mL) and Intermediate A (200 mg in 2 mL of DMSO). The mixture was heated to 120° C. for about 24 hours. The reaction mixture was poured into water and extracted with EtOAc. The organic extracts were combined, washed with brine, dried with sodium sulfate and concentrated in vacuo to give 0.8 g of brown oil. The crude material was purified by HPLC (ACN in water with gradient 10%-50% in 35 minutes) twice in order to obtain pure product. The desired fractions were combined, basified with potassium carbonate (to pH>10), extracted with EtOAc, dried with sodium sulfate and concentrated in vacuo to give pure product. The pure product was lyophilized with MeCN and water (1:1) to give 110 mg of off-white powder. ¹H NMR (400 MHz, CHLOROFORM-d) δ ppm 1.14-1.47 (m, 4H) 1.63-2.23 (m, 9H) 2.45-2.58 (m, 1H) 2.83 (t, J=5.09 Hz, 2H) 3.04 (t, J=6.26 Hz, 1H) 3.32-3.57 (m, 7H) 3.92 (t, J=5.87 Hz, 1H) 4.01 (dd, J=11.15, 3.33 Hz, 2H) 4.46 (d, J=7.83 Hz, 1H) 6.26 (s, 1H) 6.78 (d, J=3.13 Hz, 1H) 7.84 (d, J=2.74 Hz, 1H) 8.12 (s, 1H) LC/MS: 508/510 (MH⁺), retention time=0.56 min.

### Example 2

### Compound 63

### Synthesis of N2′-(trans-4-aminocyclohexyl)-5′-chloro-N5-((tetrahydro-2H-pyran-4-yl)methyl)-3,4′-bipyridine-2′,5-diamine

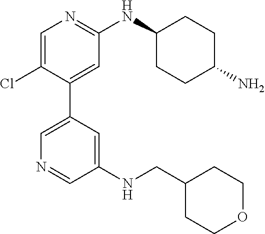 [SMILES: N[C@H]1CC[C@H](Nc2cc(-c3cncc(NCC4CCOCC4)c3)c(Cl)cn2)CC1]

### Step 1: Preparation of 5-bromo-N-((tetrahydro-2H-pyran-4-yl)methyl)pyridin-3-amine

To Pd(OAc)₂ (117 mg, 0.521 mmol) was added BINAP (389 mg, 0.625 mmol) and Dioxane (20 ml). The reaction was stirred for about 5 minutes at room temperature. To this mixture was added 3,5-dibromopyridine (2468 mg, 10.42 mmol), (tetrahydro-2H-pyran-4-yl)methanamine (600 mg, 5.21 mmol) and stirred for about 5 minutes. Finally potassium tert-butoxide (643 mg, 5.73 mmol) was added and the resulting mixture was stirred at 90-95° C. for 18 hours. The reaction was cooled to room temperature and 15 ml of ethyl acetate along with 5 ml of methanol was added. The mixture was filtered and concentrated dryness. The crude material was purified by silica gel chromatography (40 g column, eluting with 20-80% ethyl acetate in heptane). The desired fractions were combined and concentrated to yield 550 mg of the title compound as an off-white solid which was used without further purification. LCMS (m/z): 271.1/273.1 (MH⁺), retention time=0.44 min.

### Step 2: Preparation of 5′-chloro-2′-fluoro-N-((tetrahydro-2H-pyran-4-yl)methyl)-3,4′-bipyridin-5-amine

To 5-bromo-N-((tetrahydro-2H-pyran-4-yl)methyl)pyridin-3-amine (550 mg, 2.028 mmol) was added 5-chloro-2-fluoropyridin-4-ylboronic acid (711 mg, 4.06 mmol), PdCl₂(dppf).CH₂Cl₂ adduct (166 mg, 0.203 mmol), DME (9 ml) and sodium carbonate solution (2M aqueous, 3.04 ml, 6.09 mmol). The reaction was stirred at 100-105° C. for 2 hours and monitored by LCMS. The reaction was cooled to room temperature and 15 ml of ethyl acetate along with 15 ml of methanol was added. The resulting mixture was filtered and concentrated to dryness. The crude material was purified by silica gel chromatography (40 g column eluting with 20-80% ethyl acetate in heptane). The desired fractions were concentrated to yield 416 mg of the title compound as an off-white solid which was used without further purification. LCMS (m/z): 322.2 (MH⁺), retention time=0.53 min.

### Step 3: Preparation of N2′-(trans-4-aminocyclohexyl)-5′-chloro-N5-((tetrahydro-2H-pyran-4-yl)methyl)-3,4′-bipyridine-2′,5-diamine

To 5′-chloro-2′-fluoro-N-((tetrahydro-2H-pyran-4-yl)methyl)-3,4′-bipyridin-5-amine (240 mg, 0.746 mmol) was added DMSO (3 ml) and cyclohexane-trans-1,4-diamine (767 mg, 6.71 mmol). The reaction mixture was stirred at 100° C. for 18 hours. The reaction was cooled to room temperature and 2.0 ml of DMSO was added. The resulting mixture was filtered and purified by HPLC and lyophilized to yield the pure material as TFA salt. The product was free based by adding 350 ml of ethyl acetate and washed with saturated sodium bicarbonate solution (1×). The basic water layer was back extracted with ethyl acetate (2×). The organic layers were combined and washed with water (3×), brine (1×), dried with sodium sulfate, filtered and concentrate to dryness. The residue was dissolved in 1:1 ACN/water and lyophilized to yield 168 mg of the title compound as an off-white solid. LCMS (m/z): 416.2 (MH⁺), retention time=0.40 min. 1H NMR (300 MHz, METHANOL-d4, 25° C.) δ ppm 1.20-1.48 (m, 6H) 1.75 (d, J=12.89 Hz, 2H) 1.82-1.91 (m, 1H) 1.91-2.01 (m, 2H) 2.09 (d, J=9.67 Hz, 2H) 2.66-2.83 (m, 1H) 3.05 (d, J=6.74 Hz, 2H) 3.41 (td, J=11.72, 1.47 Hz, 2H) 3.58-3.74 (m, 1H) 3.96 (dd, J=11.28, 3.37 Hz, 2H) 6.47 (s, 1H) 7.03 (d, J=2.05 Hz, 1H) 7.74 (d, J=1.76 Hz, 1H) 7.95 (d, J=2.64 Hz, 1H) 7.99 (s, 1H).

### Example 3

### Compound 24

### Synthesis of N2′-(trans-4-aminocyclohexyl)-5′-chloro-N5-(3-fluorobenzyl)-3,4′-bipyridine-2′,5-diamine

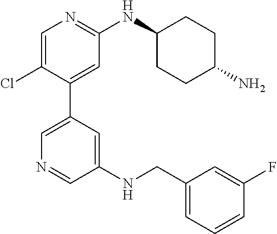 [SMILES: N[C@H]1CC[C@H](Nc2cc(-c3cncc(NCc4cccc(F)c4)c3)c(Cl)cn2)CC1]

### Step 1: Preparation of 5-bromo-N-(3-fluorobenzyl)pyridin-3-amine

To 3-bromo-5-fluoropyridine (600 mg, 3.41 mmol) was added DMSO (5 ml), (3-fluorophenyl)methanamine (1280 mg, 10.23 mmol) and TEA (0.570 ml, 4.09 mmol). The reaction mixture was microwaved at 205° C. for 30 minutes. The reaction was cooled to room temperature and 200 ml of ethyl acetate was added. The organic layer was separated, washed with saturated sodium bicarbonate solution (1×), water (2×), brine, dried with sodium sulfate, filtered and concentrated to dryness. The crude residue was purified by silica gel chromatography (40 g column eluting with 0-35% ethyl acetate in heptane). The desired fractions were concentrated to yield 370 mg of the title compound as an off-white solid. This was used without further purification. LCMS (m/z): 281.1/283.1 (MH⁺), retention time=0.63 min.

### Step 2: Preparation of 5′-chloro-2′-fluoro-N-(3-fluorobenzyl)-3,4′-bipyridin-5-amine

To 5-bromo-N-(3-fluorobenzyl)pyridin-3-amine (450 mg, 1.601 mmol) was added 5-chloro-2-fluoropyridin-4-ylboronic acid (505 mg, 2.88 mmol), PdCl₂(dppf).CH₂Cl₂ adduct (131 mg, 0.160 mmol), DME (7 ml) and 2M sodium carbonate solution (2.401 ml, 4.80 mmol). The reaction mixture was stirred at 100° C. for 2 hours. The reaction was cooled to room temperature and 25 ml of ethyl acetate along with 10 ml of methanol was added. The mixture was filtered and concentrated to dryness. The crude material was purified by silica gel chromotography (40 g column eluting with 10-50% ethyl acetate in heptanes). The desired fractions were concentrated to yield 449 mg of the title compound as an off-white solid. This was used without further purification. LCMS (m/z): 332.1 (MH⁺), retention time=0.69 min.

### Step 3: Preparation of N2′-(trans-4-aminocyclohexyl)-5′-chloro-N5-(3-fluorobenzyl)-3,4′-bipyridine-2′,5-diamine

To 5′-chloro-2′-fluoro-N-(3-fluorobenzyl)-3,4′-bipyridin-5-amine (180 mg, 0.543 mmol) was added cyclohexane-trans-1,4-diamine (558 mg, 4.88 mmol) and DMSO (2.8 ml). The reaction mixture was stirred at 100° C. for 18 hours. The reaction was cooled to room temperature, filtered and purified by HPLC and lyophilized to give desired pure product as TFA salt. The product was free based by adding 500 ml of ethyl acetate and washed with saturated sodium bicarbonate solution (1×). The basic water layer was back extracted with ethyl acetate. The organic layers were combined and washed with water (3×) and brine (1×), dried with sodium sulfate, filtered and concentrate to dryness. The residue was dissolved in 1:1 ACN/water and lyophilize to yield 162 mg of the title compound as an off-white solid. LCMS (m/z): 426.2 (MH⁺), retention time=0.52 min. 1H NMR (300 MHz, METHANOL-d4, 25° C.) δ ppm 1.16-1.44 (m, 4H) 1.93 (d, J=8.79 Hz, 2H) 2.06 (d, J=8.79 Hz, 2H) 2.62-2.79 (m, 1H) 3.53-3.71 (m, 1H) 4.40 (s, 2H) 6.41 (s, 1H) 6.91-7.03 (m, 2H) 7.11 (d, J=9.96 Hz, 1H) 7.19 (d, J=7.62 Hz, 1H) 7.27-7.40 (m, 1H) 7.76 (d, J=1.47 Hz, 1H) 7.90-8.00 (m, 2H).

### Example 4

### Compound 14

### Synthesis of N2′-(trans-4-aminocyclohexyl)-5′-chloro-N5-(3-(trifluoromethoxy)benzyl)-3,4′-bipyridine-2′,5-diamine

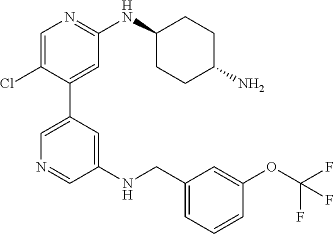 [SMILES: N[C@H]1CC[C@H](Nc2cc(-c3cncc(NCc4cccc(OC(F)(F)F)c4)c3)c(Cl)cn2)CC1]

### Step 1: Preparation of 5-bromo-5′-chloro-2′-fluoro-3,4′-bipyridine

To 3,5-dibromopyridine (811 mg, 3.42 mmol) was added 5-chloro-2-fluoropyridin-4-ylboronic acid (300 mg, 1.711 mmol), PdCl₂(dppf).CH₂Cl₂ adduct (140 mg, 0.171 mmol), DME (7 ml) and sodium carbonate aqueous solution (2 M, 2.57 ml, 5.13 mmol). The reaction was stirred at 85-90° C. for 2 hours. The reaction was cooled to room temperature and 20 ml of ethyl acetate was added. The mixture was filtered and concentrated to dryness. The crude material was purified by silica gel chromatography (40 g column eluting 0-25% ethyl acetate in hexane). The desired fractions were concentrated to yield 212 mg of the title compound as free base which was used without further purification. LCMS (m/z): 287.0/289.1 (MR), retention time=0.94 min.

### Step 2: Preparation of trans-N1-(5-bromo-5′-chloro-3,4′-bipyridin-2′-yl)cyclohexane-1,4-diamine

To 5-bromo-5′-chloro-2′-fluoro-3,4′-bipyridine (206 mg, 0.716 mmol) was added trans-cyclohexane-1,4-diamine (655 mg, 5.73 mmol), DMSO (2.5 ml) and TEA (0.120 ml, 0.860 mmol). The reaction mixture was stirred at 100° C. for 18 hours. The reaction was cooled to room temperature and 300 ml of ethyl acetate was added. The mixture was washed with saturated sodium bicarbonate (2×), water (2×), brine, dried with sodium sulfate, filtered and concentrated to yield 254 mg of the title compound as free base which was used without further purification. LCMS (m/z): 381.1/383.2 (MH⁺), retention time=0.54 min.

### Step 3: Preparation of N2′-(trans-4-aminocyclohexyl)-5′-chloro-N5-(3-(trifluoromethoxy)benzyl)-3,4′-bipyridine-2′,5-diamine

To Pd(OAc)₂ (2.470 mg, 0.011 mmol) was added BINAP (8.56 mg, 0.014 mmol), N1-(5-bromo-5′-chloro-3,4′-bipyridin-2′-yl)cyclohexane-trans-1,4-diamine (21 mg, 0.055 mmol), and dioxane (0.5 ml). The reaction mixture was stirred for 5 minutes at room temperature. To this mixture was added (3-(trifluoromethoxy)phenyl)methanamine (63.1 mg, 0.330 mmol) and stirred for about 5 minutes. Lastly potassium tert-butoxide (24.69 mg, 0.220 mmol) was added and the reaction was stirred at 95° C. for 45 minutes. The reaction was cooled to room temperature and 3 ml of ethyl acetate was added. The mixture was filtered and concentrated to dryness. The residue was dissolved in 1.0 ml of DMSO, filtered and purified by HPLC. After lypophilization, 6.5 mg of the title compound as an off-white solid, as a TFA salt was obtained. LCMS (m/z): 492.3 (MH⁺), retention time=0.61 min. ¹H NMR (300 MHz, METHANOL-d4, 25° C.) δ ppm 1.26-1.46 (m, 2H) 1.45-1.66 (m, 2H) 2.15 (d, J=16.70 Hz, 4H) 3.03-3.19 (m, 1H) 3.66-3.81 (m, 1H) 4.54 (s, 2H) 6.56 (s, 1H) 7.21 (d, J=7.62 Hz, 1H) 7.32 (br. s., 1H) 7.45 (dt, J=15.31, 7.73 Hz, 2H) 7.61 (br. s., 1H) 8.00-8.11 (m, 3H).

### Example 5

### Compound 16

### Synthesis of 2,5′-dichloro-N2′-(trans-4-((2-methoxyethyl)(methyl)amino)cyclohexyl)-N5-((tetrahydro-2H-pyran-4-yl)methyl)-3,4′-bipyridine-2′,5-diamine

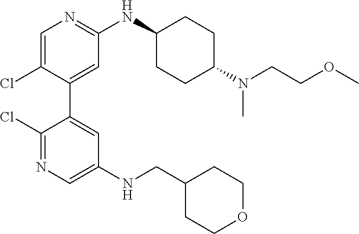 [SMILES: COCCN(C)[C@H]1CC[C@H](Nc2cc(-c3cc(NCC4CCOCC4)cnc3Cl)c(Cl)cn2)CC1]

### Step 1: Preparation of trans-4-(2,5′-dichloro-5-((tetrahydro-2H-pyran-4-yl)methyl)amino-3,4′-bipyridin-2′-ylamino)cyclohexanol

To 2,5′-dichloro-2′-fluoro-N-((tetrahydro-2H-pyran-4-yl)methyl)-3,4′-bipyridin-5-amine (Intermediate E) (250 mg, 0.702 mmol) was added trans-4-aminocyclohexanol (283 mg, 2.456 mmol), DMSO (2 ml) and then TEA (0.783 ml, 5.61 mmol). The reaction mixture was stirred at 95° C. for 72 hours. The reaction was cooled to room temperature and 1 ml of DMSO was added. The mixture was filtered, purified by HPLC and lyophilized to yield pure product as TFA salt. The product was free based with a solid support cartridge (PS bound NaHCO₃), flushed with methanol, and concentrated to yield 125 mg of the title compound as free base. LCMS (m/z): 451.2 (MH⁺), retention time=0.62 min.

### Step 2: Preparation of trans-4-(2,5′-dichloro-5-((tetrahydro-2H-pyran-4-yl)methyl)amino-3,4′-bipyridin-2′-ylamino)cyclohexyl methanesulfonate

To trans-4-(2,5′-dichloro-5-((tetrahydro-2H-pyran-4-yl)methyl)amino-3,4′-bipyridin-2′-ylamino)cyclohexanol (125 mg, 0.277 mmol) was added DCM (2 ml), TEA (0.058 ml, 0.415 mmol) and the mixture was cooled to 0° C. To the above mixture with stirring was added methanesulfonyl chloride (0.030 ml, 0.388 mmol). The reaction mixture was allowed to warm to room temperature and stirred for 2 hours. To the reaction was added 150 ml of ethyl acetate. The mixture was washed with saturated sodium bicarbonate (1×), water (2×), filtered through silica gel plug (1×1 inch) and concentrated to yield 145 mg of the title compound as free base which was used without further purification. LCMS (m/z): 529.2 (MH⁺), retention time=0.72 min.

### Step 3: Preparation of 2,5′-dichloro-N2′-(trans-4-((2-methoxyethyl)(methyl)amino)cyclohexyl)-N5-((tetrahydro-2H-pyran-4-yl)methyl)-3,4′-bipyridine-2′,5-diamine

To trans-4-(2,5′-dichloro-5-((tetrahydro-2H-pyran-4-yl)methyl)amino-3,4′-bipyridin-2′-ylamino)cyclohexyl methanesulfonate (62 mg, 0.117 mmol) was added t-butanol (0.45 ml) and 2-methoxy-N-methylethanamine (313 mg, 3.51 mmol). The reaction mixture was stirred at 95° C. for 5 hours. The reaction was cooled to room temperature and 12 ml of ethyl acetate was added. The mixture was washed with saturated sodium bicarbonate (1×), water (2×) and concentrated to dryness. The crude residue was dissolved in 1 ml of DMSO, filtered and purified by HPLC. After lyophilization, 7.8 mg of the title compound (an off-white solid), as a TFA salt was obtained. LCMS (m/z): 522.1 (MH⁺), retention time=0.58 min. ¹H NMR (300 MHz, METHANOL-d4, 25° C.) δ ppm 1.22-1.52 (m, 4H) 1.73 (d, J=13.19 Hz, 4H) 1.80-1.93 (m, 1H) 2.02-2.20 (m, 2H) 2.24 (br. s., 2H) 2.87 (s, 3H) 3.01 (d, J=6.45 Hz, 2H) 3.15-3.22 (m, 1H) 3.22-3.27 (m, 1H) 3.36-3.46 (m, 5H) 3.47-3.58 (m, 1H) 3.63-3.79 (m, 3H) 3.95 (dd, J=11.14, 3.22 Hz, 2H) 6.55 (s, 1H) 6.92 (d, J=2.93 Hz, 1H) 7.79 (d, J=2.93 Hz, 1H) 8.03 (s, 1H).

### Example 6

### Compound 26

### Synthesis of N2′-(trans-4-aminocyclohexyl)-5′-chloro-6-morpholino-N5-((tetrahydro-2H-pyran-4-yl)methyl)-3,4′-bipyridine-2′,5-diamine

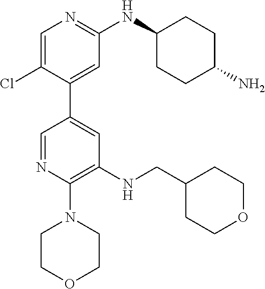 [SMILES: N[C@H]1CC[C@H](Nc2cc(-c3cnc(N4CCOCC4)c(NCC4CCOCC4)c3)c(Cl)cn2)CC1]

### Step 1: Preparation 5-bromo-2-morpholinopyridin-3-amine

To 5-bromo-2-fluoropyridin-3-amine (400 mg, 2.094 mmol) was added DMSO (2.5 ml) and morpholine (912 mg, 10.47 mmol). The reaction mixture was stirred at 110-115° C. for 40 hours. The reaction was cooled to room temperature and 200 ml of ethyl acetate was added. The mixture was washed with saturated sodium bicarbonate (2×), water (1×), brine (1×), dried with sodium sulfate, filtered and concentrated to yield 535 mg of the title compound as free base which was used without further purification. LCMS (m/z): 258.0/260.0 (MH⁺), retention time=0.52 min.

### Step 2: Preparation tert-butyl 5-bromo-2-morpholinopyridin-3-ylcarbamate

To 5-bromo-2-morpholinopyridin-3-amine (517 mg, 2.003 mmol) in DMF (6 ml) at 0° C. was sodium hydride (60% in mineral oil, 88 mg, 2.203 mmol). The ice bath was removed and the crude mixture was stirred for 20 minutes at room temperature. Then to the crude mixture was added di-tert-butyl dicarbonate (0.465 ml, 2.003 mmol) and the reaction mixture was stirred at 45° C. for about 16 hours. Additional sodium hydride (60% in mineral oil, 88 mg, 2.203 mmol) and di-tert-butyl dicarbonate (0.465 ml, 2.003 mmol) were added and the reaction mixture was stirred at 65° C. for 24 hours. The reaction was cooled to room temperature and 200 ml of ethyl acetated was added. The mixture was washed with saturated sodium bicarbonate (2×), water (2×) and brine (1×), dried with sodium sulfate, filtered and concentrated to dryness. The crude residue was purified by silica gel chromotography (40 g column eluting with 0-30% ethyl acetate in heptane). The desired fractions were combined and concentrated to yield 204 mg of the title compound as free base which was used without further purification. LCMS (m/z): 357.9/359.9 (MH⁺), retention time=1.10 min.

### Step 3: Preparation tert-butyl 5′-chloro-2′-fluoro-6-morpholino-3,4′-bipyridin-5-ylcarbamate

To tert-butyl 5-bromo-2-morpholinopyridin-3-ylcarbamate (200 mg, 0.558 mmol) was added 5-chloro-2-fluoropyridin-4-ylboronic acid (196 mg, 1.117 mmol), PdCl₂(dppf).CH₂Cl₂ adduct (45.6 mg, 0.056 mmol), DME (2.5 ml) and 2M sodium carbonate (0.837 ml, 1.675 mmol). The reaction was stirred at 110° C. for 1 hour. The reaction was cooled to room temperature and 15 ml of ethyl acetate along with 15 ml of methanol was added. The mixture was filtered and concentrated to dryness. The crude material was purified by silica gel chromatography (24 g ISCO column eluting with 0-30% ethyl acetate in heptane). The desired fractions were concentrated to yield 200 mg of the title compound as free base which was used without further purification. LCMS (m/z): 409.1 (MH⁺), retention time=1.04 min.

### Step 4: Preparation 5′-chloro-2′-fluoro-6-morpholino-N-((tetrahydro-2H-pyran-4-yl)methyl)-3,4′-bipyridin-5-amine

To tert-butyl 5′-chloro-2′-fluoro-6-morpholino-3,4′-bipyridin-5-ylcarbamate (120 mg, 0.294 mmol) in DMF (1.8 ml) was added sodium hydride (60% in mineral oil, 14.09 mg, 0.352 mmol). The resulting mixture was stirred for 20 minutes at room temperature. Then to the mixture was added (tetrahydro-2H-pyran-4-yl)methyl 4-methylbenzenesulfonate (95 mg, 0.352 mmol) and the reaction mixture was stirred at room temperature for 18 hours. To the resulting reaction mixture was added 50 ml of ethyl acetated. The mixture was then washed with saturated sodium bicarbonate (1×), water (2×) and brine (1×), dried with sodium sulfate, filtered and concentrated to give the crude intermediate which was used as is. To the intermediate was added 4M HCl in dioxane (3 ml, 12.00 mmol) and the reaction mixture was stirred at room temperature for 1 hour. This mixture was concentrated to dryness, dissolved in DMSO, filtered and purified by HPLC. After lyophilization, 50 mg of the title compound, as a TFA salt was obtained. LCMS (m/z): 407.1 (MH⁺), retention time=0.75 min.

### Step 5: Preparation N2′-(trans-4-aminocyclohexyl)-5′-chloro-6-morpholino-N5-((tetrahydro-2H-pyran-4-yl)methyl)-3,4′-bipyridine-2′,5-diamine

To 5′-chloro-2′-fluoro-6-morpholino-N-((tetrahydro-2H-pyran-4-yl)methyl)-3,4′-bipyridin-5-amine (18 mg, 0.044 mmol) was added DMSO (0.5 ml) and cyclohexane-trans-1,4-diamine (45.5 mg, 0.398 mmol). The reaction mixture was stirred at 100-105° C. for 18 hours. The reaction was cooled to room temperature and 0.5 ml of DMSO was added. The mixture was filtered and purified by HPLC. After lyophilization, 10.8 mg of the title compound, as a TFA salt was obtained as an off-white solid. LCMS (m/z): 501.3 (MH⁺), retention time=0.52 min. ¹H NMR (300 MHz, METHANOL-d4, 25° C.) δ ppm 1.28-1.49 (m, 4H) 1.50-1.65 (m, 2H) 1.68 (br. s., 2H) 1.87-2.03 (m, 1H) 2.16 (br. s., 4H) 3.11 (d, J=7.03 Hz, 2H) 3.13-3.22 (m, 5H) 3.35-3.49 (m, 2H) 3.63-3.78 (m, 1H) 3.85-3.92 (m, 4H) 3.96 (dd, J=11.28, 3.66 Hz, 2H) 6.67 (s, 1H) 7.12 (d, J=1.76 Hz, 1H) 7.64 (d, J=2.05 Hz, 1H) 8.04 (s, 1H).

### Example 7

### Compound 25

### Synthesis of 2,5′-dichloro-N5-((tetrahydro-2H-pyran-4-yl)methyl)-N2′-(trans-4-(((R)-tetrahydrofuran-2-yl)methylamino)cyclohexyl)-3,4′-bipyridine-2′,5-diamine

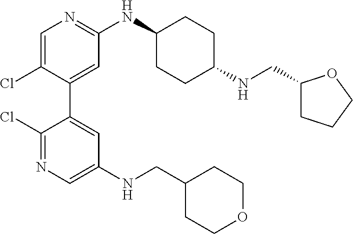 [SMILES: Clc1cnc(N[C@H]2CC[C@H](NC[C@H]3CCCO3)CC2)cc1-c1cc(NCC2CCOCC2)cnc1Cl]

### Step 1: Preparation (R)-(tetrahydrofuran-2-yl)methyl methanesulfonate

To (R)-(tetrahydrofuran-2-yl)methanol (600 mg, 5.87 mmol) was added DCM (35 ml), TEA (0.983 ml, 7.05 mmol) and then methanesulfonyl chloride (0.467 ml, 5.99 mmol) dropwise. The reaction mixture was stirred at room temperature for 5 hours. The resulting mixture was washed with saturated sodium bicarbonate (1×), water (2×), filtered and concentrate to yield 980 mg of the title compound as free base which was used without further purification. LCMS (m/z): 181.0 (MH⁺), retention time=0.40 min.

### Step 2: Preparation of 2,5′-dichloro-N5-((tetrahydro-2H-pyran-4-yl)methyl)-N2′-(trans-4-(((R)-tetrahydrofuran-2-yl)methylamino)cyclohexyl)-3,4′-bipyridine-2′,5-diamine

To N2′-(trans-4-aminocyclohexyl)-2,5′-dichloro-N5-((tetrahydro-2H-pyran-4-yl)methyl)-3,4′-bipyridine-2′,5-diamine (Intermediate F) (40 mg, 0.089 mmol) was added potassium carbonate (30.7 mg, 0.222 mmol), DMSO (0.4 ml) and then (R)-(tetrahydrofuran-2-yl)methyl methanesulfonate (24.01 mg, 0.133 mmol). The reaction was stirred at 100° C. for 3 hours. Additional (R)-(tetrahydrofuran-2-yl)methyl methanesulfonate (24.01 mg, 0.133 mmol) was added and the reaction continued at 100° C. for 3 additional hours, for a total of 6 hours. The reaction was cooled to room temperature and 0.5 ml of DMSO was added. The mixture was filtered and purified by HPLC. After lyophilization, 12.2 mg of the title compound, as a TFA salt was obtained as an off-white solid. LCMS (m/z): 534.2 (MH⁺), retention time=0.61 min. ¹H NMR (300 MHz, METHANOL-d4, 25° C.) δ ppm 1.24-1.45 (m, 4H) 1.48-1.67 (m, 3H) 1.73 (d, J=13.19 Hz, 2H) 1.79-1.91 (m, 1H) 1.91-2.05 (m, 2H) 2.06-2.31 (m, 5H) 2.94-3.05 (m, 3H) 3.13-3.22 (m, 2H) 3.41 (t, J=11.28 Hz, 2H) 3.72 (t, J=11.28 Hz, 1H) 3.77-3.87 (m, 1H) 3.87-4.01 (m, 3H) 4.06-4.19 (m, 1H) 6.52 (s, 1H) 6.91 (d, J=2.93 Hz, 1H) 7.78 (d, J=2.93 Hz, 1H) 8.02 (s, 1H).

### Example 8

### Compound 31

### Synthesis of 5′-chloro-N2′-(trans-4-(dimethylamino)cyclohexyl)-N5-(3-fluorobenzyl)-3,4′-bipyridine-2′,5-diamine

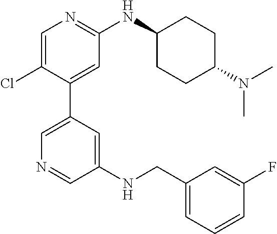 [SMILES: CN(C)[C@H]1CC[C@H](Nc2cc(-c3cncc(NCc4cccc(F)c4)c3)c(Cl)cn2)CC1]

### Step 1: Preparation of 5′-chloro-N2′-(trans-4-(dimethylamino)cyclohexyl)-N5-(3-fluorobenzyl)-3,4′-bipyridine-2′,5-diamine

To N2′-(trans-4-aminocyclohexyl)-5′-chloro-N5-(3-fluorobenzyl)-3,4′-bipyridine-2′,5-diamine (Example 3) (10 mg, 0.023 mmol) was added MeOH (0.5 ml), acetic acid (0.040 ml, 0.704 mmol) and formaldehyde 37% in water (0.021 ml, 0.282 mmol). The reaction mixture was stirred at room temperature for 5 minutes then sodium triacetoxyborohydride (24.88 mg, 0.117 mmol) was added. After 3 hours, additional sodium triacetoxyborohydride (24.88 mg, 0.117 mmol) was added and the reaction continued at room temperature for total of 24 hours. The solvent was removed in vacuo. The resulting residue was dissolved in 1.0 ml of DMSO, filtered and purified by HPLC. After lyophilization, 7.4 mg of the title compound, as a TFA salt was obtained as an off-white solid. LCMS (m/z): 454.1 (MH⁺), retention time=0.54 min. ¹H NMR (300 MHz, METHANOL-d4, 25° C.) δ ppm 1.23-1.53 (m, 2H) 1.55-1.80 (m, 2H) 2.06-2.33 (m, 4H) 2.87 (s, 6H) 3.19-3.27 (m, 1H) 3.75 (dddd, J=15.02, 7.69, 3.96, 3.81 Hz, 1H) 4.51 (s, 2H) 6.57 (s, 1H) 7.02 (td, 1H) 7.14 (d, J=9.96 Hz, 1H) 7.22 (d, J=7.33 Hz, 1H) 7.31-7.46 (m, 1H) 7.63 (br. s., 1H) 7.99-8.14 (m, 3H).

### Example 9

### Compound 36

### Synthesis of 1-(4-((2′-(trans-4-aminocyclohexylamino)-5′-chloro-3,4′-bipyridin-5-ylamino)methyl)piperidin-1-yl)ethanone

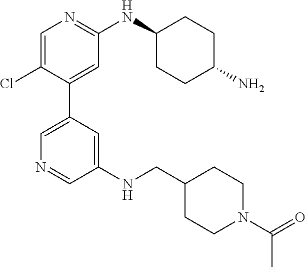 [SMILES: CC(=O)N1CCC(CNc2cncc(-c3cc(N[C@H]4CC[C@H](N)CC4)ncc3Cl)c2)CC1]

### Step 1: Preparation of tert-butyl 4-((2′-(trans-4-aminocyclohexylamino)-5′-chloro-3,4′-bipyridin-5-ylamino)methyl)piperidine-1-carboxylate

To Pd(OAc)₂ (13.23 mg, 0.059 mmol) was added BINAP (44.0 mg, 0.071 mmol) and dioxane (1.1 ml) the reaction was stirred 5 minutes at room temperature. Then to the mixture was added N1-(5-bromo-5′-chloro-3,4′-bipyridin-2′-yl)cyclohexane-trans-1,4-diamine (90 mg, 0.236 mmol) and tert-butyl 4-(aminomethyl)piperidine-1-carboxylate (177 mg, 0.825 mmol). The mixture was stirred for 3-5 minutes then lastly potassium tert-butoxide (79 mg, 0.707 mmol) was added. The reaction mixture was stirred at 95° C. for 1 hour. The reaction was cooled to room temperature and 3 ml of ethyl acetate along with 1 ml of methanol was added. The mixture was filtered and concentrated to dryness. The residue was dissolved in DMSO, filtered and purified by HPLC. After lyophilization, 40 mg of the title compound, as a TFA salt was obtained. LCMS (m/z): 515.2 (MH⁺), retention time=0.60 min.

### Step 2: Preparation of benzyl trans-4-(5′-chloro-5-(piperidin-4-ylmethylamino)-3,4′-bipyridin-2′-ylamino)cyclohexylcarbamate

To tert-butyl 4-((2′-(trans-4-aminocyclohexylamino)-5′-chloro-3,4′-bipyridin-5-ylamino)methyl)piperidine-1-carboxylate (40 mg, 0.078 mmol) was added DCM (0.75 ml), TEA (0.022 ml, 0.155 mmol) and benzyl 2,5-dioxopyrrolidin-1-yl carbonate (29.0 mg, 0.116 mmol). The reaction mixture was stirred at room temperature for 1 hour. The solution was concentrated and 50 ml of ethyl acetate was added. The resulting solution was washed with saturated sodium bicarbonate (2×), water (1×), brine (1×), dried with sodium sulfate, filtered through silica gel plug, and concentrated to give the intermediate which was used as is. To the obtained intermediate was added 10% TFA in DCM (6 ml, 7.79 mmol). The mixture was stirred at room temperature for 1 hour. The reaction was concentrated to dryness to yield the title compound as TFA salt, assumed in quantitative yield which was used without further purification. LCMS (m/z): 549.3 (MH⁺), retention time=0.63 min.

### Step 3: Preparation of benzyl trans-4-(5-((1-acetylpiperidin-4-yl)methylamino)-5′-chloro-3,4′-bipyridin-2′-ylamino)cyclohexylcarbamate

To benzyl trans-4-(5′-chloro-5-(piperidin-4-ylmethylamino)-3,4′-bipyridin-2′-ylamino)cyclohexylcarbamate (20 mg, 0.036 mmol) was added DCM (1 ml), TEA (0.020 ml, 0.146 mmol) and acetic anhydride (6.87 μl, 0.073 mmol). The reaction mixture was stirred at room temperature for 1 hour. The solvent was removed in vacuo. The resulting residue was dissolved in 1.0 ml of DMSO, filtered and purified by HPLC. After lyophilization, 14 mg of the title compound, as a TFA salt was obtained. LCMS (m/z): 591.3 (MH⁺), retention time=0.68 min.

### Step 4: Preparation of 1-(4-((2′-(trans-4-aminocyclohexylamino)-5′-chloro-3,4′-bipyridin-5-ylamino)methyl)piperidin-1-yl)ethanone

To benzyl trans-4-(5-((1-acetylpiperidin-4-yl)methylamino)-5′-chloro-3,4′-bipyridin-2′-ylamino)cyclohexylcarbamate (14 mg, 0.024 mmol) was added ACN (1.5 ml) and then TMSI (trimethylsilyl iodide, 6.45 μl, 0.047 mmol). The reaction mixture was stirred at room temperature for 20 minutes. The solvent was removed in vacuo. The resulting residue was dissolved in 0.8 ml of DMSO, filtered and purified by HPLC. After lyophilization, 5.4 mg of the title compound, as a TFA salt was obtained as an off-white solid. LCMS (m/z): 457.2 (MH⁺), retention time=0.40 min. ¹H NMR (300 MHz, METHANOL-d4, 25° C.) δ ppm 1.09-1.47 (m, 4H) 1.54 (t, J=12.31 Hz, 2H) 1.80-2.00 (m, 3H) 2.03-2.25 (m, 7H) 2.56-2.70 (m, 1H) 3.05-3.20 (m, 4H) 3.68-3.84 (m, 1H) 3.96 (d, J=13.77 Hz, 1H) 4.55 (d, J=13.48 Hz, 1H) 6.59 (s, 1H) 7.67 (s, 1H) 8.02 (s, 1H) 8.05-8.12 (m, 2H).

### Example 10

### Compound 39

### Synthesis of N2′-(trans-4-(aminomethyl)cyclohexyl)-5′-chloro-N5-(3-fluorobenzyl)-3,4′-bipyridine-2′,5-diamine

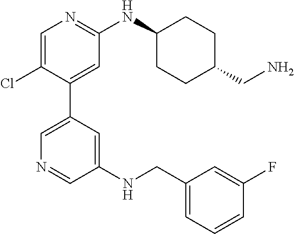 [SMILES: NC[C@H]1CC[C@H](Nc2cc(-c3cncc(NCc4cccc(F)c4)c3)c(Cl)cn2)CC1]

### Step 1: Preparation of N2′-(trans-4-(aminomethyl)cyclohexyl)-5′-chloro-N5-(3-fluorobenzyl)-3,4′-bipyridine-2′,5-diamine

To 5′-chloro-2′-fluoro-N-(3-fluorobenzyl)-3,4′-bipyridin-5-amine (Example 3, step 2) (21 mg, 0.063 mmol) was added DMSO (0.4 ml), TEA (0.018 ml, 0.127 mmol) and tert-butyl (trans-4-aminocyclohexyl)methylcarbamate (57.8 mg, 0.253 mmol). The mixture was flushed with argon and heated at 100° C. for 40 hours. The resulting mixture was concentrated in vacuo to remove the excess amine and afford an intermediate which was used as is. To this intermediate was added HCl in dioxane (4M, 1.0 mL, 4.00 mmol) and the mixture was stirred at room temperature for 90 minutes. The solvent was removed in vacuo. The resulting residue was dissolved in (0.75 ml of DMSO with 0.075 ml of water), filtered and purified by HPLC. After lyophilization, 15.3 mg of the title compound, as a TFA salt was obtained (an off-white solid). LCMS (m/z): 440.2 (MH⁺), retention time=0.53 min.

### Example 11

### Compound 55

### Synthesis of N2′-(trans-4-aminocyclohexyl)-5′-chloro-6-fluoro-N5-((tetrahydro-2H-pyran-4-yl)methyl)-3,4′-bipyridine-2′,5-diamine

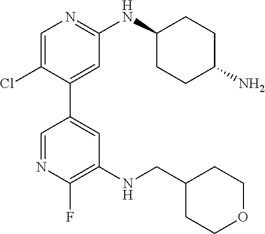 [SMILES: N[C@H]1CC[C@H](Nc2cc(-c3cnc(F)c(NCC4CCOCC4)c3)c(Cl)cn2)CC1]

### Step 1: Preparation of 5-bromo-2-fluoro-N-((tetrahydro-2H-pyran-4-yl)methyl)pyridin-3-amine. (tetrahydro-2H-pyran-4-yl)methyl 4-methylbenzenesulfonate

To DMF (1.5 ml) was added NaH (60% in mineral oil, 46.1 mg, 1.152 mmol) and then 5-bromo-2-fluoropyridin-3-amine (200 mg, 1.047 mmol). The reaction mixture was stirred at room temperature for 15 minutes. Then (tetrahydro-2H-pyran-4-yl)methyl 4-methylbenzenesulfonate (283 mg, 1.047 mmol) was added and stirred at 40° C. for 40 hours. The reaction was cooled to room temperature and 100 ml of ethyl acetate was added. The resulting mixture was washed with saturated sodium bicarbonate (2×), water (2×), brine, dried sodium sulfate, filtered and concentrated to dryness. The residue was purified by silica gel chromatography (40 g column eluting with 0-40% ethyl acetate in heptane). The desired fractions were concentrated to yield 104 mg of the title compound as free base. LCMS (m/z): 288.9/290.9 (MH⁺), retention time=0.88 min.

### Step 2: Preparation of 5′-chloro-2′,6-difluoro-N-((tetrahydro-2H-pyran-4-yl)methyl)-3,4′-bipyridin-5-amine

To 5-bromo-2-fluoro-N-((tetrahydro-2H-pyran-4-yl)methyl)pyridin-3-amine (92 mg, 0.318 mmol) was added 5-chloro-2-fluoropyridin-4-ylboronic acid (167 mg, 0.955 mmol), PdCl₂(dppf).CH₂Cl₂ adduct (26.0 mg, 0.032 mmol), DME (2.1 ml) and last 2M sodium carbonate (0.636 ml, 1.273 mmol). The reaction mixture was stirred at 100° C. for 2 hours. The reaction was cooled to room temperature and 10 ml of ethyl acetate along with 5 ml of methanol was added. The mixture was filtered and concentrated to dryness. The residue was purified by silica gel chromatography (12 g column eluting with 0-35% ethyl acetate in heptane). The desired fractions were concentrated to constant mass, giving 55 mg of the title compound as free base. LCMS (m/z): 340.0 (MH⁺), retention time=0.92 min.

### Step 3: Preparation of N2′-(trans-4-aminocyclohexyl)-5′-chloro-6-fluoro-N5-((tetrahydro-2H-pyran-4-yl)methyl)-3,4′-bipyridine-2′,5-diamine

To 5′-chloro-2′,6-difluoro-N-((tetrahydro-2H-pyran-4-yl)methyl)-3,4′-bipyridin-5-amine (26 mg, 0.077 mmol) was added DMSO (0.5 ml) and trans-cyclohexane-1,4-diamine (79 mg, 0.689 mmol). The reaction was stirred at 85-90° C. for 6 hours. The reaction was cooled to room temperature and 0.5 ml of DMSO was added. The mixture was filtered and purified by HPLC. After lyophilization, 19.0 mg of the title compound, as a TFA salt was obtained (an off-white solid). LCMS (m/z): 434.1 (MH⁺), retention time=0.55 min. ¹H NMR (300 MHz, METHANOL-d4, 25° C.) δ ppm 1.22-1.49 (m, 4H) 1.48-1.65 (m, 2H) 1.72 (d, J=13.19 Hz, 2H) 1.92 (dddd, J=11.03, 7.36, 4.10, 3.88 Hz, 1H) 2.03-2.25 (m, 4H) 3.10 (d, J=7.03 Hz, 2H) 3.12-3.21 (m, 1H) 3.39 (t, J=11.14 Hz, 2H) 3.64-3.79 (m, J=11.10, 7.44, 3.77, 3.77 Hz, 1H) 3.95 (dd, J=11.14, 3.52 Hz, 2H) 6.63 (s, 1H) 7.08-7.19 (m, 1H) 7.33 (s, 1H) 8.03 (s, 1H)

### Example 12

### Compound 44

### Synthesis of N2′-(trans-4-aminocyclohexyl)-N5-(3-fluorobenzyl)-3,4′-bipyridine-2′,5-diamine

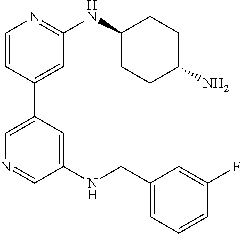 [SMILES: N[C@H]1CC[C@H](Nc2cc(-c3cncc(NCc4cccc(F)c4)c3)ccn2)CC1]

### Step 1: Preparation of 5-bromo-N-(3-fluorobenzyl)pyridin-3-amine

To 3-fluoro-5-bromo-pyridine (300 mg, 1.705 mmol) was added DMSO (3 ml), Amine (853 mg, 6.82 mmol) and TEA (0.285 ml, 2.046 mmol). The reaction was stirred at 105° C. for 72 hours followed by LCMS. The excess amine was partially concentrated off. The crude solution was filtered and purified by HPLC. After lyophilization, 45.0 mg of the title compound, as a TFA salt was obtained. LCMS (m/z): 281.0/283.1 (MH⁺), retention time=0.67 min.

### Step 2: Preparation of 2′-fluoro-N-(3-fluorobenzyl)-3,4′-bipyridin-5-amine

To 5-bromo-N-(3-fluorobenzyl)pyridin-3-amine (35 mg, 0.125 mmol) was added 2-fluoropyridin-4-ylboronic acid (31.6 mg, 0.224 mmol), PdCl₂(dppf).CH₂Cl₂ adduct (15.25 mg, 0.019 mmol), DME (0.9 ml), ethanol (0.2 ml) and then 2M sodium carbonate (0.249 ml, 0.498 mmol). The reaction mixture was stirred at 85° C. for 2 hours. The reaction was cooled to room temperature and 3 ml of ethyl acetate along with 1 ml of methanol was added. The mixture was filtered and concentrated to dryness. The residue was dissolved in 1.2 ml of DMSO, filtered and purified by HPLC. After lyophilization, 27 mg of the title compound, as a TFA salt was obtained. LCMS (m/z): 298.1 (MH⁺), retention time=0.63 min.

### Step 3: Preparation of N2′-(trans-4-aminocyclohexyl)-N5-(3-fluorobenzyl)-3,4′-bipyridine-2′,5-diamine

To 2′-fluoro-N-(3-fluorobenzyl)-3,4′-bipyridin-5-amine (21 mg, 0.071 mmol) was added DMSO (0.6 ml) and cyclohexane-trans-1,4-diamine (64.5 mg, 0.565 mmol). The reaction mixture was stirred at 105° C. for 22 hours. The reaction was cooled to room temperature and 0.5 ml of DMSO was added. The mixture was filtered and purified by HPLC. After lyophilization, 17.1 mg of the title compound, as a TFA salt was obtained (an off-white solid). LCMS (m/z): 392.2 (MH⁺), retention time=0.44 min. ¹H NMR (300 MHz, METHANOL-d4, 25° C.) δ ppm 1.40-1.74 (m, 4H) 2.17 (t, J=13.63 Hz, 4H) 3.10-3.25 (m, 1H) 3.65-3.81 (m, 1H) 4.52 (s, 2H) 7.01 (td, J=8.42, 1.90 Hz, 1H) 7.08 (dd, J=6.74, 1.47 Hz, 1H) 7.14 (d, J=9.96 Hz, 1H) 7.18-7.28 (m, 2H) 7.31-7.44 (m, 1H) 7.58 (br. S., 1H) 7.95 (d, J=6.45 Hz, 1H) 8.10 (d, J=2.34 Hz, 1H) 8.22 (s, 1H)

### Example 13

### Compound 77

N2′-(trans-4-aminocyclohexyl)-5′,6-dichloro-N5-((tetrahydro-2H-pyran-4-yl)methyl)-3,4′-bipyridine-2′,5-diamine

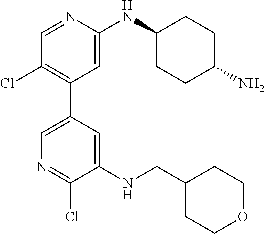 [SMILES: N[C@H]1CC[C@H](Nc2cc(-c3cnc(Cl)c(NCC4CCOCC4)c3)c(Cl)cn2)CC1]

### Step 1. Preparation of 5-bromo-2-chloro-N-((tetrahydro-2H-pyran-4-yl)methyl)pyridin-3-amine

To a scintillation vial containing 5-bromo-2-chloropyridin-3-amine (1.3 g, 6.27 mmol) was added DMF (20 ml) and NaH (0.301 g, 7.52 mmol). After 20 min stirring at room temperature, (tetrahydro-2H-pyran-4-yl)methyl 4-methylbenzenesulfonate (1.694 g, 6.27 mmol) was added. The reaction mixture was stirred at room temperature for 58 hours. The reaction mixture was diluted with EtOAc and washed with H₂O and brine. The organic layer was dried Na₂SO₄, filtered and concentrated. The resulting residue was purified by column chromatography on silica gel (22% EtOAc/Hexane) to yield 5-bromo-2-chloro-N-((tetrahydro-2H-pyran-4-yl)methyl)pyridin-3-amine (1.27 g, 4.16 mmol, 66.3% yield) as brown oil. LCMS (m/z): 305.0 (MH⁺), retention time=0.89 min.

### Step 2: Preparation of 5′,6-dichloro-2′-fluoro-N-((tetrahydro-2H-pyran-4-yl)methyl)-3,4′-bipyridin-5-amine

To a suspension of 5-bromo-2-chloro-N-((tetrahydro-2H-pyran-4-yl)methyl)pyridin-3-amine (1 g, 3.27 mmol), Na₂CO₃ (4.25 ml, 8.51 mmol) and 5-chloro-2-fluoropyridin-4-ylboronic acid (0.975 g, 5.56 mmol) in DME (20 ml) was added PdCl₂(dppf).CH₂Cl₂ adduct (0.214 g, 0.262 mmol). The reaction mixture was capped and heated to 100° C. for 4 hours with an oil bath. The reaction mixture was diluted with EtOAc and washed with H₂O, then brine. The organic layer was dried Na₂SO₄, filtered and concentrated. The crude material was purified by column chromatography on silica gel (25% EtOAc/Hexane) to yield 5′,6-dichloro-2′-fluoro-N-((tetrahydro-2H-pyran-4-yl)methyl)-3,4′-bipyridin-5-amine (693 mg, 1.945 mmol, 59.5% yield). LCMS (m/z): 356.0 (MH⁺), retention time=0.96 min.

### Step 3: Preparation of N2′-(trans-4-aminocyclohexyl)-5′,6-dichloro-N5-((tetrahydro-2H-pyran-4-yl)methyl)-3,4′-bipyridine-2′,5-diamine

To a scintillation vial containing 5′,6-dichloro-2′-fluoro-N-((tetrahydro-2H-pyran-4-yl)methyl)-3,4′-bipyridin-5-amine (477 mg, 1.339 mmol) and TEA (0.373 ml, 2.68 mmol) was added DMSO (5 ml) and cyclohexane-trans-1,4-diamine (1529 mg, 13.39 mmol). The homogenous reaction mixture was capped and heated to 100° C. in an oil bath for 3 hours. The reaction mixture was diluted with DCM and washed with water, then brine. The organic layer was dried Na₂SO₄, filtered and concentrated. The crude material was purified by reverse phase preparative HPLC. The collected fractions were combined and concentrated to one third of the original volume. The solution was neutralized with sat. NaHCO₃ solution and extracted with DCM. The organic layer was washed with brine, dried over Na₂SO₄ and concentrated to dryness. The resulting pure product was dissolved in 20 ml MeCN and 20 ml water and lyophilized to yield N2′-(trans-4-aminocyclohexyl)-5′,6-dichloro-N5-((tetrahydro-2H-pyran-4-yl)methyl)-3,4′-bipyridine-2′,5-diamine (400 mg, 0.888 mmol, 66.3% yield) as a white power. LCMS (m/z): 450.1 (MH⁺), retention time=0.57 min. ¹H NMR (400 MHz, CHLOROFORM-d) δ ppm 1.17-1.33 (m, 4H), 1.34-1.47 (m, 2H), 1.68-1.78 (m, 1H), 1.91 (br. S., 1H), 2.07-2.18 (m, 1H), 2.67-2.78 (m, 1H), 3.11 (t, J=6.26 Hz, 2H), 3.36-3.46 (m, 2H), 3.48-3.61 (m, 1H), 3.98-4.07 (m, 2H), 4.38-4.46 (m, 1H), 4.54 (t, J=5.67 Hz, 1H), 6.29 (s, 1H), 6.97 (d, J=1.57 Hz, 1H), 7.73 (d, J=1.56 Hz, 1H), 8.12 (s, 1H).

### Example 14

### Compound 76

### 2,5′-dichloro-N2′-(trans-4-(pyrrolidin-1-yl)cyclohexyl)-N5-((tetrahydro-2H-pyran-4-yl)methyl)-3,4′-bipyridine-2′,5-diamine

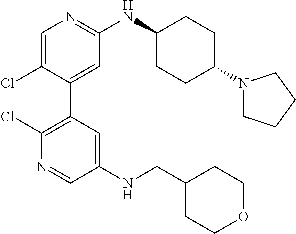 [SMILES: Clc1cnc(N[C@H]2CC[C@H](N3CCCC3)CC2)cc1-c1cc(NCC2CCOCC2)cnc1Cl]

### Step 1. Preparation of 2,5′-dichloro-N2′-(trans-4-(pyrrolidin-1-yl)cyclohexyl)-N5-((tetrahydro-2H-pyran-4-yl)methyl)-3,4′-bipyridine-2′,5-diamine

To a scintillation vial containing N2′-(trans-4-aminocyclohexyl)-2,5′-dichloro-N5-((tetrahydro-2H-pyran-4-yl)methyl)-3,4′-bipyridine-2′,5-diamine (16 mg, 0.028 mmol) (Intermediate F) and K₂CO₃ (3.92 mg, 0.028 mmol) was added DMF (1 ml) and 1,4-dibromobutane (3.36 μl, 0.028 mmol). The reaction mixture was capped and heated to 60° C. for 3 hours. The crude solution was concentrated and purified by reverse phase preparative HPLC to yield 2,5′-dichloro-N2′-(trans-4-(pyrrolidin-1-yl)cyclohexyl)-N5-((tetrahydro-2H-pyran-4-yl)methyl)-3,4′-bipyridine-2′,5-diamine as an off-white solid (4.9 mg, 7.92 μmol, 27.9% yield), LCMS (m/z): 504.2 (MH⁺), retention time=0.58 min as a TFA salt after lyophilyzing. ¹H NMR (400 MHz, METHANOL-d4) δ ppm 1.27-1.48 (m, 4H) 1.73 (d, J=12.91 Hz, 4H) 1.79-1.92 (m, 1H) 2.01 (t, J=6.26 Hz, 2H) 2.10-2.33 (m, 6H) 3.01 (d, J=6.65 Hz, 2H) 3.09-3.23 (m, 3H) 3.36-3.46 (m, 2H) 3.59-3.78 (m, 3H) 3.95 (dd, J=11.35, 3.13 Hz, 2H) 6.57 (s, 1H) 6.92 (d, J=3.13 Hz, 1H) 7.79 (d, J=2.74 Hz, 1H) 8.01-8.05 (m, 1H).

### Example 15

### Compound 78

### 5′,6-dichloro-N2′-(trans-4-(2-methoxyethylamino)cyclohexyl)-N5-((tetrahydro-2H-pyran-4-yl)methyl)-3,4′-bipyridine-2′,5-diamine

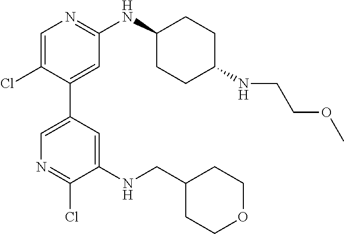 [SMILES: COCCN[C@H]1CC[C@H](Nc2cc(-c3cnc(Cl)c(NCC4CCOCC4)c3)c(Cl)cn2)CC1]

### Step 1. Preparation of 5′,6-dichloro-N2′-(trans-4-(2-methoxyethylamino)cyclohexyl)-N5-((tetrahydro-2H-pyran-4-yl)methyl)-3,4′-bipyridine-2′,5-diamine

To a scintillation vial containing N2′-(trans-4-aminocyclohexyl)-5′,6-dichloro-N5-((tetrahydro-2H-pyran-4-yl)methyl)-3,4′-bipyridine-2′,5-diamine (Example 13, 16 mg, 0.036 mmol) and K₂CO₃ (4.91 mg, 0.036 mmol) was added DMF (1 ml) and 1-bromo-2-methoxyethane (4.9 mg, 0.036 mmol). The reaction mixture was capped and heated to 60° C. for 3 hours. The crude solution was concentrated and purified by reverse phase preparative HPLC to yield 5′,6-dichloro-N2′-(trans-4-(2-methoxyethylamino)cyclohexyl)-N5-((tetrahydro-2H-pyran-4-yl)methyl)-3,4′-bipyridine-2′,5-diamine an off-white solid (6.0 mg, 0.012 mmol, 33.2% yield), LCMS (m/z): 508.2 (MH⁺), retention time=0.60 min as a TFA salt after lyophilyzing. ¹H NMR (400 MHz, METHANOL-d4) δ ppm 1.27-1.45 (m, 5H) 1.50-1.65 (m, 3H) 1.70 (d, J=12.91 Hz, 3H) 1.86-1.99 (m, 1H) 2.21 (d, J=10.96 Hz, 5H) 3.15 (d, J=6.65 Hz, 4H) 3.21-3.27 (m, 3H) 3.42 (s, 7H), 3.61-3.68 (m, 3H) 3.68-3.79 (m, 1H) 3.91-3.99 (m, 3H) 6.60 (s, 1H) 7.08-7.14 (m, 1H) 7.56-7.63 (m, 1H) 8.00-8.07 (m, 1H).

### Example 16

### Compound 87

### Synthesis of 2′-(trans-4-aminocyclohexylamino)-5′-chloro-5-((tetrahydro-2H-pyran-4-yl)methyl)amino-3,4′-bipyridin-6-ol

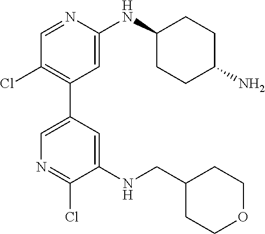 [SMILES: N[C@H]1CC[C@H](Nc2cc(-c3cnc(Cl)c(NCC4CCOCC4)c3)c(Cl)cn2)CC1]

### Step 1. Preparation of N2′-(trans-4-aminocyclohexyl)-5′-chloro-6-methoxy-N5-((tetrahydro-2H-pyran-4-yl)methyl)-3,4′-bipyridine-2′,5-diamine

Following the synthesis sequence in Example 13, using 5-bromo-2-methoxypyridin-3-amine as starting material. LCMS (m/z): 446.1 (MH⁺), retention time=0.58 min.

### Step 2. Preparation of 2′-(trans-4-aminocyclohexylamino)-5′-chloro-5-((tetrahydro-2H-pyran-4-yl)methyl)amino-3,4′-bipyridin-6-ol

To a scintillation vial containing N2′-(trans-4-aminocyclohexyl)-5′-chloro-6-methoxy-N5-((tetrahydro-2H-pyran-4-yl)methyl)-3,4′-bipyridine-2′,5-diamine (26 mg, 0.046 mmol) was added water (0.2 ml), and HCl (1 ml, 0.717 mmol). The reaction mixture was stirred at 80° C. for 8 hours. The resulting mixture was purified by reverse phase preparative HPLC to yield 2′-(trans-4-aminocyclohexylamino)-5′-chloro-5-((tetrahydro-2H-pyran-4-yl)methyl)amino-3,4′-bipyridin-6-ol an off-white solid (7 mg, 0.013 mmol, 27.6% yield), LCMS (m/z): 432.3 (MH⁺), retention time=0.43 min as a TFA salt after lyophilyzing. ¹H NMR (400 MHz, METHANOL-d₄) δ ppm 1.27-1.50 (m, 4H) 1.52-1.66 (m, 2H) 1.66-1.76 (m, 2H) 1.87-2.00 (m, 1H) 2.06-2.23 (m, 4H) 3.06 (d, J=6.65 Hz, 2H) 3.09-3.21 (m, 1H) 3.35-3.47 (m, 2H) 3.63-3.74 (m, 1H) 3.95 (dd, J=10.96, 3.52 Hz, 2H) 6.42-6.48 (m, 1H) 6.72 (s, 1H) 6.87-6.94 (m, 1H), 8.00 (s, 1H).

### Example 17

### Compound 72

### Synthesis of 2,5′-dichloro-N5-((tetrahydro-2H-pyran-4-yl)methyl)-N2′-(trans-4-((R)-3,3,3-trifluoro-2-methoxypropylamino)cyclohexyl)-3,4′-bipyridine-2′,5-diamine

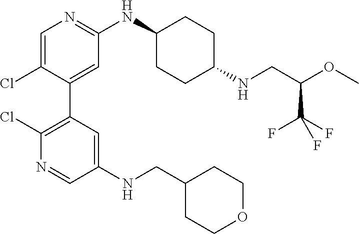 [SMILES: CO[C@H](CN[C@H]1CC[C@H](Nc2cc(-c3cc(NCC4CCOCC4)cnc3Cl)c(Cl)cn2)CC1)C(F)(F)F]

### Preparation of 2,5′-dichloro-N5-((tetrahydro-2H-pyran-4-yl)methyl)-N2′-(trans-4-((R)-3,3,3-trifluoro-2-methoxypropylamino)cyclohexyl)-3,4′-bipyridine-2′,5-diamine

To a solution of Intermediate E (40 mg, 0.11 mmol) in DMSO (0.3 mL) was added N1-((R)-3,3,3-trifluoro-2-methoxypropyl)cyclohexane-trans-1,4-diamine (Intermediate H, 81 mg, 0.34 mmol) and 2,6-lutidine (0.039 mL, 0.23 mmol). The mixture was stirred at 135° C. for 3 hours. The cooled reaction mixture was purified by reverse phase HPLC and lyophilized to give 41 mg of 2,5′-dichloro-N5-((tetrahydro-2H-pyran-4-yl)methyl)-N2′-(trans-4-((R)-3,3,3-trifluoro-2-methoxypropylamino)cyclohexyl)-3,4′-bipyridine-2′,5-diamine as its TFA salt (an off-white solid). LCMS (m/z): 576.4 (MH⁺), retention time=0.65 min.; ¹H NMR (400 MHz, DMSO-d6): δ ppm 1.08-1.32 (m, 4H) 1.37-1.58 (m, 2H) 1.63 (d, J=12.52 Hz, 2H) 1.68-1.84 (m, J=10.86, 7.24, 3.86, 3.86 Hz, 1H) 1.93-2.20 (m, 4H) 2.93 (d, J=6.65 Hz, 2H) 3.15 (d, J=9.00 Hz, 2H) 3.20-3.36 (m, 4H) 3.57 (s, 3H) 3.83 (dd, J=11.35, 2.74 Hz, 2H) 4.24-4.38 (m, 1H) 6.40 (s, 1H) 6.80 (br. s., 1H) 6.89 (d, J=2.74 Hz, 1H) 7.80 (d, J=2.74 Hz, 1H) 8.05 (s, 1H) 8.77 (br. s., 1H) 8.86 (br. s., 1H).

### Example 18

### Compound 74

### (R)-3-(trans-4-(2,5′-dichloro-5-((tetrahydro-2H-pyran-4-yl)methyl)amino-3,4′-bipyridin-2′-ylamino)cyclohexylamino)-1,1,1-trifluoropropan-2-ol

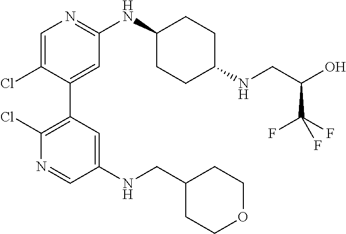 [SMILES: O[C@H](CN[C@H]1CC[C@H](Nc2cc(-c3cc(NCC4CCOCC4)cnc3Cl)c(Cl)cn2)CC1)C(F)(F)F]

### Preparation of (R)-3-(trans-4-(2,5′-dichloro-5-((tetrahydro-2H-pyran-4-yl)methyl)amino-3,4′-bipyridin-2′-ylamino)cyclohexylamino)-1,1,1-trifluoropropan-2-ol

To a solution of Intermediate F (31 mg, 0.069 mmol) in 2-propanol (0.4 mL) was added (R)-(+)-3,3,3-trifluoro-1,2-epoxypropane (7.7 mg, 0.069 mmol). The mixture was stirred at 70° C. for 2 hours. The reaction mixture was concentrated. The residue was purified by reverse phase HPLC and lyophilized to give 26 mg of (R)-3-(trans-4-(2,5′-dichloro-5-((tetrahydro-2H-pyran-4-yl)methyl)amino-3,4′-bipyridin-2′-ylamino)cyclohexylamino)-1,1,1-trifluoropropan-2-ol as its TFA salt (an off-white solid). LCMS (m/z): 562.3 (MH⁺), rt=0.60 min; ¹H NMR (400 MHz, DMSO-d6): 6 ppm 1.06-1.31 (m, 4H) 1.35-1.58 (m, 2H) 1.63 (d, J=12.91 Hz, 2H) 1.68-1.84 (m, 1H) 2.05-2.2 (m, 4H) 2.93 (d, J=6.26 Hz, 2H) 3.00-3.19 (m, 2H) 3.25 (t, J=10.76 Hz, 3H) 3.83 (dd, J=11.35, 2.74 Hz, 2H) 4.40 (d, J=6.65 Hz, 1H) 6.39 (s, 1H) 6.72-6.86 (m, 1H) 6.89 (d, J=2.74 Hz, 1H) 7.24 (br. s., 1H) 7.80 (d, J=3.13 Hz, 1H) 8.05 (s, 1H) 8.73 (br. s., 1H).

### Example 19

### Compound 1

### Synthesis of aacemic 2,5′-dichloro-N5-((tetrahydro-2H-pyran-4-yl)methyl)-N2′-(trans-4-(tetrahydrofuran-3-ylamino)cyclohexyl)-3,4′-bipyridine-2′,5-diamine

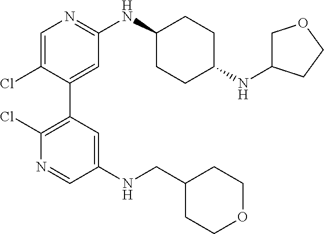 [SMILES: Clc1cnc(N[C@H]2CC[C@H](NC3CCOC3)CC2)cc1-c1cc(NCC2CCOCC2)cnc1Cl]

### Step 1. Preparation of racemic benzyl trans-4-(tetrahydrofuran-3-ylamino)cyclohexylcarbamate

To a stirred solution of benzyl trans-4-aminocyclohexylcarbamate (396 mg, 1.595 mmol) in CH₂Cl₂ (9 ml) was added dihydrofuran-3(2H)-one (151 mg, 1.754 mmol) followed by acetic acid (150 μL, 2.62 mmol) and sodium triacetoxyborohydride (439 mg, 2.073 mmol) under argon. The reaction was stirred at 25° C. for 16 hours, then concentrated in vacuo. The residue was partitioned between EtOAc and 1M NaOH. The organics were combined, then washed with 1M NaOH (×2), water (×2), brine (×2), then dried (Na₂SO₄), filtered and evaporated under reduced pressure to give racemic benzyl trans-4-(tetrahydrofuran-3-ylamino)cyclohexylcarbamate (495 mg). The residue was used in next step without further purification.

### Step 2. Preparation of racemic tert-butyl trans-4-aminocyclohexyl(tetrahydrofuran-3-yl)carbamate

To a stirred solution of racemic benzyl trans-4-(tetrahydrofuran-3-ylamino)cyclohexylcarbamate (495 mg, 1.555 mmol) in CH₂Cl₂ (5 ml) was added BOC-anhydride (0.397 ml, 1.710 mmol). The reaction was stirred at 25° C. under argon for 21 hours. The mixture was evaporated under reduced pressure and purified by flash column chromatography (silica gel; 15% to 25% EtOAc/hexane). A solution of the resultant Boc protected intermediate (135 mg, 0.323 mmol) in MeOH (5 mL) was hydrogenated under an atmosphere of hydrogen in the presence of 10% Pd/C (24 mg, 0.226 mmol) for 18 hours. The mixture was then filtered through celite and the filtrate was evaporated under reduced pressure to yield racemic tert-butyl trans-4-aminocyclohexyl(tetrahydrofuran-3-yl)carbamate (100 mg). The residue was used in next step without further purification.

### Step 3. Preparation of racemic 2,5′-dichloro-N5-((tetrahydro-2H-pyran-4-yl)methyl)-N2′-(trans-4-(tetrahydrofuran-3-ylamino)cyclohexyl)-3,4′-bipyridine-2′,5-diamine

To a scintillation vial was added 2,5′-dichloro-2′-fluoro-N-((tetrahydro-2H-pyran-4-yl)methyl)-3,4′-bipyridin-5-amine (18 mg, 0.051 mmol), racemic tert-butyl trans-4-aminocyclohexyl(tetrahydrofuran-3-yl)carbamate (21 mg, 0.074 mmol), DIPEA (17.6 μl, 0.101 mmol) and NMP (0.1 ml). This mixture was heated at 110° C. for 48 hours. The mixture was diluted with EtOAc and washed with water (×2), brine (×2), then dried (Na₂SO₄), filtered and evaporated under reduced pressure. The resultant residue was dissolved in CH₂Cl₂ (0.4 mL) and treated with TFA (100 μl, 1.298 mmol). After 1 hour, the mixture was concentrated in vacuo and the residue was purified by reverse phase preparative HPLC and then lyophilized to yield racemic 2,5′-dichloro-N6-((tetrahydro-2H-pyran-4-yl)methyl)-N2′-(trans-4-(tetrahydrofuran-3-ylamino)cyclohexyl)-2,4′-bipyridine-2′,6-diamine (6.3 mg, an off-white solid), LCMS (m/z): 520.1/522.0/524.2 (bis-chloro isotopic signature for MH⁺), retention time=0.57 min as a TFA salt.

### Example 20

### Compound 3

### Synthesis of N^{2′}-(trans-4-aminocyclohexyl)-5′-chloro-N⁵-((1,1-dioxo-tetrahydro-2H-1-thiopyran-4-yl)methyl)-3,4′-bipyridine-2′,5-diamine

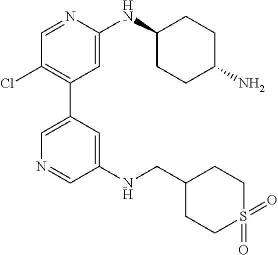 [SMILES: N[C@H]1CC[C@H](Nc2cc(-c3cncc(NCC4CCS(=O)(=O)CC4)c3)c(Cl)cn2)CC1]

### Step 1: Preparation of toluene-4-sulfonic acid 1,1-dioxo-hexahydro-1-thiopyran-4-yl-methyl ester

A 100 ml round bottom flask equipped with magnetic stir bar was charged with (1,1-dioxo-tetrahydro-2H-1-thiopyran-4-yl)-methanol (2.5 g, 15.22 mmol, synthesized according to procedures described in the literature: Organic Process Research & Development 2008, 12, 892-895.), pyridine (25 ml) and toluenesulfonyl chloride (2.90 g, 15.22 mmol). The reaction mixture was stirred for 18 hours at 50° C. The reaction mixture was concentrated in vacuo to dryness. The crude material was purified by flash chromatograph (0-70% ethyl acetate in heptane) to yield 3.78 g of the title compound. LCMS (m/z): 319.0 (MH⁺), retention time=0.71 min.

### Step 2: Preparation of 5-bromo-N-((1,1-dioxo-tetrahydro-2H-1-thiopyran-4-yl)methyl)pyridin-3-amine

To DMF (5.0 ml) was added NaH (60% in mineral oil, 111 mg, 4.62 mmol) and 5-bromopyridin-3-amine (400 mg, 2.31 mmol). The reaction mixture was stirred at room temperature for 15 minutes. Then (1,1-dioxo-tetrahydro-2H-thiopyran-4-yl)methyl 4-methylbenzenesulfonate (736 mg, 2.31 mmol) was added and stirred at 50° C. for 18 hours. The reaction was cooled to room temperature and 100 ml of ethyl acetate was added. The resulting mixture was washed with saturated sodium bicarbonate (2×), water (2×), brine, dried with sodium sulfate, filtered and concentrated to dryness. The residue was purified by silica gel chromatography (40 g column eluting with 0-70% ethyl acetate in heptane). The desired fractions were concentrated to yield 270 mg of the title compound as free base. LCMS (m/z): 318.9/320.9 (MH⁺), retention time=0.41 min.

### Step 3: Preparation of 5′-chloro-2′-fluoro-N-((1,1-dioxo-tetrahydro-2H-thiopyran-4-yl)methyl)-3,4′-bipyridin-5-amine

To 5-bromo-N-((1,1-dioxo-tetrahydro-2H-1-thiopyran-4-yl)methyl)pyridin-3-amine (180 mg, 0.564 mmol) was added 5-chloro-2-fluoropyridin-4-ylboronic acid (198 mg, 1.128 mmol), PdCl₂(dppf).CH₂Cl₂ adduct (36.8 mg, 0.045 mmol), THF (3.0 ml) and last 2M sodium carbonate aqueous solution (0.733 ml, 1.466 mmol). The reaction mixture was stirred at 80° C. for 6 hours. The reaction was cooled to room temperature and 10 ml of ethyl acetate along with 5 ml of methanol was added. The mixture was filtered and concentrated to dryness. The residue was purified by silica gel chromatography (12 g column eluting with 0-35% ethyl acetate in heptane). The desired fractions were concentrated to constant mass, giving 65 mg of the title compound as free base. LCMS (m/z): 370.0 (MH⁺), retention time=0.49 min.

### Step 4: Preparation of N²-(trans-4-aminocyclohexyl)-5′-chloro-N⁵-((1,1-dioxo-tetrahydro-2H-1-thiopyran-4-yl)methyl)-3,4′-bipyridine-2′,5-diamine

To a solution of 5′-chloro-2′-fluoro-N-((1,1-dioxo-tetrahydro-2H-thiopyran-4-yl)methyl)-3,4′-bipyridin-5-amine (40 mg, 0.108 mmol) in DMSO (0.4 ml) was added trans-cyclohexane-1,4-diamine (124 mg, 1.082 mmol). The mixture was stir at 100° C. for 2 hours. The reaction was cooled to room temperature and 0.5 ml of DMSO was added. The mixture was filtered and purified by HPLC. After lyophilization, 10 mg of the title compound, as a TFA salt was obtained (an off-white solid). LCMS (m/z): 464.1 (MH⁺), retention time=0.38 min.

### Example 21

### Synthesis of trans-N1-(5′-chloro-5-(3-fluorobenzyloxy)-3,4′-bipyridin-2′-yl)cyclohexane-1,4-diamine

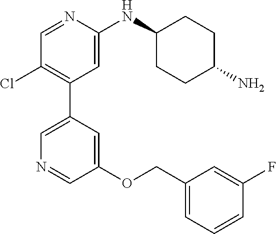 [SMILES: N[C@H]1CC[C@H](Nc2cc(-c3cncc(OCc4cccc(F)c4)c3)c(Cl)cn2)CC1]

### Step 1: Preparation of 3-bromo-5-(3-fluorobenzyloxy)pyridine

To 5-bromopyridin-3-ol (125 mg, 0.718 mmol) was added (3-fluorophenyl)methanol (181 mg, 1.437 mmol), THF (1.0 mL), triphenylphosphine (377 mg, 1.437 mmol) and stirred to dissolve. Then DEAD (0.227 mL, 1.437 mmol) was added. The reaction mixture was stirred at room temperature for 1 hr. The reaction mixture was concentrated under reduced pressure. The residue was purified by silica gel chromatography using a 12 g column eluting with 0%-30% ethyl acetate in hexane. The desired fractions were concentrated under reduced pressure giving 150 mg of titled compound. LCMS (m/z): 282.0/284.0 [M+H]+; Retention time=0.99 min.

### Step 2: Preparation of 5′-chloro-2′-fluoro-5-(3-fluorobenzyloxy)-3,4′-bipyridine

To 3-bromo-5-(3-fluorobenzyloxy)pyridine (144 mg, 0.510 mmol) was added 5-chloro-2-fluoropyridin-4-ylboronic acid (134 mg, 0.766 mmol), PdCl₂(dppf) CH₂Cl₂ adduct (50.0 mg, 0.061 mmol), DME (3 mL) and last 2M aqueous sodium carbonate solution (1.02 mL, 2.042 mmol). The reaction mixture was stirred at 100° C. for 2 hrs. The reaction mixture was cooled to room temperature, 10 mL of ethyl acetate was added, filtered and concentrated to crude product. The crude was purified by silica gel chromatography using 12 g column eluting with 0%-35% ethyl acetate in hexane. The desired fractions were concentrated under reduced pressure, giving 108 mg of titled compound. LCMS (m/z): 333.1 [M+H]+; Retention time=0.94 min.

### Step 3: Preparation of trans-N1-(5′-chloro-5-(3-fluorobenzyloxy)-3,4′-bipyridin-2′-yl)cyclohexane-1,4-diamine

To 5′-chloro-2′-fluoro-5-(3-fluorobenzyloxy)-3,4′-bipyridine (33 mg, 0.099 mmol) was added DMSO (0.8 mL), trans-cyclohexane-1,4-diamine (79 mg, 0.694 mmol) and TEA (0.028 mL, 0.198 mmol). The reaction mixture was stirred at 105° C. for 20 hrs. The reaction mixture was allowed to cool to room temperature, 0.25 mL of DMSO was added, filtered and purified by HPLC. After lypohilization, 37.8 mg of the title compound, as a trifluoroacetic acid salt was obtained.

LCMS (m/z): 427.3 [M+H]+; Retention time=0.63 min. ¹H NMR (400 MHz, chloroform-d3) δ ppm 1.29-1.43 (m, 2H) 1.55 (qd, J=12.65, 3.13 Hz, 2H) 2.08 (d, J=12.13 Hz, 2H) 2.17 (d, J=11.35 Hz, 2H) 3.05-3.17 (m, 1H) 3.71 (tt, J=11.35, 3.72 Hz, 1H) 5.25 (s, 2H) 6.57 (s, 1H) 7.06 (td, J=8.51, 2.15 Hz, 1H) 7.22 (d, J=9.78 Hz, 1H) 7.27 (d, J=7.83 Hz, 1H) 7.40 (td, J=7.83, 5.87 Hz, 1H) 7.59-7.64 (m, 1H) 8.03 (s, 1H) 8.22 (d, J=1.57 Hz, 1H) 8.41 (d, J=2.74 Hz, 1H).

### Example 22

### Synthesis of N-(trans-4-(aminomethyl)cyclohexyl)-5′-chloro-5-(3-fluorobenzyloxy)-3,4′-bipyridin-2′-amine

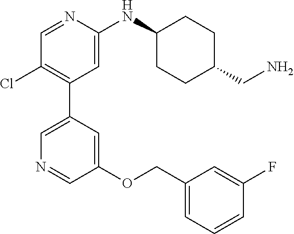 [SMILES: NC[C@H]1CC[C@H](Nc2cc(-c3cncc(OCc4cccc(F)c4)c3)c(Cl)cn2)CC1]

### Step 1: Preparation of N-(trans-4-(aminomethyl)cyclohexyl)-5′-chloro-5-(3-fluorobenzyloxy)-3,4′-bipyridin-2′-amine

To 5′-chloro-2′-fluoro-5-(3-fluorobenzyloxy)-3,4′-bipyridine (33 mg, 0.099 mmol) was added DMSO (0.8 mL), TEA (0.028 mL, 0.198 mmol) and tert-butyl (trans-4-aminocyclohexyl)methylcarbamate (45.3 mg, 0.198 mmol). The reaction mixture was flushed with argon and heated at 100-105° C. for 40 hrs. Most of the DMSO was removed under reduced pressure. To the crude material was added 4M HCl in dioxane (1.5 mL, 6.0 mmol) and the mixture was stirred at room temperature for 90 min. The solvent was concentrated off under reduced pressure, the residue was dissolved in 1.0 mL of DMSO with 0.075 mL of water and purified by HPLC. After lypohilization, 45.4 mg of the title compound, as a trifluoroacetic acid salt was obtained. LCMS (m/z): 441.3 [M+H]+; Retention time=0.64 min. ¹H NMR (400 MHz, chloroform d3) δ ppm 1.13-1.39 (m, 4H) 1.64 (ddd, J=11.05, 7.53, 3.72 Hz, 1H) 1.89 (d, J=12.52 Hz, 2H) 2.13 (d, J=12.13 Hz, 2H) 2.82 (d, J=7.04 Hz, 2H) 3.59-3.75 (m, 2H) 5.25 (s, 2H) 6.61 (s, 1H) 7.06 (td, J=8.41, 2.35 Hz, 1H) 7.22 (d, J=9.78 Hz, 1H) 7.27 (d, J=7.83 Hz, 1H) 7.35-7.45 (m, 1H) 7.58-7.63 (m, 1H) 8.02 (s, 1H) 8.22 (d, J=1.57 Hz, 1H) 8.42 (d, J=2.35 Hz, 1H).

### Example 23

### Synthesis of (1R,2R)- and (1S,2S)-2-((trans-4-((2,5′-dichloro-5-(((tetrahydro-2H-pyran-4-yl)methyl)amino)-[3,4′-bipyridin]-2′-yl)amino)cyclohexyl)amino)cyclohexanol

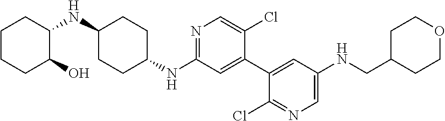 [SMILES: O[C@H]1CCCC[C@@H]1N[C@H]1CC[C@H](Nc2cc(-c3cc(NCC4CCOCC4)cnc3Cl)c(Cl)cn2)CC1]

N2′-(trans-4-aminocyclohexyl)-2,5′-dichloro-N5-((tetrahydro-2H-pyran-4-yl)methyl)-[3,4′-bipyridine]-2′,5-diamine trifluoroacetic acid salt (32 mg, 0.071 mmol) was dissolved in acetonitrile (1 mL). Si-carbonate (˜500 mg; Silicycle; particle size: 40-63 mikroM; loading: 0.8 mmol/g; lot#: 37446; cat#: R66030B) was added and the mixture was stirred for 30 min. The mixture was filtered through a syringe filter. Rinsed with 0.5 mL of acetonitrile. 7-Oxabicyclo[4.1.0]heptane (70 mg, 0.713 mmol) and lithium perchlorate (200 mg, 1.880 mmol) were added and the mixture was heated in a sealed tube under argon at 53° C. for ˜16 hrs. Additional 7-Oxabicyclo[4.1.0]heptane (70 mg, 0.713 mmol) and lithium perchlorate (200 mg, 1.880 mmol) were added and heating was continued for additional 1 hr. The mixture was allowed to cool to room temperature, diluted with ˜0.5 mL of water and 1 mL of methanol and concentrated under reduced pressure. The residue was dissolved in DMSO, filtered through a syringe filter and purified by preparative HPLC providing a mixture of (1R,2R)- and (1S,2S)-2-((trans-4-((2,5′-dichloro-5-(((tetrahydro-2H-pyran-4-yl)methyl)amino)-[3,4′-bipyridin]-2′-yl)amino)cyclohexyl)amino)cyclohexanol as its trifluoroacetic acid salt (6.8 mg).

Slightly yellowish solid. LCMS (m/z): 548.2 [M+H]+; Retention time=0.70 min.

Table 1 depicts compounds of the invention that were made by methods described herein and provides some physical property data consistent with the assigned structures. Where the word ‘chiral’ appears with the structure, it indicates that the compound was tested as one isomer; where the structure illustrates absolute stereochemistry but the word ‘chiral’ is not present with the structure, the structure depicts relative stereochemistry of the chiral centers but the tested compound was not optically active.

TABLE 1

| | | | | retention |
|---|---|---|---|---|
| Compnd | | | | time |
| No. | MOLSTRUCTURE | Method | M + 1(m/z) | (min.) |
| 1 | 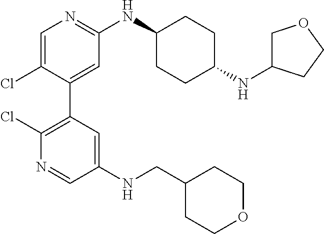 [SMILES: Clc1cnc(N[C@H]2CC[C@H](NC3CCOC3)CC2)cc1-c1cc(NCC2CCOCC2)cnc1Cl] | Example 19 | 520.1/522.0/ 524.2 (bis- chloro isotopic signature for MH⁺) | 0.57 |
| 2 | 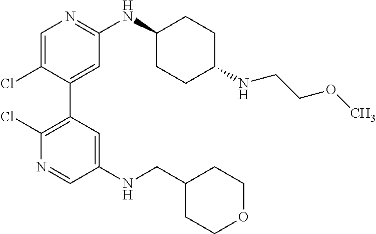 [SMILES: COCCN[C@H]1CC[C@H](Nc2cc(-c3cc(NCC4CCOCC4)cnc3Cl)c(Cl)cn2)CC1] | Example 1 | 508/510 | 0.56 |
| 3 | 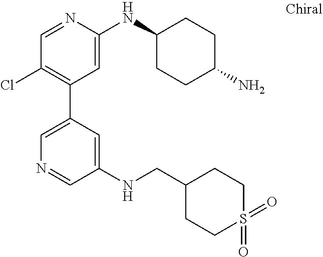 [SMILES: N[C@H]1CC[C@H](Nc2cc(-c3cncc(NCC4CCS(=O)(=O)CC4)c3)c(Cl)cn2)CC1] | Example 20 | 464.1/466.1 | 0.37 |
| 4 | 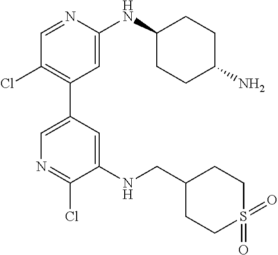 [SMILES: N[C@H]1CC[C@H](Nc2cc(-c3cnc(Cl)c(NCC4CCS(=O)(=O)CC4)c3)c(Cl)cn2)CC1] | Example 20 (using 5-bromo- 2- chloropyri- din-3- amine as the staring material) | 498.0/499.9 | 0.53 |
| 5 | 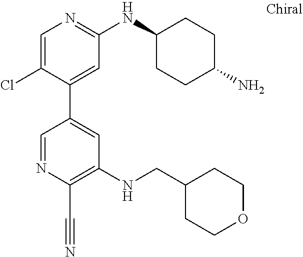 [SMILES: N#Cc1ncc(-c2cc(N[C@H]3CC[C@H](N)CC3)ncc2Cl)cc1NCC1CCOCC1] | Example 1, Intermediate G | 441.2 | 0.56 |
| 6 | 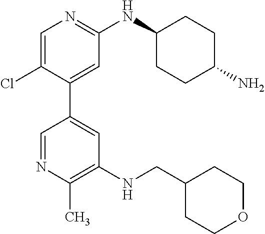 [SMILES: Cc1ncc(-c2cc(N[C@H]3CC[C@H](N)CC3)ncc2Cl)cc1NCC1CCOCC1] | Example 1 | 430.1 | 0.42 |
| 7 | 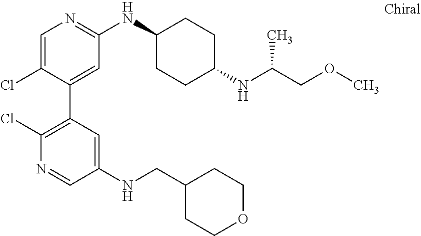 [SMILES: COC[C@@H](C)N[C@H]1CC[C@H](Nc2cc(-c3cc(NCC4CCOCC4)cnc3Cl)c(Cl)cn2)CC1] | Example 1, intermediate C | 522/524 | 0.61 |
| 8 | 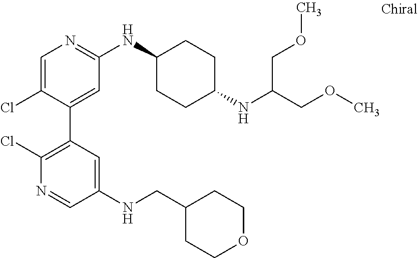 [SMILES: COCC(COC)N[C@H]1CC[C@H](Nc2cc(-c3cc(NCC4CCOCC4)cnc3Cl)c(Cl)cn2)CC1] | Example 1, intermediate D | 552/554 | 0.61 |
| 9 | 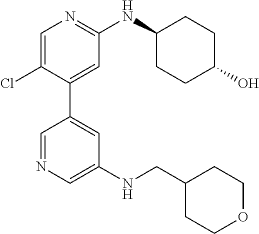 [SMILES: O[C@H]1CC[C@H](Nc2cc(-c3cncc(NCC4CCOCC4)c3)c(Cl)cn2)CC1] | Example 2 | 417.3 | 0.43 |
| 10 | 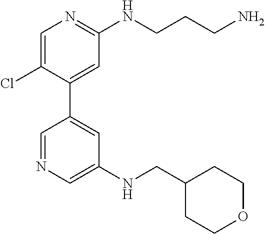 [SMILES: NCCCNc1cc(-c2cncc(NCC3CCOCC3)c2)c(Cl)cn1] | Example 2 | 376.3 | 0.39 |
| 11 | 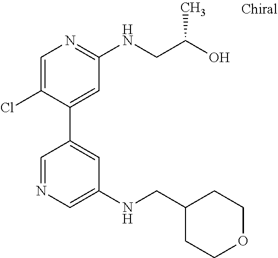 [SMILES: C[C@H](O)CNc1cc(-c2cncc(NCC3CCOCC3)c2)c(Cl)cn1] | Example 2 | 377.2 | 0.42 |
| 12 | 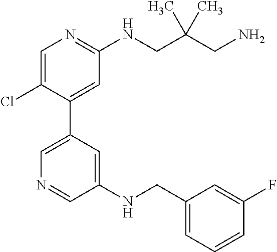 [SMILES: CC(C)(CN)CNc1cc(-c2cncc(NCc3cccc(F)c3)c2)c(Cl)cn1] | Example 3 | 414.2 | 0.54 |
| 13 | 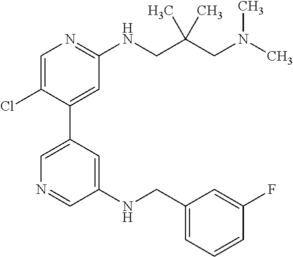 [SMILES: CN(C)CC(C)(C)CNc1cc(-c2cncc(NCc3cccc(F)c3)c2)c(Cl)cn1] | Example 3 | 442.3 | 0.56 |
| 14 | 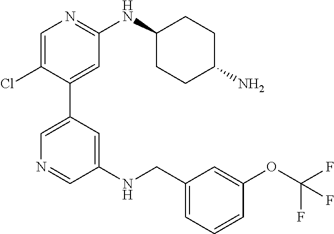 [SMILES: N[C@H]1CC[C@H](Nc2cc(-c3cncc(NCc4cccc(OC(F)(F)F)c4)c3)c(Cl)cn2)CC1] | Example 4 | 492.3 | 0.61 |
| 15 | 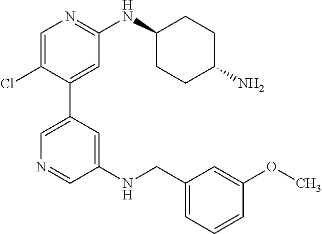 [SMILES: COc1cccc(CNc2cncc(-c3cc(N[C@H]4CC[C@H](N)CC4)ncc3Cl)c2)c1] | Example 4 | 438.3 | 0.5 |
| 16 | 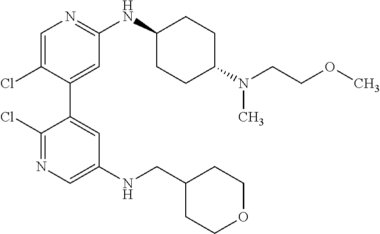 [SMILES: COCCN(C)[C@H]1CC[C@H](Nc2cc(-c3cc(NCC4CCOCC4)cnc3Cl)c(Cl)cn2)CC1] | Example 5 | 522.1 | 0.58 |
| 17 | 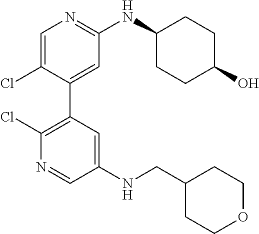 [SMILES: O[C@H]1CC[C@@H](Nc2cc(-c3cc(NCC4CCOCC4)cnc3Cl)c(Cl)cn2)CC1] | Example 5 | 451.2 | 0.62 |
| 18 | 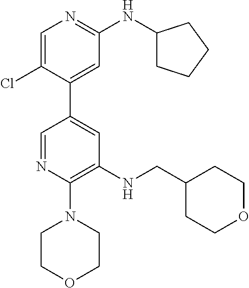 [SMILES: Clc1cnc(NC2CCCC2)cc1-c1cnc(N2CCOCC2)c(NCC2CCOCC2)c1] | Example 6 | 472.2 | 0.69 |
| 19 | 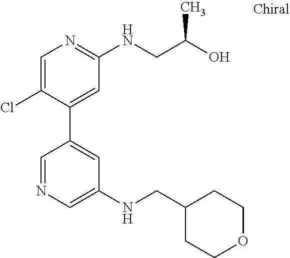 [SMILES: C[C@@H](O)CNc1cc(-c2cncc(NCC3CCOCC3)c2)c(Cl)cn1] | Example 2 | 377.2 | 0.42 |
| 20 | 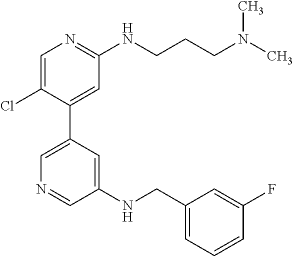 [SMILES: CN(C)CCCNc1cc(-c2cncc(NCc3cccc(F)c3)c2)c(Cl)cn1] | Example 3 | 414.2 | 0.51 |
| 21 | 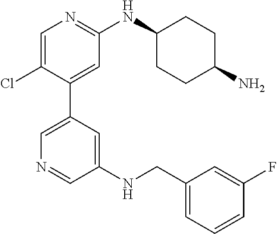 [SMILES: N[C@H]1CC[C@@H](Nc2cc(-c3cncc(NCc4cccc(F)c4)c3)c(Cl)cn2)CC1] | Example 3 | 426.3 | 0.53 |
| 22 | 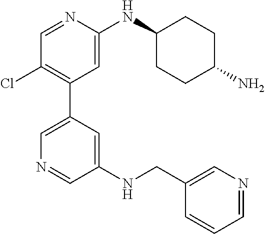 [SMILES: N[C@H]1CC[C@H](Nc2cc(-c3cncc(NCc4cccnc4)c3)c(Cl)cn2)CC1] | Example 3 | 409.2 | 0.29 |
| 23 | 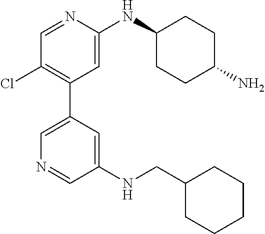 [SMILES: N[C@H]1CC[C@H](Nc2cc(-c3cncc(NCC4CCCCC4)c3)c(Cl)cn2)CC1] | Example 3 | 414.3 | 0.59 |
| 24 | 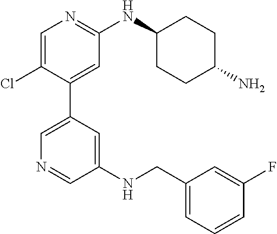 [SMILES: N[C@H]1CC[C@H](Nc2cc(-c3cncc(NCc4cccc(F)c4)c3)c(Cl)cn2)CC1] | Example 3 | 426.2 | 0.52 |
| 25 | 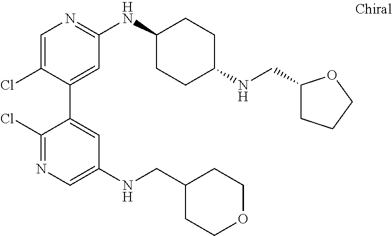 [SMILES: Clc1cnc(N[C@H]2CC[C@H](NC[C@H]3CCCO3)CC2)cc1-c1cc(NCC2CCOCC2)cnc1Cl] | Example 7 | 534.2 | 0.61 |
| 26 | 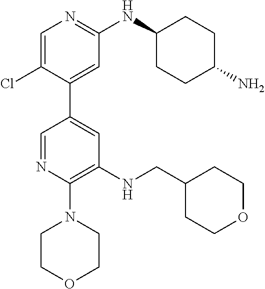 [SMILES: N[C@H]1CC[C@H](Nc2cc(-c3cnc(N4CCOCC4)c(NCC4CCOCC4)c3)c(Cl)cn2)CC1] | Example 6 | 501.3 | 0.52 |
| 27 | 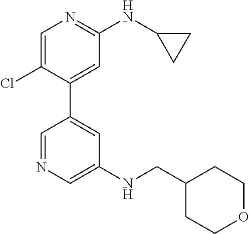 [SMILES: Clc1cnc(NC2CC2)cc1-c1cncc(NCC2CCOCC2)c1] | Example 2 | 359.3 | 0.43 |
| 28 | 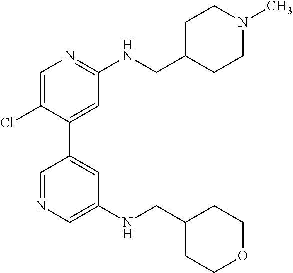 [SMILES: CN1CCC(CNc2cc(-c3cncc(NCC4CCOCC4)c3)c(Cl)cn2)CC1] | Example 2 | 430.2 | 0.41 |
| 29 | 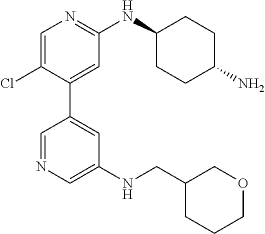 [SMILES: N[C@H]1CC[C@H](Nc2cc(-c3cncc(NCC4CCCOC4)c3)c(Cl)cn2)CC1] | Example 3 | 416.3 | 0.42 |
| 30 | 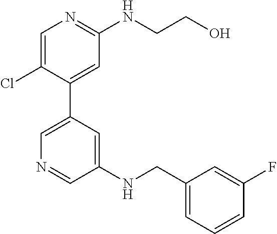 [SMILES: OCCNc1cc(-c2cncc(NCc3cccc(F)c3)c2)c(Cl)cn1] | Example 3 | 373.2 | 0.52 |
| 31 | 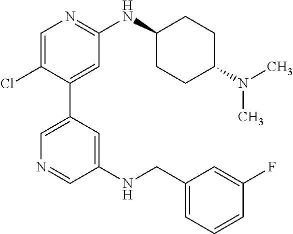 [SMILES: CN(C)[C@H]1CC[C@H](Nc2cc(-c3cncc(NCc4cccc(F)c4)c3)c(Cl)cn2)CC1] | Example 8 | 454.1 | 0.54 |
| 32 | 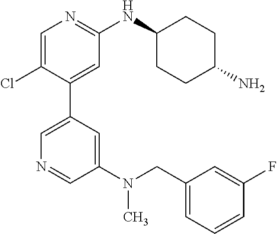 [SMILES: CN(Cc1cccc(F)c1)c1cncc(-c2cc(N[C@H]3CC[C@H](N)CC3)ncc2Cl)c1] | Example 4 | 440.3 | 0.54 |
| 33 | 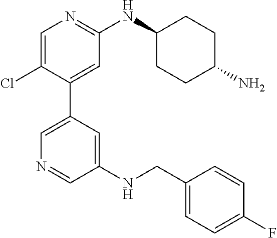 [SMILES: N[C@H]1CC[C@H](Nc2cc(-c3cncc(NCc4ccc(F)cc4)c3)c(Cl)cn2)CC1] | Example 4 | 426.3 | 0.51 |
| 34 | 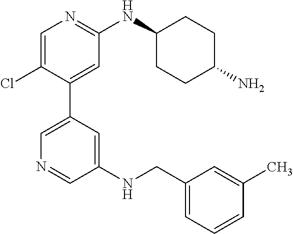 [SMILES: Cc1cccc(CNc2cncc(-c3cc(N[C@H]4CC[C@H](N)CC4)ncc3Cl)c2)c1] | Example 4 | 422.3 | 0.55 |
| 35 | 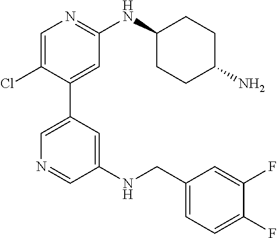 [SMILES: N[C@H]1CC[C@H](Nc2cc(-c3cncc(NCc4ccc(F)c(F)c4)c3)c(Cl)cn2)CC1] | Example 4 | 444.3 | 0.53 |
| 36 | 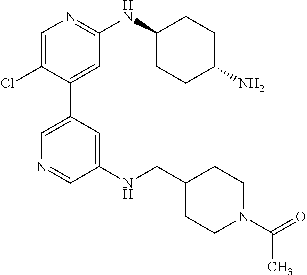 [SMILES: CC(=O)N1CCC(CNc2cncc(-c3cc(N[C@H]4CC[C@H](N)CC4)ncc3Cl)c2)CC1] | Example 9 | 457.2 | 0.4 |
| 37 | 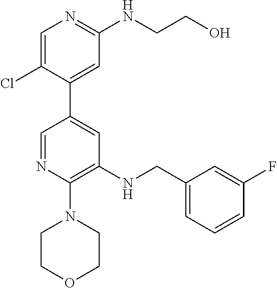 [SMILES: OCCNc1cc(-c2cnc(N3CCOCC3)c(NCc3cccc(F)c3)c2)c(Cl)cn1] | Example 6 | 458.2 | 0.65 |
| 38 | 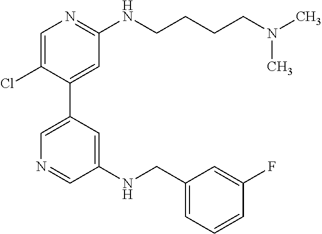 [SMILES: CN(C)CCCCNc1cc(-c2cncc(NCc3cccc(F)c3)c2)c(Cl)cn1] | Example 3 | 428.3 | 0.51 |
| 39 | 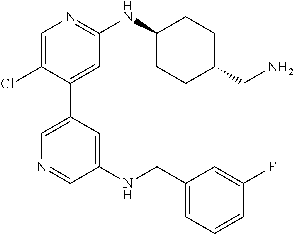 [SMILES: NC[C@H]1CC[C@H](Nc2cc(-c3cncc(NCc4cccc(F)c4)c3)c(Cl)cn2)CC1] | Example 10 | 440.2 | 0.53 |
| 40 | 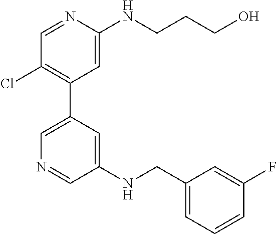 [SMILES: OCCCNc1cc(-c2cncc(NCc3cccc(F)c3)c2)c(Cl)cn1] | Example 3 | 387.2 | 0.53 |
| 41 | 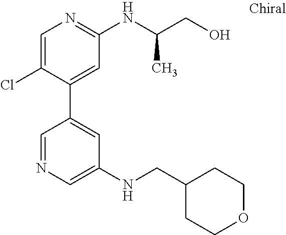 [SMILES: C[C@H](CO)Nc1cc(-c2cncc(NCC3CCOCC3)c2)c(Cl)cn1] | Example 2 | 377.2 | 0.42 |
| 42 | 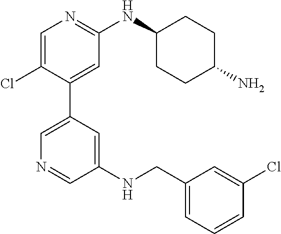 [SMILES: N[C@H]1CC[C@H](Nc2cc(-c3cncc(NCc4cccc(Cl)c4)c3)c(Cl)cn2)CC1] | Example 4 | 442.2 | 0.55 |
| 43 | 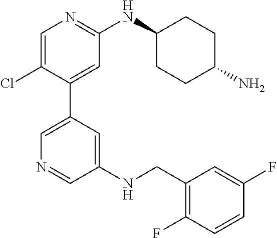 [SMILES: N[C@H]1CC[C@H](Nc2cc(-c3cncc(NCc4cc(F)ccc4F)c3)c(Cl)cn2)CC1] | Example 4 | 444.3 | 0.51 |
| 44 | 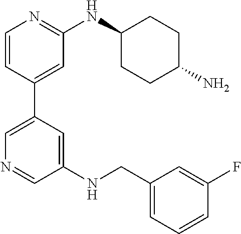 [SMILES: N[C@H]1CC[C@H](Nc2cc(-c3cncc(NCc4cccc(F)c4)c3)ccn2)CC1] | Example 12 | 392.2 | 0.44 |
| 45 | 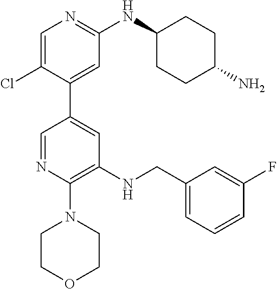 [SMILES: N[C@H]1CC[C@H](Nc2cc(-c3cnc(N4CCOCC4)c(NCc4cccc(F)c4)c3)c(Cl)cn2)CC1] | Example 6 | 511.2 | 0.64 |
| 46 | 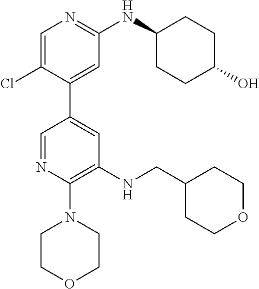 [SMILES: O[C@H]1CC[C@H](Nc2cc(-c3cnc(N4CCOCC4)c(NCC4CCOCC4)c3)c(Cl)cn2)CC1] | Example 6 | 502.2 | 0.56 |
| 47 | 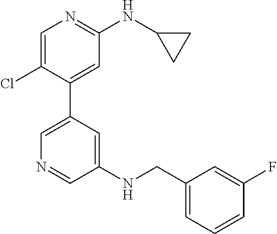 [SMILES: Fc1cccc(CNc2cncc(-c3cc(NC4CC4)ncc3Cl)c2)c1] | Example 3 | 369.2 | 0.57 |
| 48 | 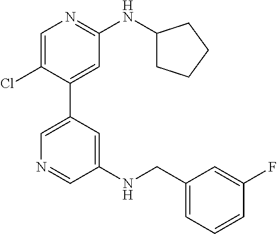 [SMILES: Fc1cccc(CNc2cncc(-c3cc(NC4CCCC4)ncc3Cl)c2)c1] | Example 3 | 397.3 | 0.68 |
| 49 | 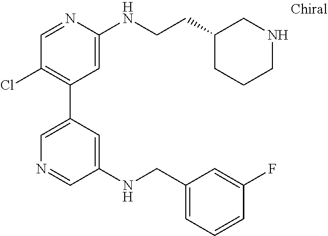 [SMILES: Fc1cccc(CNc2cncc(-c3cc(NCC[C@H]4CCCNC4)ncc3Cl)c2)c1] | Example 3 | 440.2 | 0.53 |
| 50 | 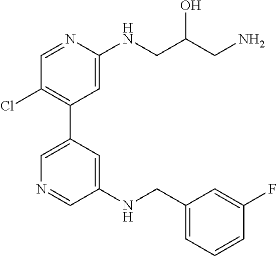 [SMILES: NCC(O)CNc1cc(-c2cncc(NCc3cccc(F)c3)c2)c(Cl)cn1] | Example 3 | 402.1 | 0.49 |
| 51 | 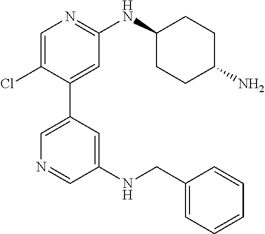 [SMILES: N[C@H]1CC[C@H](Nc2cc(-c3cncc(NCc4ccccc4)c3)c(Cl)cn2)CC1] | Example 4 | 408.3 | 0.5 |
| 52 | 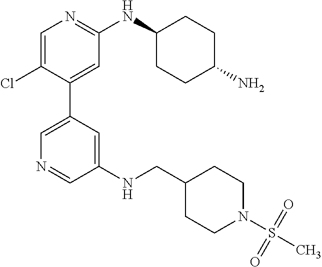 [SMILES: CS(=O)(=O)N1CCC(CNc2cncc(-c3cc(N[C@H]4CC[C@H](N)CC4)ncc3Cl)c2)CC1] | Example 9 | 493.2 | 0.44 |
| 53 | 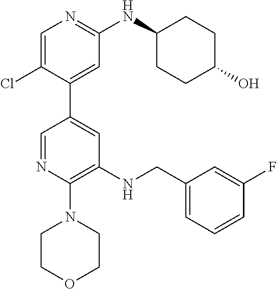 [SMILES: O[C@H]1CC[C@H](Nc2cc(-c3cnc(N4CCOCC4)c(NCc4cccc(F)c4)c3)c(Cl)cn2)CC1] | Example 6 | 512.2 | 0.69 |
| 54 | 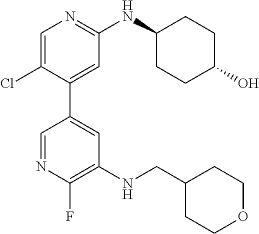 [SMILES: O[C@H]1CC[C@H](Nc2cc(-c3cnc(F)c(NCC4CCOCC4)c3)c(Cl)cn2)CC1] | Example 11 | 435.2 | 0.61 |
| 55 | 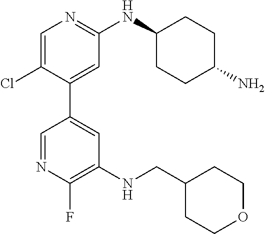 [SMILES: N[C@H]1CC[C@H](Nc2cc(-c3cnc(F)c(NCC4CCOCC4)c3)c(Cl)cn2)CC1] | Example 11 | 434.1 | 0.55 |
| 56 | 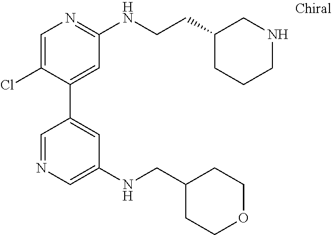 [SMILES: Clc1cnc(NCC[C@H]2CCCNC2)cc1-c1cncc(NCC2CCOCC2)c1] | Example 10 | 430.3 | 0.42 |
| 57 | 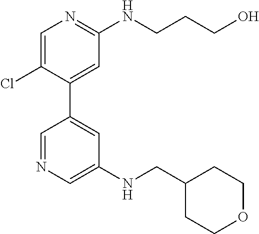 [SMILES: OCCCNc1cc(-c2cncc(NCC3CCOCC3)c2)c(Cl)cn1] | Example 2 | 377.2 | 0.4 |
| 58 | 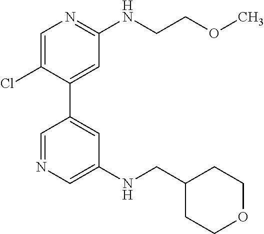 [SMILES: COCCNc1cc(-c2cncc(NCC3CCOCC3)c2)c(Cl)cn1] | Example 2 | 377.2 | 0.46 |
| 59 | 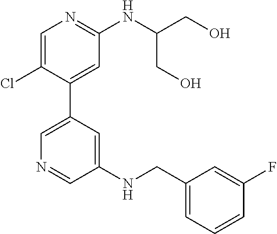 [SMILES: OCC(CO)Nc1cc(-c2cncc(NCc3cccc(F)c3)c2)c(Cl)cn1] | Example 3 | 403.2 | 0.5 |
| 60 | 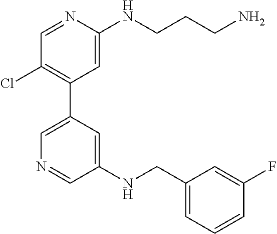 [SMILES: NCCCNc1cc(-c2cncc(NCc3cccc(F)c3)c2)c(Cl)cn1] | Example 3 | 386.2 | 0.5 |
| 61 | 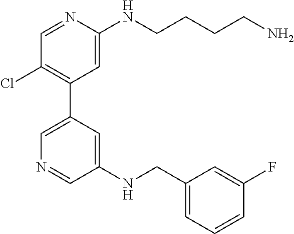 [SMILES: NCCCCNc1cc(-c2cncc(NCc3cccc(F)c3)c2)c(Cl)cn1] | Example 3 | 400.2 | 0.49 |
| 62 | 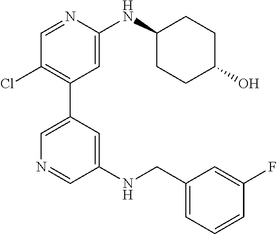 [SMILES: O[C@H]1CC[C@H](Nc2cc(-c3cncc(NCc4cccc(F)c4)c3)c(Cl)cn2)CC1] | Example 3 | 427.2 | 0.55 |
| 63 | 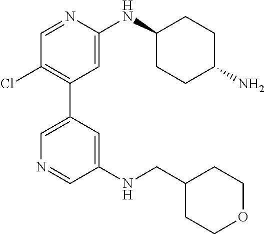 [SMILES: N[C@H]1CC[C@H](Nc2cc(-c3cncc(NCC4CCOCC4)c3)c(Cl)cn2)CC1] | Example 2 | 416.2 | 0.4 |
| 64 | 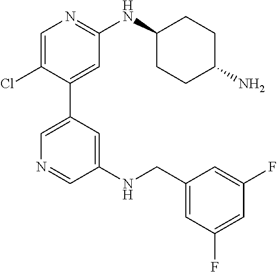 [SMILES: N[C@H]1CC[C@H](Nc2cc(-c3cncc(NCc4cc(F)cc(F)c4)c3)c(Cl)cn2)CC1] | Example 4 | 444.3 | 0.53 |
| 65 | 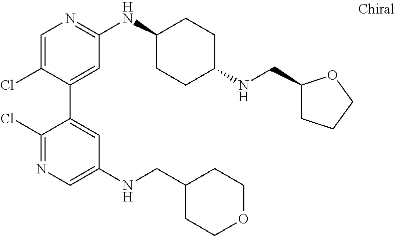 [SMILES: Clc1cnc(N[C@H]2CC[C@H](NC[C@@H]3CCCO3)CC2)cc1-c1cc(NCC2CCOCC2)cnc1Cl] | Example 5 | 534.2 | 0.59 |
| 66 | 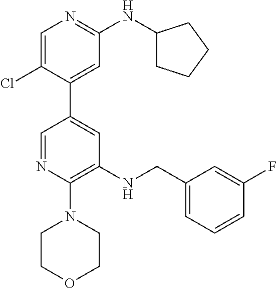 [SMILES: Fc1cccc(CNc2cc(-c3cc(NC4CCCC4)ncc3Cl)cnc2N2CCOCC2)c1] | Example 6 | 482.2 | 0.83 |
| 67 | 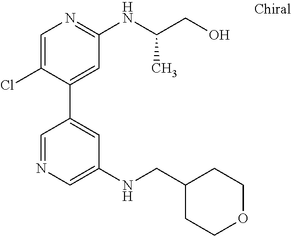 [SMILES: C[C@@H](CO)Nc1cc(-c2cncc(NCC3CCOCC3)c2)c(Cl)cn1] | Example 2 | 377.2 | 0.42 |
| 68 | 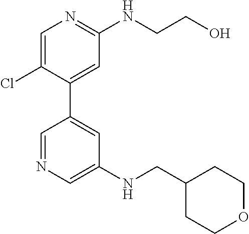 [SMILES: OCCNc1cc(-c2cncc(NCC3CCOCC3)c2)c(Cl)cn1] | Example 2 | 363.2 | 0.39 |
| 69 | 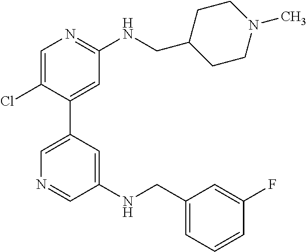 [SMILES: CN1CCC(CNc2cc(-c3cncc(NCc4cccc(F)c4)c3)c(Cl)cn2)CC1] | Example 3 | 440.3 | 0.52 |
| 70 | 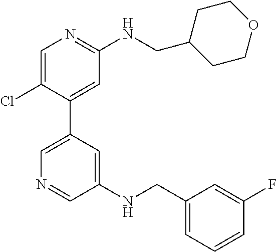 [SMILES: Fc1cccc(CNc2cncc(-c3cc(NCC4CCOCC4)ncc3Cl)c2)c1] | Example 3 | 427.2 | 0.61 |
| 71 | 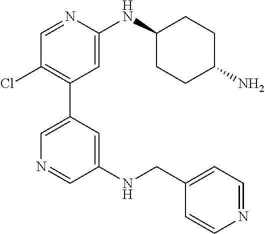 [SMILES: N[C@H]1CC[C@H](Nc2cc(-c3cncc(NCc4ccncc4)c3)c(Cl)cn2)CC1] | Example 3 | 409.3 | 0.29 |
| 72 | 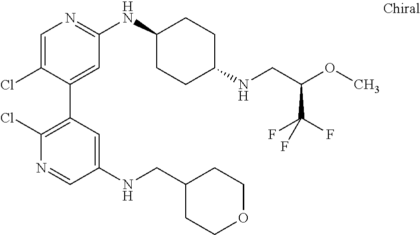 [SMILES: CO[C@H](CN[C@H]1CC[C@H](Nc2cc(-c3cc(NCC4CCOCC4)cnc3Cl)c(Cl)cn2)CC1)C(F)(F)F] | Example 17 | 576.4 | 0.65 |
| 73 | 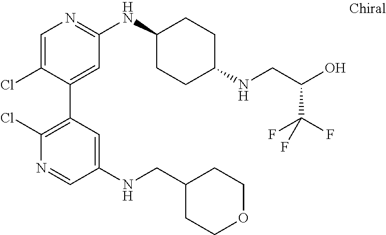 [SMILES: O[C@@H](CN[C@H]1CC[C@H](Nc2cc(-c3cc(NCC4CCOCC4)cnc3Cl)c(Cl)cn2)CC1)C(F)(F)F] | Example 18 | 562.3 | 0.6 |
| 74 | 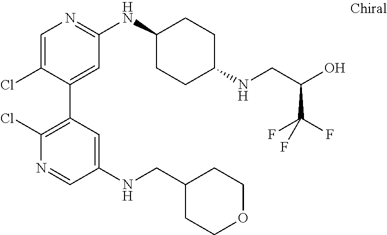 [SMILES: O[C@H](CN[C@H]1CC[C@H](Nc2cc(-c3cc(NCC4CCOCC4)cnc3Cl)c(Cl)cn2)CC1)C(F)(F)F] | Example 18 | 562.3 | 0.6 |
| 75 | 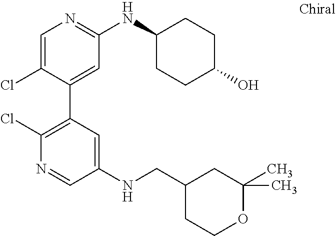 [SMILES: CC1(C)CC(CNc2cnc(Cl)c(-c3cc(N[C@H]4CC[C@H](O)CC4)ncc3Cl)c2)CCO1] | Example 3, Intermediate I | 451.2 | 0.65 |
| 76 | 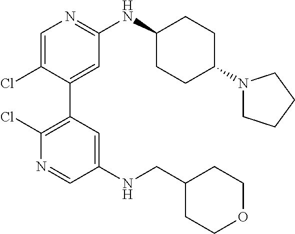 [SMILES: Clc1cnc(N[C@H]2CC[C@H](N3CCCC3)CC2)cc1-c1cc(NCC2CCOCC2)cnc1Cl] | Example 14 | 504.1 | 0.58 |
| 77 | 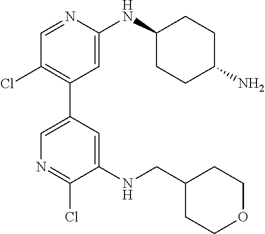 [SMILES: N[C@H]1CC[C@H](Nc2cc(-c3cnc(Cl)c(NCC4CCOCC4)c3)c(Cl)cn2)CC1] | Example 13 | 450.1 | 0.59 |
| 78 | 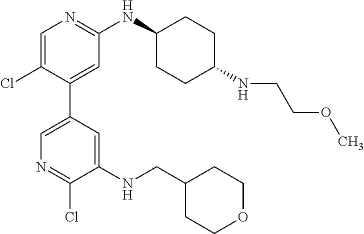 [SMILES: COCCN[C@H]1CC[C@H](Nc2cc(-c3cnc(Cl)c(NCC4CCOCC4)c3)c(Cl)cn2)CC1] | Example 15 | 508.2 | 0.6 |
| 79 | 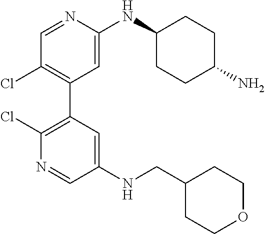 [SMILES: N[C@H]1CC[C@H](Nc2cc(-c3cc(NCC4CCOCC4)cnc3Cl)c(Cl)cn2)CC1] | Example 1 | 450 | 0.59 |
| 80 | 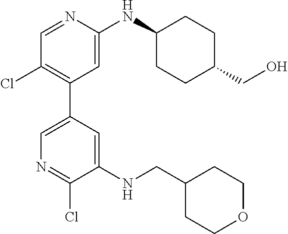 [SMILES: OC[C@H]1CC[C@H](Nc2cc(-c3cnc(Cl)c(NCC4CCOCC4)c3)c(Cl)cn2)CC1] | Example 13 | 465.1 | 0.67 |
| 81 | 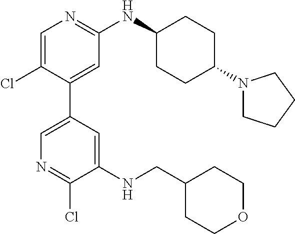 [SMILES: Clc1cnc(N[C@H]2CC[C@H](N3CCCC3)CC2)cc1-c1cnc(Cl)c(NCC2CCOCC2)c1] | Example 14 | 504.2 | 0.7 |
| 82 | 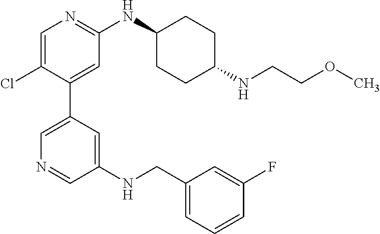 [SMILES: COCCN[C@H]1CC[C@H](Nc2cc(-c3cncc(NCc4cccc(F)c4)c3)c(Cl)cn2)CC1] | Example 15 | 484.1 | 0.53 |
| 83 | 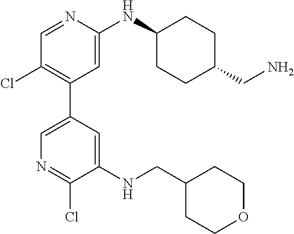 [SMILES: NC[C@H]1CC[C@H](Nc2cc(-c3cnc(Cl)c(NCC4CCOCC4)c3)c(Cl)cn2)CC1] | Example 13 | 464.1 | 0.58 |
| 84 | 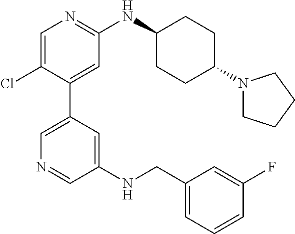 [SMILES: Fc1cccc(CNc2cncc(-c3cc(N[C@H]4CC[C@H](N5CCCC5)CC4)ncc3Cl)c2)c1] | Example 14 | 480.3 | 0.53 |
| 85 | 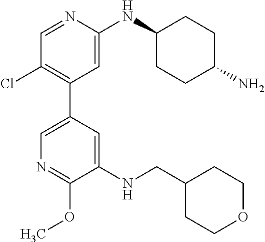 [SMILES: COc1ncc(-c2cc(N[C@H]3CC[C@H](N)CC3)ncc2Cl)cc1NCC1CCOCC1] | Example 13 | 446.1 | 0.58 |
| 86 | 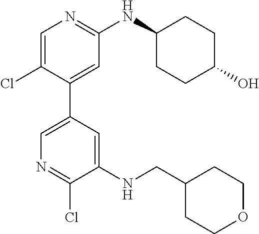 [SMILES: O[C@H]1CC[C@H](Nc2cc(-c3cnc(Cl)c(NCC4CCOCC4)c3)c(Cl)cn2)CC1] | Example 13 | 451.1 | 0.65 |
| 87 | 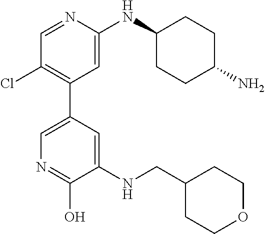 [SMILES: N[C@H]1CC[C@H](Nc2cc(-c3cnc(O)c(NCC4CCOCC4)c3)c(Cl)cn2)CC1] | Example 16 | 432.3 | 0.43 |
| 88 | 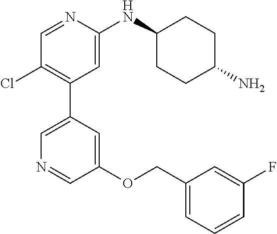 [SMILES: N[C@H]1CC[C@H](Nc2cc(-c3cncc(OCc4cccc(F)c4)c3)c(Cl)cn2)CC1] | | 427.3 | 0.63 |
| 89 | 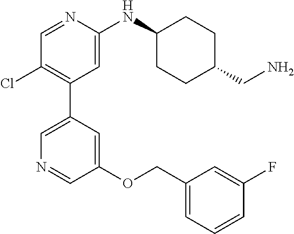 [SMILES: NC[C@H]1CC[C@H](Nc2cc(-c3cncc(OCc4cccc(F)c4)c3)c(Cl)cn2)CC1] | | 441.3 | 0.64 |
| 90 | 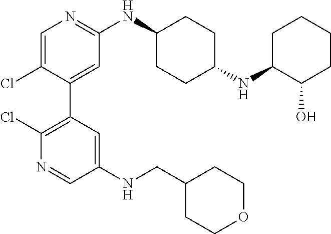 [SMILES: O[C@H]1CCCC[C@@H]1N[C@H]1CC[C@H](Nc2cc(-c3cc(NCC4CCOCC4)cnc3Cl)c(Cl)cn2)CC1] | | 548.2 | 0.70 |
| 91 | 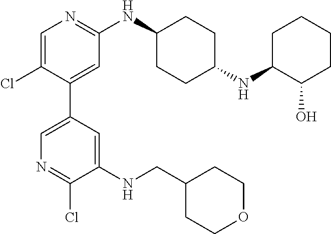 [SMILES: O[C@H]1CCCC[C@@H]1N[C@H]1CC[C@H](Nc2cc(-c3cnc(Cl)c(NCC4CCOCC4)c3)c(Cl)cn2)CC1] | | 548.2 | 0.72 |
| 92 | 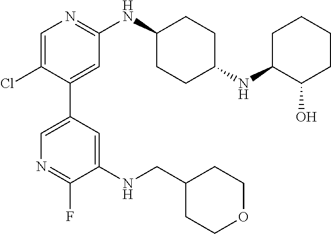 [SMILES: O[C@H]1CCCC[C@@H]1N[C@H]1CC[C@H](Nc2cc(-c3cnc(F)c(NCC4CCOCC4)c3)c(Cl)cn2)CC1] | | 532.2 | 0.70 |

Additional compounds of the invention that can be made using combinations and variations of the methods described above include the compounds shown in Table 1B.

TABLE 1B

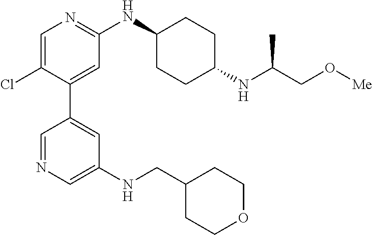 [SMILES: COC[C@H](C)N[C@H]1CC[C@H](Nc2cc(-c3cncc(NCC4CCOCC4)c3)c(Cl)cn2)CC1]

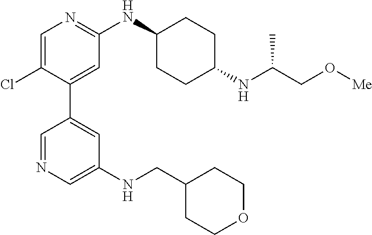 [SMILES: COC[C@@H](C)N[C@H]1CC[C@H](Nc2cc(-c3cncc(NCC4CCOCC4)c3)c(Cl)cn2)CC1]

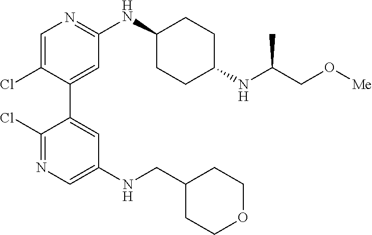 [SMILES: COC[C@H](C)N[C@H]1CC[C@H](Nc2cc(-c3cc(NCC4CCOCC4)cnc3Cl)c(Cl)cn2)CC1]

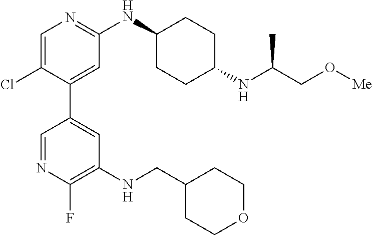 [SMILES: COC[C@H](C)N[C@H]1CC[C@H](Nc2cc(-c3cnc(F)c(NCC4CCOCC4)c3)c(Cl)cn2)CC1]

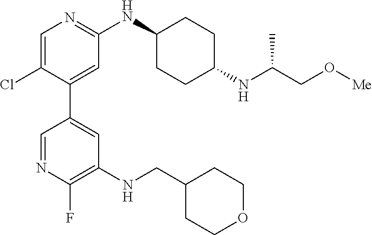 [SMILES: COC[C@@H](C)N[C@H]1CC[C@H](Nc2cc(-c3cnc(F)c(NCC4CCOCC4)c3)c(Cl)cn2)CC1]

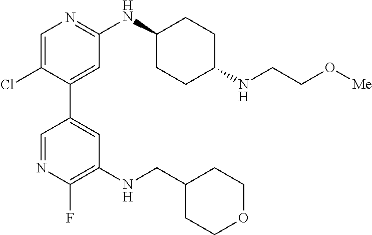 [SMILES: COCCN[C@H]1CC[C@H](Nc2cc(-c3cnc(F)c(NCC4CCOCC4)c3)c(Cl)cn2)CC1]

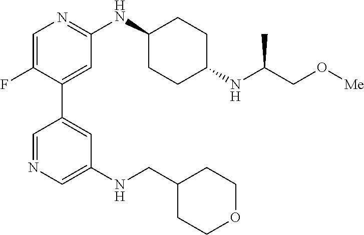 [SMILES: COC[C@H](C)N[C@H]1CC[C@H](Nc2cc(-c3cncc(NCC4CCOCC4)c3)c(F)cn2)CC1]

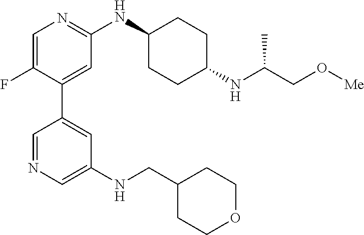 [SMILES: COC[C@@H](C)N[C@H]1CC[C@H](Nc2cc(-c3cncc(NCC4CCOCC4)c3)c(F)cn2)CC1]

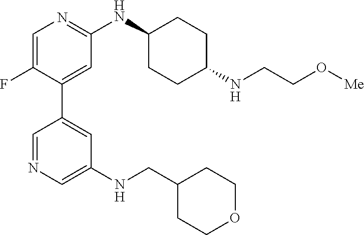 [SMILES: COCCN[C@H]1CC[C@H](Nc2cc(-c3cncc(NCC4CCOCC4)c3)c(F)cn2)CC1]

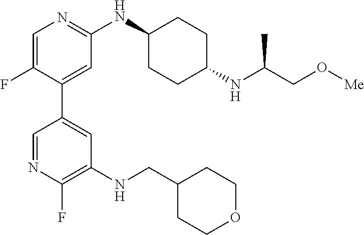 [SMILES: COC[C@H](C)N[C@H]1CC[C@H](Nc2cc(-c3cnc(F)c(NCC4CCOCC4)c3)c(F)cn2)CC1]

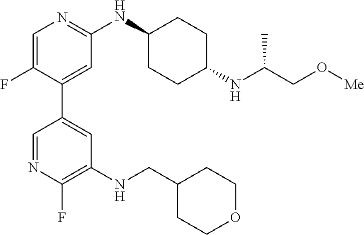 [SMILES: COC[C@@H](C)N[C@H]1CC[C@H](Nc2cc(-c3cnc(F)c(NCC4CCOCC4)c3)c(F)cn2)CC1]

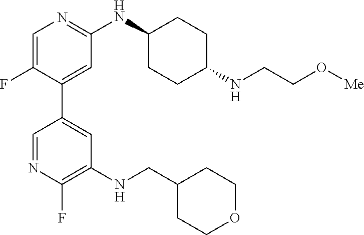 [SMILES: COCCN[C@H]1CC[C@H](Nc2cc(-c3cnc(F)c(NCC4CCOCC4)c3)c(F)cn2)CC1]

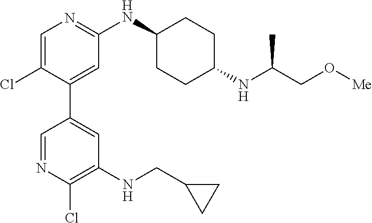 [SMILES: COC[C@H](C)N[C@H]1CC[C@H](Nc2cc(-c3cnc(Cl)c(NCC4CC4)c3)c(Cl)cn2)CC1]

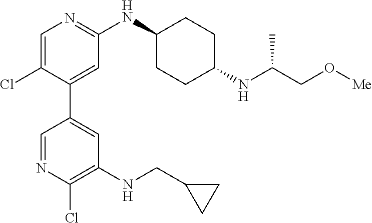 [SMILES: COC[C@@H](C)N[C@H]1CC[C@H](Nc2cc(-c3cnc(Cl)c(NCC4CC4)c3)c(Cl)cn2)CC1]

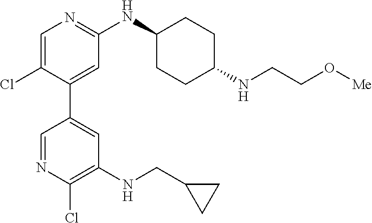 [SMILES: COCCN[C@H]1CC[C@H](Nc2cc(-c3cnc(Cl)c(NCC4CC4)c3)c(Cl)cn2)CC1]

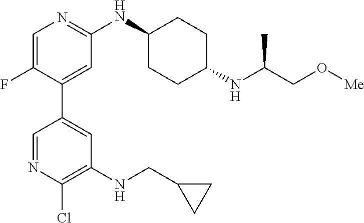 [SMILES: COC[C@H](C)N[C@H]1CC[C@H](Nc2cc(-c3cnc(Cl)c(NCC4CC4)c3)c(F)cn2)CC1]

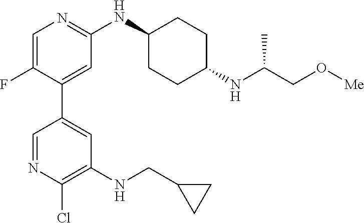 [SMILES: COC[C@@H](C)N[C@H]1CC[C@H](Nc2cc(-c3cnc(Cl)c(NCC4CC4)c3)c(F)cn2)CC1]

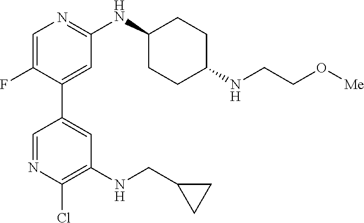 [SMILES: COCCN[C@H]1CC[C@H](Nc2cc(-c3cnc(Cl)c(NCC4CC4)c3)c(F)cn2)CC1]

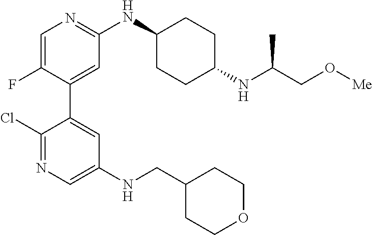 [SMILES: COC[C@H](C)N[C@H]1CC[C@H](Nc2cc(-c3cc(NCC4CCOCC4)cnc3Cl)c(F)cn2)CC1]

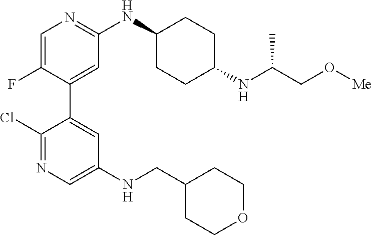 [SMILES: COC[C@@H](C)N[C@H]1CC[C@H](Nc2cc(-c3cc(NCC4CCOCC4)cnc3Cl)c(F)cn2)CC1]

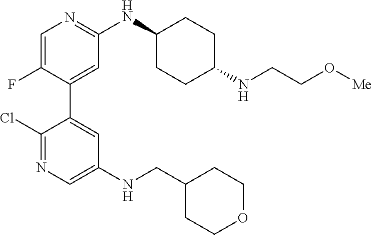 [SMILES: COCCN[C@H]1CC[C@H](Nc2cc(-c3cc(NCC4CCOCC4)cnc3Cl)c(F)cn2)CC1]

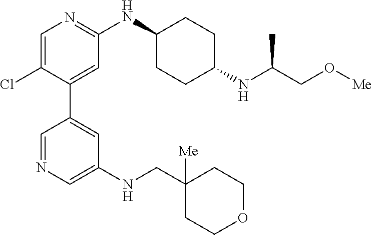 [SMILES: COC[C@H](C)N[C@H]1CC[C@H](Nc2cc(-c3cncc(NCC4(C)CCOCC4)c3)c(Cl)cn2)CC1]

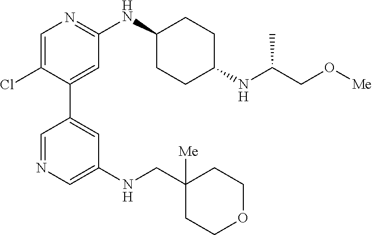 [SMILES: COC[C@@H](C)N[C@H]1CC[C@H](Nc2cc(-c3cncc(NCC4(C)CCOCC4)c3)c(Cl)cn2)CC1]

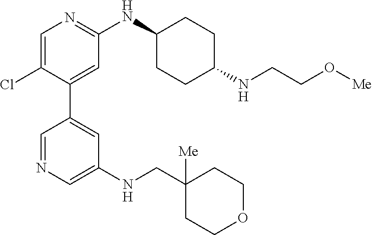 [SMILES: COCCN[C@H]1CC[C@H](Nc2cc(-c3cncc(NCC4(C)CCOCC4)c3)c(Cl)cn2)CC1]

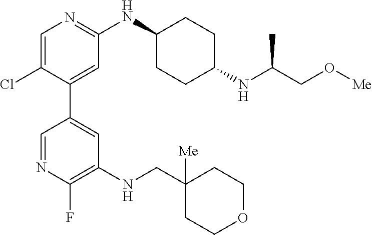 [SMILES: COC[C@H](C)N[C@H]1CC[C@H](Nc2cc(-c3cnc(F)c(NCC4(C)CCOCC4)c3)c(Cl)cn2)CC1]

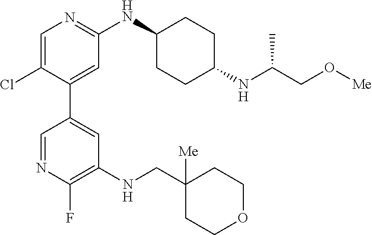 [SMILES: COC[C@@H](C)N[C@H]1CC[C@H](Nc2cc(-c3cnc(F)c(NCC4(C)CCOCC4)c3)c(Cl)cn2)CC1]

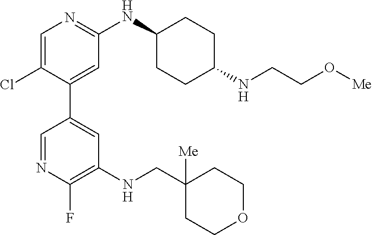 [SMILES: COCCN[C@H]1CC[C@H](Nc2cc(-c3cnc(F)c(NCC4(C)CCOCC4)c3)c(Cl)cn2)CC1]

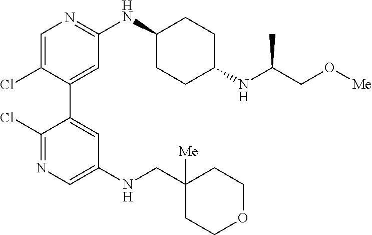 [SMILES: COC[C@H](C)N[C@H]1CC[C@H](Nc2cc(-c3cc(NCC4(C)CCOCC4)cnc3Cl)c(Cl)cn2)CC1]

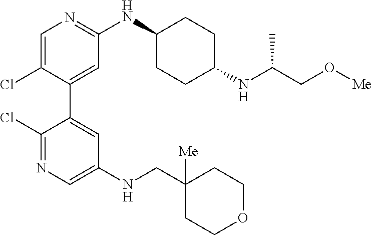 [SMILES: COC[C@@H](C)N[C@H]1CC[C@H](Nc2cc(-c3cc(NCC4(C)CCOCC4)cnc3Cl)c(Cl)cn2)CC1]

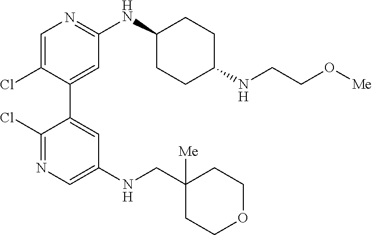 [SMILES: COCCN[C@H]1CC[C@H](Nc2cc(-c3cc(NCC4(C)CCOCC4)cnc3Cl)c(Cl)cn2)CC1]

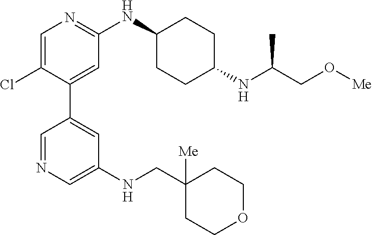 [SMILES: COC[C@H](C)N[C@H]1CC[C@H](Nc2cc(-c3cncc(NCC4(C)CCOCC4)c3)c(Cl)cn2)CC1]

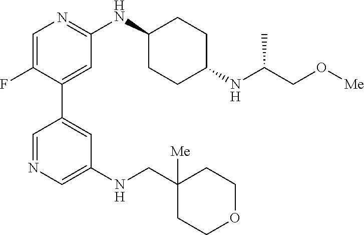 [SMILES: COC[C@@H](C)N[C@H]1CC[C@H](Nc2cc(-c3cncc(NCC4(C)CCOCC4)c3)c(F)cn2)CC1]

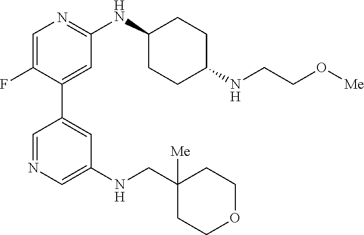 [SMILES: COCCN[C@H]1CC[C@H](Nc2cc(-c3cncc(NCC4(C)CCOCC4)c3)c(F)cn2)CC1]

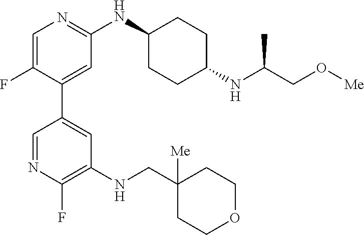 [SMILES: COC[C@H](C)N[C@H]1CC[C@H](Nc2cc(-c3cnc(F)c(NCC4(C)CCOCC4)c3)c(F)cn2)CC1]

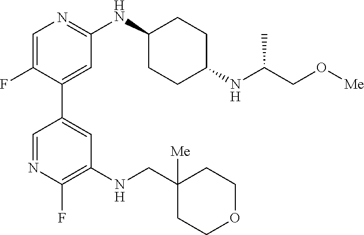 [SMILES: COC[C@@H](C)N[C@H]1CC[C@H](Nc2cc(-c3cnc(F)c(NCC4(C)CCOCC4)c3)c(F)cn2)CC1]

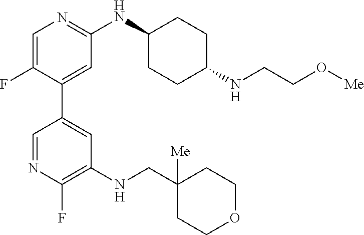 [SMILES: COCCN[C@H]1CC[C@H](Nc2cc(-c3cnc(F)c(NCC4(C)CCOCC4)c3)c(F)cn2)CC1]

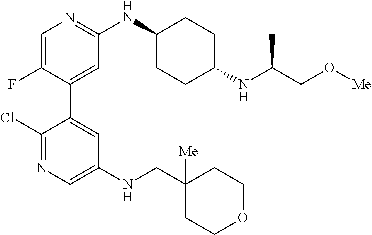 [SMILES: COC[C@H](C)N[C@H]1CC[C@H](Nc2cc(-c3cc(NCC4(C)CCOCC4)cnc3Cl)c(F)cn2)CC1]

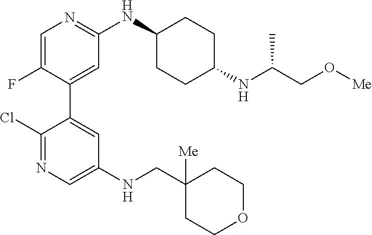 [SMILES: COC[C@@H](C)N[C@H]1CC[C@H](Nc2cc(-c3cc(NCC4(C)CCOCC4)cnc3Cl)c(F)cn2)CC1]

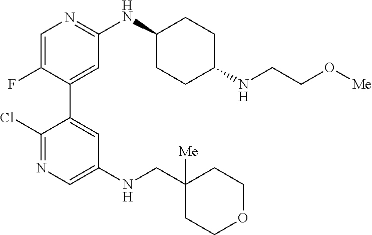 [SMILES: COCCN[C@H]1CC[C@H](Nc2cc(-c3cc(NCC4(C)CCOCC4)cnc3Cl)c(F)cn2)CC1]

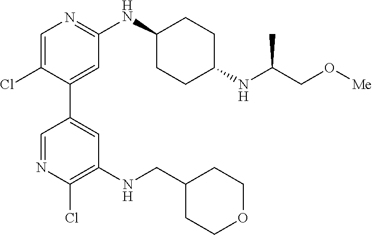 [SMILES: COC[C@H](C)N[C@H]1CC[C@H](Nc2cc(-c3cnc(Cl)c(NCC4CCOCC4)c3)c(Cl)cn2)CC1]

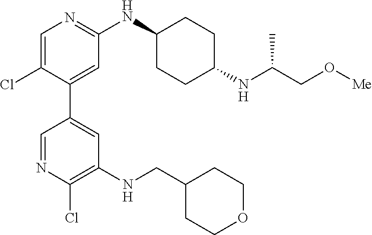 [SMILES: COC[C@@H](C)N[C@H]1CC[C@H](Nc2cc(-c3cnc(Cl)c(NCC4CCOCC4)c3)c(Cl)cn2)CC1]

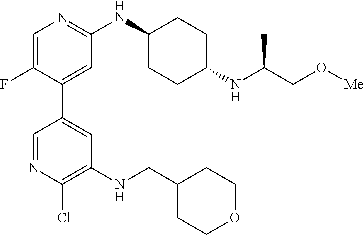 [SMILES: COC[C@H](C)N[C@H]1CC[C@H](Nc2cc(-c3cnc(Cl)c(NCC4CCOCC4)c3)c(F)cn2)CC1]

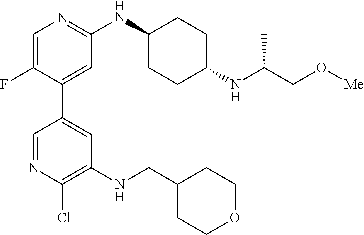 [SMILES: COC[C@@H](C)N[C@H]1CC[C@H](Nc2cc(-c3cnc(Cl)c(NCC4CCOCC4)c3)c(F)cn2)CC1]

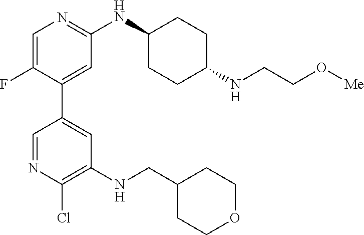 [SMILES: COCCN[C@H]1CC[C@H](Nc2cc(-c3cnc(Cl)c(NCC4CCOCC4)c3)c(F)cn2)CC1]

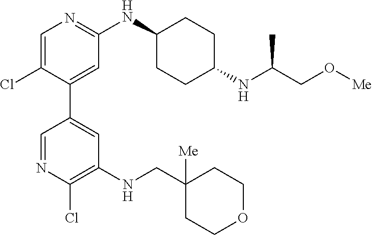 [SMILES: COC[C@H](C)N[C@H]1CC[C@H](Nc2cc(-c3cnc(Cl)c(NCC4(C)CCOCC4)c3)c(Cl)cn2)CC1]

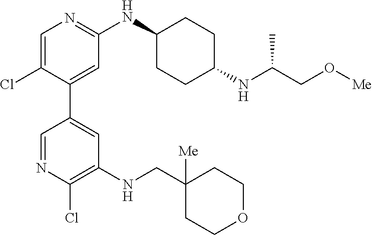 [SMILES: COC[C@@H](C)N[C@H]1CC[C@H](Nc2cc(-c3cnc(Cl)c(NCC4(C)CCOCC4)c3)c(Cl)cn2)CC1]

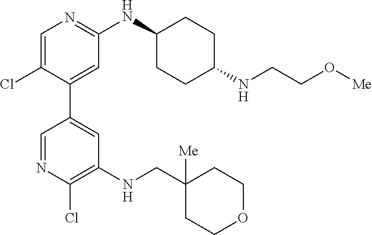 [SMILES: COCCN[C@H]1CC[C@H](Nc2cc(-c3cnc(Cl)c(NCC4(C)CCOCC4)c3)c(Cl)cn2)CC1]

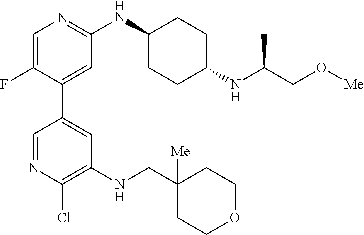 [SMILES: COC[C@H](C)N[C@H]1CC[C@H](Nc2cc(-c3cnc(Cl)c(NCC4(C)CCOCC4)c3)c(F)cn2)CC1]

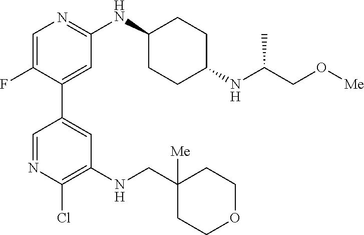 [SMILES: COC[C@@H](C)N[C@H]1CC[C@H](Nc2cc(-c3cnc(Cl)c(NCC4(C)CCOCC4)c3)c(F)cn2)CC1]

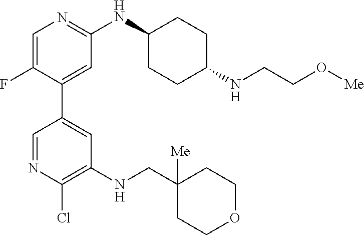 [SMILES: COCCN[C@H]1CC[C@H](Nc2cc(-c3cnc(Cl)c(NCC4(C)CCOCC4)c3)c(F)cn2)CC1]

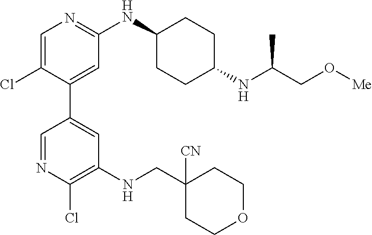 [SMILES: COC[C@H](C)N[C@H]1CC[C@H](Nc2cc(-c3cnc(Cl)c(NCC4(C#N)CCOCC4)c3)c(Cl)cn2)CC1]

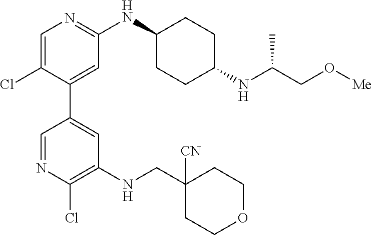 [SMILES: COC[C@@H](C)N[C@H]1CC[C@H](Nc2cc(-c3cnc(Cl)c(NCC4(C#N)CCOCC4)c3)c(Cl)cn2)CC1]

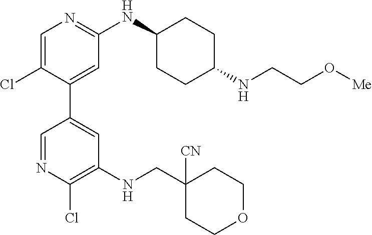 [SMILES: COCCN[C@H]1CC[C@H](Nc2cc(-c3cnc(Cl)c(NCC4(C#N)CCOCC4)c3)c(Cl)cn2)CC1]

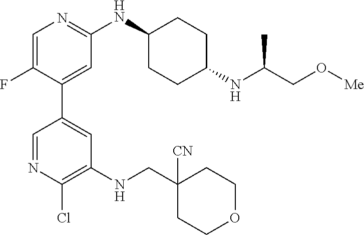 [SMILES: COC[C@H](C)N[C@H]1CC[C@H](Nc2cc(-c3cnc(Cl)c(NCC4(C#N)CCOCC4)c3)c(F)cn2)CC1]

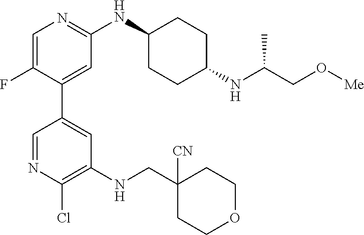 [SMILES: COC[C@@H](C)N[C@H]1CC[C@H](Nc2cc(-c3cnc(Cl)c(NCC4(C#N)CCOCC4)c3)c(F)cn2)CC1]

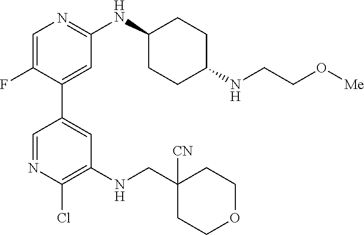 [SMILES: COCCN[C@H]1CC[C@H](Nc2cc(-c3cnc(Cl)c(NCC4(C#N)CCOCC4)c3)c(F)cn2)CC1]

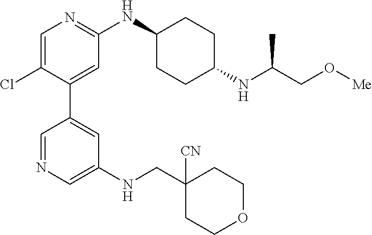 [SMILES: COC[C@H](C)N[C@H]1CC[C@H](Nc2cc(-c3cncc(NCC4(C#N)CCOCC4)c3)c(Cl)cn2)CC1]

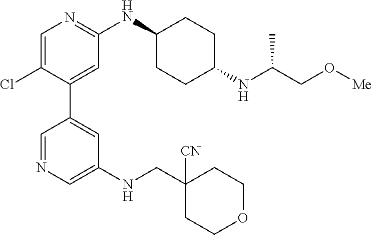 [SMILES: COC[C@@H](C)N[C@H]1CC[C@H](Nc2cc(-c3cncc(NCC4(C#N)CCOCC4)c3)c(Cl)cn2)CC1]

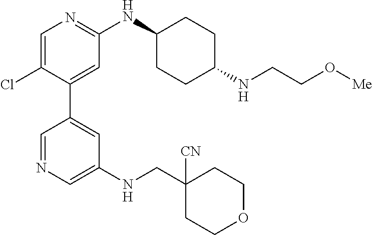 [SMILES: COCCN[C@H]1CC[C@H](Nc2cc(-c3cncc(NCC4(C#N)CCOCC4)c3)c(Cl)cn2)CC1]

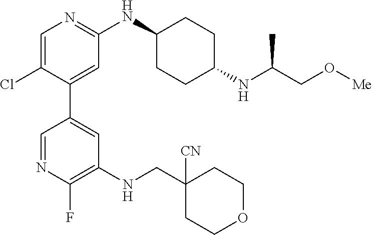 [SMILES: COC[C@H](C)N[C@H]1CC[C@H](Nc2cc(-c3cnc(F)c(NCC4(C#N)CCOCC4)c3)c(Cl)cn2)CC1]

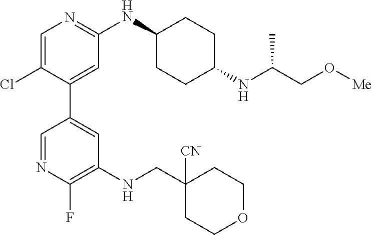 [SMILES: COC[C@@H](C)N[C@H]1CC[C@H](Nc2cc(-c3cnc(F)c(NCC4(C#N)CCOCC4)c3)c(Cl)cn2)CC1]

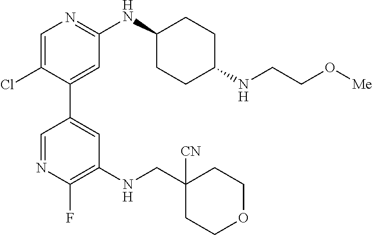 [SMILES: COCCN[C@H]1CC[C@H](Nc2cc(-c3cnc(F)c(NCC4(C#N)CCOCC4)c3)c(Cl)cn2)CC1]

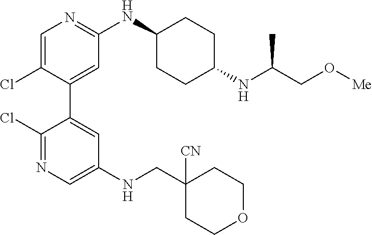 [SMILES: COC[C@H](C)N[C@H]1CC[C@H](Nc2cc(-c3cc(NCC4(C#N)CCOCC4)cnc3Cl)c(Cl)cn2)CC1]

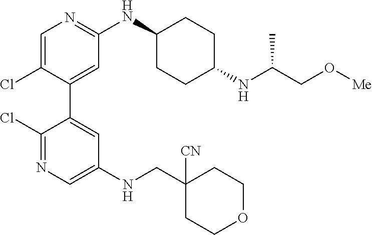 [SMILES: COC[C@@H](C)N[C@H]1CC[C@H](Nc2cc(-c3cc(NCC4(C#N)CCOCC4)cnc3Cl)c(Cl)cn2)CC1]

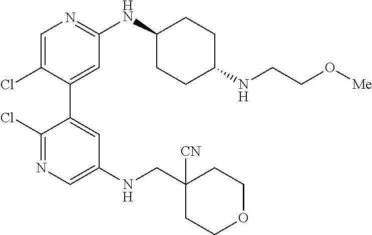 [SMILES: COCCN[C@H]1CC[C@H](Nc2cc(-c3cc(NCC4(C#N)CCOCC4)cnc3Cl)c(Cl)cn2)CC1]

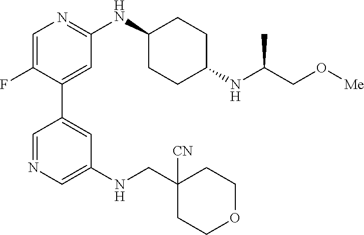 [SMILES: COC[C@H](C)N[C@H]1CC[C@H](Nc2cc(-c3cncc(NCC4(C#N)CCOCC4)c3)c(F)cn2)CC1]

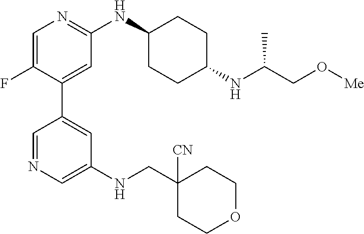 [SMILES: COC[C@@H](C)N[C@H]1CC[C@H](Nc2cc(-c3cncc(NCC4(C#N)CCOCC4)c3)c(F)cn2)CC1]

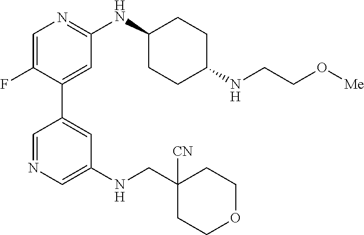 [SMILES: COCCN[C@H]1CC[C@H](Nc2cc(-c3cncc(NCC4(C#N)CCOCC4)c3)c(F)cn2)CC1]

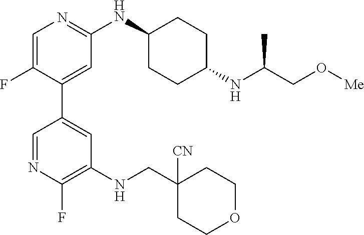 [SMILES: COC[C@H](C)N[C@H]1CC[C@H](Nc2cc(-c3cnc(F)c(NCC4(C#N)CCOCC4)c3)c(F)cn2)CC1]

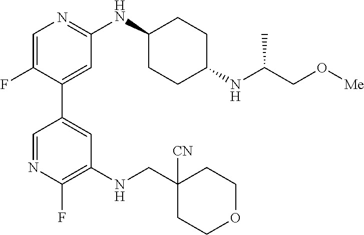 [SMILES: COC[C@@H](C)N[C@H]1CC[C@H](Nc2cc(-c3cnc(F)c(NCC4(C#N)CCOCC4)c3)c(F)cn2)CC1]

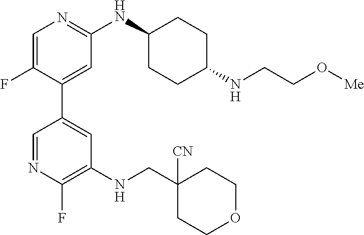 [SMILES: COCCN[C@H]1CC[C@H](Nc2cc(-c3cnc(F)c(NCC4(C#N)CCOCC4)c3)c(F)cn2)CC1]

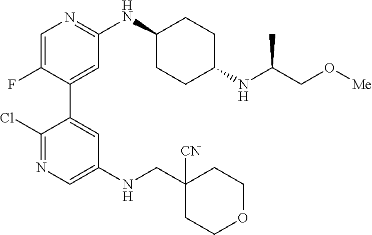 [SMILES: COC[C@H](C)N[C@H]1CC[C@H](Nc2cc(-c3cc(NCC4(C#N)CCOCC4)cnc3Cl)c(F)cn2)CC1]

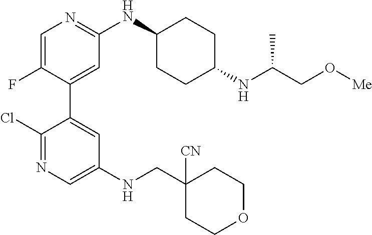 [SMILES: COC[C@@H](C)N[C@H]1CC[C@H](Nc2cc(-c3cc(NCC4(C#N)CCOCC4)cnc3Cl)c(F)cn2)CC1]

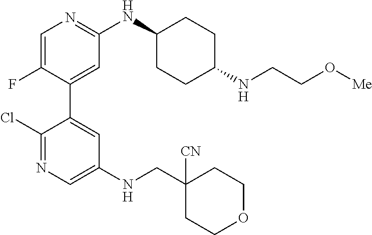 [SMILES: COCCN[C@H]1CC[C@H](Nc2cc(-c3cc(NCC4(C#N)CCOCC4)cnc3Cl)c(F)cn2)CC1]

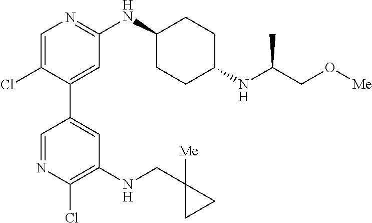 [SMILES: COC[C@H](C)N[C@H]1CC[C@H](Nc2cc(-c3cnc(Cl)c(NCC4(C)CC4)c3)c(Cl)cn2)CC1]

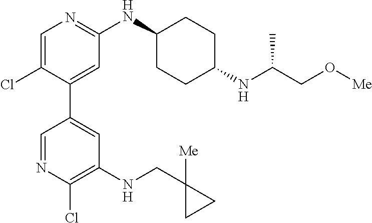 [SMILES: COC[C@@H](C)N[C@H]1CC[C@H](Nc2cc(-c3cnc(Cl)c(NCC4(C)CC4)c3)c(Cl)cn2)CC1]

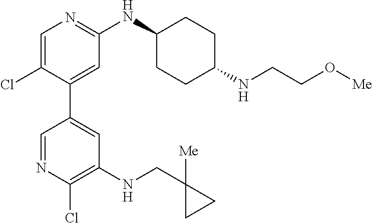 [SMILES: COCCN[C@H]1CC[C@H](Nc2cc(-c3cnc(Cl)c(NCC4(C)CC4)c3)c(Cl)cn2)CC1]

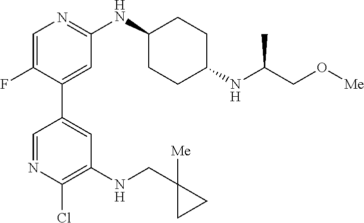 [SMILES: COC[C@H](C)N[C@H]1CC[C@H](Nc2cc(-c3cnc(Cl)c(NCC4(C)CC4)c3)c(F)cn2)CC1]

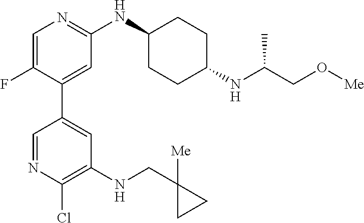 [SMILES: COC[C@@H](C)N[C@H]1CC[C@H](Nc2cc(-c3cnc(Cl)c(NCC4(C)CC4)c3)c(F)cn2)CC1]

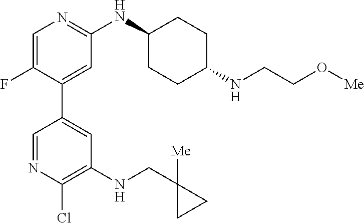 [SMILES: COCCN[C@H]1CC[C@H](Nc2cc(-c3cnc(Cl)c(NCC4(C)CC4)c3)c(F)cn2)CC1]

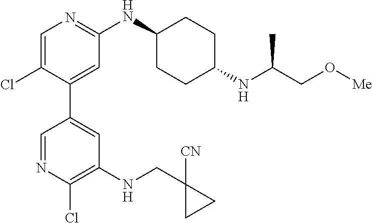 [SMILES: COC[C@H](C)N[C@H]1CC[C@H](Nc2cc(-c3cnc(Cl)c(NCC4(C#N)CC4)c3)c(Cl)cn2)CC1]

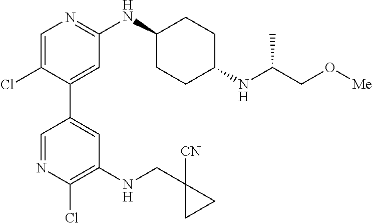 [SMILES: COC[C@@H](C)N[C@H]1CC[C@H](Nc2cc(-c3cnc(Cl)c(NCC4(C#N)CC4)c3)c(Cl)cn2)CC1]

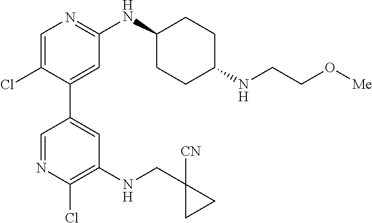 [SMILES: COCCN[C@H]1CC[C@H](Nc2cc(-c3cnc(Cl)c(NCC4(C#N)CC4)c3)c(Cl)cn2)CC1]

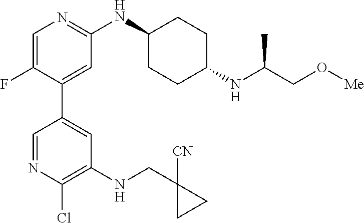 [SMILES: COC[C@H](C)N[C@H]1CC[C@H](Nc2cc(-c3cnc(Cl)c(NCC4(C#N)CC4)c3)c(F)cn2)CC1]

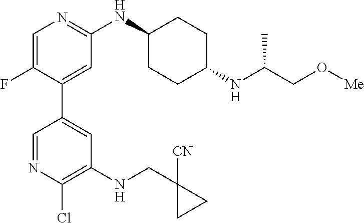 [SMILES: COC[C@@H](C)N[C@H]1CC[C@H](Nc2cc(-c3cnc(Cl)c(NCC4(C#N)CC4)c3)c(F)cn2)CC1]

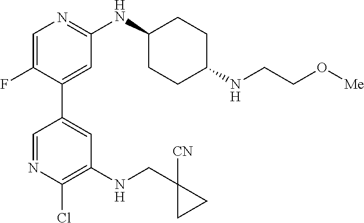 [SMILES: COCCN[C@H]1CC[C@H](Nc2cc(-c3cnc(Cl)c(NCC4(C#N)CC4)c3)c(F)cn2)CC1]

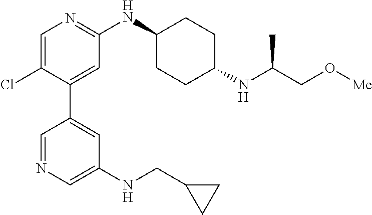 [SMILES: COC[C@H](C)N[C@H]1CC[C@H](Nc2cc(-c3cncc(NCC4CC4)c3)c(Cl)cn2)CC1]

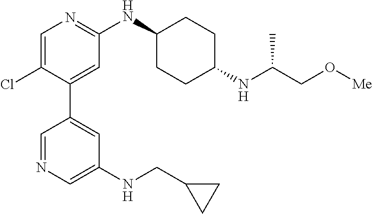 [SMILES: COC[C@@H](C)N[C@H]1CC[C@H](Nc2cc(-c3cncc(NCC4CC4)c3)c(Cl)cn2)CC1]

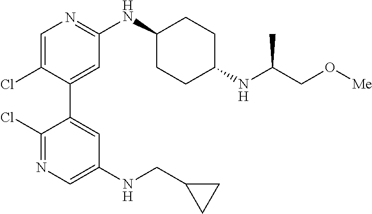 [SMILES: COC[C@H](C)N[C@H]1CC[C@H](Nc2cc(-c3cc(NCC4CC4)cnc3Cl)c(Cl)cn2)CC1]

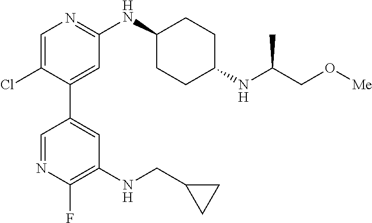 [SMILES: COC[C@H](C)N[C@H]1CC[C@H](Nc2cc(-c3cnc(F)c(NCC4CC4)c3)c(Cl)cn2)CC1]

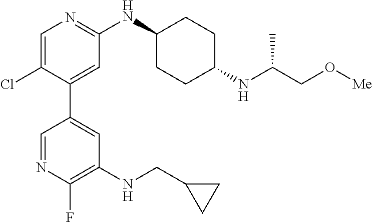 [SMILES: COC[C@@H](C)N[C@H]1CC[C@H](Nc2cc(-c3cnc(F)c(NCC4CC4)c3)c(Cl)cn2)CC1]

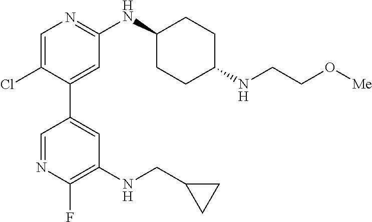 [SMILES: COCCN[C@H]1CC[C@H](Nc2cc(-c3cnc(F)c(NCC4CC4)c3)c(Cl)cn2)CC1]

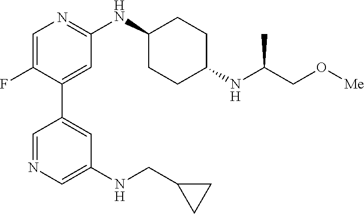 [SMILES: COC[C@H](C)N[C@H]1CC[C@H](Nc2cc(-c3cncc(NCC4CC4)c3)c(F)cn2)CC1]

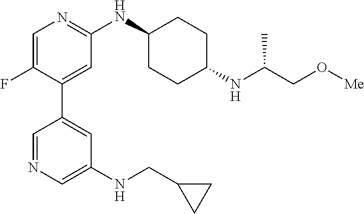 [SMILES: COC[C@@H](C)N[C@H]1CC[C@H](Nc2cc(-c3cncc(NCC4CC4)c3)c(F)cn2)CC1]

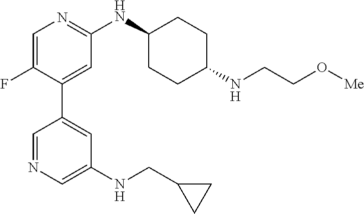 [SMILES: COCCN[C@H]1CC[C@H](Nc2cc(-c3cncc(NCC4CC4)c3)c(F)cn2)CC1]

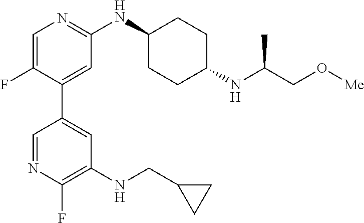 [SMILES: COC[C@H](C)N[C@H]1CC[C@H](Nc2cc(-c3cnc(F)c(NCC4CC4)c3)c(F)cn2)CC1]

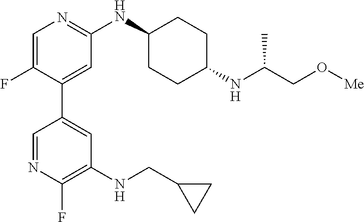 [SMILES: COC[C@@H](C)N[C@H]1CC[C@H](Nc2cc(-c3cnc(F)c(NCC4CC4)c3)c(F)cn2)CC1]

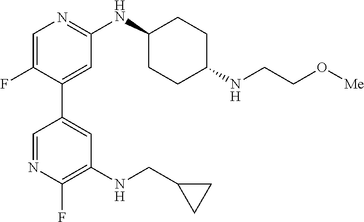 [SMILES: COCCN[C@H]1CC[C@H](Nc2cc(-c3cnc(F)c(NCC4CC4)c3)c(F)cn2)CC1]

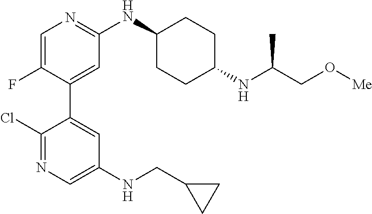 [SMILES: COC[C@H](C)N[C@H]1CC[C@H](Nc2cc(-c3cc(NCC4CC4)cnc3Cl)c(F)cn2)CC1]

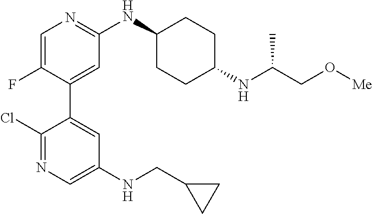 [SMILES: COC[C@@H](C)N[C@H]1CC[C@H](Nc2cc(-c3cc(NCC4CC4)cnc3Cl)c(F)cn2)CC1]

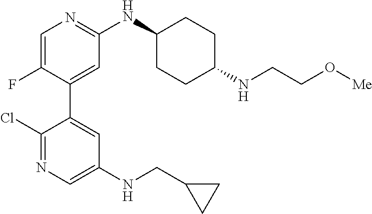 [SMILES: COCCN[C@H]1CC[C@H](Nc2cc(-c3cc(NCC4CC4)cnc3Cl)c(F)cn2)CC1]

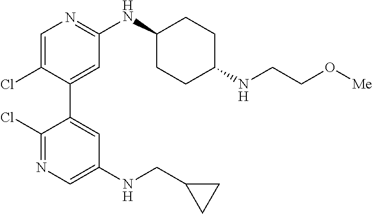 [SMILES: COCCN[C@H]1CC[C@H](Nc2cc(-c3cc(NCC4CC4)cnc3Cl)c(Cl)cn2)CC1]

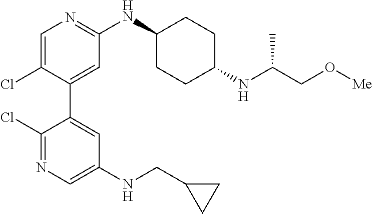 [SMILES: COC[C@@H](C)N[C@H]1CC[C@H](Nc2cc(-c3cc(NCC4CC4)cnc3Cl)c(Cl)cn2)CC1]

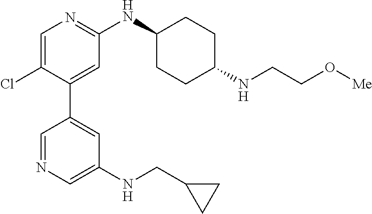 [SMILES: COCCN[C@H]1CC[C@H](Nc2cc(-c3cncc(NCC4CC4)c3)c(Cl)cn2)CC1]

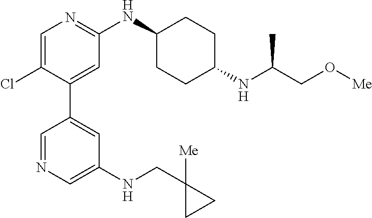 [SMILES: COC[C@H](C)N[C@H]1CC[C@H](Nc2cc(-c3cncc(NCC4(C)CC4)c3)c(Cl)cn2)CC1]

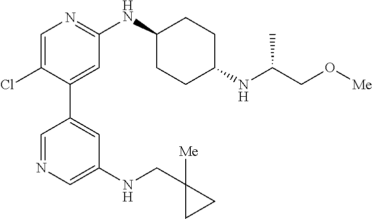 [SMILES: COC[C@@H](C)N[C@H]1CC[C@H](Nc2cc(-c3cncc(NCC4(C)CC4)c3)c(Cl)cn2)CC1]

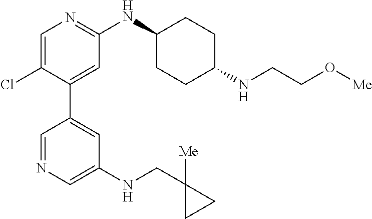 [SMILES: COCCN[C@H]1CC[C@H](Nc2cc(-c3cncc(NCC4(C)CC4)c3)c(Cl)cn2)CC1]

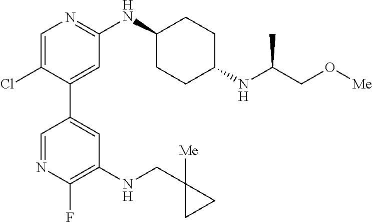 [SMILES: COC[C@H](C)N[C@H]1CC[C@H](Nc2cc(-c3cnc(F)c(NCC4(C)CC4)c3)c(Cl)cn2)CC1]

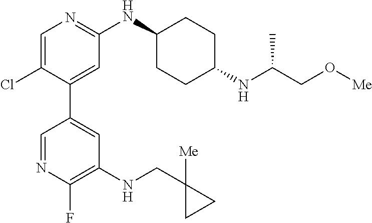 [SMILES: COC[C@@H](C)N[C@H]1CC[C@H](Nc2cc(-c3cnc(F)c(NCC4(C)CC4)c3)c(Cl)cn2)CC1]

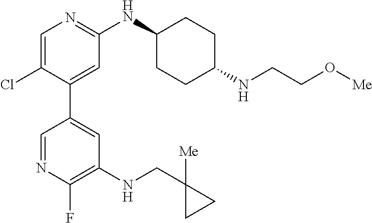 [SMILES: COCCN[C@H]1CC[C@H](Nc2cc(-c3cnc(F)c(NCC4(C)CC4)c3)c(Cl)cn2)CC1]

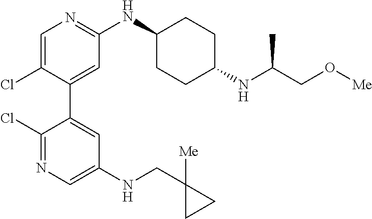 [SMILES: COC[C@H](C)N[C@H]1CC[C@H](Nc2cc(-c3cc(NCC4(C)CC4)cnc3Cl)c(Cl)cn2)CC1]

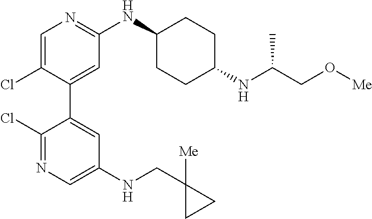 [SMILES: COC[C@@H](C)N[C@H]1CC[C@H](Nc2cc(-c3cc(NCC4(C)CC4)cnc3Cl)c(Cl)cn2)CC1]

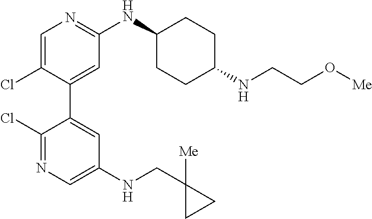 [SMILES: COCCN[C@H]1CC[C@H](Nc2cc(-c3cc(NCC4(C)CC4)cnc3Cl)c(Cl)cn2)CC1]

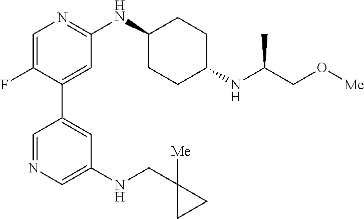 [SMILES: COC[C@H](C)N[C@H]1CC[C@H](Nc2cc(-c3cncc(NCC4(C)CC4)c3)c(F)cn2)CC1]

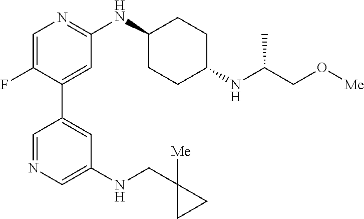 [SMILES: COC[C@@H](C)N[C@H]1CC[C@H](Nc2cc(-c3cncc(NCC4(C)CC4)c3)c(F)cn2)CC1]

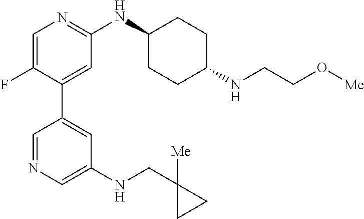 [SMILES: COCCN[C@H]1CC[C@H](Nc2cc(-c3cncc(NCC4(C)CC4)c3)c(F)cn2)CC1]

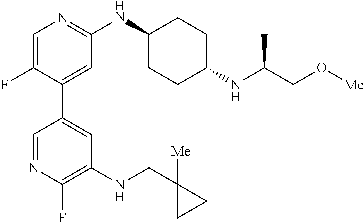 [SMILES: COC[C@H](C)N[C@H]1CC[C@H](Nc2cc(-c3cnc(F)c(NCC4(C)CC4)c3)c(F)cn2)CC1]

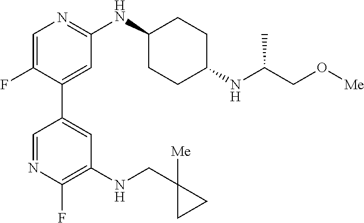 [SMILES: COC[C@@H](C)N[C@H]1CC[C@H](Nc2cc(-c3cnc(F)c(NCC4(C)CC4)c3)c(F)cn2)CC1]

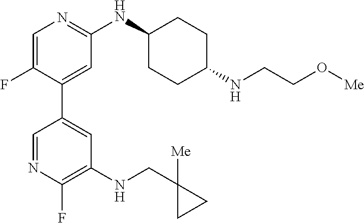 [SMILES: COCCN[C@H]1CC[C@H](Nc2cc(-c3cnc(F)c(NCC4(C)CC4)c3)c(F)cn2)CC1]

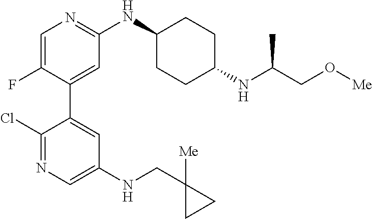 [SMILES: COC[C@H](C)N[C@H]1CC[C@H](Nc2cc(-c3cc(NCC4(C)CC4)cnc3Cl)c(F)cn2)CC1]

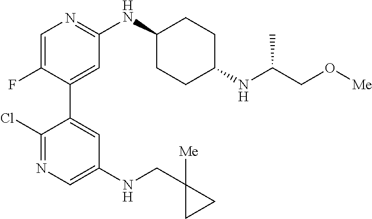 [SMILES: COC[C@@H](C)N[C@H]1CC[C@H](Nc2cc(-c3cc(NCC4(C)CC4)cnc3Cl)c(F)cn2)CC1]

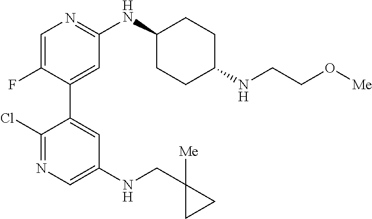 [SMILES: COCCN[C@H]1CC[C@H](Nc2cc(-c3cc(NCC4(C)CC4)cnc3Cl)c(F)cn2)CC1]

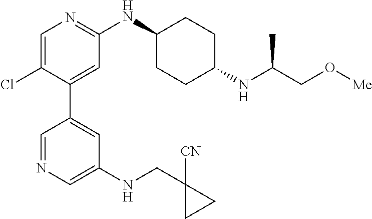 [SMILES: COC[C@H](C)N[C@H]1CC[C@H](Nc2cc(-c3cncc(NCC4(C#N)CC4)c3)c(Cl)cn2)CC1]

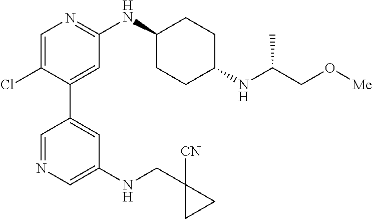 [SMILES: COC[C@@H](C)N[C@H]1CC[C@H](Nc2cc(-c3cncc(NCC4(C#N)CC4)c3)c(Cl)cn2)CC1]

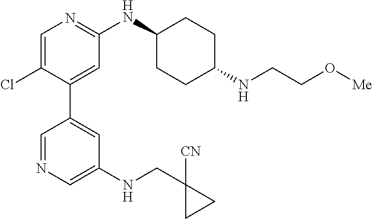 [SMILES: COCCN[C@H]1CC[C@H](Nc2cc(-c3cncc(NCC4(C#N)CC4)c3)c(Cl)cn2)CC1]

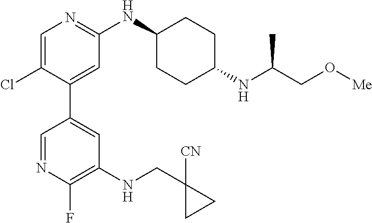 [SMILES: COC[C@H](C)N[C@H]1CC[C@H](Nc2cc(-c3cnc(F)c(NCC4(C#N)CC4)c3)c(Cl)cn2)CC1]

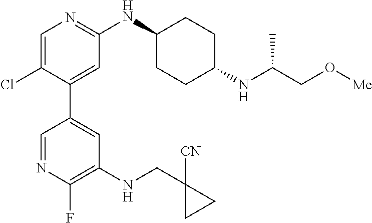 [SMILES: COC[C@@H](C)N[C@H]1CC[C@H](Nc2cc(-c3cnc(F)c(NCC4(C#N)CC4)c3)c(Cl)cn2)CC1]

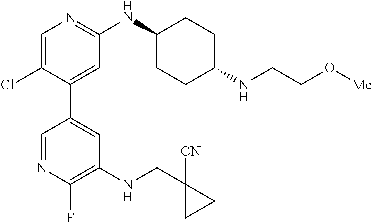 [SMILES: COCCN[C@H]1CC[C@H](Nc2cc(-c3cnc(F)c(NCC4(C#N)CC4)c3)c(Cl)cn2)CC1]

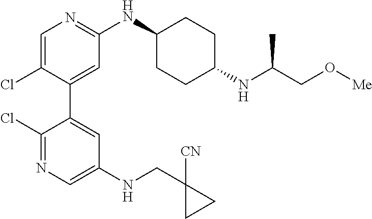 [SMILES: COC[C@H](C)N[C@H]1CC[C@H](Nc2cc(-c3cc(NCC4(C#N)CC4)cnc3Cl)c(Cl)cn2)CC1]

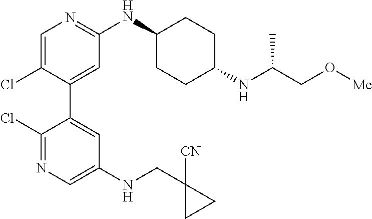 [SMILES: COC[C@@H](C)N[C@H]1CC[C@H](Nc2cc(-c3cc(NCC4(C#N)CC4)cnc3Cl)c(Cl)cn2)CC1]

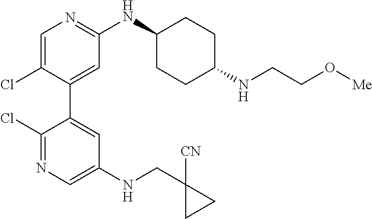 [SMILES: COCCN[C@H]1CC[C@H](Nc2cc(-c3cc(NCC4(C#N)CC4)cnc3Cl)c(Cl)cn2)CC1]

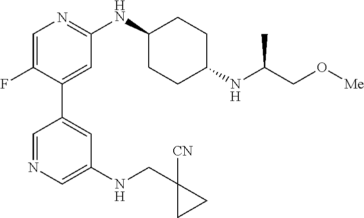 [SMILES: COC[C@H](C)N[C@H]1CC[C@H](Nc2cc(-c3cncc(NCC4(C#N)CC4)c3)c(F)cn2)CC1]

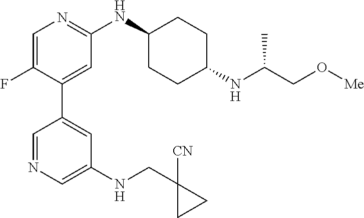 [SMILES: COC[C@@H](C)N[C@H]1CC[C@H](Nc2cc(-c3cncc(NCC4(C#N)CC4)c3)c(F)cn2)CC1]

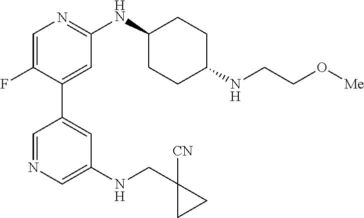 [SMILES: COCCN[C@H]1CC[C@H](Nc2cc(-c3cncc(NCC4(C#N)CC4)c3)c(F)cn2)CC1]

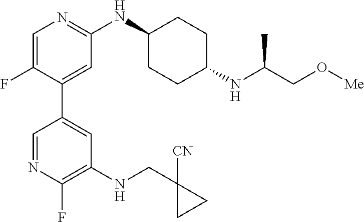 [SMILES: COC[C@H](C)N[C@H]1CC[C@H](Nc2cc(-c3cnc(F)c(NCC4(C#N)CC4)c3)c(F)cn2)CC1]

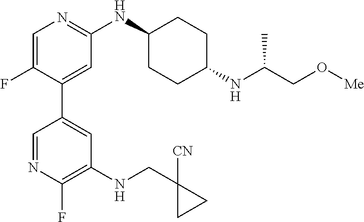 [SMILES: COC[C@@H](C)N[C@H]1CC[C@H](Nc2cc(-c3cnc(F)c(NCC4(C#N)CC4)c3)c(F)cn2)CC1]

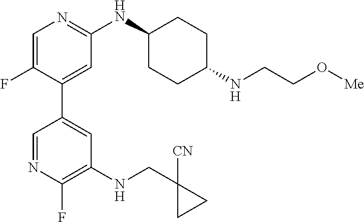 [SMILES: COCCN[C@H]1CC[C@H](Nc2cc(-c3cnc(F)c(NCC4(C#N)CC4)c3)c(F)cn2)CC1]

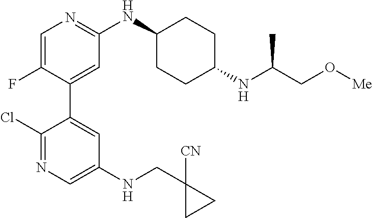 [SMILES: COC[C@H](C)N[C@H]1CC[C@H](Nc2cc(-c3cc(NCC4(C#N)CC4)cnc3Cl)c(F)cn2)CC1]

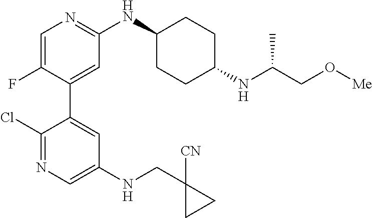 [SMILES: COC[C@@H](C)N[C@H]1CC[C@H](Nc2cc(-c3cc(NCC4(C#N)CC4)cnc3Cl)c(F)cn2)CC1]

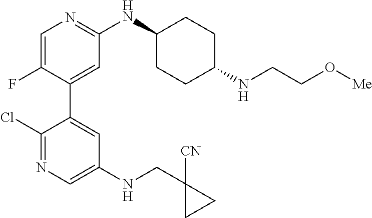 [SMILES: COCCN[C@H]1CC[C@H](Nc2cc(-c3cc(NCC4(C#N)CC4)cnc3Cl)c(F)cn2)CC1]

### Biological Methods

### Cdk9/CyclinT1 IMAP Protocol

The biological activity of the compounds of the invention can be determined using the assay described below.

Cdk9/cyclinT1 is purchased from Millipore, cat #14-685. The final total protein concentration in the assay is 4 nM. The STAMRA-cdk7tide peptide substrate, 5TAMRA-YSPTSPSYSPTSPSYSTPSPS-COOH, is purchased from Molecular Devices, cat#R7352. The final concentration of peptide substrate is 100 nM. The ATP substrate (Adenosine-5′-triphosphate) is purchased from Roche Diagnostics, cat#1140965. The final concentration of ATP substrate is 6 uM. IMAP (Immobilized Metal Assay for Phosphochemicals) Progressive Binding reagent is purchased from Molecular Devices, cat#R8139. Fluorescence polarization (FP) is used for detection. The 5TAMRA-cdk7tide peptide is phosphorylated by Cdk9/cyclinT1 kinase using the ATP substrate. The Phospho-5TAMRA-cdk7tide peptide substrate is bound to the IMAP Progressive Binding Reagent. The binding of the IMAP Progressive Binding Reagent changes the fluorescence polarization of the STAIVIRA-cdk7tide peptide which is measured at an excitation of 531 nm and FP emission of 595 nm. Assays are carried out in 100 mM Tris, pH=7.2, 10 mM MgCl₂, 0.05% NaN3, 0.01% Tween-20, 1 mM dithiothreitol and 2.5% dimethyl sulfoxide. IMAP Progressive Binding Reagent is diluted 1:800 in 100% 1× Solution A from Molecular Devices, cat#R7285.

General protocol is as follows: To 10 ul of cdk9/cyclinT1, 0.5 ul of test compound in dimethyl sulfoxide is added. STAIVIRA-cdk7tide and ATP are mixed. 10 ul of the 5TAMRA-cdk7tide/ATP mix is added to start the reaction. The reaction is allowed to proceed for 4.5 hrs. 60 uL of IMAP Progressive Binding Reagent is added. After >1 hr of incubation, plates are read on the Envision 2101 from Perkin-Elmer. The assay is run in a 384-well format using black Corning plates, cat#3573.

### Cdk9/CyclinT1 Alpha Screen Protocol

Full length wild type Cdk9/cyclin T1 is purchased from Invitrogen, cat#PV4131. The final total protein concentration in the assay is 1 nM. The cdk7tide peptide substrate, biotin-GGGGYSPTSPSYSPTSPSYSPTSPS-OH, is a custom synthesis purchased from the Tufts University Core Facility. The final concentration of cdk7tide peptide substrate is 200 nM. The ATP substrate (Adenosine-5′-triphosphate) is purchased from Roche Diagnostics. The final concentration of ATP substrate is 6 uM. Phospho-Rpb1 CTD (ser2/5) substrate antibody is purchased from Cell Signaling Technology. The final concentration of antibody is 0.67 ug/ml. The Alpha Screen Protein A detection kit containing donor and acceptor beads is purchased from PerkinElmer Life Sciences. The final concentration of both donor and acceptor beads is 15 ug/ml. Alpha Screen is used for detection. The biotinylated-cdk7tide peptide is phosphorylated by cdk9/cyclinT1 using the ATP substrate. The biotinylated-cdk7tide peptide substrate is bound to the streptavidin coated donor bead. The antibody is bound to the protein A coated acceptor bead. The antibody will bind to the phosphorylated form of the biotinylated-cdk7tide peptide substrate, bringing the donor and acceptor beads into close proximity. Laser irradiation of the donor bead at 680 nm generates a flow of short-lived singlet oxygen molecules. When the donor and acceptor beads are in close proximity, the reactive oxygen generated by the irradiation of the donor beads initiates a luminescence/fluorescence cascade in the acceptor beads. This process leads to a highly amplified signal with output in the 530-620 nm range. Assays are carried out in 50 mM Hepes, pH=7.5, 10 mM MgCl₂, 0.1% Bovine Serum Albumin, 0.01% Tween-20, 1 mM Dithiothreitol, 2.5% Dimethyl Sulfoxide. Stop and detection steps are combined using 50 mM Hepes, pH=7.5, 18 mM EDTA, 0.1% Bovine Serum Albumin, 0.01% Tween-20.

General protocol is as follows: To 5 ul of cdk9/cyclinT1, 0.25 ul of test compound in dimethyl sulfoxide is added. Cdk7tide peptide and ATP are mixed. 5 ul of the cdk7tide peptide/ATP mix is added to start the reaction. The reaction is allowed to proceed for 5 hrs. 10 uL of Ab/Alpha Screen beads/Stop-detection buffer is added. Care is taken to keep Alpha Screen beads in the dark at all times. Plates are incubated at room temperature overnight, in the dark, to allow for detection development before being read. The assay is run is a 384-well format using white polypropylene Greiner plates.

The data shown in Table 2 below were generated using the IMAP assay described above, whose output as used had a lower limit of about 0.008, which is reflected in the data as <0.008; values lower than 0.008 were measured under conditions that provide a lower dynamic range of 0.001 nM. The IC-50's are micromolar values.

TABLE 2

| Ex. No. | CDK9 IC50 |
|---|---|
| 1 | 0.032 |
| 2 | <0.008 |
| 3 | 0.013 |
| 4 | <0.008 |
| 5 | <0.008 |
| 6 | 0.043 |
| 7 | 0.001 |
| 8 | 0.001 |
| 9 | 0.015 |
| 10 | 1.858 |
| 11 | 0.386 |
| 12 | 0.032 |
| 13 | 0.13 |
| 14 | <0.008 |
| 15 | 0.008 |
| 16 | <0.008 |
| 17 | 0.025 |
| 18 | 3.438 |
| 19 | 0.469 |
| 20 | 0.045 |
| 21 | 0.011 |
| 22 | 0.009 |
| 23 | 0.01 |
| 24 | <0.008 |
| 25 | <0.008 |
| 26 | 1.96 |
| 27 | 0.057 |
| 28 | 1.966 |
| 29 | 0.016 |
| 30 | <0.008 |
| 31 | <0.008 |
| 32 | 0.016 |
| 33 | <0.008 |
| 34 | <0.008 |
| 35 | <0.008 |
| 36 | 0.032 |
| 37 | 6.917 |
| 38 | 0.021 |
| 39 | <0.008 |
| 40 | <0.008 |
| 41 | 0.325 |
| 42 | <0.008 |
| 43 | <0.008 |
| 44 | <0.008 |
| 45 | 0.61 |
| 46 | 1.834 |
| 47 | <0.008 |
| 48 | <0.008 |
| 49 | <0.008 |
| 50 | 0.01 |
| 51 | <0.008 |
| 52 | 0.035 |
| 53 | 0.66 |
| 54 | <0.008 |
| 55 | <0.008 |
| 56 | 0.068 |
| 57 | 0.199 |
| 58 | 2.26 |
| 59 | 0.13 |
| 60 | <0.008 |
| 61 | <0.008 |
| 62 | <0.008 |
| 63 | 0.018 |
| 64 | <0.008 |
| 65 | <0.008 |
| 66 | 1.486 |
| 67 | 0.335 |
| 68 | 0.82 |
| 69 | <0.008 |
| 70 | 0.009 |
| 71 | <0.008 |
| 72 | 0.007 |
| 73 | 0.002 |
| 74 | 0.001 |
| 75 | 0.001 |
| 76 | 0.035 |
| 77 | <0.008 |
| 78 | <0.008 |
| 79 | <0.008 |
| 80 | <0.008 |
| 81 | 0.045 |
| 82 | <0.008 |
| 83 | <0.008 |
| 84 | <0.008 |
| 85 | 0.096 |
| 86 | <0.008 |
| 87 | 0.42 |
| 88 | <0.001 |
| 89 | <0.001 |
| 90 | 0.0028 |
| 91 | 0.0027 |
| 92 | 0.0018 |

1. A compound of Formula (I): 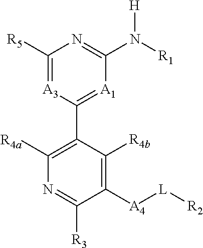
or a pharmaceutically acceptable salt or deuterated version thereof, wherein:
A₁ is CR₆;
A₃ is CR₈;
A₄ is selected from NR₉, and O;
L is an optionally substituted group selected from C₁₋₄alkyl, C₃₋₆ cycloalkyl, C₃₋₆ heterocycloalkyl, and C₂₋₄ alkenyl;
R₁ is —X—R₁₆;
X is a bond or C₁₋₄ alkyl;
R₁₆ is selected from the group consisting of C₃₋₈cycloalkyl, heterocycloalkyl, C₃₋₁₀ heterocycloalkyl, C₃₋₈-partially unsaturated cycloalkyl, C₆₋₁₀ aryl, C₆₋₁₀ aryl- or C₅₋₆-heteroaryl-fused C₅₋₇ heterocycloalkyl, and C₅₋₁₀ heteroaryl,
wherein R₁₆ is optionally substituted with up to three groups independently selected from halogen, oxo (═O), C₁₋₆alkyl, C₁₋₆haloalkyl, C₃₋₆ branched alkyl, C₃₋₆ branched haloalkyl, OH, C₁₋₆alkoxy, C₄₋₈ heterocycloalkyl, C₁₋₂alkyl-heterocycloalkyl, C₁₋₂alkyl-heteroaryl, —R₂₂—OR₁₂, —S(O)₀₋₂R₁₂, —R₂₂—S(O)₀₋₂R₁₂, —S(O)₂NR₁₃R₁₄, —R₂₂—S(O)₂NR₁₃R₁₄, —C(O)OR₁₂, —R₂₂—C(O)OR₁₂, —C(O)R₁₉, —R₂₂—C(O)R₁₉, —O—C₁₋₃ alkyl, —OC₁₋₃ haloalkyl, —OC(O)R₁₉, —R₂₂—OC(O)R₁₉, —C(O)NR₁₃R₁₄, —R₂₂—C(O)NR₁₃R₁₄, —NR₁₅S(O)₂R₁₂, —R₂₂—NR₁₅S(O)₂R₁₂, —NR₁₇R₁₈, —R₂₂—NR₁₇R₁₈, —NR₁₅C(O)R₁₉, —R₂₂—NR₁₅C(O)R₁₉, —NR₁₅C(O)OCH₂Ph, —R₂₂—NR₁₅C(O)OCH₂Ph, —NR₁₅C(O)OR₁₂, —R₂₂—NR₁₅C(O)OR₁₂, —NR₁₅C(O)NR₁₃R₁₄, and —R₂₂—NR₁₅C(O)NR₁₃R₁₄;
wherein said C₁₋₆alkyl and C₃₋₆ branched alkyl are optionally substituted with up to three R₂₀;
R₁₇ and R₁₈ are each, independently, selected from the group consisting of hydrogen, hydroxyl, C₁₋₆alkyl, C₁₋₆haloalkyl, C₃₋₆ branched alkyl, C₃₋₈ cycloalkyl, C₁₋₄-alkyl-C₃₋₈-cycloalkyl, C₃₋₈ heterocycloalkyl, C₁₋₄-alkyl-C₃₋₈ heterocycloalkyl, —R₂₂—OR₁₂, —R₂₂—S(O)₀₋₂R₁₂, —R₂₂—S(O)₂NR₁₃R₁₄, —R₂₂—C(O)OR₁₂, —R₂₂—C(O)R₁₉, —R₂₂—OC(O)R₁₉, —R₂₂—C(O)NR₁₃R₁₄, —R₂₂—NR₁₅S(O)₂R₁₂, —R₂₂—NR₂₃R₂₄, —R₂₂—NR₁₅C(O)R₁₉, —R₂₂—NR₁₅C(O)OCH₂Ph, —R₂₂—NR₁₅C(O)OR₁₂, —R₂₂—NR₁₅C(O)NR₁₃R₁₄, C₆₋₁₀ aryl, C₅₋₁₀ heteroaryl, —C₁₋₂alkyl-C₃₋₈-cycloalkyl, —C₁₋₂ alkyl-aryl, —C₁₋₂ alkyl-heterocycloalkyl and —C₁₋₂ alkyl-heteroaryl,
wherein each of said C₁₋₆alkyl, C₁₋₆haloalkyl, C₃₋₆ branched alkyl, C₁₋₄ alkyl-, C₃₋₈ heterocycloalkyl, and C₃₋₈ cycloalkyl, groups are optionally substituted with up to three R₂₀,
and each of said aryl and heteroaryl groups is optionally substituted with up to three R₂₁, halo or C₁₋₆ alkoxy;
alternatively, R₁₇ and R₁₈ along with the nitrogen atom to which they are attached to can be taken together to form a four to six, seven or eight-membered heterocyclic ring containing up to one additional N, O or S as a ring member, which can be optionally fused with a 5-6-membered optionally-substituted aryl or heteroaryl,
wherein the carbon atoms of said heterocyclic, aryl and heteroaryl rings are optionally substituted with R₂₀, and the nitrogen atom of said rings are optionally substituted with R₂₁;
R₁₉ is selected from optionally substituted alkyl, optionally substituted cycloalkyl, optionally substituted heterocycloalkyl, optionally substituted aryl, and optionally substituted heteroaryl;
R₂₀ is selected from the group consisting of halo, hydroxy, amino, CN, CONR₁₃R₁₄, oxo (═O), C₁₋₆ alkoxy, C₁₋₆ alkyl, C₂₋₄ alkenyl, C₂₋₄ alkynyl, and C₁₋₆ haloalkyl;
and two R²⁰ on the same or adjacent connected atoms can be taken together with the atoms to which they are attached to form a 3-8 membered carbocyclic or heterocyclic ring containing up to 2 heteroatoms selected from N, O and S as ring members and optionally substituted with up to two groups selected from halo, oxo, Me, OMe, CN, hydroxy, amino, and dimethylamino;
R₂₁ is selected from the group consisting of C₁₋₆alkyl, —C(O)R₁₂; C(O)OR₁₂, and —S(O)₂R₁₂;
R₂₂ is selected from the group consisting of C₁₋₆ alkyl, C₁₋₆haloalkyl, C₃₋₆ branched alkyl, C₃₋₆ branched haloalkyl;
R₂₃ and R₂₄ are each, independently, selected from the group consisting of hydrogen, C₁₋₆ alkyl, C₁₋₆haloalkyl, C₃₋₆ branched alkyl, C₃₋₆ branched haloalkyl;
R₂ is selected from C₃₋₈ cycloalkyl, C₄₋₈ heterocycloalkyl, C₆₋₁₀ aryl and C₅₋₁₀ heteroaryl wherein said C₃₋₈ cycloalkyl, and C₄₋₈ heterocycloalkyl groups are optionally substituted with up to three R₂₀, and said aryl and heteroaryl groups are optionally substituted with up to three groups selected from halo, C₁₋₆ alkoxy, and R₂₁;
R₄ₐ, R_{4b}, R₅, and R₆ are each, independently, selected from the group consisting of hydrogen, hydroxyl, cyano, halogen, C₁₋₄ alkyl, C₁₋₄haloalkyl, C₂₋₄ alkenyl, C₂₋₄ alkynyl, amino, NR₁₀R₁₁, C₁₋₄ alkoxy and C₁₋₄ haloalkoxy;
R₃ and R₈ are each, independently, selected from the group consisting of hydrogen, hydroxyl, cyano, halogen, optionally substituted C₁₋₄ alkyl, tetrazolyl, morpholino, C₁₋₄ haloalkyl, optionally substituted C₂₋₄ alkenyl, optionally substituted C₂₋₄ alkynyl, C₁₋₄ alkoxy, NR₁₀R₁₁, C(O)R₁₂; C(O)OR₁₂, C(O)NR₁₃R₁₄, S(O)₀₋₂R₁₂, S(O)₀₋₂NR₁₃R₁₄, and optionally substituted C₃₋₄ cycloalkyl;
R₉ is selected from the group consisting of hydrogen, C₁₋₄ alkyl, alkoxy, C(O)R₁₂, C(O)OR₁₅, C(O)NR₁₃R₁₄, S(O)₀₋₂R₁₂, S(O)₀₋₂NR₁₃R₁₄, optionally substituted C₃₋₄ cycloalkyl, and optionally substituted heterocycloalkyl;
R₁₀ and R₁₁ are each, independently, selected from the group consisting of hydrogen, hydroxyl, alkyl, alkoxy, C(O)R₁₂, C(O)OR₁₂, C(O)NR₁₃R₁₄, S(O)₀₋₂R₁₂, and S(O)₀₋₂NR₁₃R₁₄;
alternatively, R₁₀ and R₁₁ along with the nitrogen atom to which they are attached to can be taken together to form an optionally substituted four to six membered heteroaromatic, or a non-aromatic heterocyclic ring containing up to one additional heteroatom selected from N, O and S as a ring member;
R₁₂ and R₁₅ are each, independently selected from the group consisting of hydrogen, alkyl, branched alkyl, haloalkyl, branched haloalkyl, (CH₂)₀₋₃-cycloalkyl, (CH₂)₀₋₃-heterocycloalkyl, (CH₂)₀₋₃-aryl, and heteroaryl;
R₁₃ and R₁₄ are each, independently, selected from the group consisting of hydrogen, hydroxyl, alkyl, branched alkyl, haloalkyl, branched haloalkyl, alkoxy, cycloalkyl or heterocycloalkyl; and alternatively, R₁₃ and R₁₄ along with the nitrogen atom to which they are attached to can be taken together to form an optionally substituted four to six membered heteroaromatic, or non-aromatic heterocyclic ring that can contain an additional heteroatom selected from N, O and S as a ring member.

2-4. (canceled)

5. The compound of claim 1, wherein: R₈ is selected from halogen, CN, CF₃, O—C₁₋₃-alkyl, and C₁₋₃-alkyl.

6. (canceled)

7. The compound of claim 1, wherein R₈ is Cl or F.

8. The compound of claim 1, wherein -L-R₂ is 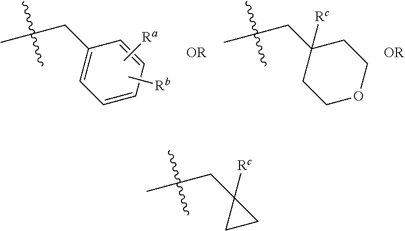
where R^{a} and R^{b} and R^{c} each independently represent H, F, Cl, —OCHF₂, —C(O)-Me, —OH, CF₃, Me, —OMe, —CN, —C≡CH, vinyl, -Ethyl, COOMe, COOH, NH₂, NMe₂, —CONH₂, or —NH—C(O)-Me.

9. The compound of claim 8, wherein -L-R₂ is a group of the formula: 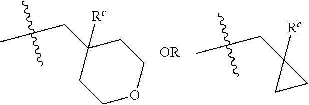
wherein R^{c} is CN, Me, H, OMe, or CF₃.

10. A compound of claim 1, wherein R₁ is substituted cyclohexyl.

11. The compound of claim 1, wherein A₄ is NH.

12. The compound of claim 1, wherein A₄ is O.

13. The compound of claim 1, wherein X is a bond.

14. (canceled)

15. A compound of claim 1, wherein R₁ is cyclohexyl substituted with —NR₁₇R₁₈,
wherein R₁₇ and R₁₈ are each, independently, selected from the group consisting of hydrogen, hydroxyl, C₁₋₆alkyl, C₁₋₆haloalkyl, C₃₋₆ branched alkyl, C₃₋₆ cycloalkyl, —R₂₂—OR₁₂, —R₂₂—S(O)₀₋₂R₁₂, —R₂₂—S(O)₂NR₁₃R₁₄, —R₂₂—C(O)OR₁₂, —R₂₂—C(O)R₁₉, —R₂₂—OC(O)R₁₉, —R₂₂—C(O)NR₁₃R₁₄, —R₂₂—NR₁₅S(O)₂R₁₂, —R₂₂—NR₂₃R₂₄, —R₂₂—NR₁₅C(O)R₁₉, —R₂₂—NR₁₅C(O)OCH₂Ph, —R₂₂—NR₁₅C(O)OR₁₂, —R₂₂—NR₁₅C(O)NR₁₃R₁₄, cycloalkyl, heterocycloalkyl and heteroaryl;
or R₁₇ and R₁₈ along with the nitrogen atom to which they are attached can be taken together to form a four to six or seven membered heterocyclic ring that can contain an additional O, N or S as a ring member, wherein the carbon atoms of said ring are optionally substituted with R₂₀, and the nitrogen atoms of said ring are optionally substituted with R₂₁.

16. The compound of claim 1, wherein:
R₁ is —X—R₁₆ wherein X is a bond; and
R₁₆ is selected from the group consisting of C₄₋₆cycloalkyl, C₄₋₈ heterocycloalkyl, phenyl, and C₅₋₁₀ heteroaryl,
wherein R₁₆ is substituted with up to three groups independently selected from halogen, C₁₋₃alkyl, C₃₋₆ branched alkyl, OH, C₁₋₂alkoxy, —R₂₂—OR₁₂, S(O)₁₋₂R₁₂, —C(O)OR₁₂, —R₂₂—C(O)OR₁₂, —C(O)R₁₉, —R₂₂—OC(O)R₁₉, —C(O)NR₁₃R₁₄, —NR₁₅S(O)₂R₁₂, —NR₁₇R₁₈, —R₂₂—NR₁₇R₁₈, —NR₁₅C(O)R₁₉, —R₂₂—NR₁₅C(O)R₁₉, and —NR₁₅C(O)OCH₂Ph.

17. A compound of claim 1, wherein R₁ is 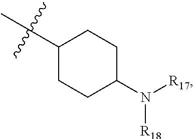
where R₁₇ is H.

18. The compound of claim 17, wherein —NR₁₇R₁₈ is a group of the formula: 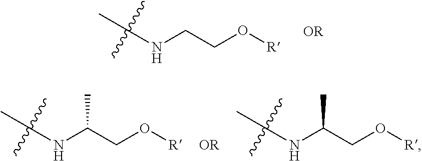
wherein R′ is H, Me, or Et.

19-21. (canceled)

22. A compound of claim 1, wherein:
X represents a bond;
R₁₆ is selected from cyclohexyl, cyclopentyl, and cyclopropyl, wherein each said cyclohexyl, cyclopentyl, and cyclopropyl group is substituted with 1 to 2 substituents selected from amino, methyl-amino, hydroxy, amino-ethyl, dimethyl-amino, —NH—(CH₂)₂—O-ethyl, —NH—SO₂-methyl, —CH₂—NH—SO₂-methyl, piperidinyl, pyrrolidinyl, —NH—CH₂—CF₃, —NH—(CH₂)₂—O-methyl, —N(CH₃)—(CH₂)₁₋₂-methoxy, —NH—CH₂—CH(CH₃)—OH, —NH—CH(CH₃)—CH₂OH, —NH—CH(CH₃)—CH₂OMe, —NH—CH₂-tetrahydrofuranyl, —NH—(CH₂)₂—OH, —NH—CH₂—CONH₂, —NH(CH₂)₂—CF₃, methylpyrrolidin-3-ol, —NH—(CH₂)₂-pyrrolidinyl, —NH—CH₂—COOH, —NH—CH₂-dioxane, —NH-oxetane, —NH-tetrahydrofuranyl, morpholinyl, —NH—(CH₂)₂—O—(CH₂)₂—OCH₃, —NH—(CH₂)₂—CONH₂, and —N(CH₂CH₂OCH₃)₂;
-L-R₂ is selected from —CH₂-fluorophenyl, —CH₂-difluorophenyl, —CH₂-chlorophenyl, —CH₂-pyridyl, —CH₂-cyclopropyl, —CH₂-cyclohexyl, —CH₂-piperidinyl, —CH₂-cyano-phenyl, 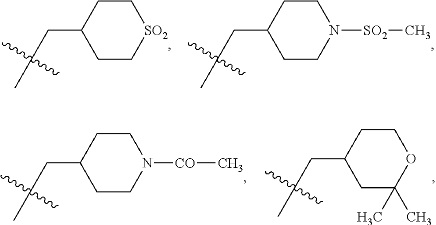
—CH₂-tetrahydropyran, benzyl, —CH₂-toluoyl, and —CH₂-methoxy-phenyl;
A₄ is NH;
R₃ is selected from H, CONH₂, hydroxyethyl, chloro, tetrazolyl, hydroxy, morpholino, cyano, fluoro, and methoxy;
R₄ₐ and R_{4b} are independently selected from H, Cl and fluoro;
R₅ represents H;
R₆ represents hydrogen; and
R₈ is selected from hydrogen, chloro and fluoro.

23. The compound of claim 1, wherein the compound is selected from the compounds of Table 1 or Table 1B.

24. A compound of Formula (II): 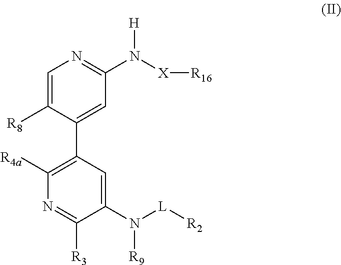
wherein:
X is a bond, —CH₂—, or —(CH₂)₂—,
R₁₆ is selected from C₃-C₆ cycloalkyl and C₁₋₄ alkyl, each of which is optionally substituted with one to three groups independently selected from C₁₋₆ haloalkyl, halo, amino, oxo, —OR, —(CH₂)₂₋₄OR, —NR—(CH₂)₂₋₄—OR, —NR—(CHR)₂₋₄—OR, —O—(CH₂)₂₋₄—OR, and C₁₋₄ aminoalkyl, wherein each R is independently C₁₋₄ alkyl or H;
L is —CH₂— or a bond;
R₈ is F or Cl;
R₄ₐ is H, F or Cl;
R₃ is H, F, Cl, OH, CN, or 4-morpholinyl;
R₉ is H or Me; and
R₂ is selected from cycloalkyl, heterocycloalkyl, heteroaryl and aryl, each of which is optionally substituted with up to three groups independently selected from halo, hydroxy, amino, CONH₂, haloalkyl, C₂₋₄ alkenyl, C₂₋₄ alkynyl, CN, C₁₋₄ alkyl, and C₁₋₄haloalkyl.

25-28. (canceled)

29. The compound of claim 24, wherein -L-R₂ is 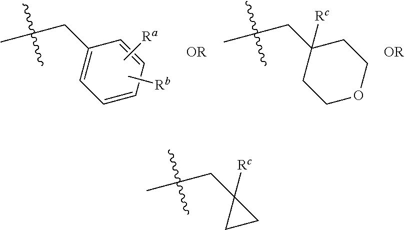
where R^{a} and R^{b} and R^{c} each independently represent H, F, Cl, CF₃, —OCHF₂, —C(O)-Me, —OH, Me, —OMe, —CN, —C≡CH-Ethyl, vinyl, —CONH₂, or —NH—C(O)-Me.

30-32. (canceled)

33. The compound of claim 29, wherein —X—R₁₆ is 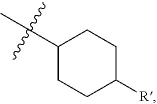
wherein R′ is selected from C₁₋₆ haloalkyl, halo, hydroxy, amino, oxo, C₁₋₄ aminoalkyl, —(CH₂)₁₋₄OR, —NR—(CH₂)₂₋₄—OR, —NR—(CHR)—CH₂—OR, and —O—(CH₂)₂₋₄—OR, wherein each R is independently C₁₋₄ alkyl or H.

34-35. (canceled)

36. A pharmaceutical composition comprising a compound according to claim 1, admixed with at least one pharmaceutically acceptable excipient.

37. The pharmaceutical composition of claim 36, wherein said compound is admixed with at least one pharmaceutically acceptable carrier and at least one additional pharmaceutically acceptable excipient.

38-39. (canceled)

40. A method to treat a cancer selected from the group consisting of bladder, head and neck, breast, stomach, ovary, colon, lung, brain, larynx, lymphatic system, hematopoietic system, genitourinary tract, gastrointestinal, ovarian, prostate, gastric, bone, small-cell lung, glioma, colorectal, and pancreatic cancer, comprising administering to a subject in need thereof a therapeutically effective amount of a compound according to claim 1, or a pharmaceutically acceptable salt thereof.

41-44. (canceled)
